(19) United States
(12) Patent Application Publication
(10) Pub. No.: US 2024/0261422 A1
(43) Pub. Date: Aug. 8, 2024

(54) ANTHRACYCLINE ANTIBODY CONJUGATES

(71) Applicant: SEAGEN INC., Bothell, WA (US)

(72) Inventors: Patrick J. Burke, Seattle, WA (US); Joseph Z. Hamilton, Seattle, WA (US)

(21) Appl. No.: 18/518,130

(22) Filed: Nov. 22, 2023

Related U.S. Application Data
(63) Continuation of application No. PCT/US2022/072570, filed on May 25, 2022.
(60) Provisional application No. 63/194,606, filed on May 28, 2021.

(51) Int. Cl. A61K 47/68; A61K 47/60; A61P 35/00

(52) U.S. Cl. CPC A61K 47/6803; A61K 47/60; A61K 47/6849; A61K 47/6889; A61P 35/00

(57) ABSTRACT

The present disclosure provides, inter alia, antibody drug conjugates that are useful in treating various diseases such as cancer.

### CROSS-REFERENCE TO RELATED APPLICATIONS

This application is a continuation of PCT Application No. PCT/US2022/072570 filed May 25, 2022, which claims the benefit of U.S. Provisional Application No. 63/194,606 filed May 28, 2021, both of which are incorporated herein by reference in their entireties.

### REFERENCE TO THE SEQUENCE LISTING

The present application is being filed along with a Sequence Listing in electronic format. The Sequence Listing is provided as a file entitled SequenceListingSGENE005C1.xml created on Nov. 22, 2023, which is approximately 386.5 kB in size; and further updated by a file entitled 2024-03-11_SequenceListing_SGENE005C1.xml, created on Mar. 11, 2024, which is approximately 1,127,139 bytes in size. The information in the electronic format of the Sequence Listing is incorporated herein by reference in its entirety.

### BACKGROUND

Anthracyclines are cytotoxic compounds that have been used in anticancer therapy for more than 40 years. See Mattarollo, et al., Cancer Res. 2011; Vol. 71, pp. 4809-20. Anthracyclines primarily exert their cytoxotic activity through interfering with DNA topoisomerase II, which is also the mechanism of anthracycline-induced cardiotoxicity. See Dal Ben, et al., Curr. Pharm. Des. 2007; Vol. 13, No. 27, pp. 2766-80. While these compounds may be useful in the treatment of cancer and other diseases, their therapeutic utility is often limited by their dose-dependent toxicity. Anthracycline chemotherapy causes dose-related cardiomyocyte injury and death leading to left ventricular dysfunction. Clinical heart failure may ensue in up to 5% of high-risk patients. See Henriksen, Heart, 2018; Vol. 104, No. 12, pp. 971-77. These off target effects are particularly problematic for more recently developed, highly cytotoxic anthracyclines such as nemorubicin.

The use of antibody-drug conjugates (ADCs) for the local delivery of cytotoxic compounds provides targeted delivery of these drugs directly into tumor cells, or in proximity to tumor cells, whereas systemic administration of these drugs may result in unacceptable levels of toxicity to normal cells. See, e.g., Lambert, Curr. Opin. Pharmacol. 2005; Vol. 5, pp. 543-49 and Doronina, et al., Bioconj. Chem. 2006; Vol. 17, pp. 114-24. Anthracycline ADCs, such as conjugates of doxorubicin and daunorubicin have been studied, but none have been approved for clinical use. See, e.g., Nagy, et al., Proc. Natl. Acad. Sci. 2000; Vol. 97, pp. 829-34 and Dubowchik, et al., Bioorg. Med. Chem. Lett. 2002; Vol 12, pp. 1529-32. Thus, there remains a need for targeted delivery of highly-potent anthracycline compounds to localize the cytotoxic effect to the desired cells while minimizing off-target effects.

### SUMMARY

Some embodiments provide an antibody drug conjugate (ADC) having the structure:

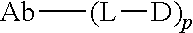

or a salt thereof;
   wherein:
   Ab is an antibody;
   wherein each L is covalently attached to Ab via a sulfur atom of a cysteine residue or an ϵ-amino group of a lysine residue in Ab;
   subscript p is an integer from 1 to 16;
   each D is an anthracycline;
   each L is a linker that has the formula -M-(A)ₐ-(W)_{w}—(Y)_{y}—(X)—, wherein:
   M is a succinimide, a hydrolyzed succinimide, an amide, or a triazole, wherein M is covalently attached to Ab;
   subscript a is 0 or 1;
   subscript y is 0 or 1;
   subscript w is 0 or 1;
   A is a C₂₋₁₀ alkylene optionally substituted with 1-3 R^{a1}; or a 3 to 20 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
   W is from 1-6 amino acids; or
   W has the structure:

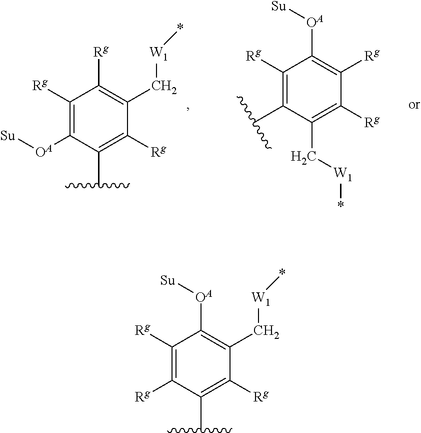

wherein Su is a Sugar moiety;
—O^{A}— represents the oxygen atom of a glycosidic bond;
each R⁹ is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
represents covalent attachment to Y or X;
Y is a self-immolative moiety, a non-self-immolative releasable moiety, or a non-cleavable moiety;
X is from 1-10 amino acids; or
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
L is optionally substituted with a PEG Unit from PEG1 to PEG72.

Some embodiments provide an antibody drug conjugate (ADC) having the structure:

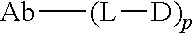

or a salt thereof;
   wherein:
   Ab is an antibody;
   wherein each L is covalently attached to Ab via a sulfur atom of a cysteine residue or an ϵ-amino group of a lysine residue in Ab;
   subscript p is an integer from 1 to 16;
   each D is an anthracycline;
   each L is a linker that has the formula -M-(A)ₐ-(W)_{w}—(Y)_{y}—(X)—, wherein:
   M is a succinimide, a hydrolyzed succinimide, an amide, or a triazole, wherein M is covalently attached to Ab;
   subscript a is 0 or 1;
   subscript y is 0 or 1;
   subscript w is 0 or 1;
   A is a C₂₋₁₀ alkylene optionally substituted with 1-3 R^{a1}; or a 3 to 20 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
   W is from 1-6 amino acids; or
   W has the structure:

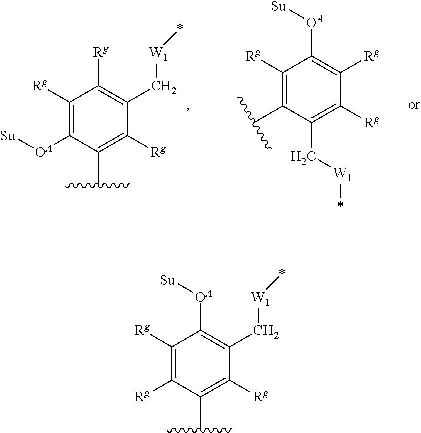

wherein Su is a Sugar moiety;
—O^{A}— represents the oxygen atom of a glycosidic bond;
each R⁹ is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
* represents covalent attachment to Y or X;
Y is a self-immolative moiety, a non-self-immolative releasable moiety, or a non-cleavable moiety;
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
L is optionally substituted with a PEG Unit from PEG1 to PEG72.

Some embodiments provide an antibody drug conjugate (ADC) having the structure:

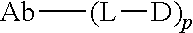

or a salt thereof;
   wherein:
   Ab is an antibody;
   wherein each L is covalently attached to Ab via a sulfur atom of a cysteine residue or an ϵ-amino group of a lysine residue in Ab;
   subscript p is an integer from 1 to 16;
   each D is:

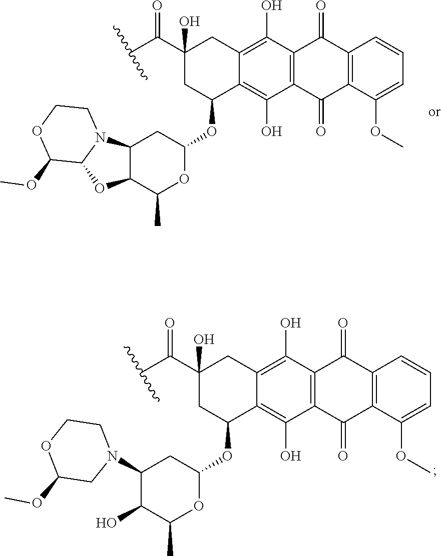

wherein [symbol] represents covalent attachment to L;
each L is a linker that has the formula -M-(A)ₐ-(W)_{w}—(Y)_{y}—(X)—, wherein:
M is a succinimide, a hydrolyzed succinimide, an amide, or a triazole, wherein M is covalently attached to Ab;
subscript a is 0 or 1;
subscript y is 0 or 1;
subscript w is 0 or 1;
A is a C₂₋₁₀ alkylene optionally substituted with 1-3 R^{a1}; or a 3 to 20 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
W is from 1-6 amino acids; or
W has the structure:

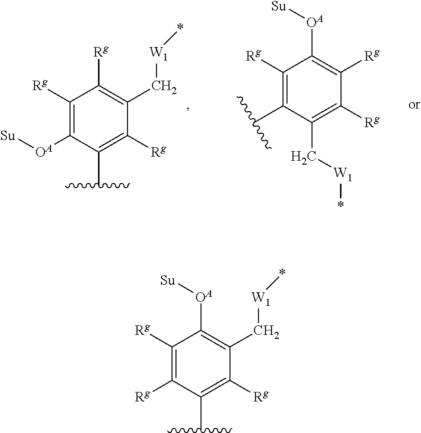

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
* represents covalent attachment to Y or X;
Y is a self-immolative moiety, a non-self-immolative releasable moiety, or a non-cleavable moiety;
X is from 1-10 amino acids; or
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
   L is optionally substituted with a PEG Unit from PEG1 to PEG72.

Some embodiments provide an antibody drug conjugate (ADC) having the structure:

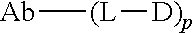

or a salt thereof;
   wherein:
   Ab is an antibody;
   wherein each L is covalently attached to Ab via a sulfur atom of a cysteine residue or an ϵ-amino group of a lysine residue in Ab;
   subscript p is an integer from 1 to 16;
   each D is:

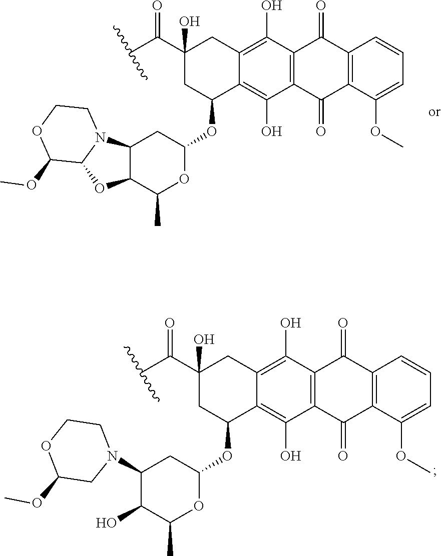

wherein [symbol] represents covalent attachment to L;
each L is a linker that has the formula -M-(A)ₐ-(W)_{w}—(Y)_{y}—(X)—, wherein:
M is a succinimide, a hydrolyzed succinimide, an amide, or a triazole, wherein M is covalently attached to Ab;
subscript a is 0 or 1;
subscript y is 0 or 1;
subscript w is 0 or 1;
A is a C₂₋₁₀ alkylene optionally substituted with 1-3 R^{a1}; or a 3 to 20 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl); each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
W is from 1-6 amino acids; or
W has the structure:

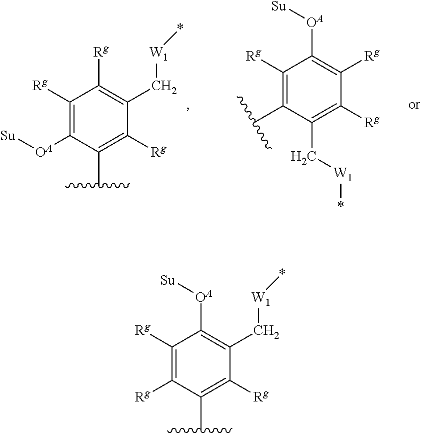

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
* represents covalent attachment to Y or X;
Y is a self-immolative moiety, a non-self-immolative releasable moiety, or a non-cleavable moiety;
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
L is optionally substituted with a PEG Unit from PEG1 to PEG72.

Some embodiments provide an ADC composition comprising a distribution of ADCs, as described herein. In some embodiments, the composition further comprises at least one pharmaceutically acceptable carrier.

Some embodiments provide a method of treating cancer in a subject in need thereof, comprising administering to the subject a therapeutically effective amount of an ADC, as described herein.

Some embodiments provide a method of treating cancer in a subject in need thereof, comprising administering to the subject a therapeutically effective amount of an ADC composition, as described herein.

Some embodiments provide a method of treating an autoimmune disorder in a subject in need thereof, comprising administering to the subject a therapeutically effective amount of an ADC, as described herein.

Some embodiments provide a method of treating an autoimmune disorder in a subject in need thereof, comprising administering to the subject a therapeutically effective amount of an ADC composition, as described herein.

### BRIEF DESCRIPTION OF THE FIGURES

FIG. 1 illustrates the activity of cAC10-PNU antibody-drug conjugates in a SCID mouse xenograft model with L540cy CD30+ Hodgkin lymphoma.

FIG. 2 illustrates the activity of cAC10-PNU antibody-drug conjugates in a SCID mouse xenograft model with DEL/BVR MDR+, CD30+ anaplastic large cell lymphoma.

### DETAILED DESCRIPTION

Provided herein are antibody anthracycline-drug conjugates (ADCs) that can elicit a localized cytotoxic response to target cells, and hence, improved activity and reduced off-target toxicity. For example, the ADCs provided herein can elicit reduced off-target toxicity, such as neutropenia, alopecia, and cardiotoxicity, as compared to the toxicity often observed with systemic administration of anthracyclines. See, e.g., Plosker, Adis Drug Eval. 2008; Vol. 68, pp. 2535-51. Indeed, since their introduction in the 1960s, anthracycline-induced cardiotoxicity has been a primary limiting factor in the use of these compounds in the clinic. See Cardinale, et al., Front. Cardiovasc. Med. 2020; Vol. 7, No. 26, pp. 1-14. The present disclosure provides targeted delivery of anthracyclines to maximize damage to target cells, while avoiding systemic administration of these compounds and their concomitant adverse effects.

### Definitions

Unless otherwise defined, all technical and scientific terms used herein have the same meaning as commonly understood by one of ordinary skill in the art to which this disclosure belongs. Methods and materials are described herein for use in the present application; other, suitable methods and materials known in the art in some aspects of this disclosure are also used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control. When trade names are used herein, the trade name includes the product formulation, the generic drug, and the active pharmaceutical ingredient(s) of the trade name product, unless otherwise indicated by context.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a linker” includes reference to one or more such linkers, and reference to “the cell” includes reference to a plurality of such cells.

The term “about” when referring to a number or a numerical range means that the number or numerical range referred to is an approximation, for example, within experimental variability and/or statistical experimental error, and thus the number or numerical range may vary up to ±10% of the stated number or numerical range. In reference to an ADC composition comprising a distribution of ADCs as described herein, the average number of conjugated anthracycline compounds to an antibody in the composition can be an integer or a non-integer, particularly when the antibody is to be partially loaded. Thus, the term “about” recited prior to an average drug loading value is intended to capture the expected variations in drug loading within an ADC composition.

The term “antibody” as used herein covers intact monoclonal antibodies, polyclonal antibodies, monospecific antibodies, multispecific antibodies (e.g., bispecific antibodies), including intact antibodies and antigen binding antibody fragments, and reduced forms thereof in which one or more of the interchain disulfide bonds are disrupted, that exhibit the desired biological activity and provided that the antigen binding antibody fragments have the requisite number of attachment sites for the desired number of attached groups, such as a linker (L), as described herein. In some embodiments, the linkers are attached via a succinimide or hydrolyzed succinimide to the sulfur atoms of cysteine residues of reduced interchain disulfide bonds and/or cysteine residues introduced by genetic engineering. The native form of an antibody is a tetramer and consists of two identical pairs of immunoglobulin chains, each pair having one light chain and one heavy chain. In each pair, the light and heavy chain variable domains (VL and VH) are together primarily responsible for binding to an antigen. The light chain and heavy chain variable domains consist of a framework region interrupted by three hypervariable regions, also called “complementarity determining regions” or “CDRs.” The light chain and heavy chains also contain constant regions that may be recognized by and interact with the immune system. (see, e.g., Janeway et al., 2001, Immuno. Biology, 5th Ed., Garland Publishing, New York). An antibody includes any isotype (e.g., IgG, IgE, IgM, IgD, and IgA) or subclass (e.g., IgG1, IgG2, IgG3, IgG4, IgA1 and IgA2) thereof. The antibody is derivable from any suitable species. In some embodiments, the antibody is of human or murine origin, and in some embodiments the antibody is a human, humanized or chimeric antibody. Antibodies can be fucosylated to varying extents or afucosylated.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

An “intact antibody” is one which comprises an antigen-binding variable region as well as light chain constant domains (C_{L}) and heavy chain constant domains, C_{H}1, C_{H}2, C_{H}3 and C_{H}4, as appropriate for the antibody class. The constant domains are either native sequence constant domains (e.g., human native sequence constant domains) or amino acid sequence variants thereof.

An “antibody fragment” comprises a portion of an intact antibody, comprising the antigen-binding or variable region thereof. Antibody fragments of the present disclosure include at least one cysteine residue (natural or engineered) that provides a site for attachment of a linker and/or linker-drug compound. In some embodiments, an antibody fragment includes Fab, Fab′, or F(ab′)₂.

An “antigen” is an entity to which an antibody specifically binds.

As used herein the term “engineered cysteine residue” or “eCys residue” refers to a cysteine amino acid or a derivative thereof that is incorporated into an antibody. In those embodiments one or more eCys residues can be incorporated into an antibody, and typically, the eCys residues are incorporated into either the heavy chain or the light chain of an antibody. Generally, incorporation of an eCys residue into an antibody is performed by mutagenizing a nucleic acid sequence of a parent antibody to encode for one or more amino acid residues with a cysteine or a derivative thereof. Suitable mutations include replacement of a desired residue in the light or heavy chain of an antibody with a cysteine or a derivative thereof, incorporation of an additional cysteine or a derivative thereof at a desired location in the light or heavy chain of an antibody, as well as adding an additional cysteine or a derivative thereof to the N- and/or C-terminus of a desired heavy or light chain of an amino acid. Further information can be found in U.S. Pat. No. 9,000,130, the contents of which are incorporated herein in its entirety. Derivatives of cysteine (Cys) include but are not limited to beta-2-Cys, beta-3-Cys, homocysteine, and N-methyl cysteine.

In some embodiments, the antibodies of the present disclosure include those having one or more engineered cysteine (eCys) residues. In some embodiments, derivatives of cysteine (Cys) include, but are not limited to beta-2-Cys, beta-3-Cys, homocysteine, and N-methyl cysteine.

In some embodiments, the antibodies of the present disclosure include those having one or more engineered lysine (eLys) residues. In some embodiments, one or more native lysine and/or eLys residues are activated prior to conjugation with a drug-linker intermediate (to form an ADC, as described herein). In some embodiments, the activation comprises contacting the antibody with a compound comprising a succinimydyl ester and a functional group selected from the group consisting of: maleimido, pyridyldisulfidem and iodoacetamido.

The terms “specific binding” and “specifically binds” mean that the antibody or antibody fragment thereof will bind, in a selective manner, with its corresponding target antigen and not with a multitude of other antigens. Typically, the antibody or antibody fragment binds with an affinity of at least about 1×10⁻⁷ M, for example, 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “amino acid” as used herein, refers to natural, non-natural, non-classical, and proteogenic amino acids. Exemplary amino acids include, but are not limited to alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, ornithine, β-alanine, citrulline, ornithine, serine methyl ether, aspartate methyl ester, glutamate methyl ester, homoserine methyl ether, N,N-dimethyl lysine, methionine sulfoxide, γ-carboxy-glutamic acid, α-aminobutyric acid, α-aminoisobutyric acid, norvaline, naphthylalanine, O-allyl tyrosine, propargylglycine, 2-aminobut-3-ynoic acid, and selenomethionine.

“Natural amino acid” as used herein refers to a naturally occurring amino acid, namely, arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine or a residue thereof, in the L or D-configuration.

“Non-natural amino acid” as used herein refers to an alpha-amino-containing acid or residue thereof, which has the backbone structure of a natural amino acid, but has a side chain group attached to the alpha carbon that is not present in natural amino acids.

“Non-classical amino acid” as used herein refers to an amine-containing acid compound that does not have its amine substituent bonded to the carbon alpha to the carboxylic acid and therefore is not an alpha-amino acid. Non-classical amino acids include 3-amino acids in which a methylene is inserted between the carboxylic acid and amino functional groups in a natural amino acid or a non-natural amino acid.

“Peptide” as used herein refers to a polymer of two or more amino acids wherein the carboxylic acid group of one amino acid forms an amide bond with the alpha-amino group of the next amino acid in the peptide sequence. Peptides may be comprised of naturally occurring amino acids in the L- or D-configuration and/or non-natural and/or non-classical amino acids.

Peptides may be comprised of naturally occurring amino acids in the L- or D-configuration or unnatural or non-classical amino acids, which include, but are not limited to, ornithine, citrulline, diaminobutyric acid, norleucine, pyrylalanine, thienylalanine, naphthylalanine and phenylglycine. Other examples of non-natural and non-classical amino acids are alpha and alpha-disubstituted amino acids, N-alkyl amino acids, lactic acid, halide derivatives of natural amino acids such as trifluorotyrosine, p-Cl-phenylalanine, p-Br-phenylalanine, p-F-phenylalanine, L-allyl-glycine, beta-alanine, L-alpha-amino butyric acid, L-gamma-amino butyric acid, L-alpha-amino isobutyric acid, L-epsilon-amino caproic acid, 7-amino heptanoic acid, L-methionine sulfone, L-norleucine, L-norvaline, p-nitro-L-phenylalanine, L-hydroxyproline, L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminopropionic acid, L-Phe (4-benzyl), 2,4-diaminobutyric acid, 4-aminobutyric acid (gamma-Abu), 2-amino butyric acid (alpha-Abu), 6-amino hexanoic acid (epsilon-Ahx), 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, fluoroamino acids, beta-methyl amino acids, alpha-methyl amino acids, N-methyl amino acids, naphthyl alanine, and the like.

A “sortase enzyme recognition motif” as used herein, refers to a sequence of natural amino acids recognized by one or more sortase enzymes as a site for transpeptidation. In some embodiments, the recognition motif includes the sequence LPXTG, wherein “X” refers to any natural amino acid. In some embodiments, the recognition motif is as described in any of Puorger, et al., Biochemistry, 2017; Vol. 56, No. 21, pp. 2641-50; Antos, et al., Curr. Protoc. Prot. Sci. 2009; Ch. 15, Unit 15-3; Guimares, et al., Nat. Protoc. 2013; Vol. 8, pp. 1787-99; or U.S. Pat. No. 10,960,083, each of which is incorporated by reference herein solely for purposes of disclosing sortase recognition motifs.

A “sugar moiety” as used herein, refers to a monovalent monosaccharide group, for example, a pyranose or a furanose. A sugar moiety may comprise a hemiacetal or a carboxylic acid (from oxidation of the pendant —CH₂OH group). In some embodiments, the sugar moiety is in the β-D conformation. In some embodiments, the sugar moiety is a glucose, glucuronic acid, or mannose group.

The term “inhibit” or “inhibition of” means to reduce by a measurable amount, or to prevent entirely (e.g., 100% inhibition).

The term “therapeutically effective amount” refers to an amount of an ADC, or a salt thereof (as described herein), that is effective to treat a disease or disorder in a mammal. In the case of cancer, the therapeutically effective amount of the ADC or the compound provides one or more of the following biological effects: reduction of the number of cancer cells; reduction of tumor size; inhibition of cancer cell infiltration into peripheral organs; inhibition of tumor metastasis; inhibition, to some extent, of tumor growth; and/or relief, to some extent, of one or more of the symptoms associated with the cancer. For cancer therapy, efficacy, in some aspects, is measured by assessing the time to disease progression (TTP) and/or determining the response rate (RR).

The term “substantial” or “substantially” refers to a majority, i.e. >50% of a population, of a mixture, or a sample, typically more than 50%, 55%, 60%, 65%, 70%, 75%, 80%, 85%, 90%, 91%, 92%, 93%, 94%, 95%, 96%, 97%, 98%, or 99%.

The terms “intracellularly cleaved” and “intracellular cleavage” refer to a metabolic process or reaction occurring inside a cell, in which the cellular machinery acts on the ADC or a fragment thereof, to intracellularly release free drug from the ADC, or other degradant products thereof. The moieties resulting from that metabolic process or reaction are thus intracellular metabolites.

The terms “cancer” and “cancerous” refer to or describe the physiological condition or disorder in mammals that is typically characterized by unregulated cell growth. A “tumor” comprises multiple cancerous cells.

An “autoimmune disorder” as used herein refers to a disease or disorder arising from and directed against an individual's own tissues or proteins.

“Subject” as used herein refers to an individual to which an ADC or ADC composition, as described herein, is administered. Examples of a “subject” include, but are not limited to, a mammal such as a human, rat, mouse, guinea pig, non-human primate, pig, goat, cow, horse, dog, cat, bird and fowl. Typically, a subject is a rat, mouse, dog, non-human primate, or human. In some embodiments, the subject is a human.

The terms “treat” or “treatment,” refer to therapeutic treatment and prophylactic measures to prevent relapse, wherein the object is to inhibit an undesired physiological change or disorder, such as, for example, the development or spread of cancer. For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” in some aspects also means prolonging survival as compared to expected survival if not receiving treatment.

In the context of cancer, the term “treating” includes any or all of: inhibiting growth of cancer cells or of a tumor; inhibiting replication of cancer cells, lessening of overall tumor burden or decreasing the number of cancer cells, and ameliorating one or more symptoms associated with the disease.

In the context of an autoimmune disorder, the term “treating” includes any or all of: inhibiting replication of cells associated with an autoimmune disorder state including, but not limited to, cells that produce an autoimmune antibody, lessening the autoimmune-antibody burden and ameliorating one or more symptoms of an autoimmune disorder.

The term “salt,” as used herein, refers to organic or inorganic salts of a compound, such as a Drug Unit (D) (e.g., an anthracycline), a linker, a drug-linker intermediate, or an ADC, such as those described herein. In some embodiments, the compound contains at least one amino group, and accordingly acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion, or other counterion. The counterion may be any organic or inorganic moiety that stabilizes the charge on the parent compound. Furthermore, a salt has one or more than one charged atom in its structure. In instances where there are multiple charged atoms as part of the salt, multiple counter ions can be present. Hence, a salt can have one or more charged atoms and/or one or more counterions. A “pharmaceutically acceptable salt” is one that is suitable for administration to a subject as described herein and in some aspects includes salts as described by P. H. Stahl and C. G. Wermuth, editors, Handbook of Pharmaceutical Salts: Properties, Selection and Use, Weinheim/Ziirich: Wiley-VCH/VHCA, 2002, the list for which is specifically incorporated by reference in its entirety. In some embodiments, the ADCs described herein are present in the form of a pharmaceutically acceptable salt. In some embodiments, the compounds described herein are present in the form of a pharmaceutically acceptable salt.

The term “anthracycline,” as used herein refers to a class of compounds that contain a fused tetracyclic ring system and are isolated from certain types of Streptomyces bacteria, such as S. peucetius. This term also includes derivatives (e.g., semisynthetic derivatives) and metabolites of isolated anthracyclines. Anthracyclines include, but are not limited to doxorubicin, daunorubicin, nemorubicin, idrarubicin, epirubicin, aclarubicin, amrubicin, pirarubicin, valrubicin, doxazolidine, carubicin, mitoxantrone, and PNU-159682.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “optionally substituted,” refers to an indicated group being either substituted or unsubstituted.

The term “alkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent unsubstituted saturated branched or straight chain hydrocarbon of the stated number of carbon atoms (e.g., a C₁-C₆alkylene has from 1 to 6 carbon atoms) and having two monovalent centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene groups include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoromethylene (—CF₂—), tetrafluoroethylene (—CF₂CF₂—), and the like.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “heteroalkylene” refers to a bivalent unsubstituted straight or branched group derived from heteroalkyl (as defined herein). Examples of heteroalkylene groups include, but are not limited to, —NH—CH₂—CH₂—NH—, —NH—CH₂—CH₂—CH₂—NH—, —NH—CH₂—CH₂—CH₂—CH₂—NH—, —CH₂—CH₂—O—CH₂—, —CH₂—CH₂—O—CF₂—, —CH₂—CH₂—NH—CH₂—, —C(═O)—NH—CH₂—CH₂—NH—CH₂—C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—, —O—CH₂—CH₂—CH₂—N(CH₃)—CH₂—, —O—CH₂—CH₂—CH₂—NH—C(═O)—CH₂—CH₂—, —O—CH₂—CH₂—CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—, —CH₂—CH₂—N(CH₃)—CH₂—, —CH₂—CH₂—N⁺(CH₃)₂—, —NH—CH₂—CH₂(NH₂)—CH₂—, and —NH—CH₂—CH₂(NHCH₃)—CH₂—. A bivalent polyethylene glycol (PEG) moiety is a type of heteroalkylene group. In some embodiments, heteroalkylene groups do not include poly-glycine chains, such as di-glycine, tri-glycine, tetra-glycine, and higher order polyglycine peptides. In some embodiments, heteroalkylene groups are straight chain groups derived from heteroalkyl (as defined herein), and do not included branched groups derived from heteroalkyl (as defined herein).

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “haloalkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, trifluoromethyl, 2,2,2-trifluoroethyl, and 1-chloroisopropyl.

The term “cycloalkyl” refers to a cyclic, saturated or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C₃₋₆ cycloalkyl” groups include, cyclopropyl, cyclobutyl, cyclopentyl, and cyclohexyl. Cycloalkenyl and cycloalkynyl are types of cycloalkyl groups having at least one double bond, or at least one triple bond, respectively.

The term “aryl” refers to an unsubstituted monovalent carbocyclic aromatic hydrocarbon group of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The terms “heterocycle” and “heterocyclyl” are used interchangeably herein and refer to a saturated or partially unsaturated ring or a multiple condensed ring system, including bridged, fused, and spiro ring systems, in which one or more ring atoms is a heteroatom (e.g., oxygen, nitrogen, and sulfur). Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl and piperidinyl. The terms “heterocycle” and “heterocyclyl” also include multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated π system where all atoms contributing to the conjugated π system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

The term “hydroxyl” refers to an —OH group.

The term “cyano” refers to a —CN group.

The term “oxo” refers to a ═O group.

The term “acyl” refers to an alkyl, haloalkyl, alkenyl, alkynyl, aryl cycloalkyl, heteroaryl, or heterocyclyl group, as defined herein, connected to the remainder of the compound by a C═O (carbonyl) group.

The term “carboxamido” refers to a —C(═O)NRR′ group, wherein R and R′ are independently selected from the group consisting of hydrogen, alkyl, alkenyl, alkynyl, aryl cycloalkyl, heteroaryl, and heterocyclyl, as defined herein.

It will be appreciated by those skilled in the art that compounds described herein having a chiral center may exist in and be isolated in optically active and racemic forms.

As used herein, the term “free drug” refers to a biologically active species that is not covalently attached to an antibody. Accordingly, free drug refers to a compound as it exists immediately upon cleavage from the ADC. The release mechanism can be via a cleavable linker in the ADC, or via intracellular conversion or metabolism of the ADC. The free drug is a pharmacologically active species which is capable of exerting the desired biological effect. In some embodiments, the pharamacologically active species is the parent drug alone. In some embodiments, the pharamacologically active species is the parent drug bonded to a component or vestige of the ADC (e.g., a component of the linker, succinimide, hydrolyzed succinimide, and/or antibody that has not undergone subsequent intracellular metabolism). In some embodiments, free drug refers to an anthracycline compound, or a salt thereof, as described herein, for example, wherein one or more of X, Y, W, A, and M are absent. In some embodiments, free drug refers to PNU-159682, or a salt thereof.

As used herein, the term “Drug Unit” refers to the free drug that is conjugated to an antibody in an ADC, as described herein.

### Antibody Drug Conjugates (ADCs)

Some embodiments provide an antibody drug conjugate (ADC) having the structure:

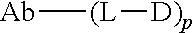

or a salt thereof;
   wherein:
   Ab is an antibody;
   wherein each L is covalently attached to Ab via a sulfur atom of a cysteine residue or an ϵ-amino group of a lysine residue in Ab;
   subscript p is an integer from 1 to 16;
   each D is an anthracycline;
   each L is a linker that has the formula -M-(A)ₐ-(W)_{w}—(Y)_{y}—(X)—, wherein:
   M is a succinimide, a hydrolyzed succinimide, an amide, or a triazole, wherein M is covalently attached to Ab;
   subscript a is 0 or 1;
   subscript y is 0 or 1;
   subscript w is 0 or 1;
   A is a C₂₋₁₀ alkylene optionally substituted with 1-3 R^{a1}; or a 3 to 20 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
   W is from 1-6 amino acids; or
   W has the structure:

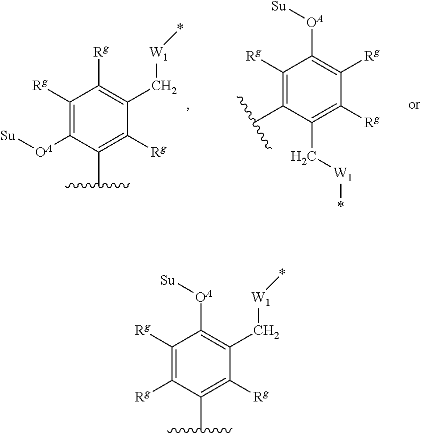

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
* represents covalent attachment to Y or X;
Y is a self-immolative moiety, a non-self-immolative releasable moiety, or a non-cleavable moiety;
X is from 1-10 amino acids; or
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
L is optionally substituted with a PEG Unit from PEG1 to PEG72.

Some embodiments provide an antibody drug conjugate (ADC) having the structure:

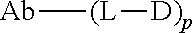

or a salt thereof;
   wherein:
   Ab is an antibody;
wherein each L is covalently attached to Ab via a sulfur atom of a cysteine residue or an ϵ-amino group of a lysine residue in Ab;
   subscript p is an integer from 1 to 16;
   each D is an anthracycline;
   each L is a linker that has the formula -M-(A)ₐ-(W)_{w}—(Y)_{y}—(X)—, wherein:
   M is a succinimide, a hydrolyzed succinimide, an amide, or a triazole, wherein M is covalently attached to Ab;
   subscript a is 0 or 1;
   subscript y is 0 or 1;
   subscript w is 0 or 1;
   A is a C₂₋₁₀ alkylene optionally substituted with 1-3 R^{a1}; or a 3 to 20 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
   W is from 1-6 amino acids; or
   W has the structure:

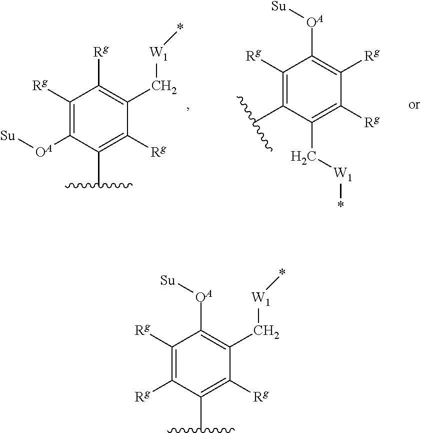

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
* represents covalent attachment to Y or X;
Y is a self-immolative moiety, a non-self-immolative releasable moiety, or a non-cleavable moiety;
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and L is optionally substituted with a PEG Unit from PEG1 to PEG72.

In some embodiments, the anthracycline is selected from the group consisting of doxorubicin, daunorubicin, nemorubicin, idrarubicin, epirubicin, aclarubicin, amrubicin, pirarubicin, valrubicin, doxazolidine, carubicin, mitoxantrone, and PNU-159682.

In some embodiments, each D is selected from the group consisting of:

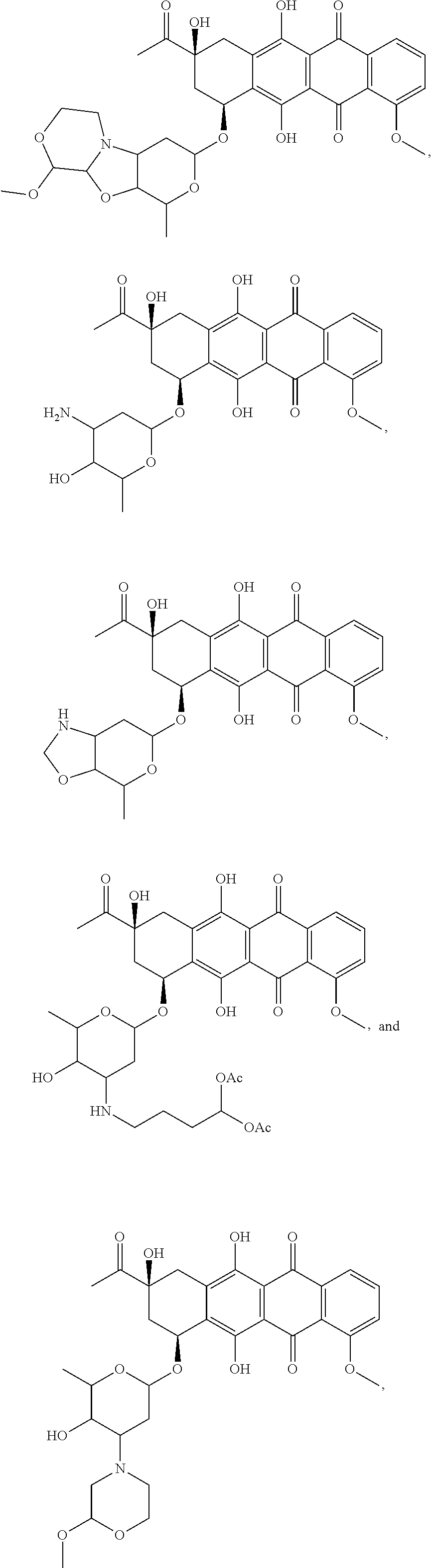

wherein [symbol] represents covalent attachment to L.

In some embodiments, each D-X is selected from the group consisting of:

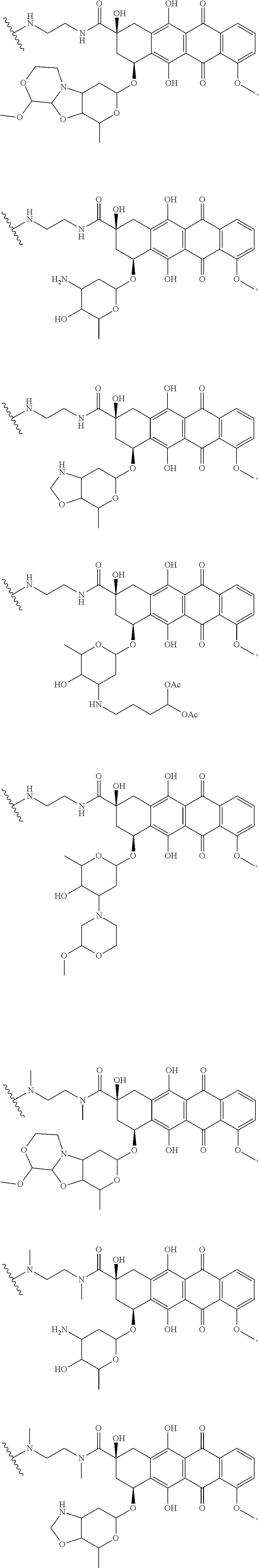

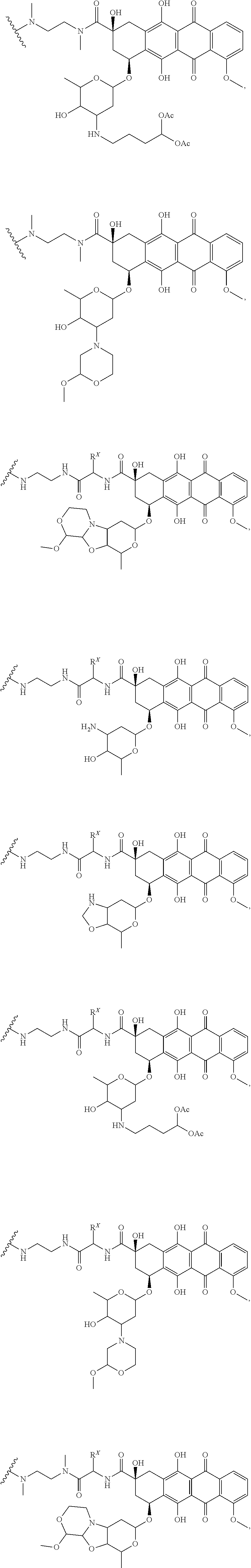

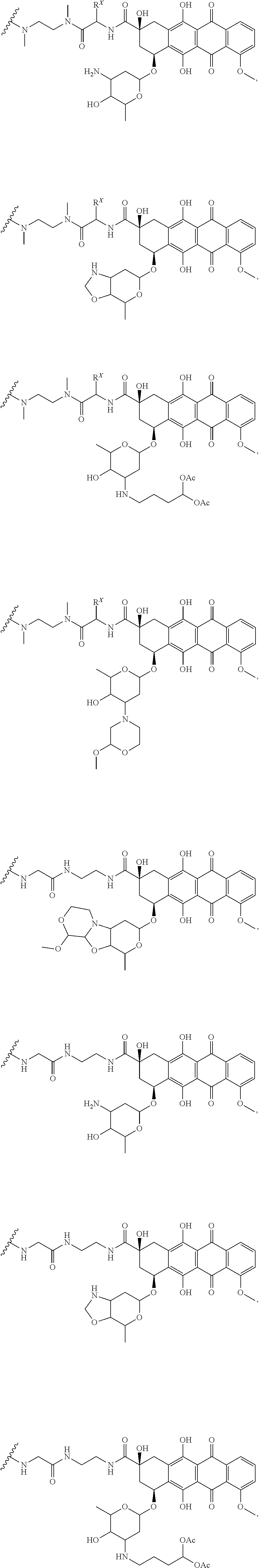

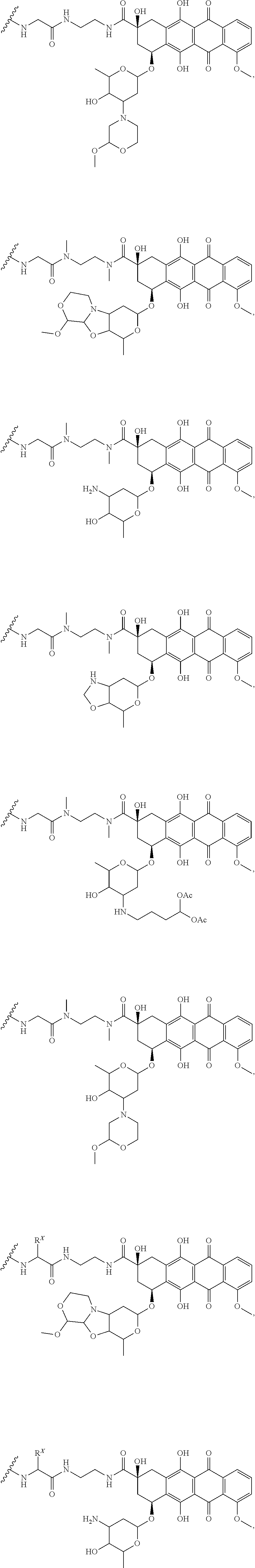

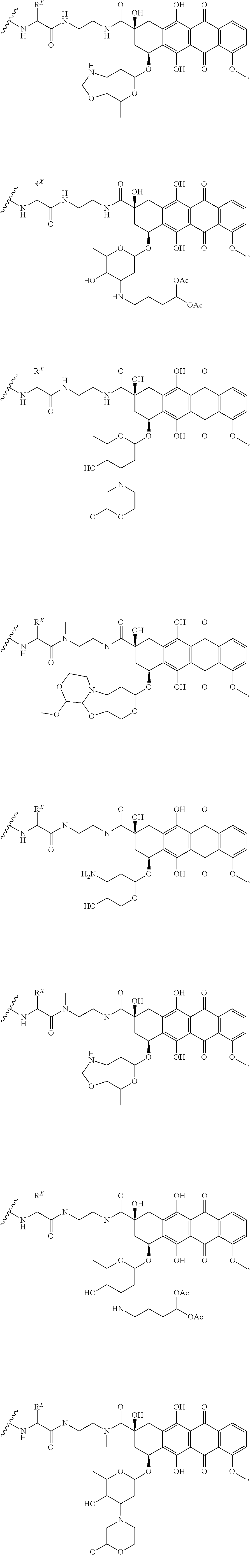

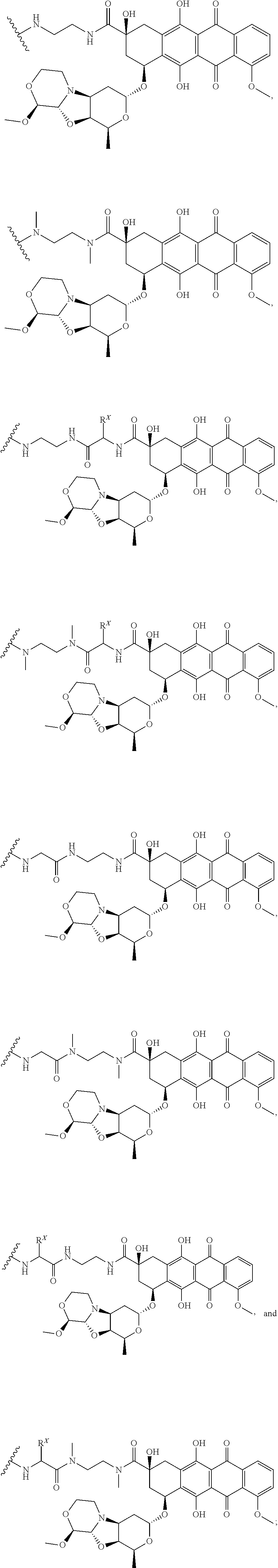

wherein R^{X} is as described herein, and wherein [symbol] represents covalent attachment to Y, W, A, or M.

Some embodiments provide an antibody drug conjugate (ADC) having the structure:

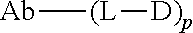

or a salt thereof;
   wherein:
   Ab is an antibody;
   wherein each L is covalently attached to Ab via a sulfur atom of a cysteine residue or an ϵ-amino group of a lysine residue in Ab;
   subscript p is an integer from 1 to 16;
   each D is:

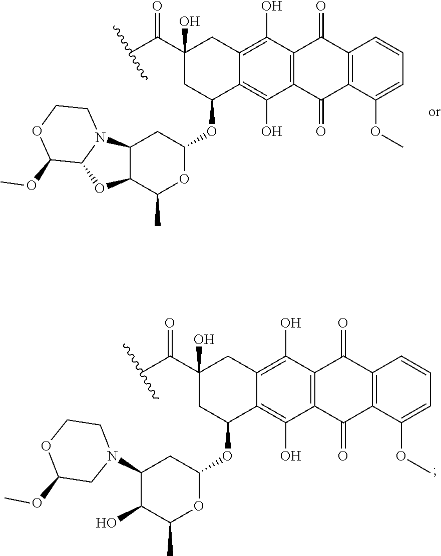

wherein [symbol] represents covalent attachment to L;
each L is a linker that has the formula -M-(A)ₐ-(W)_{w}—(Y)_{y}—(X)—, wherein:
   M is a succinimide, a hydrolyzed succinimide, an amide, or a triazole, wherein M is covalently attached to Ab;
   subscript a is 0 or 1;
   subscript y is 0 or 1;
   subscript w is 0 or 1;
   A is a C₂₋₁₀ alkylene optionally substituted with 1-3 R^{a1}; or a 3 to 20 membered heteroalkylene optionally substituted with 1-3 R^{b1};
   each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
   each R^{d1} and R^{e1} are independently hydrogen or C₁₋₃ alkyl;
   W is from 1-6 amino acids; or
   W has the structure:

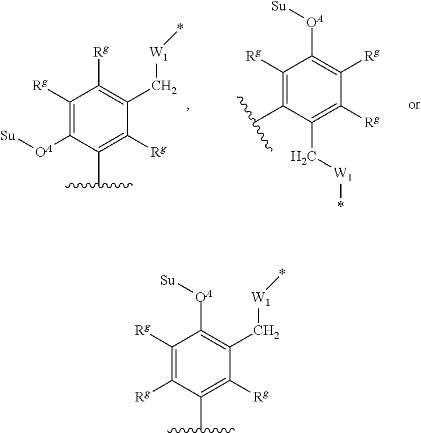

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
* represents covalent attachment to Y or X;
Y is a self-immolative moiety, a non-self-immolative releasable moiety, or a non-cleavable moiety;
X is from 1-10 amino acids; or
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
L is optionally substituted with a PEG Unit from PEG1 to PEG72.

Some embodiments provide an antibody drug conjugate (ADC) having the structure:

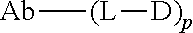

or a salt thereof;
   wherein:
   Ab is an antibody;
   wherein each L is covalently attached to Ab via a sulfur atom of a cysteine residue or an ϵ-amino group of a lysine residue in Ab;
   subscript p is an integer from 1 to 16;
   each D is:

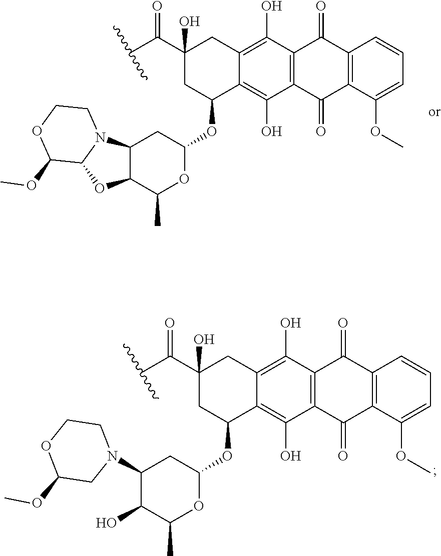

wherein [symbol] represents covalent attachment to L;
each L is a linker that has the formula -M-(A)ₐ-(W)_{w}—(Y)_{y}—(X)—, wherein:
M is a succinimide, a hydrolyzed succinimide, an amide, or a triazole, wherein M is covalently attached to Ab;
subscript a is 0 or 1;
subscript y is 0 or 1;
subscript w is 0 or 1;
A is a C₂₋₁₀ alkylene optionally substituted with 1-3 R^{a1}; or a 3 to 20 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
W is from 1-6 amino acids; or
W has the structure:

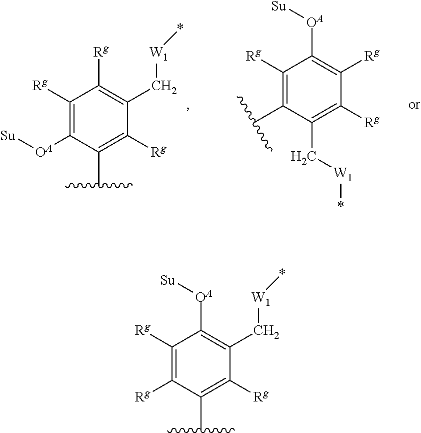

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—;
[symbol] represents covalent attachment to A or M;
* represents covalent attachment to Y or X;
Y is a self-immolative moiety, a non-self-immolative releasable moiety, or a non-cleavable moiety;
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
L is optionally substituted with a PEG Unit from PEG1 to PEG72.

In some embodiments, the ADC has the structure:

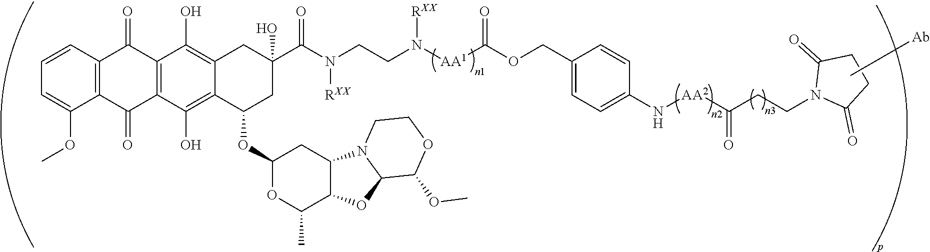

wherein:
each R^{XX} is independently hydrogen or Cia alkyl;
n1 is an integer from 0 to 4;
n2 is an integer from 1 to 4;
n3 is an integer from 1 to 4;
each AA¹ is independently selected from the group consisting of alanine, glycine, lysine, serine, aspartic acid, aspartate methyl ester, N,N-dimethyl-lysine, phenylalanine, citrulline, valine, asparagine, homoserine methyl ether, isoleucine, leucine, glutamic acid, histidine, arginine, threonine, O-methylserine, O-methylaspartic acid, O-methylglutamic acid, N-methyllysine, O-methyltyrosine, O-methylhistidine, and O-methylthreonine;
each AA² is independently selected from from the group consisting of alanine, glycine, lysine, serine, aspartic acid, aspartate methyl ester, N,N-dimethyl-lysine, phenylalanine, citrulline, valine, asparagine, homoserine methyl ether, isoleucine, leucine, glutamic acid, histidine, arginine, threonine, O-methylserine, O-methylaspartic acid, O-methylglutamic acid, N-methyllysine, O-methyltyrosine, O-methylhistidine, and O-methylthreonine;
Ab is an antibody; and
p is an integer from 1 to 16.

In some embodiments, each AA¹ is independently selected from the group consisting of alanine, glycine, valine, and serine.

In some embodiments, n1 is 0. In some embodiments, n1 is 1. In some embodiments, n1 is 2. In some embodiments, n1 is 3.

In some embodiments, each AA² is independently selected from the group consisting of alanine, glycine, valine, serine, leucine, and aspartic acid. In some embodiments, each AA² is independently selected from the group consisting of alanine and valine.

In some embodiments, n2 is 2. In some embodiments, (AA²)ₙ₂ is -Ala-Val-.

In some embodiments, n3 is 1.

In some embodiments, the ADC has the structure:

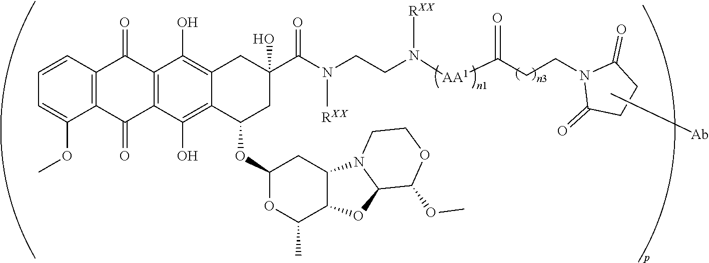

wherein:
each R^{XX} is independently hydrogen or C₁₋₃ alkyl;
n1 is an integer from 0 to 4;
each AA¹ is independently selected from the group consisting of alanine, glycine, lysine, serine, aspartic acid, aspartate methyl ester, N,N-dimethyl-lysine, phenylalanine, citrulline, valine, asparagine, homoserine methyl ether, isoleucine, leucine, glutamic acid, histidine, arginine, threonine, O-methylserine, O-methylaspartic acid, O-methylglutamic acid, N-methyllysine, O-methyltyrosine, O-methylhistidine, and O-methylthreonine;
n3 is an integer from 1 to 4;
Ab is an antibody; and
p is an integer from 1 to 16.

In some embodiments, n1 is 0. In some embodiments, n1 is 1. In some embodiments, n1 is 2. In some embodiments, n1 is 3. In some embodiments, when n1 is 3, at least one AA¹ is not glycine.

In some embodiments, each AA¹ is independently selected from the group consisting of alanine, glycine, valine, serine, leucine, arginine, and aspartic acid. In some embodiments, each AA¹ is independently selected from the group consisting of alanine, glycine, valine, and serine.

In some embodiments, n1 is 3; each AA¹ is independently selected from the group consisting of alanine, glycine, valine, serine, leucine, arginine, and aspartic acid; and wherein at least one AA¹ is not glycine.

In some embodiments, n3 is 1.

In some embodiments, the ADC has the structure:

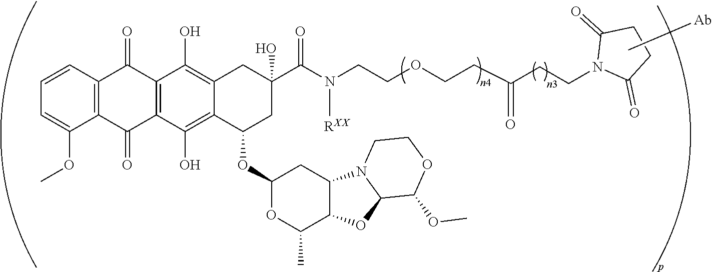

wherein:
R^{XX} is hydrogen or C₁₋₃ alkyl;
n4 is an integer from 2 to 8;
n3 is an integer from 1 to 4;
Ab is an antibody; and
p is an integer from 1 to 16.

In some embodiments, n4 is an integer from 3 to 6.

In some embodiments, n3 is 1.

In some embodiments, the ADC has a structure selected from the group consisting of:

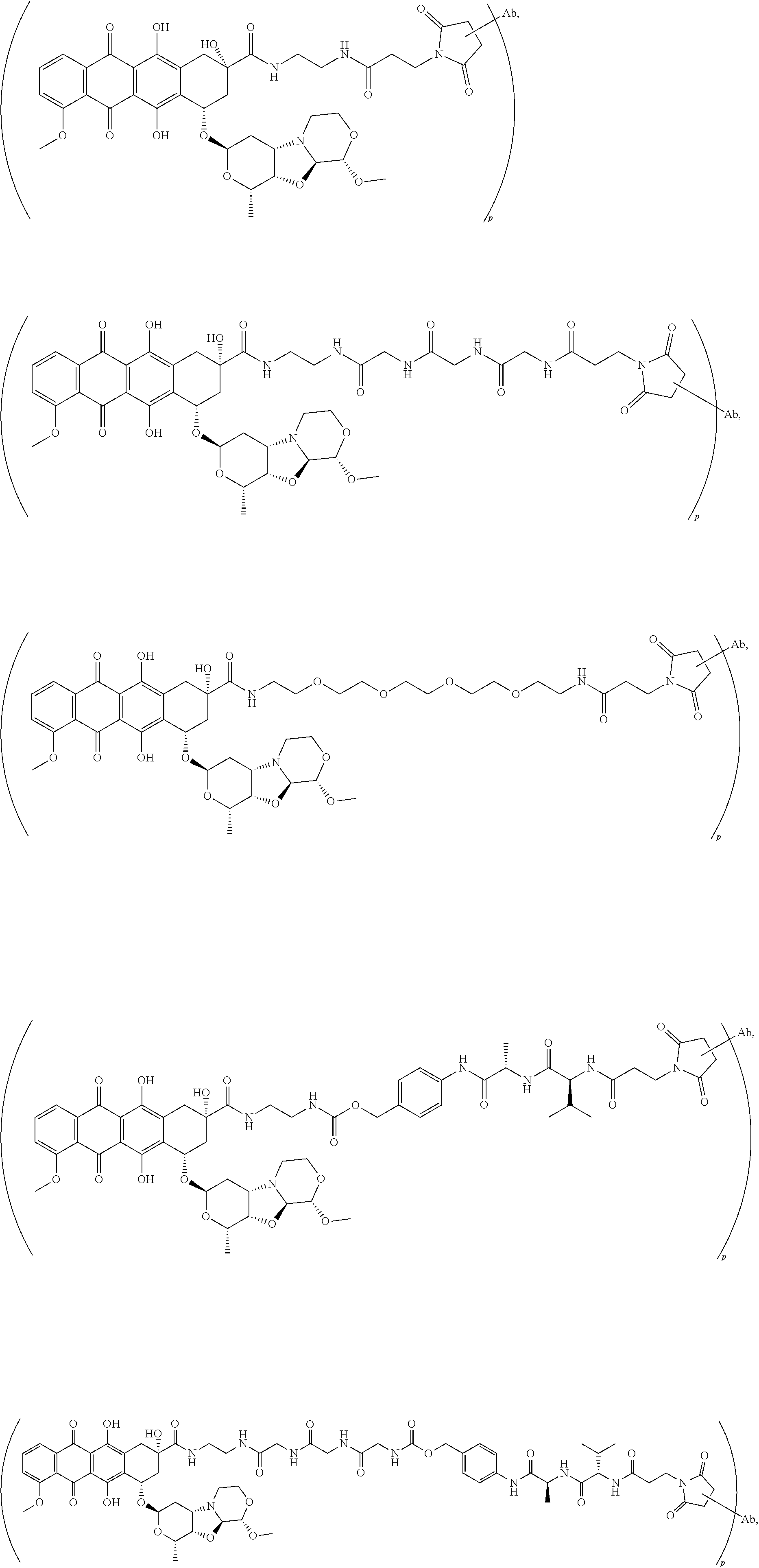

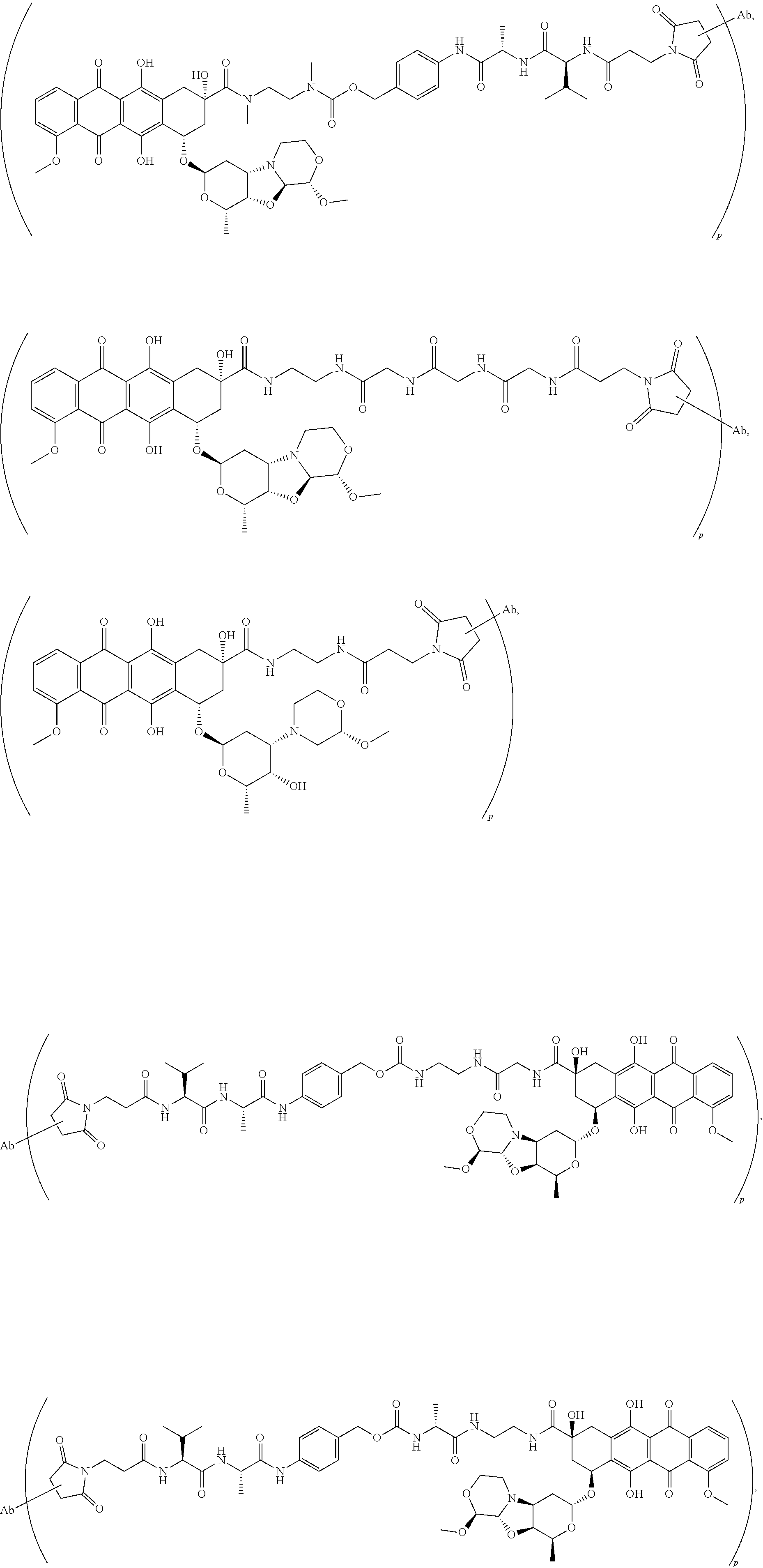

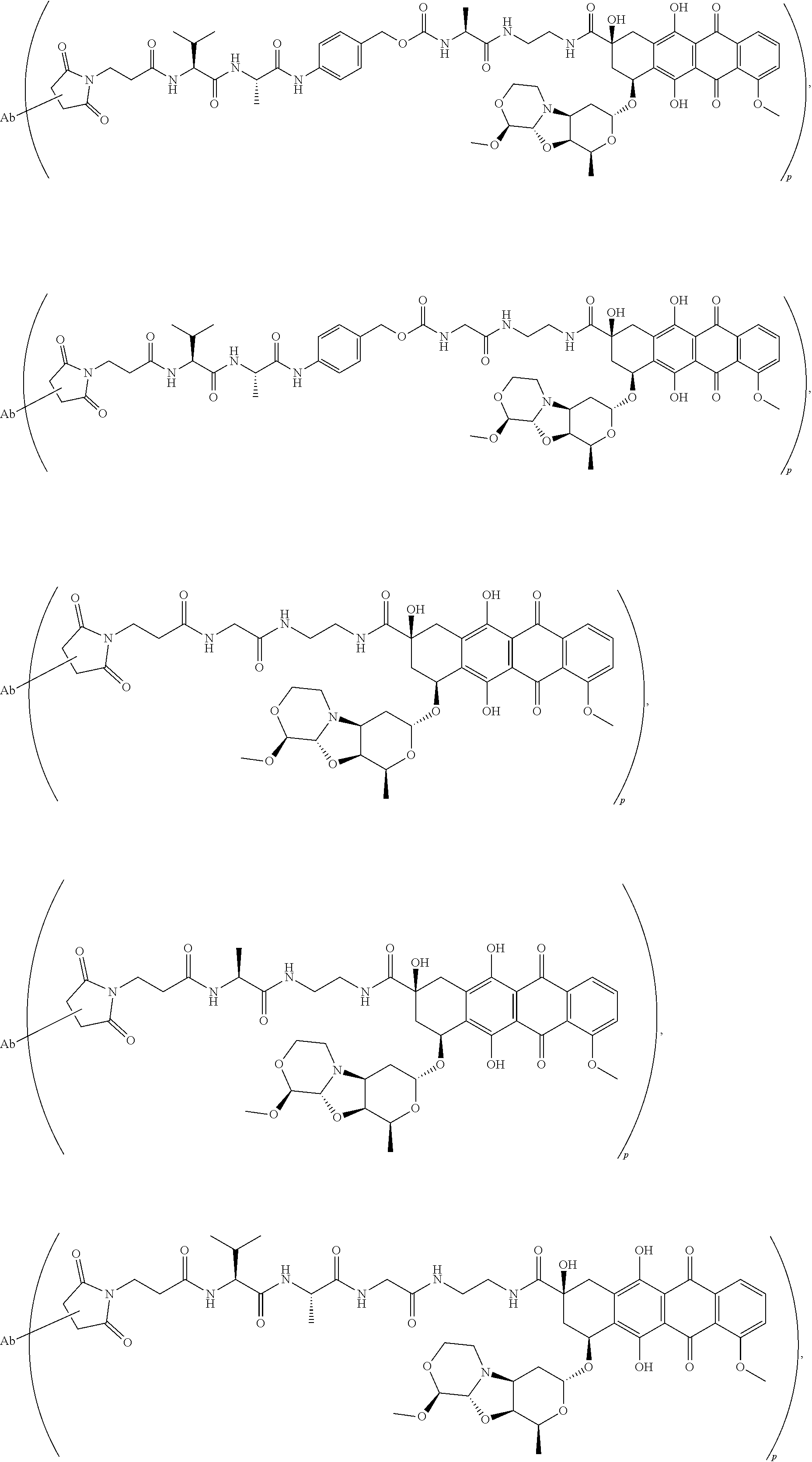

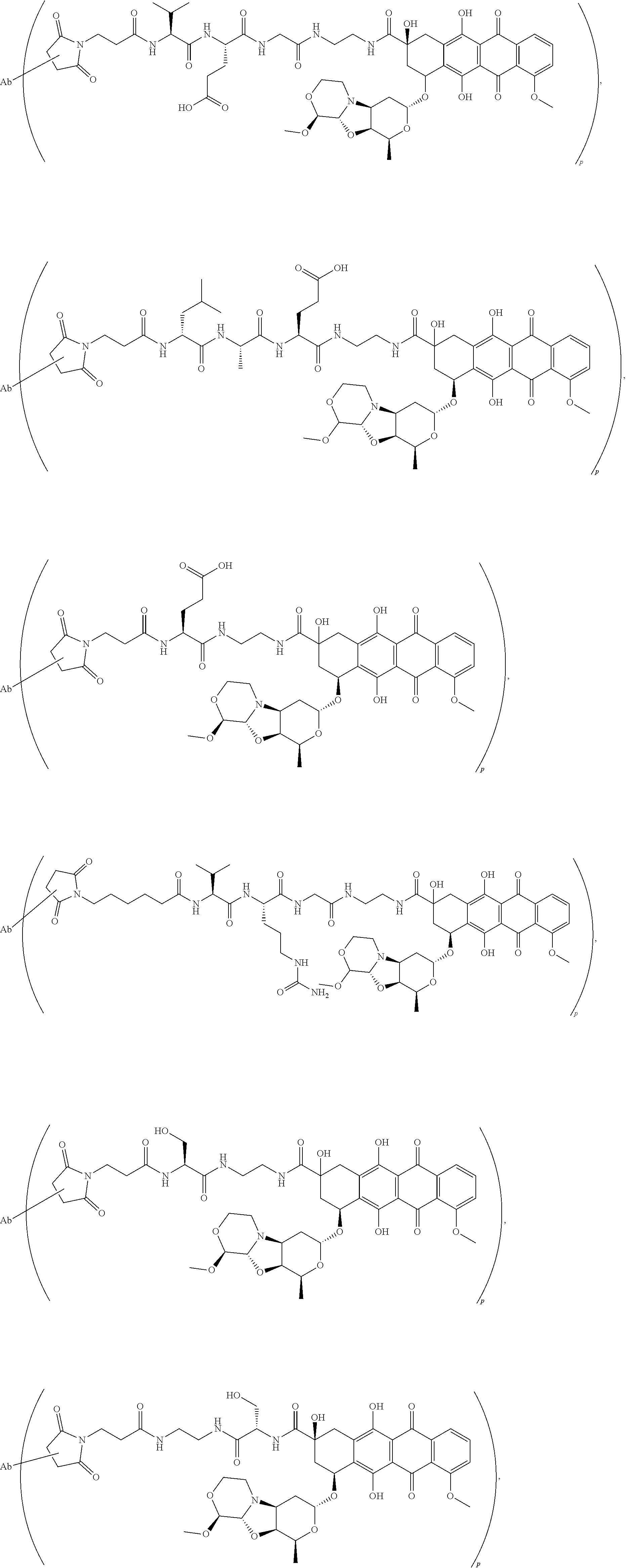

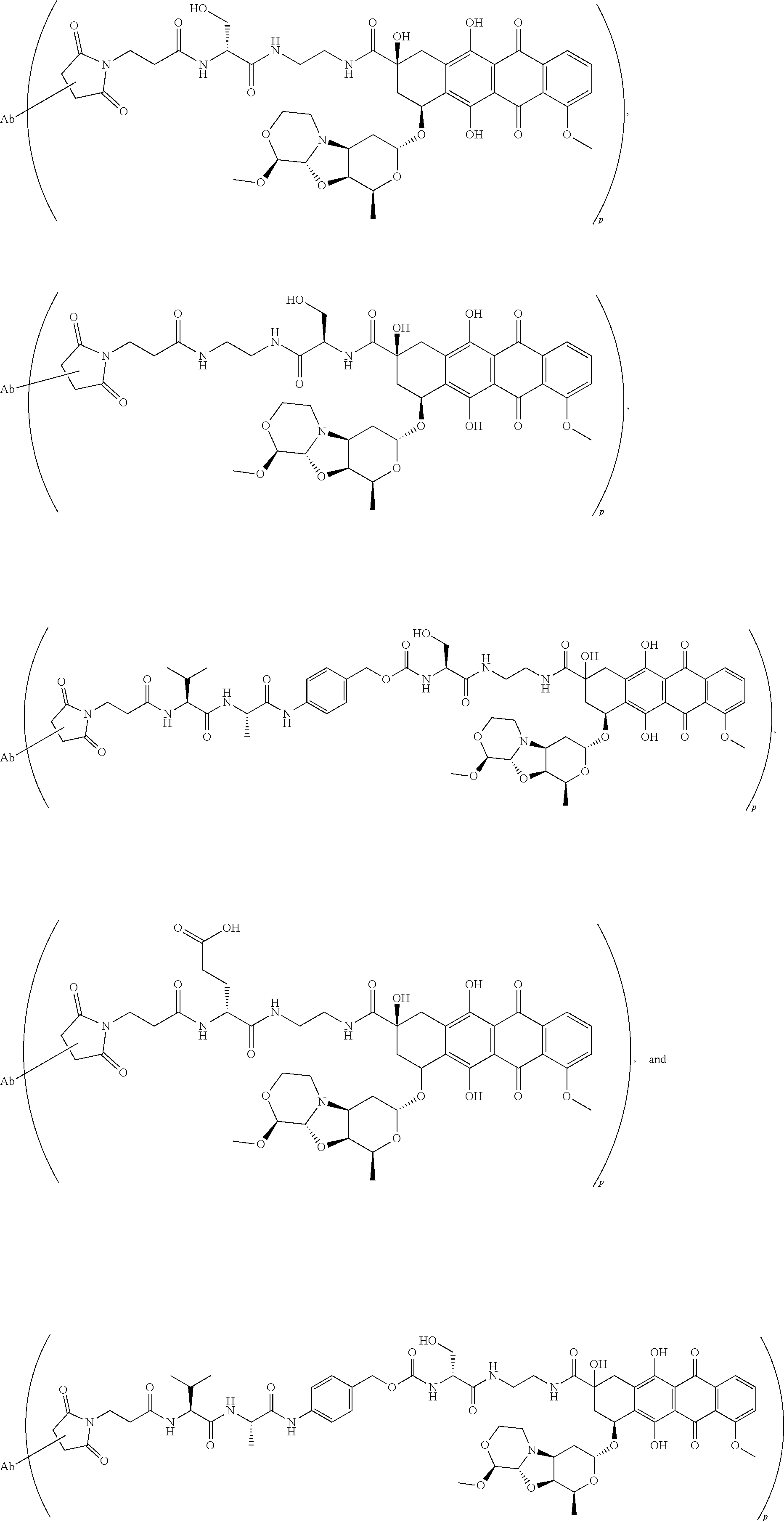

or a pharmaceutically acceptable salts of any of the foregoing.

In some embodiments, the ADCs described herein are present in the form of a salt. In some embodiments, the salt is a pharmaceutically acceptable salt.

In some embodiments subscript p is an integer from 1 to 8; from 4 to 12; or from 8 to 16. In some embodiments, subscript p is an even number. In some embodiments, subscript p is 2, 4, 6, 8, 10, 12, 14, or 16. In some embodiments, subscript p is 2, 4, 6, or 8.

In some embodiments, each L is covalently attached to Ab via a sulfur atom of a cysteine residue. In some embodiments, one or more of the cysteine residues is an engineered cysteine residue. In some embodiments, each cysteine residue is an engineered cysteine residue. In some embodiments, one or more of the cysteine residues is a native cysteine residue. In some embodiments, each cysteine residue is a native cysteine residue. In some embodiments, each sulfur atom is from a cysteine residue from a reduced interchain disulfide bond.

In some embodiments, each L is covalently attached to Ab via an ϵ-amino group of a lysine residue.

In some embodiments, the ADC is capable of releasing (i) a component of the linker bound to D; (ii) a component of antibody that has not undergone subsequent intracellular metabolism bound to L-D; and/or (iii) the parent compound D, as the free drug (as defined herein). In some embodiments, the free drug is released at the intended site of action targeted by the antibody. In some embodiments, the free drug is released within the intended site of action targeted by the antibody.

### Antibodies

In some embodiments, an antibody is a polyclonal antibody. In some embodiments, an antibody is a monoclonal antibody. In some embodiments, an antibody is chimeric. In some embodiments, an antibody is humanized. In some embodiments, an antibody is an antigen binding fragment.

Useful polyclonal antibodies are heterogeneous populations of antibody molecules derived from the sera of immunized animals. Useful monoclonal antibodies are homogeneous populations of antibodies to a particular antigenic determinant (e.g., a cancer or immune cell antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

In some embodiments, an antibody includes a functionally active fragment, derivative or analog of an antibody that binds specifically to target cells (e.g., cancer cell antigens) or other antibodies bound to cancer cells or matrix. In this regard, “functionally active” means that the fragment, derivative or analog is able to bind specifically to target cells. To determine which CDR sequences bind the antigen, synthetic peptides containing the CDR sequences are typically used in binding assays with the antigen by any binding assay method known in the art (e.g., the Biacore assay) (See, e.g., Kabat et al., 1991, Sequences of Proteins of Immunological Interest, Fifth Edition, National Institute of Health, Bethesda, Md; Kabat E et al., 1980, J. Immunology 125(3):961-969).

Additionally, recombinant antibodies, such as chimeric and humanized monoclonal antibodies, comprising both human and non-human portions, which are typically obtained using standard recombinant DNA techniques, are useful antibodies. A chimeric antibody is a molecule in which different portions are derived from different animal species, such as for example, those having a variable region derived from a murine monoclonal and a constant region derived from a human immunoglobulin. See, e.g., U.S. Pat. Nos. 4,816,567; and 4,816,397, which are incorporated herein by reference in their entireties. Humanized antibodies are antibody molecules from non-human species having one or more CDRs from the non-human species and a framework region from a human immunoglobulin molecule. See, e.g., U.S. Pat. No. 5,585,089, which is incorporated herein by reference in its entirety. Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321: 522-525; Verhoeyan et al., 1988, Science 239:1534; and Beidler et al., 1988, J. Immunol. 141:4053-4060; each of which is incorporated herein by reference in its entirety.

In some embodiments, an antibody is a completely human antibody. In some embodiments, an antibody is produced using transgenic mice that are incapable of expressing endogenous immunoglobulin heavy and light chain genes, but which are capable of expressing human heavy and light chain genes.

In some embodiments, an antibody is an intact or fully-reduced antibody. The term ‘fully-reduced’ is meant to refer to an antibody in which all four inter-chain disulfide linkages have been reduced to provide eight thiols that can be attached to a linker (L).

Attachment to an antibody can be via thioether linkages from native and/or engineered cysteine residues, or from an amino acid residue engineered to participate in a cycloaddition reaction (such as a click reaction) with the corresponding linker intermediate. See, e.g., Maerle, et al., PLOS One 2019: 14(1); e0209860. In some embodiments, an antibody is an intact or fully-reduced antibody, or is an antibody bearing engineered an cysteine group that is modified with a functional group that can participate in, for example, click chemistry or other cycloaddition reactions for attachment of other components of the ADC as described herein (e.g., Diels-Alder reactions or other [3+2] or [4+2] cycloadditions). See, e.g., Agard, et al., J. Am. Chem. Soc. Vol. 126, pp. 15046-15047 (2004); Laughlin, et al., Science, Vol. 320, pp. 664-667 (2008); Beatty, et al., ChemBioChem, Vol. 11, pp. 2092-2095 (2010); and Van Geel, et al., Bioconjug. Chem. Vol. 26, pp. 2233-2242 (2015).

Antibodies that bind specifically to a cancer or immune cell antigen are available commercially or produced by any method known to one of skill in the art such as, e.g., chemical synthesis or recombinant expression techniques. The nucleotide sequences encoding antibodies that bind specifically to a cancer or immune cell antigen are obtainable, e.g., from the GenBank database or similar database, literature publications, or by routine cloning and sequencing.

In some embodiments, the antibody can be used for the treatment of a cancer (e.g., an antibody approved by the FDA and/or EMA). In some embodiments, the antibody described herein is selected from the group consisting of avelumab, durvalumab, daratumumab, elotuzumab, necitumumab, atezolizumab, nivolumab, dinutuximab, bevacizumab, pembrolizumab, ramucirumab, alemtuzumab, pertuzumab, obinutuzumab, ipilimumab, denosumab, ofatumumab, catumaxomab, panitumumab, bevacizumab, cetuximab, tositumomab, alemtuzumab, trastuzumab, rituximab, sintilimab, tislelizumab, camrelizumab, huJ591, JS001, hu3S193, TRC093, TRC105, AGEN1181, AGEN2034, MEDI4736, NEO-102, MK-0646, ZKAB001, TB-403, GLS-010, CT-011, INCMGA00012, AGEN1884, MK-3475, GC1118, DS-8201a, CC-95251, Sym004, CS1001, and REGN2810. In some embodiments, the antibody described herein is selected from the group consisting of rituximab, obinutuzumab, ofatumumab, trastuzumab, alemtuzumab, mogamulizumab, cetuximab, and dinutuximab. In some embodiments, the antibody described herein is rituximab. In some embodiments, the antibody described herein is obinutuzumab. In some embodiments, the antibody described herein is ofatumumab. In some embodiments, the antibody described herein is trastuzumab. In some embodiments, the antibody described herein is alemtuzumab. In some embodiments, the antibody described herein is mogamulizumab. In some embodiments, the antibody described herein is cetuximab. In some embodiments, the antibody described herein is dinutuximab.

Antibodies that bind specifically to a cancer or immune cell antigen are available commercially or produced by any method known to one of skill in the art such as, e.g., recombinant expression techniques. The nucleotide sequences encoding antibodies that bind specifically to a cancer or immune cell antigen are obtainable, e.g., from the GenBank database or similar database, literature publications, or by routine cloning and sequencing.

In some embodiments, an antibody can bind specifically to a receptor or a receptor complex expressed on lymphocytes. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein or other immune cell expressed surface receptor.

In some embodiments, an antibody can bind specifically to a cancer cell antigen. In some embodiments, an antibody can bind specifically to an immune cell antigen. It will be understood that the antibody component in an ADC is an antibody in residue form such that “Ab” in the ADC structures described herein incorporates the structure of the antibody.

Non-limiting examples of antibodies that can be used for treatment of cancer and antibodies that bind specifically to tumor associated antigens are disclosed in Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998, each of which is hereby incorporated by reference in its entirety.

Exemplary antigens are provided below. Exemplary antibodies that bind the indicated antigen are shown in parentheses.

In some embodiments, the antigen is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD147 (exemplary antibodies include gavilimomab and metuzumab), CD19, CD20 (exemplary antibodies include divozilimab and ibritumomab tiuxetan), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CLPTM1L, DPP4, EGFR, ERVMER34-1, FASL, FSHR, FZD5, FZD8, GUCY2C (exemplary antibodies include indusatumab), IFNAR1 (exemplary antibodies include faralimomab), IFNAR2, LMP2, MLANA, SIT1, TLR2/4/1 (exemplary antibodies include tomaralimab), TM4SF5, TMEM132A, TMEM40, UPK1B, VEGF, and VEFGR2 (exemplary antibodies include gentuximab).

In some embodiments, the tumor-associated antigen is a transmembrane transport protein. For example, the following antigens are transmembrane transport proteins: ASCT2 (exemplary antibodies include idactamab), MFSD13A, Mincle, NOX1, SLC10A2, SLC12A2, SLC17A2, SLC38A1, SLC39A5, SLC39A6 also known as LIV1 (exemplary antibodies include ladiratuzumab), SLC44A4, SLC6A15, SLC6A6, SLC7A11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-1 (exemplary antibodies include alemtuzumab), carcinoembryonic antigen (exemplary antibodies include arcitumomab, cergutuzumab, amunaleukin, and labetuzumab), CD112, CD155, CD24, CD247, CD37 (exemplary antibodies include lilotomab), CD38 (exemplary antibodies include felzartamab), CD3D, CD3E (exemplary antibodies include foralumab and teplizumab), CD3G, CD96, CDCP1, CDH17, CDH3, CDH6, CEACAMI, CEACAM6, CLDN1, CLDN16, CLDN18.1 (exemplary antibodies include zolbetuximab), CLDN18.2 (exemplary antibodies include zolbetuximab), CLDN19, CLDN2, CLEC12A (exemplary antibodies include tepoditamab), DPEP1, DPEP3, DSG2, endosialin (exemplary antibodies include ontuxizumab), ENPP1, EPCAM (exemplary antibodies include adecatumumab), FN, FN1, Gp100, GPA33, gpNMB (exemplary antibodies include glembatumumab), ICAM1, LlCAM, LAMP1, MELTF also known as CD228, NCAM1, Nectin-4 (exemplary antibodies include enfortumab), PDPN, PMSA, PROM1, PSCA, PSMA, Siglecs 1-16, SIRPa, SIRPg, TACSTD2, TAG-72, Tenascin, Tissue Factor also known as TF (exemplary antibodies include tisotumab), and ULBP1/2/3/4/5/6.

In some embodiments, the tumor-associated antigen is a transmembrane or membrane-associated receptor kinase. For example, the following antigens are transmembrane or membrane-associated receptor kinases: ALK, Axl (exemplary antibodies include tilvestamab), BMPR2, DCLK1, DDR1, EPHA receptors, EPHA2, ERBB2 also known as HER2 (exemplary antibodies include trastuzumab, bevacizumab, pertuzumab, and margetuximab), ERBB3, FLT3, PDGFR-B (exemplary antibodies include rinucumab), PTK7 (exemplary antibodies include cofetuzumab), RET, ROR1 (exemplary antibodies include cirmtuzumab), ROR2, ROS1, andTie3.

In some embodiments, the tumor-associated antigen is a membrane-associated or membrane-localized protein. For example, the following antigens are membrane-associated or membrane-localized proteins: ALPP, ALPPL2, ANXA1, FOLR1 (exemplary antibodies include farletuzumab), IL13Ra2, ILIRAP (exemplary antibodies include nidanilimab), NT5E, OX40, Ras mutant, RGS5, RhoC, SLAMF7 (exemplary antibodies include elotuzumab), and VSIR.

In some embodiments, the tumor-associated antigen is a transmembrane G-protein coupled receptor (GPCR). For example, the following antigens are GPCRs: CALCR, CD97, GPR87, and KISS1R.

In some embodiments, the tumor-associated antigen is cell-surface-associated or a cell-surface receptor. For example, the following antigens are cell-surface-associated and/or cell-surface receptors: B7-DC, BCMA, CD137, CD 244, CD3 (exemplary antibodies include otelixizumab and visilizumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), Gpc3 (exemplary antibodies include ragifilimab), HAVCR2, HLA-E, HLA-F, HLA-G, LAG-3 (exemplary antibodies include encelimab), LY6G6D, LY9, MICA, MICB, MSLN, MUC1, MUC5AC, NY-ESO-1, OY-TES1, PVRIG, Sialyl-Thomsen-Nouveau Antigen, Sperm protein 17, TNFRSF12, and uPAR.

In some embodiments, the tumor-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab and omburtamab), B7-H4, B7-H6, and B7-H7.

In some embodiments, the tumor-associated antigen is a transcription factor or a DNA-binding protein. For example, the following antigens are transcription factors: ETV6-AML, MYCN, PAX3, PAX5, and WT1. The following protein is a DNA-binding protein: BORIS.

In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: alpha v beta 6, ITGAV (exemplary antibodies include abituzumab), ITGB6, and ITGB8.

In some embodiments, the tumor-associated antigen is a glycolipid. For example, the following are glycolipid antigens: FucGM1, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormone receptor. For example, the following antigens are cell-surface hormone receptors: AMHR2 and androgen receptor.

In some embodiments, the tumor-associated antigen is a transmembrane or membrane-associated protease. For example, the following antigens are transmembrane or membrane-associated proteases: ADAM12, ADAM9, TMPRSSI1D, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C₅ complement, CCNB1, CSPG4, CYP1B1, De2-7 EGFR, EGF, Fas-related antigen 1, FBP, G250, GAGE, HAS3, HPV E6 E7, hTERT, IDO1, LCK, Legumain, LYPD1, MAD-CT-1, MAD-CT-2, MAGEA3, MAGEA4, MAGEC2, MerTk, ML-IAP, NA17, NY-BR-1, p53, p⁵³ mutant, PAP, PLAVI, polysialic acid, PR1, PSA, Sarcoma translocation breakpoints, SART3, sLe, SSX2, Survivin, Tn, TRAIL, TRAIL1, TRP-2, and XAGE1.

In some embodiments, the antigen is an immune-cell-associated antigen. In some embodiments, the immune-cell-associated antigen is a transmembrane protein. For example, the following antigens are transmembrane proteins: BAFF-R, CD163, CD19, CD20 (exemplary antibodies include rituximab, ocrelizumab, divozilimab; ibritumomab tiuxetan), CD25 (exemplary antibodies include basiliximab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-1 (exemplary antibodies include ipilimumab, nivolumab, pembrolizumab, balstilimab, budigalimab, geptanolimab, toripalimab, and pidilizumabsf), SIT1, and TLR2/4/1 (exemplary antibodies include tomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CD112, CD155, CD24, CD247, CD28, CD30L, CD37 (exemplary antibodies include lilotomab), CD38 (exemplary antibodies include felzartamab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: Axl (exemplary antibodies include tilvestamab) and FLT3.

In some embodiments, the immune-cell-associated antigen is a membrane-associated or membrane-localized protein. For example, the following antigens are membrane-associated or membrane-localized proteins: CD83, IL1RAP (exemplary antibodies include nidanilimab), OX40, SLAMF7 (exemplary antibodies include elotuzumab), and VSIR.

In some embodiments, the immune-cell-associated antigen is a transmembrane G-protein coupled receptor (GPCR). For example, the following antigens are GPCRs: CCR4 (exemplary antibodies include mogamulizumab-kpkc), CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD 244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab), Trem2, and uPAR.

In some embodiments, the immune-cell-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR4 (exemplary antibodies include ulocuplumab), IL-21R, and IL-5R (exemplary antibodies include benralizumab).

In some embodiments, the immune-cell-associated antigen is a co-stimulatory, surface-expressed protein. For example, the following antigens are co-stimulatory, surface-expressed proteins: B7-H 3 (exemplary antibodies include enoblituzumab and omburtamab), B7-H4, B7-H6, and B7-H7.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C₅ complement, IDO1, LCK, MerTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR1 (exemplary antibodies include faralimomab), and IFNAR2.

In some embodiments, the antigen is CD30. In some embodiments, the antibody is an antibody or antigen-binding fragment that binds to CD30, such as described in International Patent Publication No. WO 02/43661. In some embodiments, the anti-CD30 antibody is cAC10, which is described in International Patent Publication No. WO 02/43661. cAC10 is also known as brentuximab. In some embodiments, the anti-CD30 antibody comprises the CDRs of cAC10. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at last 98%, at least 99%, or 100% identical to the amino acid sequence of SEQ ID NO: 7 and a light chain variable region comprising an amino acid sequence that is at least 95% at least 96%, at least 97%, at last 98%, at least 99%, or 100% identical to the amino acid sequence of SEQ ID NO: 8. In some embodiments, the anti-CD30 antibody comprises a heavy chain comprising the amino acid sequence of SEQ ID NO: 9 or SEQ ID NO: 10 and a light chain comprising the amino acid sequence of SEQ ID NO: 11.

In some embodiments, the antigen is CD70. In some embodiments, the antibody is an antibody or antigen-binding fragment that binds to CD70, such as described in International Patent Publication No. WO 2006/113909. In some embodiments, the antibody is a h1F6 anti-CD70 antibody, which is described in International Patent Publication No. WO 2006/113909. h1F6 is also known as vorsetuzumab. In some embodiments, the anti-CD70 antibody comprises a heavy chain variable region comprising the three CDRs of SEQ ID NO:12 and a light chain variable region comprising the three CDRs of SEQ ID NO:13. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD70 antibody comprises a heavy chain variable region comprising an amino acid sequence that is at least 95%, at least 96%, at least 97%, at last 98%, at least 99%, or 100% identical to the amino acid sequence of SEQ ID NO: 12 and a light chain variable region comprising an amino acid sequence that is at least 95% at least 96%, at least 97%, at last 98%, at least 99%, or 100% identical to the amino acid sequence of SEQ ID NO: 13. In some embodiments, the anti-CD30 antibody comprises a heavy chain comprising the amino acid sequence of SEQ ID NO: 14 and a light chain comprising the amino acid sequence of SEQ ID NO: 15.

In some embodiments, the antigen is interleukin-1 receptor accessory protein (IL1RAP). IL1RAP is a co-receptor of the IL1 receptor (IL1R1) and is required for interleukin-1 (IL1) signaling. IL1 has been implicated in the resistance to certain chemotherapy regimens. IL1RAP is overexpressed in various solid tumors, both on cancer cells and in the tumor microenvironment, but has low expression on normal cells. IL1RAP is also overexpressed in hematopoietic stem and progenitor cells, making it a candidate to target for chronic myeloid leukemia (CML). IL1RAP has also been shown to be overexpressed in acute myeloid leukemia (AML). Antibody binding to IL1RAP could block signal transduction from IL-1 and IL-33 into cells and allow NK-cells to recognize tumor cells and subsequent killing by antibody dependent cellular cytotoxicity (ADCC).

In some embodiments, the antigen is ASCT2. ASCT2 is also known as SLC1A5. ASCT2 is a ubiquitously expressed, broad-specificity, sodium-dependent neutral amino acid exchanger. ASCT2 is involved in glutamine transport. ASCT2 is overexpressed in different cancers and is closely related to poor prognosis. Downregulating ASCT2 has been shown to suppress intracellular glutamine levels and downstream glutamine metabolism, including glutathione production. Due to its high expression in many cancers, ASCT2 is a potential therapeutic target. These effects attenuated growth and proliferation, increased apoptosis and autophagy, and increased oxidative stress and mTORC1 pathway suppression in head and neck squamous cell carcinoma (HNSCC). Additionally, silencing ASCT2 improved the response to cetuximab in HNSCC.

In some embodiments, an antibody provided herein binds to TROP2. In some embodiments, the antibody comprises CDR-H1, CDR-H2, CDR-H3, CDR-L1, CDR-L2, and CDR-L3 comprising the amino acid sequences of SEQ ID NOs: 16, 17, 18, 19, 20, and 21, respectively. In some embodiments, the antibody comprises a heavy chain variable region comprising the amino acid sequence of SEQ ID NO: 22 and a light chain variable region comprising the amino acid sequence of SEQ ID NO: 23. In some embodiments, the antibody is sacituzumab. In some embodiments, the antibody comprises CDR-H1, CDR-H2, CDR-H3, CDR-L1, CDR-L2, and CDR-L3 comprising the amino acid sequences of SEQ ID NOs: 24, 25, 26, 27, 28, and 29, respectively. In some embodiments, the antibody comprises a heavy chain variable region comprising the amino acid sequence of SEQ ID NO: 30 and a light chain variable region comprising the amino acid sequence of SEQ ID NO: 31. In some embodiments, the antibody is datopotamab.

In some embodiments, an antibody provided herein binds to MICA. In some embodiments, the antibody comprises CDR-H1, CDR-H2, CDR-H3, CDR-L1, CDR-L2, and CDR-L3 comprising the amino acid sequences of SEQ ID NOs: 32, 33, 34, 35, 36, and 37, respectively. In some embodiments, the antibody comprises a heavy chain variable region comprising the amino acid sequence of SEQ ID NO: 38 and a light chain variable region comprising the amino acid sequence of SEQ ID NO: 39. In some embodiments, the antibody is h1D5v11 hIgG1K. In some embodiments, the antibody comprises CDR-H1, CDR-H2, CDR-H3, CDR-L1, CDR-L2, and CDR-L3 comprising the amino acid sequences of SEQ ID NOs: 40, 41, 42, 43, 44, and 45, respectively. In some embodiments, the antibody comprises a heavy chain variable region comprising the amino acid sequence of SEQ ID NO: 46 and a light chain variable region comprising the amino acid sequence of SEQ ID NO: 47. In some embodiments, the antibody is MICA.36 hIgG1K G236A. In some embodiments, the antibody comprises CDR-H1, CDR-H2, CDR-H3, CDR-L1, CDR-L2, and CDR-L3 comprising the amino acid sequences of SEQ ID NOs: 48, 49, 50, 51, 52, and 53, respectively. In some embodiments, the antibody comprises a heavy chain variable region comprising the amino acid sequence of SEQ ID NO: 54 and a light chain variable region comprising the amino acid sequence of SEQ ID NO: 55. In some embodiments, the antibody is h3F9 H1L3 hIgG1K. In some embodiments, the antibody comprises CDR-H1, CDR-H2, CDR-H3, CDR-L1, CDR-L2, and CDR-L3 comprising the amino acid sequences of SEQ ID NOs: 56, 57, 58, 59, 60, and 61, respectively. In some embodiments, the antibody comprises a heavy chain variable region comprising the amino acid sequence of SEQ ID NO: 62 and a light chain variable region comprising the amino acid sequence of SEQ ID NO: 63. In some embodiments, the antibody is CM33322 Ab28 hIgG1K.

In some embodiments, an antibody provided herein binds to CD24. In some embodiments, the antibody comprises CDR-H1, CDR-H2, CDR-H3, CDR-L1, CDR-L2, and CDR-L3 comprising the amino acid sequences of SEQ ID NOs: 64, 65, 66, 67, 68, and 69, respectively. In some embodiments, the antibody comprises a heavy chain variable region comprising the amino acid sequence of SEQ ID NO: 70 and a light chain variable region comprising the amino acid sequence of SEQ ID NO: 71. In some embodiments, the antibody is SWA11.

In some embodiments, an antibody provided herein binds to ITGav. In some embodiments, the antibody comprises CDR-H1, CDR-H2, CDR-H3, CDR-L1, CDR-L2, and CDR-L3 comprising the amino acid sequences of SEQ ID NOs: 72, 73, 74, 75, 76, and 77, respectively. In some embodiments, the antibody comprises a heavy chain variable region comprising the amino acid sequence of SEQ ID NO: 78 and a light chain variable region comprising the amino acid sequence of SEQ ID NO: 79. In some embodiments, the antibody is intetumumab. In some embodiments, the antibody comprises CDR-H1, CDR-H2, CDR-H3, CDR-L1, CDR-L2, and CDR-L3 comprising the amino acid sequences of SEQ ID NOs: 80, 81, 82, 83, 84, and 85, respectively. In some embodiments, the antibody comprises a heavy chain variable region comprising the amino acid sequence of SEQ ID NO: 86 and a light chain variable region comprising the amino acid sequence of SEQ ID NO: 87. In some embodiments, the antibody is abituzumab.

In some embodiments, an antibody provided herein binds to gpA33. In some embodiments, the antibody comprises CDR-H1, CDR-H2, CDR-H3, CDR-L1, CDR-L2, and CDR-L3 comprising the amino acid sequences of SEQ ID NOs: 88, 89, 90, 91, 92, and 93, respectively. In some embodiments, the antibody comprises a heavy chain variable region comprising the amino acid sequence of SEQ ID NO: 94 and a light chain variable region comprising the amino acid sequence of SEQ ID NO: 95.

In some embodiments, an antibody provided herein binds to IL1Rap. In some embodiments, the antibody comprises CDR-H1, CDR-H2, CDR-H3, CDR-L1, CDR-L2, and CDR-L3 comprising the amino acid sequences of SEQ ID NOs: 96, 97, 98, 99, 100, and 101, respectively. In some embodiments, the antibody comprises a heavy chain variable region comprising the amino acid sequence of SEQ ID NO: 102 and a light chain variable region comprising the amino acid sequence of SEQ ID NO: 103. In some embodiments, the antibody is nidanilimab.

In some embodiments, an antibody provided herein binds to EpCAM. In some embodiments, the antibody comprises CDR-H1, CDR-H2, CDR-H3, CDR-L1, CDR-L2, and CDR-L3 comprising the amino acid sequences of SEQ ID NOs: 104, 105, 106, 017, 108, and 109, respectively. In some embodiments, the antibody comprises a heavy chain variable region comprising the amino acid sequence of SEQ ID NO: 110 and a light chain variable region comprising the amino acid sequence of SEQ ID NO: 111. In some embodiments, the antibody is adecatumumab. In some embodiments, the antibody comprises CDR-H1, CDR-H2, CDR-H3, CDR-L1, CDR-L2, and CDR-L3 comprising the amino acid sequences of SEQ ID NOs: 112, 113, 114, 115, 116, and 117, respectively. In some embodiments, the antibody comprises a heavy chain variable region comprising the amino acid sequence of SEQ ID NO: 118 and a light chain variable region comprising the amino acid sequence of SEQ ID NO: 119. In some embodiments, the antibody is Ep157305. In some embodiments, the antibody comprises CDR-H1, CDR-H2, CDR-H3, CDR-L1, CDR-L2, and CDR-L3 comprising the amino acid sequences of SEQ ID NOs: 120, 121, 122, 123, 124, and 125, respectively. In some embodiments, the antibody comprises a heavy chain variable region comprising the amino acid sequence of SEQ ID NO: 126 and a light chain variable region comprising the amino acid sequence of SEQ ID NO: 127. In some embodiments, the antibody is Ep3-171. In some embodiments, the antibody comprises CDR-H1, CDR-H2, CDR-H3, CDR-L1, CDR-L2, and CDR-L3 comprising the amino acid sequences of SEQ ID NOs: 128, 129, 130, 131, 132, and 133, respectively. In some embodiments, the antibody comprises a heavy chain variable region comprising the amino acid sequence of SEQ ID NO: 134 and a light chain variable region comprising the amino acid sequence of SEQ ID NO: 135. In some embodiments, the antibody is Ep3622w94. In some embodiments, the antibody comprises CDR-H1, CDR-H2, CDR-H3, CDR-L1, CDR-L2, and CDR-L3 comprising the amino acid sequences of SEQ ID NOs: 136, 137, 138, 139, 140, and 141, respectively. In some embodiments, the antibody comprises a heavy chain variable region comprising the amino acid sequence of SEQ ID NO: 142 and a light chain variable region comprising the amino acid sequence of SEQ ID NO: 143. In some embodiments, the antibody is EpING1. In some embodiments, the antibody comprises CDR-H1, CDR-H2, CDR-H3, CDR-L1, CDR-L2, and CDR-L3 comprising the amino acid sequences of SEQ ID NOs: 144, 145, 146, 147, 148, and 149, respectively. In some embodiments, the antibody comprises a heavy chain variable region comprising the amino acid sequence of SEQ ID NO: 150 and a light chain variable region comprising the amino acid sequence of SEQ ID NO: 151. In some embodiments, the antibody is EpAb2-6.

In some embodiments, an antibody provided herein binds to CD352. In some embodiments, the antibody comprises CDR-H1, CDR-H2, CDR-H3, CDR-L1, CDR-L2, and CDR-L3 comprising the amino acid sequences of SEQ ID NOs: 152, 153, 154, 155, 156, and 157, respectively. In some embodiments, the antibody comprises a heavy chain variable region comprising the amino acid sequence of SEQ ID NO: 158 and a light chain variable region comprising the amino acid sequence of SEQ ID NO: 159. In some embodiments, the antibody is h20F3.

In some embodiments, an antibody provided herein binds to CS1. In some embodiments, the antibody comprises CDR-H1, CDR-H2, CDR-H3, CDR-L1, CDR-L2, and CDR-L3 comprising the amino acid sequences of SEQ ID NOs: 160, 161, 162, 163, 164, and 165, respectively. In some embodiments, the antibody comprises a heavy chain variable region comprising the amino acid sequence of SEQ ID NO: 166 and a light chain variable region comprising the amino acid sequence of SEQ ID NO: 167. In some embodiments, the antibody is elotuzumab.

In some embodiments, an antibody provided herein binds to CD38. In some embodiments, the antibody comprises CDR-H1, CDR-H2, CDR-H3, CDR-L1, CDR-L2, and CDR-L3 comprising the amino acid sequences of SEQ ID NOs: 168, 169, 170, 171, 172, and 173, respectively. In some embodiments, the antibody comprises a heavy chain variable region comprising the amino acid sequence of SEQ ID NO: 174 and a light chain variable region comprising the amino acid sequence of SEQ ID NO: 175. In some embodiments, the antibody is daratumumab.

In some embodiments, an antibody provided herein binds to CD25. In some embodiments, the antibody comprises CDR-H1, CDR-H2, CDR-H3, CDR-L1, CDR-L2, and CDR-L3 comprising the amino acid sequences of SEQ ID NOs: 176, 177, 178, 179, 180, and 181, respectively. In some embodiments, the antibody comprises a heavy chain variable region comprising the amino acid sequence of SEQ ID NO: 182 and a light chain variable region comprising the amino acid sequence of SEQ ID NO: 183. In some embodiments, the antibody is daclizumab.

In some embodiments, an antibody provided herein binds to ADAM9. In some embodiments, the antibody comprises CDR-H1, CDR-H2, CDR-H3, CDR-L1, CDR-L2, and CDR-L3 comprising the amino acid sequences of SEQ ID NOs: 184, 185, 186, 187, 188, and 189, respectively. In some embodiments, the antibody comprises a heavy chain variable region comprising the amino acid sequence of SEQ ID NO: 190 and a light chain variable region comprising the amino acid sequence of SEQ ID NO: 191. In some embodiments, the antibody is chMAbA9-A. In some embodiments, the antibody comprises CDR-H1, CDR-H2, CDR-H3, CDR-L1, CDR-L2, and CDR-L3 comprising the amino acid sequences of SEQ ID NOs: 192, 193, 194, 195, 196, and 197, respectively. In some embodiments, the antibody comprises a heavy chain variable region comprising the amino acid sequence of SEQ ID NO: 198 and a light chain variable region comprising the amino acid sequence of SEQ ID NO: 199. In some embodiments, the antibody is hMAbA9-A.

In some embodiments, an antibody provided herein binds to CD59. In some embodiments, the antibody comprises CDR-H1, CDR-H2, CDR-H3, CDR-L1, CDR-L2, and CDR-L3 comprising the amino acid sequences of SEQ ID NOs: 200, 201, 202, 203, 204, and 205, respectively. In some embodiments, the antibody comprises a heavy chain variable region comprising the amino acid sequence of SEQ ID NO: 206 and a light chain variable region comprising the amino acid sequence of SEQ ID NO: 207.

In some embodiments, an antibody provided herein binds to CD25. In some embodiments, the antibody is Clone123.

In some embodiments, an antibody provided herein binds to CD229. In some embodiments, the antibody is h8A10.

In some embodiments, an antibody provided herein binds to CD19. In some embodiments, the antibody comprises CDR-H1, CDR-H2, CDR-H3, CDR-L1, CDR-L2, and CDR-L3 comprising the amino acid sequences of SEQ ID NOs: 208, 209, 210, 211, 212, and 213, respectively. In some embodiments, the antibody comprises a heavy chain variable region comprising the amino acid sequence of SEQ ID NO: 214 and a light chain variable region comprising the amino acid sequence of SEQ ID NO: 215. In some embodiments, the antibody is denintuzumab, which is also known as hBU12. See WO2009052431.

In some embodiments, an antibody provided herein binds to CD70. In some embodiments, the antibody comprises CDR-H1, CDR-H2, CDR-H3, CDR-L1, CDR-L2, and CDR-L3 comprising the amino acid sequences of SEQ ID NOs: 216, 217, 218, 219, 220, and 221, respectively. In some embodiments, the antibody comprises a heavy chain variable region comprising the amino acid sequence of SEQ ID NO: 222 and a light chain variable region comprising the amino acid sequence of SEQ ID NO: 223. In some embodiments, the antibody is vorsetuzumab.

In some embodiments, an antibody provided herein binds to B7H4. In some embodiments, the antibody comprises CDR-H1, CDR-H2, CDR-H3, CDR-L1, CDR-L2, and CDR-L3 comprising the amino acid sequences of SEQ ID NOs: 224, 225, 226, 227, 228, and 229, respectively. In some embodiments, the antibody comprises a heavy chain variable region comprising the amino acid sequence of SEQ ID NO: 230 and a light chain variable region comprising the amino acid sequence of SEQ ID NO: 231. In some embodiments, the antibody is mirzotamab.

In some embodiments, an antibody provided herein binds to CD138. In some embodiments, the antibody comprises CDR-H1, CDR-H2, CDR-H3, CDR-L1, CDR-L2, and CDR-L3 comprising the amino acid sequences of SEQ ID NOs: 232, 233, 234, 235, 236, and 237, respectively. In some embodiments, the antibody comprises a heavy chain variable region comprising the amino acid sequence of SEQ ID NO: 238 and a light chain variable region comprising the amino acid sequence of SEQ ID NO: 239. In some embodiments, the antibody is indatuxumab.

In some embodiments, an antibody provided herein binds to CD166. In some embodiments, the antibody comprises CDR-H1, CDR-H2, CDR-H3, CDR-L1, CDR-L2, and CDR-L3 comprising the amino acid sequences of SEQ ID NOs: 240, 241, 242, 243, 244, and 245, respectively. In some embodiments, the antibody comprises a heavy chain variable region comprising the amino acid sequence of SEQ ID NO: 246 and a light chain variable region comprising the amino acid sequence of SEQ ID NO: 247. In some embodiments, the antibody is praluzatamab.

In some embodiments, an antibody provided herein binds to CD51. In some embodiments, the antibody comprises CDR-H1, CDR-H2, CDR-H3, CDR-L1, CDR-L2, and CDR-L3 comprising the amino acid sequences of SEQ ID NOs: 248, 249, 250, 251, 252, and 253, respectively. In some embodiments, the antibody comprises a heavy chain variable region comprising the amino acid sequence of SEQ ID NO: 254 and a light chain variable region comprising the amino acid sequence of SEQ ID NO: 255. In some embodiments, the antibody is intetumumab.

In some embodiments, an antibody provided herein binds to CD56. In some embodiments, the antibody comprises CDR-H1, CDR-H2, CDR-H3, CDR-L1, CDR-L2, and CDR-L3 comprising the amino acid sequences of SEQ ID NOs: 256, 257, 258, 259, 260, and 261, respectively. In some embodiments, the antibody comprises a heavy chain variable region comprising the amino acid sequence of SEQ ID NO: 262 and a light chain variable region comprising the amino acid sequence of SEQ ID NO: 263. In some embodiments, the antibody is lorvotuzumab.

In some embodiments, an antibody provided herein binds to CD74. In some embodiments, the antibody comprises CDR-H1, CDR-H2, CDR-H3, CDR-L1, CDR-L2, and CDR-L3 comprising the amino acid sequences of SEQ ID NOs: 264, 265, 266, 267, 268, and 269, respectively. In some embodiments, the antibody comprises a heavy chain variable region comprising the amino acid sequence of SEQ ID NO: 270 and a light chain variable region comprising the amino acid sequence of SEQ ID NO: 271. In some embodiments, the antibody is milatuzumab.

In some embodiments, an antibody provided herein binds to CEACAM5. In some embodiments, the antibody comprises CDR-H1, CDR-H2, CDR-H3, CDR-L1, CDR-L2, and CDR-L3 comprising the amino acid sequences of SEQ ID NOs: 272, 273 274, 275, 276, and 277, respectively. In some embodiments, the antibody comprises a heavy chain variable region comprising the amino acid sequence of SEQ ID NO: 278 and a light chain variable region comprising the amino acid sequence of SEQ ID NO: 279. In some embodiments, the antibody is labetuzumab.

In some embodiments, an antibody provided herein binds to CanAg. In some embodiments, the antibody comprises CDR-H1, CDR-H2, CDR-H3, CDR-L1, CDR-L2, and CDR-L3 comprising the amino acid sequences of SEQ ID NOs: 280, 281, 282, 283, 284, and 285, respectively. In some embodiments, the antibody comprises a heavy chain variable region comprising the amino acid sequence of SEQ ID NO: 286 and a light chain variable region comprising the amino acid sequence of SEQ ID NO: 287. In some embodiments, the antibody is cantuzumab.

In some embodiments, an antibody provided herein binds to DLL-3. In some embodiments, the antibody comprises CDR-H1, CDR-H2, CDR-H3, CDR-L1, CDR-L2, and CDR-L3 comprising the amino acid sequences of SEQ ID NOs: 288, 289, 290, 291, 292, and 293, respectively. In some embodiments, the antibody comprises a heavy chain variable region comprising the amino acid sequence of SEQ ID NO: 294 and a light chain variable region comprising the amino acid sequence of SEQ ID NO: 295. In some embodiments, the antibody is rovalpituzumab.

In some embodiments, an antibody provided herein binds to DPEP-3. In some embodiments, the antibody comprises CDR-H1, CDR-H2, CDR-H3, CDR-L1, CDR-L2, and CDR-L3 comprising the amino acid sequences of SEQ ID NOs: 296, 297, 298, 299, 300, and 301, respectively. In some embodiments, the antibody comprises a heavy chain variable region comprising the amino acid sequence of SEQ ID NO: 302 and a light chain variable region comprising the amino acid sequence of SEQ ID NO: 303. In some embodiments, the antibody is tamrintamab.

In some embodiments, an antibody provided herein binds to EGFR. In some embodiments, the antibody comprises CDR-H1, CDR-H2, CDR-H3, CDR-L1, CDR-L2, and CDR-L3 comprising the amino acid sequences of SEQ ID NOs: 304, 305, 306, 307, 308, and 309, respectively. In some embodiments, the antibody comprises a heavy chain variable region comprising the amino acid sequence of SEQ ID NO: 310 and a light chain variable region comprising the amino acid sequence of SEQ ID NO: 311. In some embodiments, the antibody is laprituximab. In some embodiments, the antibody comprises CDR-H1, CDR-H2, CDR-H3, CDR-L1, CDR-L2, and CDR-L3 comprising the amino acid sequences of SEQ ID NOs: 312, 313, 314, 315, 316, and 317, respectively. In some embodiments, the antibody comprises a heavy chain variable region comprising the amino acid sequence of SEQ ID NO: 318 and a light chain variable region comprising the amino acid sequence of SEQ ID NO: 319. In some embodiments, the antibody is losatuxizumab. In some embodiments, the antibody comprises CDR-H1, CDR-H2, CDR-H3, CDR-L1, CDR-L2, and CDR-L3 comprising the amino acid sequences of SEQ ID NOs: 320, 321, 322, 323, 324, and 325, respectively. In some embodiments, the antibody comprises a heavy chain variable region comprising the amino acid sequence of SEQ ID NO: 326 and a light chain variable region comprising the amino acid sequence of SEQ ID NO: 327. In some embodiments, the antibody is serclutamab. In some embodiments, the antibody comprises CDR-H1, CDR-H2, CDR-H3, CDR-L1, CDR-L2, and CDR-L3 comprising the amino acid sequences of SEQ ID NOs: 328, 329, 330, 331, 332, and 333, respectively. In some embodiments, the antibody comprises a heavy chain variable region comprising the amino acid sequence of SEQ ID NO: 334 and a light chain variable region comprising the amino acid sequence of SEQ ID NO: 335. In some embodiments, the antibody is cetuximab.

In some embodiments, an antibody provided herein binds to FRa. In some embodiments, the antibody comprises CDR-H1, CDR-H2, CDR-H3, CDR-L1, CDR-L2, and CDR-L3 comprising the amino acid sequences of SEQ ID NOs: 336, 337, 338, 339, 340, and 341, respectively. In some embodiments, the antibody comprises a heavy chain variable region comprising the amino acid sequence of SEQ ID NO: 342 and a light chain variable region comprising the amino acid sequence of SEQ ID NO: 343. In some embodiments, the antibody is mirvetuximab. In some embodiments, the antibody comprises CDR-H1, CDR-H2, CDR-H3, CDR-L1, CDR-L2, and CDR-L3 comprising the amino acid sequences of SEQ ID NOs: 344, 345, 346, 347, 348, and 349, respectively. In some embodiments, the antibody comprises a heavy chain variable region comprising the amino acid sequence of SEQ ID NO: 350 and a light chain variable region comprising the amino acid sequence of SEQ ID NO: 351. In some embodiments, the antibody is farletuzumab.

In some embodiments, an antibody provided herein binds to MUC-1. In some embodiments, the antibody comprises CDR-H1, CDR-H2, CDR-H3, CDR-L1, CDR-L2, and CDR-L3 comprising the amino acid sequences of SEQ ID NOs: 352, 353, 354, 355, 356, and 357, respectively. In some embodiments, the antibody comprises a heavy chain variable region comprising the amino acid sequence of SEQ ID NO: 358 and a light chain variable region comprising the amino acid sequence of SEQ ID NO: 359. In some embodiments, the antibody is gatipotuzumab.

In some embodiments, an antibody provided herein binds to mesothelin. In some embodiments, the antibody comprises CDR-H1, CDR-H2, CDR-H3, CDR-L1, CDR-L2, and CDR-L3 comprising the amino acid sequences of SEQ ID NOs: 360, 361, 362, 363, 364, and 365, respectively. In some embodiments, the antibody comprises a heavy chain variable region comprising the amino acid sequence of SEQ ID NO: 366 and a light chain variable region comprising the amino acid sequence of SEQ ID NO: 367. In some embodiments, the antibody is anetumab.

In some embodiments, an antibody provided herein binds to ROR-1. In some embodiments, the antibody comprises CDR-H1, CDR-H2, CDR-H3, CDR-L1, CDR-L2, and CDR-L3 comprising the amino acid sequences of SEQ ID NOs: 368, 369, 370, 371, 372, and 373, respectively. In some embodiments, the antibody comprises a heavy chain variable region comprising the amino acid sequence of SEQ ID NO: 374 and a light chain variable region comprising the amino acid sequence of SEQ ID NO: 375. In some embodiments, the antibody is zilovertamab.

In some embodiments, an antibody provided herein binds to ASCT2. In some embodiments, an antibody provided herein binds to B7H4. In some embodiments, the antibody comprises CDR-H1, CDR-H2, CDR-H3, CDR-L1, CDR-L2, and CDR-L3 comprising the amino acid sequences of SEQ ID NOs: 376, 377, 378, 379, 380, and 381, respectively. In some embodiments, the antibody comprises a heavy chain variable region comprising the amino acid sequence of SEQ ID NO: 382 and a light chain variable region comprising the amino acid sequence of SEQ ID NO: 383. In some embodiments, the antibody is 20502. See WO2019040780.

In some embodiments, an antibody provided herein binds to B7-H3. In some embodiments, the antibody comprises CDR-H1, CDR-H2, CDR-H3, CDR-L1, CDR-L2, and CDR-L3 comprising the amino acid sequences of SEQ ID NOs: 384, 385, 386, 387, 388, and 389, respectively. In some embodiments, the antibody comprises a heavy chain variable region comprising the amino acid sequence of SEQ ID NO: 390 and a light chain variable region comprising the amino acid sequence of SEQ ID NO: 391. In some embodiments, the antibody is chAb-A (BRCA84D). In some embodiments, the antibody comprises CDR-H1, CDR-H2, CDR-H3, CDR-L1, CDR-L2, and CDR-L3 comprising the amino acid sequences of SEQ ID NOs: 392, 393, 394, 395, 396, and 397, respectively. In some embodiments, the antibody comprises a heavy chain variable region comprising the amino acid sequence of SEQ ID NO: 398 and a light chain variable region comprising the amino acid sequence of SEQ ID NO: 399. In some embodiments, the antibody is hAb-B. In some embodiments, the antibody comprises CDR-H1, CDR-H2, CDR-H3, CDR-L1, CDR-L2, and CDR-L3 comprising the amino acid sequences of SEQ ID NOs: 400, 401, 402, 403, 404, and 405, respectively. In some embodiments, the antibody comprises a heavy chain variable region comprising the amino acid sequence of SEQ ID NO: 406 and a light chain variable region comprising the amino acid sequence of SEQ ID NO: 407. In some embodiments, the antibody is hAb-C. In some embodiments, the antibody comprises CDR-H1, CDR-H2, CDR-H3, CDR-L1, CDR-L2, and CDR-L3 comprising the amino acid sequences of SEQ ID NOs: 408, 409, 410, 411, 412, and 413, respectively. In some embodiments, the antibody comprises a heavy chain variable region comprising the amino acid sequence of SEQ ID NO: 414 and a light chain variable region comprising the amino acid sequence of SEQ ID NO: 415. In some embodiments, the antibody is hAb-D. In some embodiments, the antibody comprises CDR-H1, CDR-H2, CDR-H3, CDR-L1, CDR-L2, and CDR-L3 comprising the amino acid sequences of SEQ ID NOs: 416, 417, 418, 419, 420, and 421, respectively. In some embodiments, the antibody comprises a heavy chain variable region comprising the amino acid sequence of SEQ ID NO: 422 and a light chain variable region comprising the amino acid sequence of SEQ ID NO: 423. In some embodiments, the antibody is chM30. In some embodiments, the antibody comprises CDR-H1, CDR-H2, CDR-H3, CDR-L1, CDR-L2, and CDR-L3 comprising the amino acid sequences of SEQ ID NOs: 424, 425, 426, 427, 428, and 429, respectively. In some embodiments, the antibody comprises a heavy chain variable region comprising the amino acid sequence of SEQ ID NO: 430 and a light chain variable region comprising the amino acid sequence of SEQ ID NO: 431. In some embodiments, the antibody is hM30-H1-L4. In some embodiments, the antibody comprises CDR-H1, CDR-H2, CDR-H3, CDR-L1, CDR-L2, and CDR-L3 comprising the amino acid sequences of SEQ ID NOs: 432, 433, 434, 435, 436, and 437, respectively. In some embodiments, the antibody comprises a heavy chain variable region comprising the amino acid sequence of SEQ ID NO: 438 and a light chain variable region comprising the amino acid sequence of SEQ ID NO: 439. In some embodiments, the antibody is AbV_huAb18-v4. In some embodiments, the antibody comprises CDR-H1, CDR-H2, CDR-H3, CDR-L1, CDR-L2, and CDR-L3 comprising the amino acid sequences of SEQ ID NOs: 440, 441, 442, 443, 444, and 445, respectively. In some embodiments, the antibody comprises a heavy chain variable region comprising the amino acid sequence of SEQ ID NO: 446 and a light chain variable region comprising the amino acid sequence of SEQ ID NO: 447. In some embodiments, the antibody is AbV_huAb3-v6. In some embodiments, the antibody comprises CDR-H1, CDR-H2, CDR-H3, CDR-L1, CDR-L2, and CDR-L3 comprising the amino acid sequences of SEQ ID NOs: 448, 449, 450, 451, 452, and 453, respectively. In some embodiments, the antibody comprises a heavy chain variable region comprising the amino acid sequence of SEQ ID NO: 454 and a light chain variable region comprising the amino acid sequence of SEQ ID NO: 455. In some embodiments, the antibody is AbV_huAb3-v2.6. In some embodiments, the antibody comprises CDR-H1, CDR-H2, CDR-H3, CDR-L1, CDR-L2, and CDR-L3 comprising the amino acid sequences of SEQ ID NOs: 456, 457, 458, 459, 460, and 461, respectively. In some embodiments, the antibody comprises a heavy chain variable region comprising the amino acid sequence of SEQ ID NO: 462 and a light chain variable region comprising the amino acid sequence of SEQ ID NO: 463. In some embodiments, the antibody is AbV_huAb13-v1-CR. In some embodiments, the antibody comprises CDR-H1, CDR-H2, CDR-H3, CDR-L1, CDR-L2, and CDR-L3 comprising the amino acid sequences of SEQ ID NOs: 464, 465, 466, 467, 468, and 469, respectively. In some embodiments, the antibody comprises a heavy chain variable region comprising the amino acid sequence of SEQ ID NO: 470 and a light chain variable region comprising the amino acid sequence of SEQ ID NO: 471. In some embodiments, the antibody is 8H9-6m. In some embodiments, the antibody comprises a heavy chain variable region comprising the amino acid sequence of SEQ ID NO: 472 and a light chain variable region comprising the amino acid sequence of SEQ ID NO: 473. In some embodiments, the antibody is m8517. In some embodiments, the antibody comprises CDR-H1, CDR-H2, CDR-H3, CDR-L1, CDR-L2, and CDR-L3 comprising the amino acid sequences of SEQ ID NOs: 474, 475, 476, 477, 478, and 479, respectively. In some embodiments, the antibody comprises a heavy chain variable region comprising the amino acid sequence of SEQ ID NO: 480 and a light chain variable region comprising the amino acid sequence of SEQ ID NO: 481. In some embodiments, the antibody is TPP-5706. In some embodiments, the antibody comprises a heavy chain variable region comprising the amino acid sequence of SEQ ID NO: 482 and a light chain variable region comprising the amino acid sequence of SEQ ID NO: 483. In some embodiments, the antibody is TPP-6642. In some embodiments, the antibody comprises a heavy chain variable region comprising the amino acid sequence of SEQ ID NO: 484 and a light chain variable region comprising the amino acid sequence of SEQ ID NO: 485. In some embodiments, the antibody is TPP-6850.

In some embodiments, an antibody provided herein binds to CDCP1. In some embodiments, the antibody is 10D7.

In some embodiments, an antibody provided herein binds to HER3. In some embodiments, the antibody comprises a heavy chain comprising the amino acid sequence of SEQ ID NO: 486 and a light chain comprising the amino acid sequence of SEQ ID NO: 487. In some embodiments, the antibody is patritumab. In some embodiments, the antibody comprises a heavy chain comprising the amino acid sequence of SEQ ID NO: 488 and a light chain comprising the amino acid sequence of SEQ ID NO: 489. In some embodiments, the antibody is seribantumab. In some embodiments, the antibody comprises a heavy chain comprising the amino acid sequence of SEQ ID NO: 490 and a light chain comprising the amino acid sequence of SEQ ID NO: 491. In some embodiments, the antibody is elgemtumab. In some embodiments, the antibody comprises a heavy chain the amino acid sequence of SEQ ID NO: 492 and a light chain comprising the amino acid sequence of SEQ ID NO: 493. In some embodiments, the antibody is lumretuzumab.

In some embodiments, an antibody provided herein binds to RON. In some embodiments, the antibody is Zt/g4.

In some embodiments, an antibody provided herein binds to claudin-2.

In some embodiments, an antibody provided herein binds to HLA-G.

In some embodiments, an antibody provided herein binds to PTK7. In some embodiments, the antibody comprises CDR-H1, CDR-H2, CDR-H3, CDR-L1, CDR-L2, and CDR-L3 comprising the amino acid sequences of SEQ ID NOs: 494, 495, 496, 497, 498, and 499, respectively. In some embodiments, the antibody comprises a heavy chain variable region comprising the amino acid sequence of SEQ ID NO: 500 and a light chain variable region comprising the amino acid sequence of SEQ ID NO: 501. In some embodiments, the antibody is PTK7 mab 1. In some embodiments, the antibody comprises CDR-H1, CDR-H2, CDR-H3, CDR-L1, CDR-L2, and CDR-L3 comprising the amino acid sequences of SEQ ID NOs: 502, 503, 504, 505, 506, and 507, respectively. In some embodiments, the antibody comprises a heavy chain variable region comprising the amino acid sequence of SEQ ID NO: 508 and a light chain variable region comprising the amino acid sequence of SEQ ID NO: 509. In some embodiments, the antibody is PTK7 mab 2. In some embodiments, the antibody comprises CDR-H1, CDR-H2, CDR-H3, CDR-L1, CDR-L2, and CDR-L3 comprising the amino acid sequences of SEQ ID NOs: 510, 511, 512, 513, 514, and 515, respectively. In some embodiments, the antibody comprises a heavy chain variable region comprising the amino acid sequence of SEQ ID NO: 516 and a light chain variable region comprising the amino acid sequence of SEQ ID NO: 517. In some embodiments, the antibody is PTK7 mab 3.

In some embodiments, an antibody provided herein binds to LIV1. In some embodiments, the antibody comprises CDR-H1, CDR-H2, CDR-H3, CDR-L1, CDR-L2, and CDR-L3 comprising the amino acid sequences of SEQ ID NOs: 518, 519, 520, 521, 522, and 523, respectively. In some embodiments, the antibody comprises a heavy chain variable region comprising the amino acid sequence of SEQ ID NO: 524 and a light chain variable region comprising the amino acid sequence of SEQ ID NO: 525. In some embodiments, the antibody is ladiratuzumab, which is also known as hLIV22 and hglg. See WO2012078668.

In some embodiments, an antibody provided herein binds to avb6. In some embodiments, the antibody comprises CDR-H1, CDR-H2, CDR-H3, CDR-L1, CDR-L2, and CDR-L3 comprising the amino acid sequences of SEQ ID NOs: 526, 527, 528, 529, 530, and 531, respectively. In some embodiments, the antibody comprises a heavy chain variable region comprising the amino acid sequence of SEQ ID NO: 532 and a light chain variable region comprising the amino acid sequence of SEQ ID NO: 533. In some embodiments, the antibody is h2A2. In some embodiments, the antibody comprises CDR-H1, CDR-H2, CDR-H3, CDR-L1, CDR-L2, and CDR-L3 comprising the amino acid sequences of SEQ ID NOs: 534, 535, 536, 537, 538, and 539, respectively. In some embodiments, the antibody comprises a heavy chain variable region comprising the amino acid sequence of SEQ ID NO: 540 and a light chain variable region comprising the amino acid sequence of SEQ ID NO: 541. In some embodiments, the antibody is h15H3.

In some embodiments, an antibody provided herein binds to CD48. In some embodiments, the antibody comprises CDR-H1, CDR-H2, CDR-H3, CDR-L1, CDR-L2, and CDR-L3 comprising the amino acid sequences of SEQ ID NOs: 542, 543, 544, 545, 546, and 547, respectively. In some embodiments, the antibody comprises a heavy chain variable region comprising the amino acid sequence of SEQ ID NO: 548 and a light chain variable region comprising the amino acid sequence of SEQ ID NO: 549. In some embodiments, the antibody is hMEM102. See WO2016149535.

In some embodiments, an antibody provided herein binds to PD-L1. In some embodiments, the antibody comprises CDR-H1, CDR-H2, CDR-H3, CDR-L1, CDR-L2, and CDR-L3 comprising the amino acid sequences of SEQ ID NOs: 550, 551, 552, 553, 554, and 555, respectively. In some embodiments, the antibody comprises a heavy chain variable region comprising the amino acid sequence of SEQ ID NO: 556 and a light chain variable region comprising the amino acid sequence of SEQ ID NO: 557. In some embodiments, the antibody is SG-559-01 LALA mAb.

In some embodiments, an antibody provided herein binds to IGF-1R. In some embodiments, the antibody comprises CDR-H1, CDR-H2, CDR-H3, CDR-L1, CDR-L2, and CDR-L3 comprising the amino acid sequences of SEQ ID NOs: 558, 559, 560, 561, 562, and 563, respectively. In some embodiments, the antibody comprises a heavy chain variable region comprising the amino acid sequence of SEQ ID NO: 564 and a light chain variable region comprising the amino acid sequence of SEQ ID NO: 565. In some embodiments, the antibody is cixutumumab.

In some embodiments, an antibody provided herein binds to claudin-18.2. In some embodiments, the antibody comprises CDR-H1, CDR-H2, CDR-H3, CDR-L1, CDR-L2, and CDR-L3 comprising the amino acid sequences of SEQ ID NOs: 566, 567, 568, 569, 570, and 571, respectively. In some embodiments, the antibody comprises a heavy chain variable region comprising the amino acid sequence of SEQ ID NO: 572 and a light chain variable region comprising the amino acid sequence of SEQ ID NO: 573. In some embodiments, the antibody is zolbetuximab (175D10). In some embodiments, the antibody comprises CDR-H1, CDR-H2, CDR-H3, CDR-L1, CDR-L2, and CDR-L3 comprising the amino acid sequences of SEQ ID NOs: 574, 575, 576, 577, 578, and 579, respectively. In some embodiments, the antibody comprises a heavy chain variable region comprising the amino acid sequence of SEQ ID NO: 580 and a light chain variable region comprising the amino acid sequence of SEQ ID NO: 581. In some embodiments, the antibody is 163E12.

In some embodiments, an antibody provided herein binds to Nectin-4. In some embodiments, the antibody comprises CDR-H1, CDR-H2, CDR-H3, CDR-L1, CDR-L2, and CDR-L3 comprising the amino acid sequences of SEQ ID NOs: 582, 583, 584, 585, 586, and 587, respectively. In some embodiments, the antibody comprises a heavy chain variable region comprising the amino acid sequence of SEQ ID NO: 588 and a light chain variable region comprising the amino acid sequence of SEQ ID NO: 589. In some embodiments, the antibody is enfortumab. See WO 2012047724.

In some embodiments, an antibody provided herein binds to SLTRK6. In some embodiments, the antibody comprises CDR-H1, CDR-H2, CDR-H3, CDR-L1, CDR-L2, and CDR-L3 comprising the amino acid sequences of SEQ ID NOs: 590, 591, 592, 593, 594, and 595, respectively. In some embodiments, the antibody comprises a heavy chain variable region comprising the amino acid sequence of SEQ ID NO: 596 and a light chain variable region comprising the amino acid sequence of SEQ ID NO: 597. In some embodiments, the antibody is sirtratumab.

In some embodiments, an antibody provided herein binds to CD228. In some embodiments, the antibody comprises CDR-H1, CDR-H2, CDR-H3, CDR-L1, CDR-L2, and CDR-L3 comprising the amino acid sequences of SEQ ID NOs: 598, 599, 600, 601, 602, and 603, respectively. In some embodiments, the antibody comprises a heavy chain variable region comprising the amino acid sequence of SEQ ID NO: 604 and a light chain variable region comprising the amino acid sequence of SEQ ID NO: 605. In some embodiments, the antibody is hL49. See WO 2020/163225.

In some embodiments, an antibody provided herein binds to CD142 (tissue factor; TF). In some embodiments, the antibody comprises CDR-H1, CDR-H2, CDR-H3, CDR-L1, CDR-L2, and CDR-L3 comprising the amino acid sequences of SEQ ID NOs: 606, 607, 608, 609, 610, and 611, respectively. In some embodiments, the antibody comprises a heavy chain variable region comprising the amino acid sequence of SEQ ID NO: 612 and a light chain variable region comprising the amino acid sequence of SEQ ID NO: 613. In some embodiments, the antibody is tisotumab. See WO 2010/066803.

In some embodiments, an antibody provided herein binds to STn. In some embodiments, the antibody comprises CDR-H1, CDR-H2, CDR-H3, CDR-L1, CDR-L2, and CDR-L3 comprising the amino acid sequences of SEQ ID NOs: 614, 615, 616, 617, 618, and 619, respectively. In some embodiments, the antibody comprises a heavy chain variable region comprising the amino acid sequence of SEQ ID NO: 620 and a light chain variable region comprising the amino acid sequence of SEQ ID NO: 621. In some embodiments, the antibody is h2G12.

In some embodiments, an antibody provided herein binds to CD20. In some embodiments, the antibody comprises CDR-H1, CDR-H2, CDR-H3, CDR-L1, CDR-L2, and CDR-L3 comprising the amino acid sequences of SEQ ID NOs: 622, 623, 624, 625, 626, and 627, respectively. In some embodiments, the antibody comprises a heavy chain variable region comprising the amino acid sequence of SEQ ID NO: 628 and a light chain variable region comprising the amino acid sequence of SEQ ID NO: 629. In some embodiments, the antibody is rituximab.

In some embodiments, an antibody provided herein binds to HER2. In some embodiments, the antibody comprises CDR-H1, CDR-H2, CDR-H3, CDR-L1, CDR-L2, and CDR-L3 comprising the amino acid sequences of SEQ ID NOs: 630, 631, 632, 633, 634, and 635, respectively. In some embodiments, the antibody comprises a heavy chain variable region comprising the amino acid sequence of SEQ ID NO: 636 and a light chain variable region comprising the amino acid sequence of SEQ ID NO: 637. In some embodiments, the antibody is trastuzumab.

In some embodiments, an antibody provided herein binds to FLT3.

In some embodiments, an antibody provided herein binds to CD46.

In some embodiments, an antibody provided herein binds to GloboH.

In some embodiments, an antibody provided herein binds to AG7.

In some embodiments, an antibody provided herein binds to mesothelin.

In some embodiments, an antibody provided herein binds to FCRH5.

In some embodiments, an antibody provided herein binds to ETBR.

In some embodiments, an antibody provided herein binds to Tim-1.

In some embodiments, an antibody provided herein binds to SLC44A4.

In some embodiments, an antibody provided herein binds to ENPP3.

In some embodiments, an antibody provided herein binds to CD37.

In some embodiments, an antibody provided herein binds to CA9.

In some embodiments, an antibody provided herein binds to Notch3.

In some embodiments, an antibody provided herein binds to EphA2.

In some embodiments, an antibody provided herein binds to TRFC.

In some embodiments, an antibody provided herein binds to PSMA.

In some embodiments, an antibody provided herein binds to LRRC15.

In some embodiments, an antibody provided herein binds to 5T4.

In some embodiments, an antibody provided herein binds to CD79b. In some embodiments, the antibody comprises CDR-H1, CDR-H2, CDR-H3, CDR-L1, CDR-L2, and CDR-L3 comprising the amino acid sequences of SEQ ID NOs: 638, 639, 640, 641, 642, and 643, respectively. In some embodiments, the antibody comprises a heavy chain variable region comprising the amino acid sequence of SEQ ID NO: 644 and a light chain variable region comprising the amino acid sequence of SEQ ID NO: 645. In some embodiments, the antibody is polatuzumab.

In some embodiments, an antibody provided herein binds to NaPi2B. In some embodiments, the antibody comprises CDR-H1, CDR-H2, CDR-H3, CDR-L1, CDR-L2, and CDR-L3 comprising the amino acid sequences of SEQ ID NOs: 646, 647, 648, 649, 650, and 651, respectively. In some embodiments, the antibody comprises a heavy chain variable region comprising the amino acid sequence of SEQ ID NO: 652 and a light chain variable region comprising the amino acid sequence of SEQ ID NO: 653. In some embodiments, the antibody is lifastuzumab.

In some embodiments, an antibody provided herein binds to Muc16. In some embodiments, the antibody comprises CDR-H1, CDR-H2, CDR-H3, CDR-L1, CDR-L2, and CDR-L3 comprising the amino acid sequences of SEQ ID NOs: 654, 655, 656, 657, 658, and 659, respectively. In some embodiments, the antibody comprises a heavy chain variable region comprising the amino acid sequence of SEQ ID NO: 660 and a light chain variable region comprising the amino acid sequence of SEQ ID NO: 661. In some embodiments, the antibody is sofituzumab.

In some embodiments, an antibody provided herein binds to STEAPL. In some embodiments, the antibody comprises CDR-H1, CDR-H2, CDR-H3, CDR-L1, CDR-L2, and CDR-L3 comprising the amino acid sequences of SEQ ID NOs: 662, 663, 664, 665, 666, and 667, respectively. In some embodiments, the antibody comprises a heavy chain variable region comprising the amino acid sequence of SEQ ID NO: 668 and a light chain variable region comprising the amino acid sequence of SEQ ID NO: 669. In some embodiments, the antibody is vandortuzumab.

In some embodiments, an antibody provided herein binds to BCMA. In some embodiments, the antibody comprises CDR-H1, CDR-H2, CDR-H3, CDR-L1, CDR-L2, and CDR-L3 comprising the amino acid sequences of SEQ ID NOs: 670, 671, 672, 673, 674, and 675, respectively. In some embodiments, the antibody comprises a heavy chain variable region comprising the amino acid sequence of SEQ ID NO: 676 and a light chain variable region comprising the amino acid sequence of SEQ ID NO: 677. In some embodiments, the antibody is belantamab.

In some embodiments, an antibody provided herein binds to c-Met. In some embodiments, the antibody comprises CDR-H1, CDR-H2, CDR-H3, CDR-L1, CDR-L2, and CDR-L3 comprising the amino acid sequences of SEQ ID NOs: 678, 679, 680, 681, 682, and 683, respectively. In some embodiments, the antibody comprises a heavy chain variable region comprising the amino acid sequence of SEQ ID NO: 684 and a light chain variable region comprising the amino acid sequence of SEQ ID NO: 685. In some embodiments, the antibody is telisotuzumab.

In some embodiments, an antibody provided herein binds to EGFR. In some embodiments, the antibody comprises CDR-H1, CDR-H2, CDR-H3, CDR-L1, CDR-L2, and CDR-L3 comprising the amino acid sequences of SEQ ID NOs: 686, 687, 688, 689, 690, and 691, respectively. In some embodiments, the antibody comprises a heavy chain variable region comprising the amino acid sequence of SEQ ID NO: 692 and a light chain variable region comprising the amino acid sequence of SEQ ID NO: 693. In some embodiments, the antibody is depatuxizumab.

In some embodiments, an antibody provided herein binds to SLAMF7. In some embodiments, the antibody comprises CDR-H1, CDR-H2, CDR-H3, CDR-L1, CDR-L2, and CDR-L3 comprising the amino acid sequences of SEQ ID NOs: 694, 695, 696, 697, 698, and 699, respectively. In some embodiments, the antibody comprises a heavy chain variable region comprising the amino acid sequence of SEQ ID NO: 700 and a light chain variable region comprising the amino acid sequence of SEQ ID NO: 701. In some embodiments, the antibody is azintuxizumab.

In some embodiments, an antibody provided herein binds to SLITRK6. In some embodiments, the antibody comprises CDR-H1, CDR-H2, CDR-H3, CDR-L1, CDR-L2, and CDR-L3 comprising the amino acid sequences of SEQ ID NOs: 702, 703, 704, 705, 706, and 707, respectively. In some embodiments, the antibody comprises a heavy chain variable region comprising the amino acid sequence of SEQ ID NO: 708 and a light chain variable region comprising the amino acid sequence of SEQ ID NO: 709. In some embodiments, the antibody is sirtratumab.

In some embodiments, an antibody provided herein binds to C4.4a. In some embodiments, the antibody comprises CDR-H1, CDR-H2, CDR-H3, CDR-L1, CDR-L2, and CDR-L3 comprising the amino acid sequences of SEQ ID NOs: 710, 711, 712, 713, 714, and 715, respectively. In some embodiments, the antibody comprises a heavy chain variable region comprising the amino acid sequence of SEQ ID NO: 716 and a light chain variable region comprising the amino acid sequence of SEQ ID NO: 717. In some embodiments, the antibody is lupartumab.

In some embodiments, an antibody provided herein binds to GCC. In some embodiments, the antibody comprises CDR-H1, CDR-H2, CDR-H3, CDR-L1, CDR-L2, and CDR-L3 comprising the amino acid sequences of SEQ ID NOs: 718, 719, 720, 721, 722, and 723, respectively. In some embodiments, the antibody comprises a heavy chain variable region comprising the amino acid sequence of SEQ ID NO: 724 and a light chain variable region comprising the amino acid sequence of SEQ ID NO: 725. In some embodiments, the antibody is indusatumab.

In some embodiments, an antibody provided herein binds to Axl. In some embodiments, the antibody comprises CDR-H1, CDR-H2, CDR-H3, CDR-L1, CDR-L2, and CDR-L3 comprising the amino acid sequences of SEQ ID NOs: 726, 727, 728, 729, 730, and 731, respectively. In some embodiments, the antibody comprises a heavy chain variable region comprising the amino acid sequence of SEQ ID NO: 732 and a light chain variable region comprising the amino acid sequence of SEQ ID NO: 733. In some embodiments, the antibody is enapotamab.

In some embodiments, an antibody provided herein binds to gpNMB. In some embodiments, the antibody comprises CDR-H1, CDR-H2, CDR-H3, CDR-L1, CDR-L2, and CDR-L3 comprising the amino acid sequences of SEQ ID NOs: 734, 735, 736, 737, 738, and 739, respectively. In some embodiments, the antibody comprises a heavy chain variable region comprising the amino acid sequence of SEQ ID NO: 740 and a light chain variable region comprising the amino acid sequence of SEQ ID NO: 741. In some embodiments, the antibody is glembatumumab.

In some embodiments, an antibody provided herein binds to Prolactin receptor. In some embodiments, the antibody comprises CDR-H1, CDR-H2, CDR-H3, CDR-L1, CDR-L2, and CDR-L3 comprising the amino acid sequences of SEQ ID NOs: 742, 743, 744, 745, 746, and 747, respectively. In some embodiments, the antibody comprises a heavy chain variable region comprising the amino acid sequence of SEQ ID NO: 748 and a light chain variable region comprising the amino acid sequence of SEQ ID NO: 749. In some embodiments, the antibody is rolinsatamab.

In some embodiments, an antibody provided herein binds to FGFR2. In some embodiments, the antibody comprises CDR-H1, CDR-H2, CDR-H3, CDR-L1, CDR-L2, and CDR-L3 comprising the amino acid sequences of SEQ ID NOs: 750, 751, 752, 753, 754, and 755, respectively. In some embodiments, the antibody comprises a heavy chain variable region comprising the amino acid sequence of SEQ ID NO: 756 and a light chain variable region comprising the amino acid sequence of SEQ ID NO: 757. In some embodiments, the antibody is aprutumab.

In some embodiments, an antibody provided herein binds to CDCP1. In some embodiments, the antibody comprises CDR-H1, CDR-H2, CDR-H3, CDR-L1, CDR-L2, and CDR-L3 comprising the amino acid sequences of SEQ ID NOs: 758, 759, 760, 761, 762, and 763, respectively. In some embodiments, the antibody comprises a heavy chain variable region comprising the amino acid sequence of SEQ ID NO: 764 and a light chain variable region comprising the amino acid sequence of SEQ ID NO: 765. In some embodiments, the antibody is Humanized CUB4 #135 HC4-H. In some embodiments, the antibody comprises CDR-H1, CDR-H2, CDR-H3, CDR-L1, CDR-L2, and CDR-L3 comprising the amino acid sequences of SEQ ID NOs: 766, 767, 768, 769, 770, and 771, respectively. In some embodiments, the antibody comprises a heavy chain variable region comprising the amino acid sequence of SEQ ID NO: 772 and a light chain variable region comprising the amino acid sequence of SEQ ID NO: 773. In some embodiments, the antibody is CUB4. In some embodiments, the antibody comprises CDR-H1, CDR-H2, CDR-H3, CDR-L1, CDR-L2, and CDR-L3 comprising the amino acid sequences of SEQ ID NOs: 774, 775, 776, 777, 778, 779, respectively. In some embodiments, the antibody comprises a heavy chain variable region comprising the amino acid sequence of SEQ ID NO: 780 and a light chain variable region comprising the amino acid sequence of SEQ ID NO: 781. In some embodiments, the antibody is CP13E10-WT. In some embodiments, the antibody comprises CDR-H1, CDR-H2, CDR-H3, CDR-L1, CDR-L2, and CDR-L3 comprising the amino acid sequences of SEQ ID NOs: 782, 783, 784, 785, 786, and 787, respectively. In some embodiments, the antibody comprises a heavy chain variable region comprising the amino acid sequence of SEQ ID NO: 788 and a light chain variable region comprising the amino acid sequence of SEQ ID NO: 789. In some embodiments, the antibody is CP13E10-54HCv13-89LCv1.

In some embodiments, an antibody provided herein binds to ASCT2. In some embodiments, the antibody comprises a heavy chain variable region comprising the amino acid sequence of SEQ ID NO: 790 and a light chain variable region comprising the amino acid sequence of SEQ ID NO: 791. In some embodiments, the antibody is KM8094a. In some embodiments, the antibody comprises a heavy chain variable region comprising the amino acid sequence of SEQ ID NO: 792 and a light chain variable region comprising the amino acid sequence of SEQ ID NO: 793. In some embodiments, the antibody is KM8094b. In some embodiments, the antibody comprises CDR-H1, CDR-H2, CDR-H3, CDR-L1, CDR-L2, and CDR-L3 comprising the amino acid sequences of SEQ ID NOs: 794, 795, 796, 797, 798, and 799, respectively. In some embodiments, the antibody comprises a heavy chain variable region comprising the amino acid sequence of SEQ ID NO: 800 and a light chain variable region comprising the amino acid sequence of SEQ ID NO: 801. In some embodiments, the antibody is KM4018.

In some embodiments, an antibody provided herein binds to CD123. In some embodiments, the antibody comprises CDR-H1, CDR-H2, CDR-H3, CDR-L1, CDR-L2, and CDR-L3 comprising the amino acid sequences of SEQ ID NOs: 802, 803, 804, 805, 806, and 807, respectively. In some embodiments, the antibody comprises a heavy chain variable region comprising the amino acid sequence of SEQ ID NO: 808 and a light chain variable region comprising the amino acid sequence of SEQ ID NO: 809. In some embodiments, the antibody is h7G3. See WO 2016201065.

In some embodiments, an antibody provided herein binds to GPC3. In some embodiments, the antibody comprises CDR-H1, CDR-H2, CDR-H3, CDR-L1, CDR-L2, and CDR-L3 comprising the amino acid sequences of SEQ ID NOs: 810, 811, 812, 813, 814, and 815, respectively. In some embodiments, the antibody comprises a heavy chain variable region comprising the amino acid sequence of SEQ ID NO: 816 and a light chain variable region comprising the amino acid sequence of SEQ ID NO: 817. In some embodiments, the antibody is hGPC3-1. See WO 2019161174.

In some embodiments, an antibody provided herein binds to B6A. In some embodiments, the antibody comprises CDR-H1, CDR-H2, CDR-H3, CDR-L1, CDR-L2, and CDR-L3 comprising the amino acid sequences of SEQ ID NOs: 818, 819, 820, 821, 822, and 823, respectively. In some embodiments, the antibody comprises a heavy chain variable region comprising the amino acid sequence of SEQ ID NO: 824 and a light chain variable region comprising the amino acid sequence of SEQ ID NO: 825. In some embodiments, the antibody is h2A2. See PCT/US20/63390. In some embodiments, the antibody comprises CDR-H1, CDR-H2, CDR-H3, CDR-L1, CDR-L2, and CDR-L3 comprising the amino acid sequences of SEQ ID NOs: 826, 827, 828, 829, 830, and 831, respectively. In some embodiments, the antibody comprises a heavy chain variable region comprising the amino acid sequence of SEQ ID NO: 832 and a light chain variable region comprising the amino acid sequence of SEQ ID NO: 833. In some embodiments, the antibody is h15H3. See WO 2013/123152.

In some embodiments, an antibody provided herein binds to PD-L1. In some embodiments, the antibody comprises CDR-H1, CDR-H2, CDR-H3, CDR-L1, CDR-L2, and CDR-L3 comprising the amino acid sequences of SEQ ID NOs: 834, 835, 836, 837, 838, and 839, respectively. In some embodiments, the antibody comprises a heavy chain variable region comprising the amino acid sequence of SEQ ID NO: 840 and a light chain variable region comprising the amino acid sequence of SEQ ID NO: 841. In some embodiments, the antibody is SG-559-01. See PCT/US2020/054037.

In some embodiments, an antibody provided herein binds to TIGIT. In some embodiments, the antibody comprises CDR-H1, CDR-H2, CDR-H3, CDR-L1, CDR-L2, and CDR-L3 comprising the amino acid sequences of SEQ ID NOs: 842, 843, 844, 845, 846, and 847, respectively. In some embodiments, the antibody comprises a heavy chain variable region comprising the amino acid sequence of SEQ ID NO: 848 and a light chain variable region comprising the amino acid sequence of SEQ ID NO: 849. In some embodiments, the antibody is Clone 13 (also known as ADI-23674 or mAb13). See WO 2020041541.

In some embodiments, an antibody provided herein binds to STN. In some embodiments, the antibody comprises CDR-H1, CDR-H2, CDR-H3, CDR-L1, CDR-L2, and CDR-L3 comprising the amino acid sequences of SEQ ID NOs: 850, 851, 852, 853, 854, and 855, respectively. In some embodiments, the antibody comprises a heavy chain variable region comprising the amino acid sequence of SEQ ID NO: 856 and a light chain variable region comprising the amino acid sequence of SEQ ID NO: 857. In some embodiments, the antibody is 2G12-2B2. See WO 2017083582.

In some embodiments, an antibody provided herein binds to CD33. In some embodiments, the antibody comprises CDR-H1, CDR-H2, CDR-H3, CDR-L1, CDR-L2, and CDR-L3 comprising the amino acid sequences of SEQ ID NOs: 858, 859, 860, 861, 862, and 863, respectively. In some embodiments, the antibody comprises a heavy chain variable region comprising the amino acid sequence of SEQ ID NO: 864 and a light chain variable region comprising the amino acid sequence of SEQ ID NO: 865. In some embodiments, the antibody is h2H12. See WO2013173496.

In some embodiments, an antibody provided herein binds to NTBA (also known as CD352). In some embodiments, the antibody comprises CDR-H1, CDR-H2, CDR-H3, CDR-L1, CDR-L2, and CDR-L3 comprising the amino acid sequences of SEQ ID NOs: 866, 867, 868, 869, 870, and 871, respectively. In some embodiments, the antibody comprises a heavy chain variable region comprising the amino acid sequence of SEQ ID NO: 872 and a light chain variable region comprising the amino acid sequence of SEQ ID NO: 873. In some embodiments, the antibody is h20F3 HDLD. See WO 2017004330.

In some embodiments, an antibody provided herein binds to BCMA. In some embodiments, the antibody comprises CDR-H1, CDR-H2, CDR-H3, CDR-L1, CDR-L2, and CDR-L3 comprising the amino acid sequences of SEQ ID NOs: 874, 875, 876, 877, 878, and 879, respectively. In some embodiments, the antibody comprises a heavy chain variable region comprising the amino acid sequence of SEQ ID NO: 880 and a light chain variable region comprising the amino acid sequence of SEQ ID NO: 881. In some embodiments, the antibody is SEA-BCMA (also known as hSG16.17). See WO 2017/143069.

In some embodiments, an antibody provided herein binds to Tissue Factor (also known as TF). In some embodiments, the antibody comprises CDR-H1, CDR-H2, CDR-H3, CDR-L1, CDR-L2, and CDR-L3 comprising the amino acid sequences of SEQ ID NOs: 882, 883, 884, 885, 886, and 887, respectively. In some embodiments, the antibody comprises a heavy chain variable region comprising the amino acid sequence of SEQ ID NO: 888 and a light chain variable region comprising the amino acid sequence of SEQ ID NO: 889. In some embodiments, the antibody is tisotumab. See WO 2010/066803 and U.S. Pat. No. 9,150,658.

TABLE OF SEQUENCES

| SEQ | | |
|---|---|---|
| ID NO | Description | Sequence |
| 1 | CAC10 CDR-H1 | DYYIT |
| 2 | CAC10 CDR-H2 | WIYPGSGNTKYNEKFKG |
| 3 | CAC10 CDR-H3 | YGNYWFAY |
| 4 | CAC10 CDR-L1 | KASQSVDFDGDSYMN |
| 5 | CAC10 CDR-L2 | AASNLES |
| 6 | CAC10 CDR-L3 | QQSNEDPWT |
| 7 | CAC10 VH | QIQLQQSGPEVVKPGASVKISCKASGYTFTDYYITWVKQKPGQGLEWIGWIYPGSGNTKY |
| | | NEKFKGKATLTVDTSSSTAFMQLSSLTSEDTAVYFCANYGNYWFAYWGQGTQVTVSA |
| 8 | CAC10 VL | DIVLTQSPASLAVSLGQRATISCKASQSVDFDGDSYMNWYQQKPGQPPKVLIYAASNLES |
| | | GIPARFSGSGSGTDFTLNIHPVEEEDAATYYCQQSNEDPWTFGGGTKLEIK |
| 9 | CAC10 HC | QIQLQQSGPEVVKPGASVKISCKASGYTFTDYYITWVKQKPGQGLEWIGWIYPGSGNTKY |
| | | NEKFKGKATLTVDTSSSTAFMQLSSLTSEDTAVYFCANYGNYWFAYWGQGTQVTVSAAS |
| | | TKGPSVFPLAPSSKSTSGGTAALGCLVKDYFPEPVTVSWNSGALTSGVHTFPAVLQSS |
| | | GLYSLSSVTVPSSSLGTQTYICNVNHKPSNTKVDKKVEPKSCDKTHTCPPCPAPELLGG |
| | | PSVFLFPPKPKDTLMISRTPEVTCWWVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 10 | CAC 10 HC v2 | QIQLQQSGPEVVKPGASVKISCKASGYTFTDYYITWVKQKPGQGLEWIGWIYPGSGNTKY |
| | | NEKFKGKATLTVDTSSSTAFMQLSSLTSEDTAVYFCANYGNYWFAYWGQGTQVTVSAAS |
| | | TKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 11 | CAC10 LC | DIVLTQSPASLAVSLGQRATISCKASQSVDFDGDSYMNWYQQKPGQPPKVLIYAASNLES |
| | | GIPARFSGSGSGTDFTLNIHPVEEEDAATYYCQQSNEDPWTFGGGTKLEIKR |
| | | TVAAPSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 12 | h1F6 VH | QVQLVQSGAEVKKPGASVKVSCKASGYTFTNYGMNWVRQAPGQGLKWMGWINTYTGE |
| | | PTY |
| | | ADAFKGRVTMTRDTSISTAYMELSRLRSDDTAVYYCARDYGDYGMDYWGQGTTVTVSS |
| 13 | h1F6 VL | DIVMTQSPDSLAVSLGERATINCRASKSVSTSGYSFMHWYQQKPGQPPKLLIYLASNLES |
| | | GVPDRFSGSGSGTDFTLTISSLQAEDVAVYYCQHSREVPWTFGQGTKVEIK |
| 14 | h1F6 HC | QVQLVQSGAEVKKPGASVKVSCKASGYTFTNYGMNWVRQAPGQGLKWMGWINTYTGE |
| | | PTY |
| | | ADAFKGRVTMTRDTSISTAYMELSRLRSDDTAVYYCARDYGDYGMDYWGQGTTVTVSSA |
| | | S |
| | | TKGPSVFPLAPSSKSTSGGTAALGCLVKDYFPEPVTVSWNSGALTSGVHTFPAVLQSSGL |
| | | YSLSSWTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRWSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQ |
| | | Q |
| | | GNVFSCSVMHEALHNHYTQKSLSLSPGK |
| 15 | h1F6 LC | DIVMTQSPDSLAVSLGERATINCRASKSVSTSGYSFMHWYQQKPGQPPKLLIYLASNLES |
| | | GVPDRFSGSGSGTDFTLTISSLQAEDVAVYYCQHSREVPWTFGQGTKVEIKRTVAAPSVF |
| | | IFPPSDEQLKSGTASWVCLLNNFYPREAKVQWKVDNALQSGNSQESVTEQDSKDSTYSLS |
| | | STLTLSKADYEKHKVYACEVTHQGLSSPVTKSFNRGEC |
| 16 | TROP2 CDR-H1 | NYGMN |
| 17 | TROP2 CDR-H2 | WINTYTGEPTYTDDFKG |
| 18 | TROP2 CDR-H3 | GGFGSSYWYFDV |
| 19 | TROP2 CDR-L1 | KASQDVSIAVA |
| 20 | TROP2 CDR-L2 | SASYRYT |
| 21 | TROP2 CDR-L3 | QQHYITPLT |
| 22 | TROP2 VH | QVQLQQSGSELKKPGASVKVSCKASGYTFTNYGMNWVKQAPGQGLKWMGWINTYTGE |
| | | PT |
| | | YTDDFKGRFAFSLDTSVSTAYLQISSLKADDTAVYFCARGGFGSSYWYFDVWGQGSLVT |
| | | VSS |
| 23 | TROP2 VL | DIQLTQSPSSLSASVGDRVSITCKASQDVSIAVAWYQQKPGKAPKLLIYSASYRYTGVP |
| | | DRFSGSGSGTDFTLTISSLQPEDFAVYYCQQHYITPLTFGAGTKVEIK |
| 24 | TROP2 CDR-H1 | TAGMQ |
| 25 | TROP2 CDR-H2 | WINTHSGVPKYAEDFKG |
| 26 | TROP2 CDR-H3 | SGFGSSYWYFDV |
| 27 | TROP2 CDR-L1 | KASQDVSTAVA |
| 28 | TROP2 CDR-L2 | SASYRYT |
| 29 | TROP2 CDR-L3 | QQHYITPLT |
| 30 | TROP2 VH | QVQLVQSGAEVKKPGASVKVSCKASGYTFTTAGMQWVRQAPGQGLEWMGWINTHSGV |
| | | PKYAEDFKGRVTISADTSTSTAYLQLSSLKSEDTAVYYCARSGFGSSYWYFDVWGQGTLV |
| | | TVSS |
| 31 | TROP2 VL | DIQMTQSPSSLSASVGDRVTITCKASQDVSTAVAWYQQKPGKAPKLLIYSASYRYTGVPS |
| | | RFSGSGSGTDFTLTISSLQPEDFAVYYCQQHYITPLTFGQGTKLEIK |
| 32 | MICA CDR-H1 | SQNIY |
| 33 | MICA CDR-H2 | YIEPYNVVPMYNPKFKG |
| 34 | MICA CDR-H3 | SGSSNFDY |
| 35 | MICA CDR-L1 | SASSSISSHYLH |
| 36 | MICA CDR-L2 | RTSNLAS |
| 37 | MICA CDR-L3 | QQGSSLPLT |
| 38 | MICA VH | EIQLVQSGAEVKKPGASVKVSCKASGYAFTSQNIYWVRQAPGQGLEWIGYIEPYNWPMY |
| | | NPKFKGRATLTVDKSTSTAYLELSSLRSEDTAVYYCARSGSSNFDYWGQGTLVTVSS |
| 39 | MICA VL | DIQLTQSPSSLSASVGDRVTITCSASSSISSHYLHWYQQKPGKSPKLLIYRTSNLASGVPSR |
| | | FSGSGSGTDYTLTISSLQPEDFATYYCQQGSSLPLTFGQGTKVEIK |
| 40 | MICA CDR-H1 | NYAMH |
| 41 | MICA CDR-H2 | LIWYDGSNKFYGDSVKG |
| 42 | MICA CDR-H3 | EGSGHY |
| 43 | MICA CDR-L1 | RASQGISSALA |
| 44 | MICA CDR-L2 | DASSLES |
| 45 | MICA CDR-L3 | QQFNSYPIT |
| 46 | MICA VH | QVQLVESGGGWVQPGRSLRLSCAASGFTFSNYAMHWVRQAPGEGLEWVALIWYDGSNK |
| | | FYGDSVKGRFTISRDNSKNTLYLQMNSLSAEDTAVYYCAREGSGHYWGQGTLVTVSS |
| 47 | MICA VL | AIQLTQSPSSLSASVGDRVTITCRASQGISSALAWYQQKPGKVPKSLIYDASSLESGVPSR |
| | | FSGSGSGTDFTLTISSLQPEDFATYYCQQFNSYPITFGQGTRLEIK |
| 48 | MICA CDR-H1 | NYAMS |
| 49 | MICA CDR-H2 | YISPGGDYIYYADSVKG |
| 50 | MICA CDR-H3 | DRRHYGSYAMDY |
| 51 | MICA CDR-L1 | RSSKSLLHSNLNTYLY |
| 52 | MICA CDR-L2 | RMSNLAS |
| 53 | MICA CDR-L3 | MQHLEYPFT |
| 54 | MICA VH | QVQLVESGGGLVKPGGSLRLSCAASGFTFSNYAMSWIRQAPGKGLEWVSYISPGGDYIY |
| | | YADSVKGRFTISRDNAKNSLYLQMNSLRAEDTAVYYCTTDRRHYGSYAMDYWGQGTLVT |
| | | VSS |
| 55 | MICA VL | DIVMTQSPLSLPVTPGEPASISCRSSKSLLHSNLNTYLYWFLQKPGQSPQILIYRMSNLASG |
| | | VPDRFSGSGSGTAFTLKISRVEAEDVGVYYCMQHLEYPFTFGPGTKLEIK |
| 56 | MICA CDR-H1 | TYAFH |
| 57 | MICA CDR-H2 | GIVPIFGTLKYAQKFQD |
| 58 | MICA CDR-H3 | AIQLEGRPFDH |
| 59 | MICA CDR-L1 | RASQGITSYLA |
| 60 | MICA CDR-L2 | AASALQS |
| 61 | MICA CDR-L3 | QQVNRGAAIT |
| 62 | MICA VH | QVQLVQSGAEVKKPGSSVRVSCRASGGSSTTYAFHWVRQAPGQGLEWMGGIVPIFGTL |
| | | KYAQKFQDRVTLTADKSTGTAYMELNSLRLDDTAVYYCARAIQLEGRPFDHWGQGTQVT |
| | | VSA |
| 63 | MICA VL | DIQLTQSPSFLSASVGDRVTITCRASQGITSYLAWYQQKPGKAPKLLIYAASALQSGVPSR |
| | | FSGRGSGTEFTLTISSLQPEDFATYYCQQVNRGAAITFGHGTRLDIK |
| 64 | CD24 CDR-H1 | TYAFH |
| 65 | CD24 CDR-H2 | GIVPIFGTLKYAQKFQD |
| 66 | CD24 CDR-H3 | AIQLEGRPFDH |
| 67 | CD24 CDR-L1 | RASQGITSYLA |
| 68 | CD24 CDR-L2 | AASALQS |
| 69 | CD24 CDR-L3 | QQVNRGAAIT |
| 70 | CD24 VH | QVQLVQSGAEVKKPGSSVRVSCRASGGSSTTYAFHWVRQAPGQGLEWMGGIVPIFGTL |
| | | KYAQKFQDRVTLTADKSTGTAYMELNSLRLDDTAVYYCARAIQLEGRPFDHWGQGTQVT |
| | | VSA |
| 71 | CD24 VL | DIQLTQSPSFLSASVGDRVTITCRASQGITSYLAWYQQKPGKAPKLLIYAASALQSGVPS |
| | | RFSGRGSGTEFTLTISSLQPEDFATYYCQQVNRGAAITFGHGTRLDIK |
| 72 | ITGav CDR-H1 | RYTMH |
| 73 | ITGav CDR-H2 | VISFDGSNKYYVDSVKG |
| 74 | ITGav CDR-H3 | EARGSYAFDI |
| 75 | ITGav CDR-L1 | RASQSVSSYLA |
| 76 | ITGav CDR-L2 | DASNRAT |
| 77 | ITGav CDR-L3 | QQRSNWPPFT |
| 78 | ITGav VH | QVQLVESGGGVVQPGRSRRLSCAASGFTFSRYTMHWVRQAPGKGLEWVAVISFDGSNK |
| | | YYVDSVKGRFTISRDNSENTLYLQVNILRAEDTAVYYCAREARGSYAFDIWGQGTMVTVS |
| | | S |
| 79 | ITGav VL | EIVLTQSPATLSLSPGERATLSCRASQSVSSYLAWYQQKPGQAPRLLIYDASNRATGIPAR |
| | | FSGSGSGTDFTLTISSLEPEDFAVYYCQQRSNWPPFTFGPGTKVDIK |
| 80 | ITGav CDR-H1 | SFWMH |
| 81 | ITGav CDR-H2 | YINPRSGYTEYNEIFRD |
| 82 | ITGav CDR-H3 | FLGRGAMDY |
| 83 | ITGav CDR-L1 | RASQDISNYLA |
| 84 | ITGav CDR-L2 | YTSKIHS |
| 85 | ITGav CDR-L3 | QQGNTFPYT |
| 86 | ITGav VH | QVQLQQSGGELAKPGASVKVSCKASGYTFSSFWMHWVRQAPGQGLEWIGYINPRSGYT |
| | | EYNEIFRDKATMTTDTSTSTAYMELSSLRSEDTAVYYCASFLGRGAMDYWGQGTTVTVSS |
| 87 | ITGav VL | DIQMTQSPSSLSASVGDRVTITCRASQDISNYLAWYQQKPGKAPKLLIYYTSKIHSGVPSR |
| | | FSGSGSGTDYTFTISSLQPEDIATYYCQQGNTFPYTFGQGTKVEIK |
| 88 | gpA33 CDR-H1 | TSSYYWG |
| 89 | gpA33 CDR-H2 | TIYYNGSTYYSPSLKS |
| 90 | gpA33 CDR-H3 | QGYDIKINIDV |
| 91 | gpA33 CDR-L1 | RASQSVSSYLA |
| 92 | gpA33 CDR-L2 | VASNRAT |
| 93 | gpA33 CDR-L3 | QQRSNWPLT |
| 94 | gpA33 VH | QLQLQESGPGLVKPSETLSLTCTVSGGSISTSSYYWGWIRQPPGKGLEWIGTIYYNGSTY |
| | | YSPSLKSRVSISVDTSKNQFSLKLSSVTAADTSVYYCARQGYDIKINIDVWGQGTTVTVSS |
| 95 | gpA33 VL | EIVLTQSPATLSLSPGERATLSCRASQSVSSYLAWYQQKPGQAPRLLIYVASNRATGIPAR |
| | | FSGSGSGTDFTLTISSLEPEDFAVYYCQQRSNWPLTFGGGTKVEIK |
| 96 | IL1Rap CDR-H1 | SSWMN |
| 97 | IL1Rap CDR-H2 | RIYPGDGNTHYAQKFQG |
| 98 | IL1Rap CDR-H3 | GYLDPMDY |
| 99 | IL1Rap CDR-L1 | QASQGINNYLN |
| 100 | IL1Rap CDR-L2 | YTSGLHA |
| 101 | IL1Rap CDR-L3 | QQYSILPWT |
| 102 | IL1Rap VH | QVQLVQSGAEVKKPGSSVKVSCKASGYAFTSSWMNWVRQAPGQGLEWMGRIYPGDGN |
| | | THYAQKFQGRVTLTADKSTSTAYMELSSLRSEDTAVYYCGEGYLDPMDYWGQGTLVTVS |
| | | S |
| 103 | IL1Rap VL | DIQMTQSPSSLSASVGDRVTITCQASQGINNYLNWYQQKPGKAPKLLIHYTSGLHAGVPS |
| | | RFSGSGSGTDYTLTISSLEPEDVATYYCQQYSILPWTFGGGTKVEIK |
| 104 | EpCAM CDR-H1 | SYGMH |
| 105 | EpCAM CDR-H2 | VISYDGSNKYYADSVKG |
| 106 | EpCAM CDR-H3 | DMG |
| 107 | EpCAM CDR-L1 | RTSQSISSYLN |
| 108 | EpCAM CDR-L2 | WASTRES |
| 109 | EpCAM CDR-L3 | QQSYDIPYT |
| 110 | EpCAM VH | EVQLLESGGGVVQPGRSLRLSCAASGFTFSSYGMHWVRQAPGKGLEWVAVISYDGSNK |
| | | YYADSVKGRFTISRDNSKNTLYLQMNSLRAEDTAVYYCAKDMGWGSGWRPYYYYGMDV |
| | | WGQGTTVTVSS |
| 111 | EpCAM VL | ELQMTQSPSSLSASVGDRVTITCRTSQSISSYLNWYQQKPGQPPKLLIYWASTRESGVPD |
| | | RFSGSGSGTDFTLTISSLQPEDSATYYCQQSYDIPYTFGQGTKLEIK |
| 112 | EpCAM CDR-H1 | NYWMS |
| 113 | EpCAM CDR-H2 | NIKQDGSEKFYADSVKG |
| 114 | EpCAM CDR-H3 | VGPSWEQDY |
| 115 | EpCAM CDR-L1 | TGSSSNIGSYYGVH |
| 116 | EpCAM CDR-L2 | SDTNRPS |
| 117 | EpCAM CDR-L3 | QSYDKGFGHRV |
| 118 | EpCAM VH | EVQLVESGGGLVQPGGSLRLSCAASGFTFSNYWMSWVRQAPGKGLEWVANIKQDGSEK |
| | | FYADSVKGRFTISRDNAKNSLYLQMNSLRAEDTAVYYCARVGPSWEQDYWGQGTLVTVS |
| | | A |
| 119 | EpCAM VL | QSVLTQPPSVSGAPGQRVTISCTGSSSNIGSYYGVHWYQQLPGTAPKLLIYSDTNRPSGV |
| | | PDRFSGSKSGTSASLAITGLQAEDEADYYCQSYD |
| 120 | EpCAM CDR-H1 | SYAIS |
| 121 | EpCAM CDR-H2 | GIIPIFGTANYAQKFQG |
| 122 | EpCAM CDR-H3 | GLLWNY |
| 123 | EpCAM CDR-L1 | RASQSVSSNLA |
| 124 | EpCAM CDR-L2 | GASTTAS |
| 125 | EpCAM CDR-L3 | QQYNNWPPAYT |
| 126 | EpCAM VH | QVQLVQSGAEVKKPGSSVKVSCKASGGTFSSYAISWVRQAPGQGLEWMGGIIPIFGTANY |
| | | AQKFQGRVTITADESTSTAYMELSSLRSEDTAVYYCARGLLWNYWGQGTLVTVSS |
| 127 | EpCAM VL | EIVMTQSPATLSVSPGERATLSCRASQSVSSNLAWYQQKPGQAPRLIIYGASTTASGIPAR |
| | | FSASGSGTDFTLTISSLQSEDFAVYYCQQYNNWPPAYTFGQGTKLEIK |
| 128 | EpCAM CDR-H1 | NYGMN |
| 129 | EpCAM CDR-H2 | WINTYTGEPTYGEDFKG |
| 130 | EpCAM CDR-H3 | FGNYVDY |
| 131 | EpCAM CDR-L1 | RSSKNLLHSNGITYLY |
| 132 | EpCAM CDR-L2 | QMSNLAS |
| 133 | EpCAM CDR-L3 | AQNLEIPRT |
| 134 | EpCAM VH | QVQLVQSGPEVKKPGASVKVSCKASGYTFTNYGMNWVRQAPGQGLEWMGWINTYTGE |
| | | PTYGEDFKGRFAFSLDTSASTAYMELSSLRSEDTAVYFCARFGNYVDYWGQGSLVTVSS |
| 135 | EpCAM VL | DIVMTQSPLSLPVTPGEPASISCRSSKNLLHSNGITYLYWYLQKPGQSPQLLIYQMSNLAS |
| | | GVPDRFSSSGSGTDFTLKISRVEAEDVGVYYCAQNLEIPRTFGQGTKVEIK |
| 136 | EpCAM CDR-H1 | KYGMN |
| 137 | EpCAM CDR-H2 | WINTYTEEPTYGDDFKG |
| 138 | EpCAM CDR-H3 | FGSAVDY |
| 139 | EpCAM CDR-L1 | RSSKSLLHSNGITYLY |
| 140 | EpCAM CDR-L2 | QMSNRAS |
| 141 | EpCAM CDR-L3 | AQNLELPRT |
| 142 | EpCAM VH | QIQLVQSGPEVKKPGESVKISCKASGYTFTKYGMNWVKQAPGQGLKWMGWINTYTEEPT |
| | | YGDDFKGRFTFTLDTSTSTAYLEISSLRSEDTATYFCARFGSAVDYWGQGTLVTVSS |
| 143 | EpCAM VL | DIVMTQSALSNPVTLGESGSISCRSSKSLLHSNGITYLYWYLQKPGQSPQLLIYQMSNRAS |
| | | GVPDRFSSSGSGTDFTLKISRVEAEDVGVYYCAQNLELPRTFGQGTKLEMKR |
| 144 | EpCAM CDR-H1 | DYSMH |
| 145 | EpCAM CDR-H2 | WINTETGEPTYADDFKG |
| 146 | EpCAM CDR-H3 | TAVY |
| 147 | EpCAM CDR-L1 | RASQEISVSLS |
| 148 | EpCAM CDR-L2 | ATSTLDS |
| 149 | EpCAM CDR-L3 | LQYASYPWT |
| 150 | EpCAM VH | QVKLQESGPELKKPGETVKISCKASGYTFTDYSMHWVKQAPGKGLKWMGWINTETGEPT |
| | | YADDFKGRFAFSLETSASTAYLQINNLKNEDTATYFCARTAVYWGQGTTVTVSS |
| 151 | EpCAM VL | DIQMTQSPSSLSASLGERVSLTCRASQEISVSLSWLQQEPDGTIKRLIYATSTLDSGVPKRF |
| | | SGSRSGSDYSLTISSLESEDFVDYYCLQYASYPWTFGGGTKLEIKR |
| 152 | CD352 CDR-H1 | NYGMN |
| 153 | CD352 CDR-H2 | WINTYSGEPRYADDFKG |
| 154 | CD352 CDR-H3 | DYGRWYFDV |
| 155 | CD352 CDR-L1 | RASSSVSHMH |
| 156 | CD352 CDR-L2 | ATSNLAS |
| 157 | CD352 CDR-L3 | QQWSSTPRT |
| 158 | CD352 VH | QIQLVQSGSELKKPGASVKVSCKASGYTFTNYGMNWVRQAPGQDLKWMGWINTYSGEP |
| | | RYADDFKGRFVFSLDKSVNTAYLQISSLKAEDTAVYYCARDYGRWYFDVWGQGTTVTVS |
| | | S |
| 159 | CD352 VL | QIVLSQSPATLSLSPGERATMSCRASSSVSHMHWYQQKPGQAPRPWIYATSNLASGVPA |
| | | RFSGSGSGTDYTLTISSLEPEDFAVYYCQQWSSTPRTFGGGTKVEIKR |
| 160 | CS1 CDR-H1 | RYWMS |
| 161 | CS1 CDR-H2 | EINPDSSTINYAPSLKD |
| 162 | CS1 CDR-H3 | PDGNYWYFDV |
| 163 | CS1 CDR-L1 | KASQDVGIAVA |
| 164 | CS1 CDR-L2 | WASTRHT |
| 165 | CS1 CDR-L3 | QQYSSYPYT |
| 166 | CS1 VH | EVQLVESGGGLVQPGGSLRLSCAASGFDFSRYWMSWVRQAPGKGLEWIGEINPDSSTIN |
| | | YAPSLKDKFIISRDNAKNSLYLQMNSLRAEDTAVYYCARPDGNYWYFDVWGQGTLVTVSS |
| 167 | CS1 VL | DIQMTQSPSSLSASVGDRVTITCKASQDVGIAVAWYQQKPGKVPKLLIYWASTRHTGVPD |
| | | RFSGSGSGTDFTLTISSLQPEDVATYYCQQYSSYPYTFGQGTKVEIKR |
| 168 | CD38 CDR-H1 | SFAMS |
| 169 | CD38 CDR-H2 | AISGSGGGTYYADSVKG |
| 170 | CD38 CDR-H3 | DKILWFGEPVFDY |
| 171 | CD38 CDR-L1 | RASQSVSSYLA |
| 172 | CD38 CDR-L2 | DASNRAT |
| 173 | CD38 CDR-L3 | QQRSNWPPT |
| 174 | CD38 VH | EVQLLESGGGLVQPGGSLRLSCAVSGFTFNSFAMSWVRQAPGKGLEWVSAISGSGGGT |
| | | YYADSVKGRFTISRDNSKNTLYLQMNSLRAEDTAVYFCAKDKILWFGEPVFDYWGQGTLV |
| | | TVSS |
| 175 | CD38 VL | EIVLTQSPATLSLSPGERATLSCRASQSVSSYLAWYQQKPGQAPRLLIYDASNRATGIPAR |
| | | FSGSGSGTDFTLTISSLEPEDFAVYYCQQRSNWPPTFGQGTKVEIKR |
| 176 | CD25 CDR-H1 | SYRMH |
| 177 | CD25 CDR-H2 | YINPSTGYTEYNQKFKD |
| 178 | CD25 CDR-H3 | GGGVFDY |
| 179 | CD25 CDR-L1 | SASSSISYMH |
| 180 | CD25 CDR-L2 | TTSNLAS |
| 181 | CD25 CDR-L3 | HQRSTYPLT |
| 182 | CD25 VH | QVQLVQSGAEVKKPGSSVKVSCKASGYTFTSYRMHWVRQAPGQGLEWIGYINPSTGYTE |
| | | YNQKFKDKATITADESTNTAYMELSSLRSEDTAVYYCARGGGVFDYWGQGTLVTVSS |
| 183 | CD25 VL | DIQMTQSPSTLSASVGDRVTITCSASSSISYMHWYQQKPGKAPKLLIYTTSNLASGVPARF |
| | | SGSGSGTEFTLTISSLQPDDFATYYCHQRSTYPLTFGQGTKVEVK |
| 184 | ADAM9 CDR-H1 | SYWM |
| 185 | ADAM9 CDR-H2 | EIIPINGHTNYNEKFKS |
| 186 | ADAM9 CDR-H3 | GGYYYYGSRDYFDY |
| 187 | ADAM9 CDR-L1 | KASQSVDYDGDSYMN |
| 188 | ADAM9 CDR-L2 | AASDLES |
| 189 | ADAM9 CDR-L3 | QQSHEDPFT |
| 190 | ADAM9 VH | QVQLQQPGAELVKPGASVKLSCKASGYTFTSYWMHWVKQRPGQGLEWIGEIIPINGHTN |
| | | YNEKFKSKATLTLDKSSSTAYMQLSSLASEDSAVYYCARGGYYYYGSRDYFDYWGQGTT |
| | | LTVSS |
| 191 | ADAM9 VL | DIVLTQSPASLAVSLGQRATISCKASQSVDYDGDSYMNWYQQIPGQPPKLLIYAASDLESG |
| | | IPARFSGSGSGTDFTLNIHPVEEEDAATYYCQQSHEDPFTFGGGTKLEIK |
| 192 | ADAM9 CDR-H1 | SYWM |
| 193 | ADAM9 CDR-H2 | EIIPIFGHTNYNEKFKS |
| 194 | ADAM9 CDR-H3 | GGYYYYPRQGFLDY |
| 195 | ADAM9 CDR-L1 | KASQSVDYDSGDSYMN |
| 196 | ADAM9 CDR-L2 | AASDLES |
| 197 | ADAM9 CDR-L3 | QQSHEDPFT |
| 198 | ADAM9 VH | EVQLVESGGGLVKPGGSLRLSCAASGFTFSSYWMHWVRQAPGKGLEWVGEIIPIFGHTN |
| | | YNEKFKSRFTISLDNSKNTLYLQMGSLRAEDTAVYYCARGGYYYYPRQGFLDYWGQGTT |
| | | VTVSS |
| 199 | ADAM9 VL | DIVMTQSPDSLAVSLGERATISCKASQSVDYSGDSYMNWYQQKPGQPPKLLIYAASDLES |
| | | GIPARFSGSGSGTDFTLTISSLEPEDFATYYCQQSHEDPFTFGQGTKLEIK |
| 200 | CD59 CDR-H1 | YGMN |
| 201 | CD59 CDR-H2 | YISSSSSTIYADSVKG |
| 202 | CD59 CDR-H3 | GPGMDV |
| 203 | CD59 CDR-L1 | KSSQSVLYSSNNKNYLA |
| 204 | CD59 CDR-L2 | WASTRES |
| 205 | CD59 CDR-L3 | QQYYSTPQLT |
| 206 | CD59 VH | QVQLQQSGGGVVQPGRSLGLSCAASFTFSSYGMNWVRQAPGKGLEWVSYISSSSSTIYA |
| | | DSVKGRFTISRDNSKNTLYLQMNSLRAEDTAVYYCARGPGMDVWGQGTTVTVS |
| 207 | CD59 VL | DIVLTQSPDSLAVSLGERATINCKSSQSVLYSSNNKNYLAWYQQKPGQPPKLLIYWASTRE |
| | | SGVPDRFSGSGSGTDFTPAISSLQAEDVAVYYCQQYYSTPQLTFGGGTKVDIK |
| 208 | CD19 CDR-H1 | TSGMGVG |
| 209 | CD19 CDR-H2 | HIWWDDDKRYNPALKS |
| 210 | CD19 CDR-H3 | MELWSYYFDY |
| 211 | CD19 CDR-L1 | SASSSVSYMH |
| 212 | CD19 CDR-L2 | DTSKLAS |
| 213 | CD19 CDR-L3 | FQGSVYPFT |
| 214 | CD19 VH | QVQLQESGPGLVKPSQTLSLTCTVSGGSISTSGMGVGWIRQHPGKGLEWIGHIWWDDDK |
| | | RYNPALKSRVTISVDTSKNQFSLKLSSVTAADTAVYYCARMELWSYYFDYWGQGTLVTVS |
| | | S |
| 215 | CD19 VL | EIVLTQSPATLSLSPGERATLSCSASSSVSYMHWYQQKPGQAPRLLIYDTSKLASGIPARF |
| | | SGSGSGTDFTLTISSLEPEDVAVYYCFQGSVYPFTFGQGTKLEIKR |
| 216 | CD70 CDR-H1 | NYGMN |
| 217 | CD70 CDR-H2 | WINTYTGEPTYADAFKG |
| 218 | CD70 CDR-H3 | DYGDYGMDY |
| 219 | CD70 CDR-L1 | RASKSVSTSGYSFMH |
| 220 | CD70 CDR-L2 | LASNLES |
| 221 | CD70 CDR-L3 | QHSREVPWT |
| 222 | CD70 VH | QVQLVQSGAEVKKPGASVKVSCKASGYTFTNYGMNWVRQAPGQGLKWMGWINTYTGE |
| | | PTYADAFKGRVTMTRDTSISTAYMELSRLRSDDTAVYYCARDYGDYGMDYWGQGTTVTV |
| | | SS |
| 223 | CD70 VL | DIVMTQSPDSLAVSLGERATINCRASKSVSTSGYSFMHWYQQKPGQPPKLLIYLASNLES |
| | | GVPDRFSGSGSGTDFTLTISSLQAEDVAVYYCQHSREVPWTFGQGTKVEIK |
| 224 | B7H4 CDR-H1 | SGYSWH |
| 225 | B7H4 CDR-H2 | YIHSSGSTNYNPSLKS |
| 226 | B7H4 CDR-H3 | YDDYFEY |
| 227 | B7H4 CDR-L1 | KASQNVGFNVA |
| 228 | B7H4 CDR-L2 | SASYRYS |
| 229 | B7H4 CDR-L3 | QQYNWYPFT |
| 230 | B7H4 VH | EVQLQESGPGLVKPSETLSLTCAVTGYSITSGYSWHWIRQFPGNGLEWMGYIHSSGSTN |
| | | YNPSLKSRISISRDTSKNQFFLKLSSVTAADTAVYYCAGYDDYFEYWGQGTTVTVSS |
| 231 | B7H4 VL | DIQMTQSPSSLSASVGDRVTITCKASQNVGFNVAWYQQKPGKSPKALIYSASYRYSGVPS |
| | | RFSGSGSGTDFTLTISSLQPEDFAEYFCQQYNWYPFTFGQGTKLEIK |
| 232 | CD138 CDR-H1 | NYWIE |
| 233 | CD138 CDR-H2 | EILPGTGRTIYNEKFKG |
| 234 | CD138 CDR-H3 | RDYYGNFYYAMDY |
| 235 | CD138 CDR-L1 | SASQGINNYLN |
| 236 | CD138 CDR-L2 | YTSTLQS |
| 237 | CD138 CDR-L3 | QQYSKLPRT |
| 238 | CD138 VH | QVQLQQSGSELMMPGASVKISCKATGYTFSNYWIEWVKQRPGHGLEWIGEILPGTGRTIY |
| | | NEKFKGKATFTADISSNTVQMQLSSLTSEDSAVYYCARRDYYGNFYYAMDYWGQGTSVT |
| | | VSS |
| 239 | CD138 VL | DIQMTQSTSSLSASLGDRVTISCSASQGINNYLNWYQQKPDGTVELLIYYTSTLQSGVP |
| | | SRFSGSGSGTDYSLTISNLEPEDIGTYYCQQYSKLPRTFGGGTKLEIK |
| 240 | CD166 CDR-H1 | TYGMGVG |
| 241 | CD166 CDR-H2 | NIWWSEDKHYSPSLKS |
| 242 | CD166 CDR-H3 | IDYGNDYAFTY |
| 243 | CD166 CDR-L1 | RSSKSLLHSNGITYLY |
| 244 | CD166 CDR-L2 | QMSNLAS |
| 245 | CD166 CDR-L3 | AQNLELPYT |
| 246 | CD166 VH | QITLKESGPTLVKPTQTLTLTCTFSGFSLSTYGMGVGWIRQPPGKALEWLANIWWSEDKH |
| | | YSPSLKSRLTITKDTSKNQWVLTITNVDPVDTATYYCVQIDYGNDYAFTYWGQGTLVTVSS |
| 247 | CD166 VL | DIVMTQSPLSLPVTPGEPASISCRSSKSLLHSNGITYLYWYLQKPGQSPQLLIYQMSNLAS |
| | | GVPDRFSGSGSGTDFTLKISRVEAEDVGVYYCAQNLELPYTFGQGTKLEIK |
| 248 | CD51 CDR-H1 | RYTMH |
| 249 | CD51 CDR-H2 | VISFDGSNKYYVDSVKG |
| 250 | CD51 CDR-H3 | EARGSYAFDI |
| 251 | CD51 CDR-L1 | RASQSVSSYLA |
| 252 | CD51 CDR-L2 | DASNRAT |
| 253 | CD51 CDR-L3 | QQRSNWPPFT |
| 254 | CD51 VH | QVQLVESGGGVVQPGRSRRLSCAASGFTFSRYTMHWVRQAPGKGLEWVAVISFDGSNK |
| | | YYVDSVKGRFTISRDNSENTLYLQVNILRAEDTAVYYCAREARGSYAFDIWGQGTMVTVS |
| | | S |
| 255 | CD51 VL | EIVLTQSPATLSLSPGERATLSCRASQSVSSYLAWYQQKPGQAPRLLIYDASNRATGIPAR |
| | | FSGSGSGTDFTLTISSLEPEDFAVYYCQQRSNWPPFTFGPGTKVDIK |
| 256 | CD56 CDR-H1 | SFGMH |
| 257 | CD56 CDR-H2 | YISSGSFTIYYADSVKG |
| 258 | CD56 CDR-H3 | MRKGYAMDY |
| 259 | CD56 CDR-L1 | RSSQIIIHSDGNTYLE |
| 260 | CD56 CDR-L2 | KVSNRFS |
| 261 | CD56 CDR-L3 | FQGSHVPHT |
| 262 | CD56 VH | QVQLVESGGGVVQPGRSLRLSCAASGFTFSSFGMHWVRQAPGKGLEWVAYISSGSFTIY |
| | | YADSVKGRFTISRDNSKNTLYLQMNSLRAEDTAVYYCARMRKGYAMDYWGQGTLVTVSS |
| 263 | CD56 VL | DVVMTQSPLSLPVTLGQPASISCRSSQIIIHSDGNTYLEWFQQRPGQSPRRLIYKVSNRFS |
| | | GVPDRFSGSGSGTDFTLKISRVEAEDVGVYYCFQGSHVPHTFGQGTKVEIK |
| 264 | CD74 CDR-H1 | NYGVN |
| 265 | CD74 CDR-H2 | WINPNTGEPTFDDDFKG |
| 266 | CD74 CDR-H3 | SRGKNEAWFAY |
| 267 | CD74 CDR-L1 | RSSQSLVHRNGNTYLH |
| 268 | CD74 CDR-L2 | TVSNRFS |
| 269 | CD74 CDR-L3 | SQSSHVPPT |
| 270 | CD74 VH | QVQLQQSGSELKKPGASVKVSCKASGYTFTNYGVNWIKQAPGQGLQWMGWINPNTGEP |
| | | TFDDDFKGRFAFSLDTSVSTAYLQISSLKADDTAVYFCSRSRGKNEAWFAYWGQGTLVTV |
| | | SS |
| 271 | CD74 VL | DIQLTQSPLSLPVTLGQPASISCRSSQSLVHRNGNTYLHWFQQRPGQSPRLLIYTVSNRFS |
| | | GVPDRFSGSGSGTDFTLKISRVEAEDVGVYFCSQSSHVPPTFGAGTRLEIK |
| 272 | CEACAM5 | TYWMS |
| | CDR-H1 | |
| 273 | CEACAM5 | EIHPDSSTINYAPSLKD |
| | CDR-H2 | |
| 274 | CEACAM5 | LYFGFPWFAY |
| | CDR-H3 | |
| 275 | CEACAM5 | KASQDVGTSVA |
| | CDR-L1 | |
| 276 | CEACAM5 | WTSTRHT |
| | CDR-L2 | |
| 277 | CEACAM5 | QQYSLYRS |
| | CDR-L3 | |
| 278 | CEACAM5 VH | EVQLVESGGGVVQPGRSLRLSCSASGFDFTTYWMSWVRQAPGKGLEWIGEIHPDSSTIN |
| | | YAPSLKDRFTISRDNAKNTLFLQMDSLRPEDTGVYFCASLYFGFPWFAYWGQGTPVTVSS |
| 279 | CEACAM5 VL | DIQLTQSPSSLSASVGDRVTITCKASQDVGTSVAWYQQKPGKAPKLLIYWTSTRHTGVPS |
| | | RFSGSGSGTDFTFTISSLQPEDIATYYCQQYSLYRSFGQGTKVEIK |
| 280 | CanAg CDR-H1 | YYGMN |
| 281 | CanAg CDR-H2 | WIDTTTGEPTYAQKFQG |
| 282 | CanAg CDR-H3 | RGPYNWYFDV |
| 283 | CanAg CDR-L1 | RSSKSLLHSNGNTYLY |
| 284 | CanAg CDR-L2 | RMSNLVS |
| 285 | CanAg CDR-L3 | LQHLEYPFT |
| 286 | CanAg VH | QVQLVQSGAEVKKPGETVKISCKASDYTFTYYGMNWVKQAPGQGLKWMGWIDTTTGEP |
| | | TYAQKFQGRIAFSLETSASTAYLQIKSLKSEDTATYFCARRGPYNWYFDVWGQGTTVTVS |
| | | S |
| 287 | CanAg VL | DIVMTQSPLSVPVTPGEPVSISCRSSKSLLHSNGNTYLYWFLQRPGQSPQLLIYRMSNLVS |
| | | GVPDRFSGSGSGTAFTLRISRVEAEDVGVYYCLQHLEYPFTFGPGTKLELK |
| 288 | DLL-3 CDR-H1 | NYGMN |
| 289 | DLL-3 CDR-H2 | WINTYTGEPTYADDFKG |
| 290 | DLL-3 CDR-H3 | IGDSSPSDY |
| 291 | DLL-3 CDR-L1 | KASQSVSNDVV |
| 292 | DLL-3 CDR-L2 | YASNRYT |
| 293 | DLL-3 CDR-L3 | QQDYTSPWT |
| 294 | DLL-3 VH | QVQLVQSGAEVKKPGASVKVSCKASGYTFTNYGMNWVRQAPGQGLEWMGWINTYTGE |
| | | PTY |
| | | ADDFKGRVTMTTDTSTSTAYMELRSLRSDDTAVYYCARIGDSSPSDYWGQGTLVTVSS |
| 295 | DLL-3 VL | EIVMTQSPATLSVSPGERATLSCKASQSVSNDVVWYQQKPGQAPRLLIYYASNRYTGIPA |
| | | RFSGSGSGTEFTLTISSLQSEDFAVYYCQQDYTSPWTFGQGTKLEIK |
| 296 | DPEP-3 CDR- | SYWIE |
| | H1 | |
| 297 | DPEP-3 CDR- | EILPGSGNTYYNERFKD |
| | H2 | |
| 298 | DPEP-3 CDR- | RAAAYYSNPEWFAY |
| | H3 | |
| 299 | DPEP-3 CDR-L1 | TASSSVNSFYLH |
| 300 | DPEP-3 CDR-L2 | STSNLAS |
| 301 | DPEP-3 CDR-L3 | HQYHRSPYT |
| 302 | DPEP-3 VH | QVQLVQSGAEVKKPGSSVKVSCKASGGTFSSYWIEWVRQAPGQGLEWMGEILPGSGNT |
| | | YYNERFKDRVTITADESTSTAYMELSSLRSEDTAVYYCARRAAAYYSNPEWFAYWGQGT |
| | | LVTVSS |
| 303 | DPEP-3 VL | EIVLTQSPATLSLSPGERATLSCTASSSVNSFYLHWYQQKPGLAPRLLIYSTSNLASGIPDR |
| | | FSGSGSGTDFTLTISRLEPEDFAVYYCHQYHRSPYTFGQGTKLEIK |
| 304 | EGFR CDR-H1 | SYWMQ |
| 305 | EGFR CDR-H2 | TIYPGDGDTTYTQKFQG |
| 306 | EGFR CDR-H3 | YDAPGYAMDY |
| 307 | EGFR CDR-L1 | RASQDINNYLA |
| 308 | EGFR CDR-L2 | YTSTLHP |
| 309 | EGFR CDR-L3 | LQYDNLLYT |
| 310 | EGFR VH | QVQLVQSGAEVAKPGASVKLSCKASGYTFTSYWMQWVKQRPGQGLECIGTIYPGDGDTT |
| | | YTQKFQGKATLTADKSSSTAYMQLSSLRSEDSAVYYCARYDAPGYAMDYWGQGTLVTVS |
| | | S |
| 311 | EGFR VL | DIQMTQSPSSLSASVGDRVTITCRASQDINNYLAWYQHKPGKGPKLLIHYTSTLHPGIPSR |
| | | FSGSGSGRDYSFSISSLEPEDIATYYCLQYDNLLYTFGQGTKLEIK |
| 312 | EGFR CDR-H1 | RDFAWN |
| 313 | EGFR CDR-H2 | YISYNGNTRYQPSLKS |
| 314 | EGFR CDR-H3 | ASRGFPY |
| 315 | EGFR CDR-L1 | HSSQDINSNIG |
| 316 | EGFR CDR-L2 | HGTNLDD |
| 317 | EGFR CDR-L3 | VQYAQFPWT |
| 318 | EGFR VH | EVQLQESGPGLVKPSQTLSLTCTVSGYSISRDFAWNWIRQPPGKGLEWMGYISYNGNTR |
| | | YQPSLKSRITISRDTSKNQFFLKLNSVTAADTATYYCVTASRGFPYWGQGTLVTVSS |
| 319 | EGFR VL | DIQMTQSPSSMSVSVGDRVTITCHSSQDINSNIGWLQQKPGKSFKGLIYHGTNLDDGVPS |
| | | RFSGSGSGTDYTLTISSLQPEDFATYYCVQYAQFPWTFGGGTKLEIK |
| 320 | EGFR CDR-H1 | RDFAWN |
| 321 | EGFR CDR-H2 | YISYNGNTRYQPSLKS |
| 322 | EGFR CDR-H3 | ASRGFPY |
| 323 | EGFR CDR-L1 | HSSQDINSNIG |
| 324 | EGFR CDR-L2 | HGTNLDD |
| 325 | EGFR CDR-L3 | VQYAQFPWT |
| 326 | EGFR VH | EVQLQESGPGLVKPSQTLSLTCTVSGYSISRDFAWNWIRQPPGKGLEWMGYISYNGNTR |
| | | YQPSLKSRITISRDTSKNQFFLKLNSVTAADTATYYCVTASRGFPYWGQGTLVTVSS |
| 327 | EGFR VL | DIQMTQSPSSMSVSVGDRVTITCHSSQDINSNIGWLQQKPGKSFKGLIYHGTNLDDGVPS |
| | | RFSGSGSGTDYTLTISSLQPEDFATYYCVQYAQFPWTFGGGTKLEIK |
| 328 | EGFR CDR-H1 | NYGVH |
| 329 | EGFR CDR-H2 | VIWSGGNTDYNTPFTS |
| 330 | EGFR CDR-H3 | ALTYYDYEFAY |
| 331 | EGFR CDR-L1 | RASQSIGTNIH |
| 332 | EGFR CDR-L2 | YASESIS |
| 333 | EGFR CDR-L3 | QQNNNWPTT |
| 334 | EGFR VH | QVQLKQSGPGLVQPSQSLSITCTVSGFSLTNYGVHWVRQSPGKGLEWLGVIWSGGNTDY |
| | | NTPFTSRLSINKDNSKSQVFFKMNSLQSNDTAIYYCARALTYYDYEFAYWGQGTLVTVSA |
| 335 | EGFR VL | DILLTQSPVILSVSPGERVSFSCRASQSIGTNIHWYQQRTNGSPRLLIKYASESISGIPSRFS |
| | | GSGSGTDFTLSINSVESEDIADYYCQQNNNWPTTFGAGTKLELK |
| 336 | FRa CDR-H1 | GYFMN |
| 337 | FRa CDR-H2 | RIHPYDGDTFYNQKFQG |
| 338 | FRa CDR-H3 | YDGSRAMDY |
| 339 | FRa CDR-L1 | KASQSVSFAGTSLMH |
| 340 | FRa CDR-L2 | RASNLEA |
| 341 | FRa CDR-L3 | QQSREYPYT |
| 342 | FRa VH | QVQLVQSGAEVVKPGASVKISCKASGYTFTGYFMNWVKQSPGQSLEWIGRIHPYDGDTF |
| | | Y |
| | | NQKFQGKATLTVDKSSNTAHMELLSLTSEDFAVYYCTRYDGSRAMDYWGQGTTVTVSS |
| 343 | FRa VL | DIVLTQSPLSLAVSLGQPAIISCKASQSVSFAGTSLMHWYHQKPGQQPRLLIYRASNLEAG |
| | | VPDRFSGSGSKTDFTLTISPVEAEDAATYYCQQSREYPYTFGGGTKLEIK |
| 344 | FRa CDR-H1 | GYGLS |
| 345 | FRa CDR-H2 | MISSGGSYTYYADSVKG |
| 346 | FRa CDR-H3 | HGDDPAWFAY |
| 347 | FRa CDR-L1 | SVSSSISSNNLH |
| 348 | FRa CDR-L2 | GTSNLAS |
| 349 | FRa CDR-L3 | QQWSSYPYMYT |
| 350 | FRa VH | EVQLVESGGGWQPGRSLRLSCSASGFTFSGYGLSWVRQAPGKGLEWVAMISSGGSYT |
| | | YY |
| | | ADSVKGRFAISRDNAKNTLFLQMDSLRPEDTGVYFCARHGDDPAWFAYWGQGTPVTVSS |
| 351 | FRa VL | DIQLTQSPSSLSASVGDRVTITCSVSSSISSNNLHWYQQKPGKAPKPWIYGTSNLASGVPS |
| | | RFSGSGSGTDYTFTISSLQPEDIATYYCQQWSSYPYMYTFGQGTKVEIK |
| 352 | MUC-1 CDR-H1 | NYWMN |
| 353 | MUC-1 CDR-H2 | EIRLKSNNYTTHYAESVKG |
| 354 | MUC-1 CDR-H3 | HYYFDY |
| 355 | MUC-1 CDR-L1 | RSSKSLLHSNGITYFF |
| 356 | MUC-1 CDR-L2 | QMSNLAS |
| 357 | MUC-1 CDR-L3 | AQNLELPPT |
| 358 | MUC-1 VH | EVQLVESGGGLVQPGGSMRLSCVASGFPFSNYWMNWVRQAPGKGLEWVGEIRLKSNN |
| | | YTTHYAESVKGRFTISRDDSKNSLYLQMNSLKTEDTAVYYCTRHYYFDYWGQGTLVTVSS |
| 359 | MUC-1 VL | DIVMTQSPLSNPVTPGEPASISCRSSKSLLHSNGITYFFWYLQKPGQSPQLLIYQMSNLAS |
| | | GVPDRFSGSGSGTDFTLRISRVEAEDVGVYYCAQNLELPPTFGQGTKVEIK |
| 360 | Mesothelin | SYWIG |
| | CDR-H1 | |
| 361 | Mesothelin | IIDPGDSRTRYSPSFQG |
| | CDR-H2 | |
| 362 | Mesothelin | GQLYGGTYMDG |
| | CDR-H3 | |
| 363 | Mesothelin | TGTSSDIGGYNSVS |
| | CDR-L1 | |
| 364 | Mesothelin | GVNNRPS |
| | CDR-L2 | |
| 365 | Mesothelin | SSYDIESATPV |
| | CDR-L3 | |
| 366 | Mesothelin VH | QVELVQSGAEVKKPGESLKISCKGSGYSFTSYWIGWVRQAPGKGLEWMGIIDPGDSRTR |
| | | YSPSFQGQVTISADKSISTAYLQWSSLKASDTAMYYCARGQLYGGTYMDGWGQGTLVTV |
| | | SS |
| 367 | Mesothelin VL | DIALTQPASVSGSPGQSITISCTGTSSDIGGYNSVSWYQQHPGKAPKLMIYGVNNRPSGV |
| | | SNRFSGSKSGNTASLTISGLQAEDEADYYCSSYDIESATPVFGGGTKLTVL |
| 368 | ROR-1 CDR-H1 | AYNIH |
| 369 | ROR-1 CDR-H2 | SFDPYDGGSSYNQKFKD |
| 370 | ROR-1 CDR-H3 | GWYYFDY |
| 371 | ROR-1 CDR-L1 | RASKSISKYLA |
| 372 | ROR-1 CDR-L2 | SGSTLQS |
| 373 | ROR-1 CDR-L3 | QQHDESPYT |
| 374 | ROR-1 VH | QVQLQESGPGLVKPSQTLSLTCTVSGYAFTAYNIHWVRQAPGQGLEWMGSFDPYDGGS |
| | | SYNQKFKDRLTISKDTSKNQWVLTMTNMDPVDTATYYCARGWYYFDYWGHGTLVTVSS |
| 375 | ROR-1 VL | DIVMTQTPLSLPVTPGEPASISCRASKSISKYLAWYQQKPGQAPRLLIYSGSTLQSGIPPRF |
| | | SGSGYGTDFTLTINNIESEDAAYYFCQQHDESPYTFGEGTKVEIK |
| 376 | B7H4 CDR-H1 | GSIKSGSYYWG |
| 377 | B7H4 CDR-H2 | NIYYSGSTYYNPSLRS |
| 378 | B7H4 CDR-H3 | AREGSYPNQFDP |
| 379 | B7H4 CDR-L1 | RASQSVSSNLA |
| 380 | B7H4 CDR-L2 | GASTRAT |
| 381 | B7H4 CDR-L3 | QQYHSFPFT |
| 382 | B7H4 VH | QLQLQESGPGLVKPSETLSLTCTVSGGSIKSGSYYWGWIRQPPGKGLEWIGNIYYSGSTY |
| | | YNPSLRSRVTISVDTSKNQFSLKLSSVTAADTAVYYCAREGSYPNQFDPWGQGTLVTVSS |
| 383 | B7H4 VL | EIVMTQSPATLSVSPGERATLSCRASQSVSSNLAWYQQKPGQAPRLLIYGASTRATGIPA |
| | | RFSGSGSGTEFTLTISSLQSEDFAVYYCQQYHSFPFTFGGGTKVEIK |
| 384 | B7-H3 CDR-H1 | SFGMH |
| 385 | B7-H3 CDR-H2 | YISSDSSAIYY |
| 386 | B7-H3 CDR-H3 | GRENIYYGSRLD |
| 387 | B7-H3 CDR-L1 | KASQNVD |
| 388 | B7-H3 CDR-L2 | SASYRYSGVPD |
| 389 | B7-H3 CDR-L3 | QQYNNYPFTFGS |
| 390 | B7-H3 VH | DVQLVESGGGLVQPGGSRKLSCAASGFTFSSFGMHWVRQAPEKGLEWVAYISSDSSAIY |
| | | Y |
| | | ADTVKGRFTISRDNPKNTLFLQMTSLRSEDTAMYYCGRGRENIYYGSRLDYWGQGTTLTV |
| | | SS |
| 391 | B7-H3 VL | DIAMTQSQKFMSTSVGDRVSVTCKASQNVDTNVAWYQQKPGQSPKALIYSASYRYSGVP |
| | | D |
| | | RFTGSGSGTDFTLTINNVQSEDLAEYFCQQYNNYPFTFGSGTKLEIK |
| 392 | B7-H3 CDR-H1 | SYWMQWVRQA |
| 393 | B7-H3 CDR-H2 | TIYPGDGDTRY |
| 394 | B7-H3 CDR-H3 | RGIPRLWYFDVM |
| 395 | B7-H3 CDR-L1 | ITCRASQDIS |
| 396 | B7-H3 CDR-L2 | YTSRLHSGVPS |
| 397 | B7-H3 CDR-L3 | QQGNTLPPFTGG |
| 398 | B7-H3 VH | DVQLVESGGGLVQPGGSRKLSCAASGFTFSSFGMHWVRQAPEKGLEWVAYISSDSSAIY |
| | | Y |
| | | ADTVKGRFTISRDNPKNTLFLQMTSLRSEDTAMYYCGRGRENIYYGSRLDYWGQGTTLTV |
| | | SS |
| 399 | B7-H3 VL | DIAMTQSQKFMSTSVGDRVSVTCKASQNVDTNVAWYQQKPGQSPKALIYSASYRYSGVP |
| | | D |
| | | RFTGSGSGTDFTLTINNVQSEDLAEYFCQQYNNYPFTFGSGTKLEIK |
| 400 | B7-H3 CDR-H1 | SYGMSWVRQA |
| 401 | B7-H3 CDR-H2 | INSGGSNTYY |
| 402 | B7-H3 CDR-H3 | HDGGAMDYW |
| 403 | B7-H3 CDR-L1 | ITCRASESIYSYLA |
| 404 | B7-H3 CDR-L2 | NTKTLPE |
| 405 | B7-H3 CDR-L3 | HHYGTPPWTFG |
| 406 | B7-H3 VH | EVQLVESGGGLVKPGGSLRLSCAASGFTFSSYGMSWVRQAPGKGLEWVATINSGGSNT |
| | | YY |
| | | PDSLKGRFTISRDNAKNSLYLQMNSLRAEDTAVYYCARHDGGAMDYWGQGTTVTVSS |
| 407 | B7-H3 VL | DIQMTQSPSSLSASVGDRVTITCRASESIYSYLAWYQQKPGKAPKLLVYNTKTLPEGVPSR |
| | | FSGSGSGTDFTLTISSLQPEDFATYYCQHHYGTPPWTFGQGTRLEIK |
| 408 | B7-H3 CDR-H1 | SFGMHWVRQA |
| 409 | B7-H3 CDR-H2 | ISSGSGTIYYADTVKGRFTI |
| 410 | B7-H3 CDR-H3 | HGYRYEGFDYWG |
| 411 | B7-H3 CDR-L1 | ITCKASQNVDTNVA |
| 412 | B7-H3 CDR-L2 | SASYRYSGVPS |
| 413 | B7-H3 CDR-L3 | QQYNNYPFTFGQ |
| 414 | B7-H3 VH | EVQLVESGGGLVQPGGSLRLSCAASGFTFSSFGMHWVRQAPGKGLEWVAYISSGSGTIY |
| | | YADTVKGRFTISRDNAKNSLYLQMNSLRAEDTAVYYCARHGYRYEGFDYWGQGTTVTVS |
| | | S |
| 415 | B7-H3 VL | DIQMTQSPSFLSASVGDRVTITCKASQNVDTNVAWYQQKPGKAPKALIYSASYRYSGVPS |
| | | RFSGSGSGTDFTLTISSLQPEDFAEYFCQQYNNYPFTFGQGTKLEIK |
| 416 | B7-H3 CDR-H1 | NYVMH |
| 417 | B7-H3 CDR-H2 | YINPYNDDVKYNEKFKG |
| 418 | B7-H3 CDR-H3 | WGYYGSPLYYFDY |
| 419 | B7-H3 CDR-L1 | RASSRLIYMH |
| 420 | B7-H3 CDR-L2 | ATSNLAS |
| 421 | B7-H3 CDR-L3 | QQWNSNPPT |
| 422 | B7-H3 VH | EVQLQQSGPELVKPGASVKMSCKASGYTFTNYVMHWVKQKPGQGLEWIGYINPYNDDV |
| | | KYNEKFKGKATQTSDKSSSTAYMELSSLTSEDSAVYYCARWGYYGSPLYYFDYWGQGTT |
| | | LTVSS |
| 423 | B7-H3 VL | QIVLSQSPTILSASPGEKVTMTCRASSRLIYMHWYQQKPGSSPKPWIYATSNLASGVPAR |
| | | FSGSGSGTSYSLTISRVEAEDAATYYCQQWNSNPPTFGTGTKLELK |
| 424 | B7-H3 CDR-H1 | NYVMH |
| 425 | B7-H3 CDR-H2 | YINPYNDDVKYNEKFKG |
| 426 | B7-H3 CDR-H3 | WGYYGSPLYYFDY |
| 427 | B7-H3 CDR-L1 | RASSRLIYMH |
| 428 | B7-H3 CDR-L2 | ATSNLAS |
| 429 | B7-H3 CDR-L3 | QQWNSNPPT |
| 430 | B7-H3 VH | QVQLVQSGAEVKKPGSSVKVSCKASGYTFTNYVMHWVRQAPGQGLEWMGYINPYNDDV |
| | | KYNE |
| | | KFKGRVTITADESTSTAYMELSSLRSEDTAVYYCARWGYYGSPLYYFDYWGQGTLVTVSS |
| 431 | B7-H3 VL | EIVLTQSPATLSLSPGERATLSCRASSRLIYMHWYQQKPGQAPRPLIYATSNLASGIPARFS |
| | | GSGSGTDFTLTISSLEPEDFAVYYCQQWNSNPPTFGQGTKVEIK |
| 432 | B7-H3 CDR-H1 | GYSFTSYTIH |
| 433 | B7-H3 CDR-H2 | YINPNSRNTDYAQKFQG |
| 434 | B7-H3 CDR-H3 | YSGSTPYWYFDV |
| 435 | B7-H3 CDR-L1 | RASSSVSYMN |
| 436 | B7-H3 CDR-L2 | ATSNLAS |
| 437 | B7-H3 CDR-L3 | QQWSSNPLT |
| 438 | B7-H3 VH | EVQLVQSGAEVKKPGSSVKVSCKASGYSFTSYTIHWVRQAPGQGLEWMGYINPNSRNTD |
| | | YAQKFQGRVTLTADKSTSTAYMELSSLRSEDTAVYYCARYSGSTPYWYFDVWGQGTTVT |
| | | VSS |
| 439 | B7-H3 VL | DIQMTQSPSSLSASVGDRVTITCKASQNVGFNVAWYQQKPGKSPKALIYSASYRYSGVPS |
| | | RFSGSGSGTDFTLTISSLQPEDFAEYFCQQYNWYPFTFGQGTKLEIK |
| 440 | B7-H3 CDR-H1 | GYTFSSYWMH |
| 441 | B7-H3 CDR-H2 | LIHPDSGSTNYNEMFKN |
| 442 | B7-H3 CDR-H3 | GGRLYFD |
| 443 | B7-H3 CDR-L1 | RSSQSLVHSNGDTYLR |
| 444 | B7-H3 CDR-L2 | KVSNRFS |
| 445 | B7-H3 CDR-L3 | SQSTHVPYT |
| 446 | B7-H3 VH | EVQLVQSGAEVKKPGSSVKVSCKASGYTFSSYWMHWVRQAPGQGLEWIGLIHPDSGST |
| | | NYNEMFKNRATLTVDRSTSTAYVELSSLRSEDTAVYFCAGGGRLYFDYWGQGTTVTVSS |
| 447 | B7-H3 VL | DWVMTQSPLSLPVTPGEPASISCRSSQSLVHSNGDTYLRWYLQKPGQSPQLLIYKVSNRF |
| | | SGVPDRFSGSGSGTDFTLKISRVEAEDVGVYYCSQSTHVPYTFGGGTKVEIK |
| 448 | B7-H3 CDR-H1 | GYTFSSYWMH |
| 449 | B7-H3 CDR-H2 | LIHPESGSTNYNEMFKN |
| 450 | B7-H3 CDR-H3 | GGRLYFDY |
| 451 | B7-H3 CDR-L1 | RSSQSLVHSNQDTYLR |
| 452 | B7-H3 CDR-L2 | KVSNRFS |
| 453 | B7-H3 CDR-L3 | SQSTHVPYT |
| 454 | B7-H3 VH | EVQLVQSGAEVKKPGSSVKVSCKASGYTFSSYWMHWVRQAPGQGLEWIGLIHPESGST |
| | | NY |
| | | NEMFKNRATLTVDRSTSTAYMELSSLRSEDTAVYYCAGGGRLYFDYWGQGTTVTVSS |
| 455 | B7-H3 VL | DIVMTQSPLSLPVTPGEPASISCRSSQSLVHSNQDTYLRWYLQKPGQSPQLLIYKVSNRF |
| | | SGVPDRFSGSGSGTDFTLKKISRVEAEDVGVYYCSQSTHVPYTFGGGTKVEIK |
| 456 | B7-H3 CDR-H1 | TGYSITSGYSWH |
| 457 | B7-H3 CDR-H2 | YIHSSGSTNYNPSLKS |
| 458 | B7-H3 CDR-H3 | YDDYFEY |
| 459 | B7-H3 CDR-L1 | KASQNVGFNVAW |
| 460 | B7-H3 CDR-L2 | SASYRYS |
| 461 | B7-H3 CDR-L3 | QQYNWYPFT |
| 462 | B7-H3 VH | EVQLQESGPGLVKPSETLSLTCAVTGYSITSGYSWHWIRQFPGNGLEWMGYIHSSGSTN |
| | | Y |
| | | NPSLKSRISISRDTSKNQFFLKLSSVTAADTAVYYCAGYDDYFEYWGQGTTVTVSS |
| 463 | B7-H3 VL | DIQMTQSPSSLSASVGDRVTITCKASQNVGGFNVAWYQQKPGKSPKALIYSASYRYSGV |
| | | PSRFSGSGSGTDFTLTISSLQPEDFAEYFCQQYNWYPFTFGQGTKLEIK |
| 464 | B7-H3 CDR-H1 | NYDIN |
| 465 | B7-H3 CDR-H2 | WIGWIFPGDDSTQYNEKFKG |
| 466 | B7-H3 CDR-H3 | QTTGTWFAY |
| 467 | B7-H3 CDR-L1 | RASQSISDYLY |
| 468 | B7-H3 CDR-L2 | YASQSIS |
| 469 | B7-H3 CDR-L3 | CQNGHSFPL |
| 470 | B7-H3 VH | QVQLVQSGAEVVKPGASVKLSCKTSGYTFTNYDINWVRQRPGQGLEWIGWIFPGDDSTQ |
| | | Y |
| | | NEKFKGKATLTTDTSTSTAYMELSSLRSEDTAVYFCARQTTGTWFAYWGQGTLVTVSS |
| 471 | B7-H3 VL | EIVMTQSPATLSVSPGERVTLSCRASQSISDYLYWYQQKSHESPRLLIKYASQSISGIPA |
| | | RFSGSGSGSEFTLTINSVEPEDVGVYYCQNGHSFPLTFGQGTKLELK |
| 472 | B7-H3 VH | QVQLQQSGAEVKKPGSSVKVSCKASGGTFSSYAISWVRQAPGQGLEWMGGIIPILGIAN |
| | | YAQKFQGRVTITADESTSTAYMELSSLRSEDTAVYYCARGGSGSYHMDVWGKGTTVTVS |
| | | S |
| 473 | B7-H3 VL | EIVLTQSPATLSLSPGERATLSCRASQSVSSYLAWYQQKPGQAPRLLIYDASNRATGIP |
| | | ARFSGSGSGTDFTLTISSLEPEDFAVYYCQQRSNWPPRITFGQGTRLEIK |
| 474 | B7-H3 CDR-H1 | IYNVH |
| 475 | B7-H3 CDR-H2 | TIFPGNGDTSYNQKFKD |
| 476 | B7-H3 CDR-H3 | WDDGNVGFAH |
| 477 | B7-H3 CDR-L1 | RASENINNYLT |
| 478 | B7-H3 CDR-L2 | HAKTLAE |
| 479 | B7-H3 CDR-L3 | QHHYGTPPT |
| 480 | B7-H3 VH | QVQLQQPGAELVKPGASVKMSCKASGYTFTIYNVHWIKQTPGQGLEWMGTIFPGNGDTS |
| | | Y |
| | | NQKFKDKATLTTDKSSKTAYMQLNSLTSEDSAVYYCARWDDGNVGFAHWGQGTLVTVS |
| | | A |
| 481 | B7-H3 VL | DIQMTQSPASLSASVGETVTITCRASENINNYLTWFQQKQGKSPQLLVYHAKTLAEGVPS |
| | | RFSGSGSGTQFSLKINSLQPEDFGSYYCQHHYGTPPTFGGGTKLEIK |
| 482 | B7-H3 VH | EVQLVQSGAEVKKPGASVKVSCKASGYTFTIYNVHWVRQAPGQGLEWMGTIFPGNGDTS |
| | | YNQKFKDKVTMTTDTSTSTAYMELSSLRSEDTAVYYCARWDDGNVGFAHWGQGTLVTV |
| | | SS |
| 483 | B7-H3 VL | DIQMTQSPSSLSASVGDRVTITCRASENINNYLTWFQQKQGKSPQLLIYHAKTLAEGVP |
| | | SRFSGSGSGTDFTLTISSLQPEDFATYYCQHHYGTPPTFGGGTKVEIK |
| 484 | B7-H3 VH | EVQLVQSGAEVKKPGASVKVSCKASGYTFTIYNVHWIRQAPGQGLEWMGTIFPGNGDTS |
| | | Y |
| | | NQKFKDRATLTTDKSTKTAYMELRSLRSDDTAVYYCARWDDGNVGFAHWGQGTLVTVS |
| | | S |
| 485 | B7-H3 VL | DIQMTQSPSSLSASVGDRVTITCRASENINNYLTWFQQKPGKAPKLLVYHAKTLAEGVPS |
| | | RFSGSGSGTQFTLTISSLQPEDFATYYCQHHYGTPPTFGQGTKLEIK |
| 486 | HER3 H | QVQLQQWGAGLLKPSETLSLTCAVYGGSFSGYYWSWIRQPPGKGLEWIGEINHSGSTNY |
| | | N |
| | | PSLKSRVTISVETSKNQFSLKLSSVTAADTAVYYCARDKWTWYFDLWGRGTLVTVSSAST |
| | | KGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKRVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRWSVLTVLHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 487 | HER3 L | DIEMTQSPDSLAVSLGERATINCRSSQSVLYSSSNRNYLAWYQQNPGQPPKLLIYWASTR |
| | | ESGVPDRFSGSGSGTDFTLTISSLQAEDVAVYYCQQYYSTPRTFGQGTKVEIKRTVAAPS |
| | | VFIFPPSDEQLKSGTASVVCLLNNFYPREAKVQWKVDNALQSGNSQESVTEQDSKDSTYS |
| | | LSSTLTLSKADYEKHKVYACEVTHQGLSSPVTKSFNRGEC |
| 488 | HER3 H | EVQLLESGGGLVQPGGSLRLSCAASGFTFSHYVMAWVRQAPGKGLEWVSSISSSGGWT |
| | | LY |
| | | ADSVKGRFTISRDNSKNTLYLQMNSLRAEDTAVYYCTRGLKMATIFDYWGQGTLVTVSSA |
| | | STKGPSVFPLAPCSRSTSESTAALGCLVKDYFPEPVTVSWNSGALTSGVHTFPAVLQSSG |
| | | LYSLSSVVTVPSSNFGTQTYTCNVDHKPSNTKVDKTVERKCCVECPPCPAPPVAGPSVFL |
| | | FPPKPKDTLMISRTPEVTCWVVDVSHEDPEVQFNWYVDGVEVHNAKTKPREEQFNSTFR |
| | | V |
| | | VSVLTWVHQDWLNGKEYKCKVSNKGLPAPIEKTISKTKGQPREPQVYTLPPSREEMTKNQ |
| | | VSLTCLVKGFYPSDIAVEWESNGQPENNYKTTPPMLDSDGSFFLYSKLTVDKSRWQQGN |
| | | V |
| | | FSCSVMHEALHNHYTQKSLSLSPGK |
| 489 | HER3 L | QSALTQPASVSGSPGQSITISCTGTSSDVGSYNVVSWYQQHPGKAPKLIIYEVSQRPSGV |
| | | SNRFSGSKSGNTASLTISGLQTEDEADYYCCSYAGSSIFVIFGGGTKVTVLGQPKAAPSVT |
| | | LFPPSSEELQANKATLVCLVSDFYPGAVTVAWKADGSPVKVGVETTKPSKQSNNKYAASS |
| | | YLSLTPEQWKSHRSYSCRVTHEGSTVEKTVAPAECS |
| 490 | HER3 H | EVQLLESGGGLVQPGGSLRLSCAASGFTFSSYAMSWVRQAPGKGLEWVSAINSQGKSTY |
| | | YADSVKGRFTISRDNSKNTLYLQMNSLRAEDTAVYYCARWGDEGFDIWGQGTLVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKRVEPKSCDKTHTCPPCPAPELLGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 491 | HER3 L | DIQMTQSPSSLSASVGDRVTITCRASQGISNWLAWYQQKPGKAPKLLIYGASSLQSGVPS |
| | | RFSGSGSGTDFTLTISSLQPEDFATYYCQQYSSFPTTFGQGTKVEIKRTVAAPSVFIFPPSD |
| | | EQLKSGTASWVCLLNNFYPREAKVQWKVDNALQSGNSQESVTEQDSKDSTYSLSSTLTLS |
| | | KADYEKHKVYACEVTHQGLSSPVTKSFNRGEC |
| 492 | HER3 H | QVQLVQSGAEVKKPGASVKVSCKASGYTFRSSYISWVRQAPGQGLEWMGWIYAGTGSP |
| | | SYNQKLQGRVTMTTDTSTSTAYMELRSLRSDDTAVYYCARHRDYYSNSLTYWGQGTLVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPG |
| 493 | HER3 L | DIVMTQSPDSLAVSLGERATINCKSSQSVLNSGNQKNYLTWYQQKPGQPPKLLIYWASTR |
| | | ESGVPDRFSGSGSGTDFTLTISSLQAEDVAVYYCQSDYSYPYTFGQGTKLEIKRTVAAPSV |
| | | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSFNRGEC |
| 494 | PTK7 CDR-H1 | TSNMGVG |
| 495 | PTK7 CDR-H2 | HIWWDDDKYYSPSLKS |
| 496 | PTK7 CDR-H3 | SNYGYAWFAY |
| 497 | PTK7 CDR-L1 | KASQDIYPYLN |
| 498 | PTK7 CDR-L2 | RTNRLLD |
| 499 | PTK7 CDR-L3 | LQYDEFPLT |
| 500 | PTK7 VH | QITLKESGPTLVKPTQTLTLTCTFSGFSLSTSNMGVGWIRQPPGKALEWLAHIWWDDDKY |
| | | YSPSLKSRLTITKDTSKNQVVLTMTNMDPVDTATYYCVRSNYGYAWFAYWGQGTLVTVS |
| | | S |
| 501 | PTK7 VL | DIQMTQSPSSLSASVGDRVTITCKASQDIYPYLNWFQQKPGKAPKTLIYRTNRLLDGVPS |
| | | RFSGSGSGTDFTFTISSLQPEDIATYYCLQYDEFPLTFGAGTKLEIK |
| 502 | PTK7 CDR-H1 | DYAVH |
| 503 | PTK7 CDR-H2 | VISTYNDYTYNNQDFKG |
| 504 | PTK7 CDR-H3 | GNSYFYALDY |
| 505 | PTK7 CDR-L1 | RASESVDSYGKSFMH |
| 506 | PTK7 CDR-L2 | RASNLES |
| 507 | PTK7 CDR-L3 | QQSNEDPWT |
| 508 | PTK7 VH | QVQLVQSGPEVKKPGASVKVSCKASGYTFTDYAVHWVRQAPGKRLEWIGVISTYNDYTY |
| | | NNQDFKGRVTMTRDTSASTAYMELSRLRSEDTAVYYCARGNSYFYALDYWGQGTSVTV |
| | | SS |
| 509 | PTK7 VL | EIVLTQSPATLSLSPGERATLSCRASESVDSYGKSFMHWYQQKPGQAPRLLIYRASNLES |
| | | GIPARFSGSGSGTDFTLTISSLEPEDFAVYYCQQSNEDPWTFGGGTKLEIK |
| 510 | PTK7 CDR-H1 | RYWMS |
| 511 | PTK7 CDR-H2 | DLNPDSSAINYVDSVKG |
| 512 | PTK7 CDR-H3 | ITTLVPYTMDF |
| 513 | PTK7 CDR-L1 | ITNTDIDDDMN |
| 514 | PTK7 CDR-L2 | EGNGLRP |
| 515 | PTK7 CDR-L3 | LQSDNLPLT |
| 516 | PTK7 VH | EVQLVESGGGLVQPGGSLRLSCAASGFDFSRYWMSWVRQAPGKGLEWIGDLNPDSSAI |
| | | NY |
| | | VDSVKGRFTISRDNAKNSLYLQMNSLRAEDTAVYYCTLITTLVPYTMDFWGQGTSVTVSS |
| 517 | PTK7 VL | ETTLTQSPAFMSATPGDKVNISCITNTDIDDDMNWYQQKPGEAAILLISEGNGLRPGIPPRF |
| | | SGSGYGTDFTLTINNIESEDAAYYFCLQSDNLPLTFGSGTKLEIK |
| 518 | LIV1 CDR-H1 | DYYMH |
| 519 | LIV1 CDR-H2 | WIDPENGDTEYGPKFQG |
| 520 | LIV1 CDR-H3 | HNAHYGTWFAY |
| 521 | LIV1 CDR-L1 | RSSQSLLHSSGNTYLE |
| 522 | LIV1 CDR-L2 | KISTRFS |
| 523 | LIV1 CDR-L3 | FQGSHVPYT |
| 524 | LIV1 VH | QVQLVQSGAEVKKPGASVKVSCKASGLTIEDYYMHWVRQAPGQGLEWMGWIDPENGDT |
| | | EY |
| | | GPKFQGRVTMTRDTSINTAYMELSRLRSDDTAVYYCAVHNAHYGTWFAYWGQGTLVTVS |
| | | S |
| 525 | LIV1 VL | DWVMTQSPLSLPVTLGQPASISCRSSQSLLHSSGNTYLEWYQQRPGQSPRPLIYKISTRFS |
| | | GVPDRFSGSGSGTDFTLKISRVEAEDVGVYYCFQGSHVPYTFGGGTKVEIK |
| 526 | avb6 CDR-H1 | DYNVN |
| 527 | avb6 CDR-H2 | VINPKYGTTRYNQKFKG |
| 528 | avb6 CDR-H3 | GLNAWDY |
| 529 | avb6 CDR-L1 | GASENIYGALN |
| 530 | avb6 CDR-L2 | GATNLED |
| 531 | avb6 CDR-L3 | QNVLTTPYT |
| 532 | avb6 VH | QFQLVQSGAEVKKPGASVKVSCKASGYSFTDYNVNWVRQAPGQGLEWIGVINPKYGTTR |
| | | Y |
| | | NQKFKGRATLTVDKSTSTAYMELSSLRSEDTAVYYCTRGLNAWDYWGQGTLVTVSS |
| 533 | avb6 VL | DIQMTQSPSSLSASVGDRVTITCGASENIYGALNWYQQKPGKAPKLLIYGATNLEDGVPS |
| | | RFSGSGSGRDYTFTISSLQPEDIATYYCQNVLTTPYTFGQGTKLEIK |
| 534 | avb6 CDR-H1 | GYFMN |
| 535 | avb6 CDR-H2 | LINPYNGDSFYNQKFKG |
| 536 | avb6 CDR-H3 | GLRRDFDY |
| 537 | avb6 CDR-L1 | KSSQSLLDSDGKTYLN |
| 538 | avb6 CDR-L2 | LVSELDS |
| 539 | avb6 CDR-L3 | WQGTHFPRT |
| 540 | avb6 VH | QVQLVQSGAEVKKPGASVKVSCKASGYSFSGYFMNWVRQAPGQGLEWMGLINPYNGD |
| | | SFY |
| | | NQKFKGRVTMTRQTSTSTVYMELSSLRSEDTAVYYCVRGLRRDFDYWGQGTLVTVSS |
| 541 | avb6 VL | DWVMTQSPLSLPVTLGQPASISCKSSQSLLDSDGKTYLNWLFQRPGQSPRRLIYLVSELD |
| | | SGVPDRFSGSGSGTDFTLKISRVEAEDVGVYYCWQGTHFPRTFGGGTKLEIK |
| 542 | CD48 CDR-H1 | DFGMN |
| 543 | CD48 CDR-H2 | WINTFTGEPSYGNVFKG |
| 544 | CD48 CDR-H3 | RHGNGNVFDS |
| 545 | CD48 CDR-L1 | RASQSIGSNIH |
| 546 | CD48 CDR-L2 | YTSESIS |
| 547 | CD48 CDR-L3 | QQSNSWPLT |
| 548 | CD48 VH | QVQLVQSGSELKKPGASVKVSCKASGYTFTDFGMNWVRQAPGQGLEWMGWINTFTGEP |
| | | SYGNVFKGRFVFSLDTSVSTAYLQISSLKAEDTAVYYCARRHGNGNVFDSWGQGTLVTVS |
| | | S |
| 549 | CD48 VL | EIVLTQSPDFQSVTPKEKVTITCRASQSIGSNIHWYQQKPDQSPKLLIKYTSESISGVPSRF |
| | | SGSGSGTDFTLTINSLEAEDAATYYCQQSNSWPLTFGGGTKVEIKR |
| 550 | PD-L1 CDR-H1 | TAAIS |
| 551 | PD-L1 CDR-H2 | GIIPIFGKAHYAQKFQG |
| 552 | PD-L1 CDR-H3 | KFHFVSGSPFGMDV |
| 553 | PD-L1 CDR-L1 | RASQSVSSYLA |
| 554 | PD-L1 CDR-L2 | DASNRAT |
| 555 | PD-L1 CDR-L3 | QQRSNWPT |
| 556 | PD-L1 VH | QVQLVQSGAEVKKPGSSVKVSCKTSGDTFSTAAISWVRQAPGQGLEWMGGIIPIFGKAHY |
| | | AQKFQGRVTITADESTSTAYMELSSLRSEDTAVYFCARKFHFVSGSPFGMDVWGQGTTV |
| | | TVSS |
| 557 | PD-L1 VL | EIVLTQSPATLSLSPGERATLSCRASQSVSSYLAWYQQKPGQAPRLLIYDASNRATGIPA |
| | | RFSGSGSGTDFTLTISSLEPEDFAVYYCQQRSNWPTFGQGTKVEIK |
| 558 | IGF-1R CDR-H1 | SYAIS |
| 559 | IGF-1R CDR-H2 | GIIPIFGTANYAQKFQG |
| 560 | IGF-1R CDR-H3 | APLRFLEWSTQDHYYYYYMDV |
| 561 | IGF-1R CDR-L1 | QGDSLRSYYAT |
| 562 | IGF-1R CDR-L2 | GENKRPS |
| 563 | IGF-1R CDR-L3 | KSRDGSGQHLV |
| 564 | IGF-1R VH | EVQLVQSGAEVKKPGSSVKVSCKASGGTFSSYAISWVRQAPGQGLEWMGGIIPIFGTANY |
| | | AQKFQGRVTITADKSTSTAYMELSSLRSEDTAVYYCARAPLRFLEWSTQDHYYYYYMDV |
| | | WGKGTTVTVSS |
| 565 | IGF-1R VL | SSELTQDPAVSVALGQTVRITCQGDSLRSYYATWYQQKPGQAPILVIYGENKRPSGIPDR |
| | | FSGSSSGNTASLTITGAQAEDEADYYCKSRDGSGQHLVFGGGTKLTVL |
| 566 | Claudin-18.2 | SYWIN |
| | CDR-H1 | |
| 567 | Claudin-18.2 | NIYPSDSYTNYNQKFKD |
| | CDR-H2 | |
| 568 | Claudin-18.2 | SWRGNSFDY |
| | CDR-H3 | |
| 569 | Claudin-18.2 | KSSQSLLNSGNQKNYLT |
| | CDR-L1 | |
| 570 | Claudin-18.2 | WASTRES |
| | CDR-L2 | |
| 571 | Claudin-18.2 | QNDYSYPFT |
| | CDR-L3 | |
| 572 | Claudin-18.2 | QVQLQQPGAELVRPGASVKLSCKASGYTFTSYWINWVKQRPGQGLEWIGNIYPSDSYTN |
| | VH | YNQKFKDKATLTVDKSSSTAYMQLSSPTSEDSAVYYCTRSWRGNSFDYWGQGTTLTVSS |
| 573 | Claudin-18.2 | DIVMTQSPSSLTVTAGEKVTMSCKSSQSLLNSGNQKNYLTWYQQKPGQPPKLLIYWASTR |
| | VL | ESGVPDRFTGSGSGTDFTLTISSVQAEDLAVYYCQNDYSYPFTFGSGTKLEIK |
| 574 | Claudin-18.2 | NYGMN |
| | CDR-H1 | |
| 575 | Claudin-18.2 | WINTNTGEPTYAEEFKG |
| | CDR-H2 | |
| 576 | Claudin-18.2 | LGFGNAMDY |
| | CDR-H3 | |
| 577 | Claudin-18.2 | KSSQSLLNSGNQKNYLT |
| | CDR-L1 | |
| 578 | Claudin-18.2 | WASTRES |
| | CDR-L2 | |
| 579 | Claudin-18.2 | QNDYSYPLT |
| | CDR-L3 | |
| 580 | Claudin-18.2 | QIQLVQSGPELKKPGETVKISCKASGYTFTNYGMNWVKQAPGKGLKWMGWINTNTGEPT |
| | VH | Y |
| | | AEEFKGRFAFSLETSASTAYLQINNLKNEDTATYFCARLGFGNAMDYWGQGTSVTVSS |
| 581 | Claudin-18.2 | DIVMTQSPSSLTVTAGEKVTMSCKSSQSLLNSGNQKNYLTWYQQKPGQPPKLLIYWASTR |
| | VL | ESGVPDRFTGSGSGTDFTLTISSVQAEDLAVYYCQNDYSYPLTFGAGTKLELK |
| 582 | Nectin-4 CDR- | SYNMN |
| | H1 | |
| 583 | Nectin-4 CDR- | YISSSSSTIYYADSVKG |
| | H2 | |
| 584 | Nectin-4 CDR- | AYYYGMDV |
| | H3 | |
| 585 | Nectin-4 CDR- | RASQGISGWLA |
| | L1 | |
| 586 | Nectin-4 CDR- | AASTLQS |
| | L2 | |
| 587 | Nectin-4 CDR- | QQANSFPPT |
| | L3 | |
| 588 | Nectin-4 VH | EVQLVESGGGLVQPGGSLRLSCAASGFTFSSYNMNWVRQAPGKGLEWVSYISSSSSTIY |
| | | Y |
| | | ADSVKGRFTISRDNAKNSLSLQMNSLRDEDTAVYYCARAYYYGMDVWGQGTTVTVSS |
| 589 | Nectin-4 VL | DIQMTQSPSSVSASVGDRVTITCRASQGISGWLAWYQQKPGKAPKFLIYAASTLQSGVPS |
| | | RFSGSGSGTDFTLTISSLQPEDFATYYCQQANSFPPTFGGGTKVEIK |
| 590 | SLTRK6 CDR- | SYGMH |
| | H1 | |
| 591 | SLTRK6 CDR- | VIWYDGSNQYYADSVKG |
| | H2 | |
| 592 | SLTRK6 CDR- | GLTSGRYGMDV |
| | H3 | |
| 593 | SLTRK6 CDR- | RSSQSLLLSHGFNYLD |
| | L1 | |
| 594 | SLTRK6 CDR- | LGSSRAS |
| | L2 | |
| 595 | SLTRK6 CDR- | MQPLQIPWT |
| | L3 | |
| 596 | SLTRK6 VH | QVQLVESGGGVVQPGRSLRLSCAASGFTFSSYGMHWVRQAPGKGLEWVAVIWYDGSN |
| | | QYY |
| | | ADSVKGRFTISRDNSKNTLFLQMHSLRAEDTAVYYCARGLTSGRYGMDVWGQGTTVTVS |
| | | S |
| 597 | SLTRK6 VL | DIVMTQSPLSLPVTPGEPASISCRSSQSLLLSHGFNYLDWYLQKPGQSPQLLIYLGSSRAS |
| | | GVPDRFSGSGSGTDFTLKISRVEAEDVGLYYCMQPLQIPWTFGQGTKVEIK |
| 598 | CD228 CDR-H1 | SGYWN |
| 599 | CD228 CDR-H2 | YISDSGITYYNPSLKS |
| 600 | CD228 CDR-H3 | RTLATYYAMDY |
| 601 | CD228 CDR-L1 | RASQSLVHSDGNTYLH |
| 602 | CD228 CDR-L2 | RVSNRFS |
| 603 | CD228 CDR-L3 | SQSTHVPPT |
| 604 | CD228 VH | QVQLQESGPGLVKPSETLSLTCTVSGDSITSGYWNWIRQPPGKGLEYIGYISDSGITYYN |
| | | PSLKSRVTISRDTSKNQYSLKLSSVTAADTAVYYCARRTLATYYAMDYWGQGTLVTVSS |
| 605 | CD228 VL | DFVMTQSPLSLPVTLGQPASISCRASQSLVHSDGNTYLHWYQQRPGQSPRLLIYRVSNRF |
| | | SGVPDRFSGSGSGTDFTLKISRVEAEDVGVYYCSQSTHVPPTFGQGTKLEIKR |
| 606 | CD142 (TF) | NYAMS |
| | CDR-H1 | |
| 607 | CD142 (TF) | SISGSGDYTYYTDSVKG |
| | CDR-H2 | |
| 608 | CD142 (TF) | SPWGYYLDS |
| | CDR-H3 | |
| 609 | CD142 (TF) | RASQGISSRLA |
| | CDR-L1 | |
| 610 | CD142 (TF) | AASSLQS |
| | CDR-L2 | |
| 611 | CD142 (TF) | QQYNSYPYT |
| | CDR-L3 | |
| 612 | CD142 (TF) VH | EVQLLESGGGLVQPGGSLRLSCAASGFTFSNYAMSWVRQAPGKGLEWVSSISGSGDYT |
| | | Y |
| | | YTDSVKGRFTISRDNSKNTLYLQMNSLRAEDTAVYYCARSPWGYYLDSWGQGTLVTVSS |
| 613 | CD142 (TF) VL | DIQMTQSPPSLSASAGDRVTITCRASQGISSRLAWYQQKPEKAPKSLIYAASSLQSGVPS |
| | | RFSGSGSGTDFTLTISSLQPEDFATYYCQQYNSYPYTFGQGTKLEIK |
| 614 | STn CDR-H1 | DHAIH |
| 615 | STn CDR-H2 | YFSPGNDDIKYNEKFRG |
| 616 | STn CDR-H3 | SLSTPY |
| 617 | STn CDR-L1 | KSSQSLLNRGNHKNYLT |
| 618 | STn CDR-L2 | WASTRES |
| 619 | STn CDR-L3 | QNDYTYPYT |
| 620 | STn VH | EVQLVQSGAEVKKPGASVKVSCKASGYTFTDHAIHWVRQAPGQGLEWMGYFSPGNDDI |
| | | KY |
| | | NEKFRGRVTMTADKSSSTAYMELRSLRSDDTAVYFCKRSLSTPYWGQGTLVTVSS |
| 621 | STn VL | DIVMTQSPDSLAVSLGERATINCKSSQSLLNRGNHKNYLTWYQQKPGQPPKLLIYWAST |
| | | RESGVPDRFSGSGSGTDFTLTISSLQAEDVAVYYCQNDYTYPYTFGQGTKVEIK |
| 622 | CD20 CDR-H1 | SYNMH |
| 623 | CD20 CDR-H2 | AIYPGNGDTSYNQKFKG |
| 624 | CD20 CDR-H3 | STYYGGDWYFNV |
| 625 | CD20 CDR-L1 | RASSSVSYIH |
| 626 | CD20 CDR-L2 | ATSNLAS |
| 627 | CD20 CDR-L3 | QQWTSNPPT |
| 628 | CD20 VH | QVQLQQPGAELVKPGASVKMSCKASGYTFTSYNMHWVKQTPGRGLEWIGAIYPGNGDT |
| | | SY |
| | | NQKFKGKATLTADKSSSTAYMQLSSLTSEDSAVYYCARSTYYGGDWYFNVWGAGTTVTV |
| | | SA |
| 629 | CD20 VL | QIVLSQSPAILSASPGEKVTMTCRASSSVSYIHWFQQKPGSSPKPWIYATSNLASGVPVR |
| | | FSGSGSGTSYSLTISRVEAEDAATYYCQQWTSNPPTFGGGTKLEIK |
| 630 | HER2 CDR-H1 | DTYIH |
| 631 | HER2 CDR-H2 | RIYPTNGYTRYADSVKG |
| 632 | HER2 CDR-H3 | WGGDGFYAMDY |
| 633 | HER2 CDR-L1 | RASQDVNTAVA |
| 634 | HER2 CDR-L2 | SASFLYS |
| 635 | HER2 CDR-L3 | QQHYTTPPT |
| 636 | HER2 VH | EVQLVESGGGLVQPGGSLRLSCAASGFNIKDTYIHWVRQAPGKGLEWVARIYPTNGYTRY |
| | | ADSVKGRFTISADTSKNTAYLQMNSLRAEDTAVYYCSRWGGDGFYAMDYWGQGTLVTV |
| | | SS |
| 637 | HER2 VL | DIQMTQSPSSLSASVGDRVTITCRASQDVNTAVAWYQQKPGKAPKLLIYSASFLYSGVPS |
| | | RFSGSRSGTDFTLTISSLQPEDFATYYCQQHYTTPPTFGQGTKVEIK |
| 638 | CD79b CDR-H1 | SYWIE |
| 639 | CD79b CDR-H2 | EILPGGGDTNYNEIFKG |
| 640 | CD79b CDR-H3 | RVPIRLDY |
| 641 | CD79b CDR-L1 | KASQSVDYEGDSFLN |
| 642 | CD79b CDR-L2 | AASNLES |
| 643 | CD79b CDR-L3 | QQSNEDPLT |
| 644 | CD79b VH | EVQLVESGGGLVQPGGSLRLSCAASGYTFSSYWIEWVRQAPGKGLEWIGEILPGGGDTN |
| | | YNEIFKGRATFSADTSKNTAYLQMNSLRAEDTAVYYCTRRVPIRLDYWGQGTLVTVSS |
| 645 | CD79b VL | DIQLTQSPSSLSASVGDRVTITCKASQSVDYEGDSFLNWYQQKPGKAPKLLIYAASNLES |
| | | GVPSRFSGSGSGTDFTLTISSLQPEDFATYYCQQSNEDPLTFGQGTKVEIK |
| 646 | NaPi2B CDR-H1 | DFAMS |
| 647 | NaPi2B CDR-H2 | TIGRVAFHTYYPDSMKG |
| 648 | NaPi2B CDR-H3 | HRGFDVGHFDF |
| 649 | NaPi2B CDR-L1 | RSSETLVHSSGNTYLE |
| 650 | NaPi2B CDR-L2 | RVSNRFS |
| 651 | NaPi2B CDR-L3 | FQGSFNPLT |
| 652 | NaPi2B VH | EVQLVESGGGLVQPGGSLRLSCAASGFSFSDFAMSWVRQAPGKGLEWVATIGRVAFHTY |
| | | Y |
| | | PDSMKGRFTISRDNSKNTLYLQMNSLRAEDTAVYYCARHRGFDVGHFDFWGQGTLVTVS |
| | | S |
| 653 | NaPi2B VL | DIQMTQSPSSLSASVGDRVTITCRSSETLVHSSGNTYLEWYQQKPGKAPKLLIYRVSNRF |
| | | SGVPSRFSGSGSGTDFTLTISSLQPEDFATYYCFQGSFNPLTFGQGTKVEIK |
| 654 | Muc16 CDR-H1 | NDYAWN |
| 655 | Muc16 CDR-H2 | YISYSGYTTYNPSLKS |
| 656 | Muc16 CDR-H3 | WTSGLDY |
| 657 | Muc16 CDR-L1 | KASDLIHNWLA |
| 658 | Muc16 CDR-L2 | GATSLET |
| 659 | Muc16 CDR-L3 | QQYWTTPFT |
| 660 | Muc16 VH | EVQLVESGGGLVQPGGSLRLSCAASGYSITNDYAWNWVRQAPGKGLEWVGYISYSGYTT |
| | | Y |
| | | NPSLKSRFTISRDTSKNTLYLQMNSLRAEDTAVYYCARWTSGLDYWGQGTLVTVSS |
| 661 | Muc 16 VL | DIQMTQSPSSLSASVGDRVTITCKASDLIHNWLAWYQQKPGKAPKLLIYGATSLETGVPSR |
| | | FSGSGSGTDFTLTISSLQPEDFATYYCQQYWTTPFTFGQGTKVEIK |
| 662 | STEAP1 CDR- | SDYAWN |
| | H1 | |
| 663 | STEAP1 CDR- | YISNSGSTSYNPSLKS |
| | H2 | |
| 664 | STEAP1 CDR- | ERNYDYDDYYYAMDY |
| | H3 | |
| 665 | STEAP1 CDR- | KSSQSLLYRSNQKNYLA |
| | L1 | |
| 666 | STEAP1 CDR- | WASTRES |
| | L2 | |
| 667 | STEAP1 CDR- | QQYYNYPRT |
| | L3 | |
| 668 | STEAP1 VH | EVQLVESGGGLVQPGGSLRLSCAVSGYSITSDYAWNWVRQAPGKGLEWVGYISNSGST |
| | | SYNPSLKSRFTISRDTSKNTLYLQMNSLRAEDTAVYYCARERNYDYDDYYYAMDYWGQG |
| | | TLVTVSS |
| 669 | STEAP1 VL | DIQMTQSPSSLSASVGDRVTITCKSSQSLLYRSNQKNYLAWYQQKPGKAPKLLIYWASTR |
| | | ESGVPSRFSGSGSGTDFTLTISSLQPEDFATYYCQQYYNYPRTFGQGTKVEIK |
| 670 | BCMA CDR-H1 | NYWMH |
| 671 | BCMA CDR-H2 | ATYRGHSDTYYNQKFKG |
| 672 | BCMA CDR-H3 | GAIYDGYDVLDN |
| 673 | BCMA CDR-L1 | SASQDISNYLN |
| 674 | BCMA CDR-L2 | YTSNLHS |
| 675 | BCMA CDR-L3 | QQYRKLPWT |
| 676 | BCMA VH | QVQLVQSGAEVKKPGSSVKVSCKASGGTFSNYWMHWVRQAPGQGLEWMGATYRGHS |
| | | DTYYNQKFKGRVTITADKSTSTAYMELSSLRSEDTAVYYCARGAIYDGYDVLDNWGQGTL |
| | | VTVSS |
| 677 | BCMA VL | DIQMTQSPSSLSASVGDRVTITCSASQDISNYLNWYQQKPGKAPKLLIYYTSNLHSGVPSR |
| | | FSGSGSGTDFTLTISSLQPEDFATYYCQQYRKLPWTFGQGTKLEIK |
| 678 | c-Met CDR-H1 | AYTMH |
| 679 | C-Met CDR-H2 | WIKPNNGLANYAQKFQG |
| 680 | c-Met CDR-H3 | SEITTEFDY |
| 681 | c-Met CDR-L1 | KSSESVDSYANSFLH |
| 682 | c-Met CDR-L2 | RASTRES |
| 683 | c-Met CDR-L3 | QQSKEDPLT |
| 684 | c-Met VH | QVQLVQSGAEVKKPGASVKVSCKASGYIFTAYTMHWVRQAPGQGLEWMGWIKPNNGLA |
| | | N |
| | | YAQKFQGRVTMTRDTSISTAYMELSRLRSDDTAVYYCARSEITTEFDYWGQGTLVTVSS |
| 685 | c-Met VL | DIVMTQSPDSLAVSLGERATINCKSSESVDSYANSFLHWYQQKPGQPPKLLIYRASTRE |
| | | SGVPDRFSGSGSGTDFTLTISSLQAEDVAVYYCQQSKEDPLTFGGGTKVEIK |
| 686 | EGFR CDR-H1 | SDFAWN |
| 687 | EGFR CDR-H2 | YISYSGNTRYQPSLKS |
| 688 | EGFR CDR-H3 | AGRGFPY |
| 689 | EGFR CDR-L1 | HSSQDINSNIG |
| 690 | EGFR CDR-L2 | HGTNLDD |
| 691 | EGFR CDR-L3 | VQYAQFPWT |
| 692 | EGFR VH | QVQLQESGPGLVKPSQTLSLTCTVSGYSISSDFAWNWIRQPPGKGLEWMGYISYSGNTR |
| | | Y |
| | | QPSLKSRITISRDTSKNQFFLKLNSVTAADTATYYCVTAGRGFPYWGQGTLVTVSS |
| 693 | EGFR VL | DIQMTQSPSSMSVSVGDRVTITCHSSQDINSNIGWLQQKPGKSFKGLIYHGTNLDDGVPS |
| | | RFSGSGSGTDYTLTISSLQPEDFATYYCVQYAQFPWTFGGGTKLEIK |
| 694 | SLAMF7 CDR- | DYYMA |
| | H1 | |
| 695 | SLAMF7 CDR- | SINYDGSSTYYVDSVKG |
| | H2 | |
| 696 | SLAMF7 CDR- | DRGYYFDY |
| | H3 | |
| 697 | SLAMF7 CDR- | RSSQSLVHSNGNTYLH |
| | L1 | |
| 698 | SLAMF7 CDR- | KVSNRFS |
| | L2 | |
| 699 | SLAMF7 CDR- | SQSTHVPPFT |
| | L3 | |
| 700 | SLAMF7 VH | EVQLVESGGGLVQPGGSLRLSCAASGFTFSDYYMAWVRQAPGKGLEWVASINYDGSST |
| | | Y |
| | | YVDSVKGRFTISRDNAKNSLYLQMNSLRAEDTAVYYCARDRGYYFDYWGQGTTVTVSS |
| 701 | SLAMF7 VL | DVVMTQTPLSLSVTPGQPASISCRSSQSLVHSNGNTYLHWYLQKPGQSPQLLIYKVSNRF |
| | | SGVPDRFSGSGSGTDFTLKISRVEAEDVGVYFCSQSTHVPPFTFGGGTKVEIK |
| 702 | SLITRK6 CDR- | SYGMH |
| | H1 | |
| 703 | SLITRK6 CDR- | VIWYDGSNQYYADSVKG |
| | H2 | |
| 704 | SLITRK6 CDR- | GLTSGRYGMDV |
| | H3 | |
| 705 | SLITRK6 CDR- | RSSQSLLLSHGFNYLD |
| | L1 | |
| 706 | SLITRK6 CDR- | LGSSRAS |
| | L2 | |
| 707 | SLITRK6 CDR- | MQPLQIPWT |
| | L3 | |
| 708 | SLITRK6 VH | QVQLVESGGGVVQPGRSLRLSCAASGFTFSSYGMHWVRQAPGKGLEWVAVIWYDGSN |
| | | QYY |
| | | ADSVKGRFTISRDNSKNTLFLQMHSLRAEDTAVYYCARGLTSGRYGMDVWGQGTTVTVS |
| | | S |
| 709 | SLITRK6 VL | DIVMTQSPLSLPVTPGEPASISCRSSQSLLLSHGFNYLDWYLQKPGQSPQLLIYLGSSRA |
| | | SGVPDRFSGSGSGTDFTLKISRVEAEDVGLYYCMQPLQIPWTFGQGTKVEIK |
| 710 | C4.4a CDR-H1 | NAWMS |
| 711 | C4.4a CDR-H2 | YISSSGSTIYYADSVKG |
| 712 | C4.4a CDR-H3 | EGLWAFDY |
| 713 | C4.4a CDR-L1 | TGSSSNIGAGYVVH |
| 714 | C4.4a CDR-L2 | DNNKRPS |
| 715 | C4.4a CDR-L3 | AAWDDRLNGPV |
| 716 | C4.4a VH | EVQLLESGGGLVQPGGSLRLSCAASGFTFSNAWMSWVRQAPGKGLEWVSYISSSGSTIY |
| | | Y |
| | | ADSVKGRFTISRDNSKNTLYLQMNSLRAEDTAVYYCAREGLWAFDYWGQGTLVTVSS |
| 717 | C4.4a VL | ESVLTQPPSVSGAPGQRVTISCTGSSSNIGAGYWHWYQQLPGTAPKLLIYDNNKRPSGV |
| | | PDRFSGSKSGTSASLAISGLRSEDEADYYCAAWDDRLNGPVFGGGTKLTVL |
| 718 | GCC CDR-H1 | GYYWS |
| 719 | GCC CDR-H2 | EINHRGNTNDNPSLKS |
| 720 | GCC CDR-H3 | ERGYTYGNFDH |
| 721 | GCC CDR-L1 | RASQSVSRNLA |
| 722 | GCC CDR-L2 | GASTRAT |
| 723 | GCC CDR-L3 | QQYKTWPRT |
| 724 | GCC VH | QVQLQQWGAGLLKPSETLSLTCAVFGGSFSGYYWSWIRQPPGKGLEWIGEINHRGNTND |
| | | N |
| | | PSLKSRVTISVDTSKNQFALKLSSVTAADTAVYYCARERGYTYGNFDHWGQGTLVTVSS |
| 725 | GCC VL | EIVMTQSPATLSVSPGERATLSCRASQSVSRNLAWYQQKPGQAPRLLIYGASTRATGIP |
| | | ARFSGSGSGTEFTLTIGSLQSEDFAVYYCQQYKTWPRTFGQGTNVEIK |
| 726 | Axl CDR-H1 | SYAMN |
| 727 | Axl CDR-H2 | TTSGSGASTYYADSVKG |
| 728 | Axl CDR-H3 | IWIAFDI |
| 729 | Axl CDR-L1 | RASQSVSSSYLA |
| 730 | Axl CDR-L2 | GASSRAT |
| 731 | Axl CDR-L3 | QQYGSSPYT |
| 732 | Axl VH | EVQLLESGGGLVQPGGSLRLSCAASGFTFSSYAMNWVRQAPGKGLEWVSTTSGSGAST |
| | | YY |
| | | ADSVKGRFTISRDNSKNTLYLQMNSLRAEDTAVYYCAKIWIAFDIWGQGTMVTVSS |
| 733 | Axl VL | EIVLTQSPGTLSLSPGERATLSCRASQSVSSSYLAWYQQKPGQAPRLLIYGASSRATGIP |
| | | DRFSGSGSGTDFTLTISRLEPEDFAVYYCQQYGSSPYTFGQGTKLEIK |
| 734 | gpNMB CDR-H1 | SFNYYWS |
| 735 | gpNMB CDR-H2 | YIYYSGSTYSNPSLKS |
| 736 | gpNMB CDR-H3 | GYNWNYFDY |
| 737 | gpNMB CDR-L1 | RASQSVDNNLV |
| 738 | gpNMB CDR-L2 | GASTRAT |
| 739 | gpNMB CDR-L3 | QQYNNWPPWT |
| 740 | gpNMB VH | QVQLQESGPGLVKPSQTLSLTCTVSGGSISSFNYYWSWIRHHPGKGLEWIGYIYYSGSTY |
| | | SNPSLKSRVTISVDTSKNQFSLTLSSVTAADTAVYYCARGYNWNYFDYWGQGTLVTVSS |
| 741 | gpNMB VL | EIVMTQSPATLSVSPGERATLSCRASQSVDNNLVWYQQKPGQAPRLLIYGASTRATGIPA |
| | | RFSGSGSGTEFTLTISSLQSEDFAVYYCQQYNNWPPWTFGQGTKVEIK |
| 742 | Prolactin | TYWMH |
| | receptor CDR- | |
| | H1 | |
| 743 | Prolactin | EIDPSDSYSNYNQKFKD |
| | receptor CDR- | |
| | H2 | |
| 744 | Prolactin | NGGLGPAWFSY |
| | receptor CDR- | |
| | H3 | |
| 745 | Prolactin | KASQYVGTAVA |
| | receptor CDR-L1 | |
| 746 | Prolactin | SASNRYT |
| | receptor CDR-L2 | |
| 747 | Prolactin | QQYSSYPWT |
| | receptor CDR-L3 | |
| 748 | Prolactin | EVQLVQSGAEVKKPGSSVKVSCKASGYTFTTYWMHWVRQAPGQGLEWIGEIDPSDSYS |
| | receptor VH | NY |
| | | NQKFKDRATLTVDKSTSTAYMELSSLRSEDTAVYYCARNGGLGPAWFSYWGQGTLVTVS |
| | | S |
| 749 | Prolactin | DIQMTQSPSSVSASVGDRVTITCKASQYVGTAVAWYQQKPGKSPKLLIYSASNRYTGVPS |
| | receptor VL | RFSDSGSGTDFTLTISSLQPEDFATYFCQQYSSYPWTFGGGTKVEIK |
| 750 | FGFR2 CDR-H1 | SYAMS |
| 751 | FGFR2 CDR-H2 | AISGSGTSTYYADSVKG |
| 752 | FGFR2 CDR-H3 | VRYNWNHGDWFDP |
| 753 | FGFR2 CDR-L1 | SGSSSNIGNNYVS |
| 754 | FGFR2 CDR-L2 | ENYNRPA |
| 755 | FGFR2 CDR-L3 | SSWDDSLNYWV |
| 756 | FGFR2 VH | EVQLLESGGGLVQPGGSLRLSCAASGFTFSSYAMSWVRQAPGKGLEWVSAISGSGTSTY |
| | | YADSVKGRFTISRDNSKNTLYLQMNSLRAEDTAVYYCARVRYNWNHGDWFDPWGQGTL |
| | | VTVSS |
| 757 | FGFR2 VL | QSVLTQPPSASGTPGQRVTISCSGSSSNIGNNYVSWYQQLPGTAPKLLIYENYNRPAGVP |
| | | DRFSGSKSGTSASLAISGLRSEDEADYYCSSWDDSLNYWVFGGGTKLTVL |
| 758 | CDCP1 CDR-H1 | SYGMS |
| 759 | CDCP1 CDR-H2 | TISSGGSYKYYVDSVKG |
| 760 | CDCP1 CDR-H3 | HPDYDGVWFAY |
| 761 | CDCP1 CDR-L1 | SVSSSVFYVH |
| 762 | CDCP1 CDR-L2 | DTSKLAS |
| 763 | CDCP1 CDR-L3 | QQWNSNPPT |
| 764 | CDCP1 VH | EVQLVESGGGLVQPGGSLRLSCAASGFTFNSYGMSWVRQAPGKGLEWVATISSGGSYK |
| | | YY |
| | | VDSVKGRFTISRDNAKNSLYLQMNSLRAEDTAVYYCARHPDYDGVWFAYWGQGTLVTVS |
| | | S |
| 765 | CDCP1 VL | DIQMTQSPSSLSASVGDRVTITCSVSSSVFYVHWYQQKPGKAPKLLIYDTSKLASSGVPS |
| | | RFSGSGSGTDFTFTISSLQPEDIATYYCQQWNSNPPTFGGGTKVEIK |
| 766 | CDCP1 CDR-H1 | SYGMS |
| 767 | CDCP1 CDR-H2 | TISSGGSYTYYPDSVKG |
| 768 | CDCP1 CDR-H3 | HPDYDGVWFAY |
| 769 | CDCP1 CDR-L1 | SVSSSVFYVH |
| 770 | CDCP1 CDR-L2 | DTSKLAS |
| 771 | CDCP1 CDR-L3 | QQWNSNPPT |
| 772 | CDCP1 VH | EVQLVESGGDLVKPGGSLKLSCAASGFTFNSYGMSWVRQTPDKRLEWVATISSGGSYTY |
| | | Y |
| | | PDSVKGRFTISRDNAKNTLYLQMSSLKSEDTAMYYCARHPDYDGVWFAYWGQGTLVTVS |
| | | A |
| 773 | CDCP1 VL | QIVLTQSPAIMASPGEKVTMTCSVSSSVFYVHWYQQKSGTSPKRWIYDTSKLASGVPARF |
| | | SGSGSGTSYSLTISSMEAEDAATYYCQQWNSNPPTFGGGTKLEIK |
| 774 | CDCP1 CDR-H1 | SYYMH |
| 775 | CDCP1 CDR-H2 | IINPSGGSTSYAQKFQG |
| 776 | CDCP1 CDR-H3 | DGVLRYFDWLLDYYYY |
| 777 | CDCP1 CDR-L1 | RASQSVGSYLA |
| 778 | CDCP1 CDR-L2 | DASNRAT |
| 779 | CDCP1 CDR-L3 | QQRANVFT |
| 780 | CDCP1 VH | EVQLVQSGAEVKKPGASVKVSCKASGYTFTSYYMHWVRQAPGQGLEWMGIINPSGGST |
| | | SY |
| | | AQKFQGRVTMTRDTSTSTVYMELSSLRSEDTAVYYCARDGVLRYFDWLLDYYYYMDVW |
| | | GKG |
| | | TTVTVSS |
| 781 | CDCP1 VL | EIVLTQSPATLSLSPGERATLSCRASQSVGSYLAWYQQRPGQAPRLLIYDASNRATGIPA |
| | | RFSGSGSGTDFTLTISSLEPEDFAVYYCQQRANVFTFGQGTKVEIK |
| 782 | CDCP1 CDR-H1 | SYYMH |
| 783 | CDCP1 CDR-H2 | IINPSGGSTSYAQKFQG |
| 784 | CDCP1 CDR-H3 | DAELRHFDHLLDYHYYMDV |
| 785 | CDCP1 CDR-L1 | RASQSVGSYLA |
| 786 | CDCP1 CDR-L2 | DASNRAT |
| 787 | CDCP1 CDR-L3 | QQRAQEFT |
| 788 | CDCP1 VH | EVQLVQSGAEVKKPGASVKVSCKASGYTFTSYYMHWVRQAPGQGLEWMGIINPSGGST |
| | | SYAQKFQGRVTMTRDTSTSTVYMELSSLRSEDTAVYYCARDAELRHFDHLLDYHYYMDV |
| | | WGQGTTVTVSS |
| 789 | CDCP1 VL | EIVMTQSPATLSLSPGERATLSCRASQSVGSYLAWYQQKPGQAPRLLIYDASNRATGIPA |
| | | RFSGSGSGTDFTLTISSLQPEDFAVYYCQQRAQEFTFGQGTKVEIK |
| 790 | ASCT2 VH | QVQLVQSGSELKKPGAPVKVSCKASGYTFSTFGMSWVRQAPGQGLKWMGWIHTYAGVP |
| | | IYGDDFKGRFVFSLDTSVSTAYLQISSLKAEDTAVYFCARRSDNYRYFFDYWGQGTTVTV |
| | | SS |
| 791 | ASCT2 VL | DIQMTQSPSSLSASLGDRVTITCRASQDIRNYLNWYQQKPGKAPKLLIYYTSRLHSGVPSR |
| | | FSGSGSGTDYTLTISSLQPEDFATYFCQQGHTLPPTFGQGTKLEIK |
| 792 | ASCT2 VH | QIQLVQSGPELKKPGAPVKISCKASGYTFTTFGMSWVKQAPGQGLKWMGWIHTYAGVPI |
| | | YGDDFKGRFVFSLDTSVSTAYLQISSVKAEDTATYFCARRSDNYRYFFDYWGQGTTLTVS |
| | | S |
| 793 | ASCT2 VL | DIQMTQSPSSLSASLGDRVTITCRASQDIRNYLNWYQQKPGKAPKLLIYYTSRLHSGVPS |
| | | RFSGSGSGTDYTLTISSLQPEDFATYFCQQGHTLPPTFGQGTKLEIK |
| 794 | ASCT2 CDR-H1 | NYYMA |
| 795 | ASCT2 CDR-H2 | SITKGGGNTYYRDSVKG |
| 796 | ASCT2 CDR-H3 | QVTIAAVSTSYFDS |
| 797 | ASCT2 CDR-L1 | KTNQKVDYYGNSYVY |
| 798 | ASCT2 CDR-L2 | LASNLAS |
| 799 | ASCT2 CDR-L3 | QQSRNLPYT |
| 800 | ASCT2 VH | EVQLVESGGGLVQSGRSIRLSCAASGFSFSNYYMAWVRQAPSKGLEWVASITKGGGNTY |
| | | YRDSVKGRFTFSRDNAKSTLYLQMDSLRSEDTATYYCARQVTIAAVSTSYFDSWGQGVM |
| | | VTVSS |
| 801 | ASCT2 VL | DIVLTQSPALAVSLGQRATISCKTNQKVDYYGNSYVYWYQQKPGQQPKLLIYLASNLASGI |
| | | PARFSGRGSGTDFTLTIDPVEADDTATYYCQQSRNLPYTFGAGTKLELK |
| 802 | CD123 CDR-H1 | DYYMK |
| 803 | CD123 CDR-H2 | DIIPSNGATFYNQKFKG |
| 804 | CD123 CDR-H3 | SHLLRASWFAY |
| 805 | CD123 CDR-L1 | KSSQSLLNSGNQKNYLT |
| 806 | CD123 CDR-L2 | WASTRES |
| 807 | CD123 CDR-L3 | QNDYSYPYT |
| 808 | CD123 VH | QVQLVQSGAEVKKPGASVKMSCKASGYTFTDYYMKWVKQAPGQGLEWIGDIIPSNGATF |
| | | YNQKFKGKATLTVDRSISTAYMHLNRLRSDDTAVYYCTRSHLLRASWFAYWGQGTLVTVS |
| | | S |
| 809 | CD123 VL | DFVMTQSPDSLAVSLGERATINCKSSQSLLNSGNQKNYLTWYLQKPGQPPKLLIYWASTR |
| | | ESGVPDRFSGSGSGTDFTLTISSLQAEDVAVYYCQNDYSYPYTFGQGTKLEIK |
| 810 | GPC3 CDR-H1 | DYEMH |
| 811 | GPC3 CDR-H2 | WIGGIDPETGGTAYNQKFKG |
| 812 | GPC3 CDR-H3 | YYSFAY |
| 813 | GPC3 CDR-L1 | RSSQSIVHSNGNTYLQ |
| 814 | GPC3 CDR-L2 | KVSNRFS |
| 815 | GPC3 CDR-L3 | FQVSHVPYT |
| 816 | GPC3 VH | EVQLVQSGAEVKKPGATVKISCKVSGYTFTDYEMHWVQQAPGKGLEWMGGIDPETGGT |
| | | AYNQKFKGRVTLTADKSTDTAYMELSSLRSEDTAVYYCGRYYSFAYWGQGTLVTVSS |
| 817 | GPC3 VL | DWVMTQSPLSLPVTLGQPASISCRSSQSIVHSNANTYLQWFQQRPGQSPRLLIYKVSNRF |
| | | SGVPDRFSGSGSGTDFTLKISRVEAEDVGVYYCFQVSHVPYTFGQGTKLEIK |
| 818 | B6A CDR-H1 | DYNVN |
| 819 | B6A CDR-H2 | VINPKYGTTRYNQKFKG |
| 820 | B6A CDR-H3 | GLNAWDY |
| 821 | B6A CDR-L1 | GASENIYGALN |
| 822 | B6A CDR-L2 | GATNLED |
| 823 | B6A CDR-L3 | QNVLTTPYT |
| 824 | B6A VH | QFQLVQSGAEVKKPGASVKVSCKASGYSFTDYNVNWVRQAPGQGLEWIGVINPKYGTTR |
| | | YNQKFKGRATLTVDKSTSTAYMELSSLRSEDTAVYYCTRGLNAWDYWGQGTLVTVSS |
| 825 | B6A VL | DIQMTQSPSSLSASVGDRVTITCGASENIYGALNWYQQKPGKAPKLLIYGATNLEDGVPSR |
| | | FSGSGSGRDYTFTISSLQPEDIATYYCQNVLTTPYTFGQGTKLEIK |
| 826 | B6A CDR-H1 | GYFMN |
| 827 | B6A CDR-H2 | LINPYNGDSFYNQKFKG |
| 828 | B6A CDR-H3 | GLRRDFDY |
| 829 | B6A CDR-L1 | KSSQSLLDSDGKTYLN |
| 830 | B6A CDR-L2 | LVSELDS |
| 831 | B6A CDR-L3 | WQGTHFPRT |
| 832 | B6A VH | QVQLVQSGAEVKKPGASVKVSCKASGYSFSGYFMNWVRQAPGQGLEWMGLINPYNGD |
| | | SFYNQKFKGRVTMTRQTSTSTVYMELSSLRSEDTAVYYCVRGLRRDFDYWGQGTLVTVS |
| | | S |
| 833 | B6A VL | DWVMTQSPLSLPVTLGQPASISCKSSQSLLDSDGKTYLNWLFQRPGQSPRRLIYLVSELDS |
| | | GVPDRFSGSGSGTDFTLKISRVEAEDVGVYYCWQGTHFPRTFGGGTKLEIK |
| 834 | PD-L1 CDR-H1 | TAAIS |
| 835 | PD-L1 CDR-H2 | GIIPIFGKAHYAQKFQG |
| 836 | PD-L1 CDR-H3 | KFHFVSGSPFGMDV |
| 837 | PD-L1 CDR-L1 | RASQSVSSYLA |
| 838 | PD-L1 CDR-L2 | DASNRAT |
| 839 | PD-L1 CDR-L3 | QQRSNWPT |
| 840 | PD-L1 VH | QVQLVQSGAEVKKPGSSVKVSCKTSGDTFSTAAISWVRQAPGQGLEWMGGIIPIFGKAHY |
| | | AQKFQGRVTITADESTSTAYMELSSLRSEDTAVYFCARKFHFVSGSPFGMDVWGQGTTV |
| | | TVSS |
| 841 | PD-L1 VL | EIVLTQSPATLSLSPGERATLSCRASQSVSSYLAWYQQKPGQAPRLLIYDASNRATGIPAR |
| | | FSGSGSGTDFTLTISSLEPEDFAVYYCQQRSNWPTFGQGTKVEIK |
| 842 | TIGIT CDR-H1 | GTFSSYAIS |
| 843 | TIGIT CDR-H2 | SIIPIFGTANYAQKFQG |
| 844 | TIGIT CDR-H3 | ARGPSEVGAILGYVWFDP |
| 845 | TIGIT CDR-L1 | RSSQSLLHSNGYNYLD |
| 846 | TIGIT CDR-L2 | LGSNRAS |
| 847 | TIGIT CDR-L3 | MQARRIPIT |
| 848 | TIGIT VH | QVQLVQSGAEVKKPGSSVKVSCKASGGTFSSYAISWVRQAPGQGLEWMGSIIPIFGTANY |
| | | AQKFQGRVTITADESTSTAYMELSSLRSEDTAVYYCARGPSEVGAILGYVWFDPWGQGTL |
| | | VTVSS |
| 849 | TIGIT VL | DIVMTQSPLSLPVTPGEPASISCRSSQSLLHSNGYNYLDWYLQKPGQSPQLLIYLGSNRAS |
| | | GVPDRFSGSGSGTDFTLKISRVEAEDVGVYYCMQARRIPITFGGGTKVEIK |
| 850 | STN CDR-H1 | GYTFTDHAIHWV |
| 851 | STN CDR-H2 | FSPGNDDIKY |
| 852 | STN CDR-H3 | KRSLSTPY |
| 853 | STN CDR-L1 | QSLLNRGNHKNY |
| 854 | STN CDR-L2 | WASTRES |
| 855 | STN CDR-L3 | QNDYTYPYT |
| 856 | STN VH | EVQLVQSGAEVKKPGASVKVSCKASGYTFTDHAIHWVRQAPGQGLEWMGYFSPGNDDI |
| | | KYNEKFRGRVTMTADKSSSTAYMELRSLRSDDTAVYFCKRSLSTPYWGQGTLVTVSS |
| 857 | STN VL | DIVMTQSPDSLAVSLGERATINCKSSQSLLNRGNHKNYLTWYQQKPGQPPKLLIYWASTR |
| | | ESGVPDRFSGSGSGTDFTLTISSLQAEDVAVYYCQNDYTYPYTFGQGTKVEIK |
| 858 | CD33 CDR-H1 | NYDIN |
| 859 | CD33 CDR-H2 | WIYPGDGSTKYNEKFKA |
| 860 | CD33 CDR-H3 | GYEDAMDY |
| 861 | CD33 CDR-L1 | KASQDINSYLS |
| 862 | CD33 CDR-L2 | RANRLVD |
| 863 | CD33 CDR-L3 | LQYDEFPLT |
| 864 | CD33 VH | QVQLVQSGAEVKKPGASVKVSCKASGYTFTNYDINWVRQAPGQGLEWIGW |
| | | IYPGDGSTKYNEKFKAKATLTADTSTSTAYMELRSLRSDDTAVYYCASGY |
| | | EDAMDYWGQGTTVTVSS |
| 865 | CD33 VL | DIQMTQSPSSLSASVGDRVTINCKASQDINSYLSWFQQKPGKAPKTLIYRANRLVDGVPS |
| | | RFSGSGSGQDYTLTISSLQPEDFATYYCLQYDEFPLTFGGGTKVE |
| 866 | NTBA CDR-H1 | NYGMN |
| 867 | NTBA CDR-H2 | WINTYSGEPRYADDFKG |
| 868 | NTBA CDR-H3 | DYGRWYFDV |
| 869 | NTBA CDR-L1 | RASSSVSHMH |
| 870 | NTBA CDR-L2 | ATSNLAS |
| 871 | NTBA CDR-L3 | QQWSSTPRT |
| 872 | NTBA VH | QIQLVQSGSELKKPGASVKVSCKASGYTFTNYGMNWVRQAPGQDLKWMGWINTYSGEP |
| | | RYADDFKGRFVFSLDKSVNTAYLQISSLKAEDTAVYYCARDYGRWYFDVWGQGTTVTVS |
| | | S |
| 873 | NTBA VL | QIVLSQSPATLSLSPGERATMSCRASSSVSHMHWYQQKPGQAPRPWIYATSNLASGVPA |
| | | RFSGSGSGTDYTLTISSLEPEDFAVYYCQQWSSTPRTFGGGTKVEIK |
| 874 | BCMA CDR-H1 | DYYIH |
| 875 | BCMA CDR-H2 | YINPNSGYTNYAQKFQG |
| 876 | BCMA CDR-H3 | YMWERVTGFFDF |
| 877 | BCMA CDR-L1 | LASEDISDDLA |
| 878 | BCMA CDR-L2 | TTSSLQS |
| 879 | BCMA CDR-L3 | QQTYKFPPT |
| 880 | BCMA VH | QVQLVQSGAEVKKPGASVKLSCKASGYTFTDYYIHWVRQAPGQGLEWIGYINPNSGYTN |
| | | YAQKFQGRATMTADKSINTAYVELSRLRSDDTAVYFCTRYMWERVTGFFDFWGQGTMVT |
| | | VSS |
| 881 | BCMA VL | DIQMTQSPSSVSASVGDRVTITCLASEDISDDLAWYQQKPGKAPKVLVYTTSSLQSGVPS |
| | | RFSGSGSGTDFTLTISSLQPEDFATYFCQQTYKFPPTFGGGTKVEIK |
| 882 | TF CDR-H1 | GFTFSNYA |
| 883 | TF CDR-H2 | ISGSGDYT |
| 884 | TF CDR-H3 | ARSPWGYYLDS |
| 885 | TF CDR-L1 | QGISSR |
| 886 | TF CDR-L2 | AAS |
| 887 | TF CDR-L3 | QQYNSYPYT |
| 888 | TF VH | EVQLLESGGGLVQPGGSLRLSCAASGFTFSNYAMSWVRQAPGKGLEWVSSISGSGDYT |
| | | YYTDSVKGRFTISRDNSKNTLYLQMNSLRAEDTAVYYCARSPWGYYLDSWGQGTLVTVS |
| | | S |
| 889 | TF VL | DIQMTQSPPSLSASAGDRVTITCRASQGISSRLAWYQQKPEKAPKSLIYAASSLQSGVPSR |
| | | FSGSGSGTDFTLTISSLQPEDFATYYCQQYNSYPYTFGQGTKLEIK |

In some embodiments, the antibody is a non-targeted antibody, for example, a non-binding or control antibody.

### Linkers

As described herein, linkers (L) are optional groups that connect D with Ab.

In some embodiments, the linker (L) has the formula -M-(A)ₐ-(W)_{w}—(Y)_{y}—(X)—, wherein:
M is a succinimide, a hydrolyzed succinimide, an amide, or a triazole;
X is from 1-10 amino acids; or
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl;
   A is a C₂₋₁₀ alkylene optionally substituted with 1-3 R^{a1}; or a 3 to 20 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
subscript a is 0 or 1;
W is from 2-6 amino acids or has the structure:

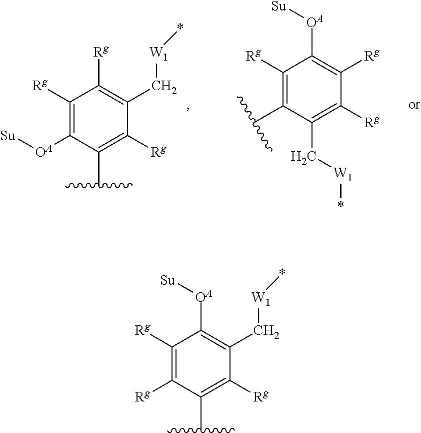

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W₁ is absent, *—C(═O)—O—, or *—O—C(═O)—;
[symbol] represents covalent attachment to A or M;
* represents covalent attachment to Y or X;
subscript w is 0 or 1;
Y is a self-immolative moiety, a non-self-immolative releasable moiety, or a non-cleavable moiety; and
subscript y is 0 or 1.

In some embodiments, subscript a is 0. In some embodiments, subscript a is 1. In some embodiments, subscript w is 0. In some embodiments, subscript w is 1. In some embodiments, subscript y is 0. In some embodiments, subscript y is 1. In some embodiments, subscripts a+y+w=1. In some embodiments, subscripts a+y+w=2. In some embodiments, subscripts a+y+w=3. In some embodiments, subscripts a+y+w=0 (i.e., the linker (L) is -M-X—).

In some embodiments, X is from 1-10 amino acids, for example, 1, 2, 3, 4, 5, 6, 7, 8, 9, or 10 independently selected amino acids. In some embodiments, X is 1 amino acid. In some embodiments, X is 2 amino acids. In some embodiments, X is 3 amino acids. In some embodiments, X is 4 amino acids. In some embodiments, X is 5, 6, or 7 amino acids. In some embodiments, X is 8, 9, or 10 amino acids.

In some embodiments, the 1-10 amino acids of X are each independently selected from natural amino acids. In some embodiments, the 1-10 amino acids of X are each independently selected from non-natural amino acids. In some embodiments, the 1-10 amino acids of X are each independently selected from non-classical amino acids. In some embodiments, the 1-10 amino acids of X are each independently selected from a combination of natural amino acids, non-natural amino acids, and/or non-classical amino acids.

In some embodiments, X does not include any glycine residues. In some embodiments, X does not include any contiguous glycine residues, such as di-glycine, tri-glycine, tetra-glycine, penta-glycine, or hexa-glycine. In some embodiments, X includes one or more non-contiguous glycine residues.

In some embodiments, X is not a sortase enzyme recognition motif.

In some embodiments, X is not -Leu-Pro-*-Thr-Gly-, -Gly-Thr-*-Pro-Leu-, -Gly-Ser-*-Pro-Leu-, -Gly-Thr-*-Ala-Leu-, -Gly-Thr-*-Pro-Leu-, -Gly-Ser-*-Pro-Leu-, -Gly-Thr-*-Ala-Leu-, -Thr-*-Pro-Leu-, -Ser-*-Pro-Leu-, -Thr-*-Ala-Leu-, -Thr-*-Pro-Leu-, -Ser-*-Pro-Leu-, -Thr-*-Ala-Leu-, -Gln-Pro-Gln-Thr-Asp-; wherein * is any natural amino acid.

In some embodiments, X is not -Lys-Pro-Gly-Thr-Gly- or -Asp-Pro-Gln-Thr-Gln-.

In some embodiments, X is a 4-16 membered heteroalkylene optionally substituted with 1-3 independently selected R^{X}. In some embodiments, X is a 4-12 membered heteroalkylene optionally substituted with 1-3 independently selected R^{X}. In some embodiments, X is a 4-8 membered heteroalkylene optionally substituted with 1-3 independently selected R^{X}.

In some embodiments, X is a 4-16 membered heteroalkylene substituted with 1-3 independently selected R^{X}. In some embodiments, X is a 4-12 membered heteroalkylene substituted with 1-3 independently selected R^{X}. In some embodiments, X is a 4-8 membered heteroalkylene substituted with 1-3 independently selected R^{X}.

In some embodiments, X is a 4-16 membered heteroalkylene substituted with 3 independently selected R^{X}. In some embodiments, X is a 4-12 membered heteroalkylene substituted with 3 independently selected R^{X}. In some embodiments, X is a 4-8 membered heteroalkylene substituted with 3 independently selected R^{X}.

In some embodiments, X is a 4-16 membered heteroalkylene substituted with 1 or 2 independently selected R^{X}. In some embodiments, X is a 4-12 membered heteroalkylene substituted with 1 or 2 independently selected R^{X}. In some embodiments, X is a 4-8 membered heteroalkylene substituted with 1 or 2 independently selected R^{X}.

In some embodiments, X is a 4-16 membered heteroalkylene substituted with 2 independently selected R^{X}. In some embodiments, X is a 4-12 membered heteroalkylene substituted with 2 independently selected R^{X}. In some embodiments, X is a 4-8 membered heteroalkylene substituted with 2 independently selected R^{X}.

In some embodiments, X is a 4-16 membered heteroalkylene substituted with 1 R^{X}. In some embodiments, X is a 4-12 membered heteroalkylene substituted with 1 R^{X}. In some embodiments, X is a 4-8 membered heteroalkylene substituted with 1 R^{X}.

In some embodiments, each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; and each R^{X1} and R^{X2} are independently hydrogen or C₁₋₆ alkyl.

In some embodiments, one R^{X} is a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl.

In some embodiments, one R^{X} is a C₁-C₆ alkyl group substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl.

In some embodiments, one R^{X} is a C₁-C₆ alkyl group substituted with hydroxyl.

In some embodiments, one R^{X} is a C₁-C₆ alkyl group substituted with guanidino.

In some embodiments, one R^{X} is a C₁-C₆ alkyl group substituted with 1 or 2 —CO₂H groups.

In some embodiments, one R^{X} is a C₁-C₆ alkyl group substituted with 1 —CO₂H group.

In some embodiments, one R^{X} is a C₁-C₆ alkyl group substituted with 2 —CO₂H groups.

In some embodiments, one R^{X} is a C₁-C₆ alkyl group substituted with urea.

In some embodiments, one R^{X} is a C₁-C₆ alkyl group substituted with phenyl.

In some embodiments, one R^{X} is a C₁-C₆ alkyl group substituted with naphthyl.

In some embodiments, one R^{X} is a C₁-C₆ alkyl group substituted with indolyl.

In some embodiments, one R^{X} is a C₁-C₆ alkyl group substituted with imidazolyl.

In some embodiments, one R^{X} is a C₁-C₆ alkyl group substituted with —SH, —SCH₃, or —SeCH₃.

In some embodiments, one R^{X} is a C₁-C₆ alkyl group substituted with 4-hydroxyphenyl optionally substituted with C₂-C₆ alkenyl.

In some embodiments, one R^{X} is a C₁-C₆ alkyl group substituted with —C(═O)NR^{X1}R^{X2}.

In some embodiments, one R^{X} is a C₁-C₆ alkyl group substituted with —NR^{X1}R^{X2}.

In some embodiments, one R^{X} is —NR^{X1}R^{X2}.

In some embodiments, R^{X1} and R^{X2} are each independently C₁₋₆ alkyl. In some embodiments, R^{X1} and R^{X2} are each methyl. In some embodiments, R^{X1} and R² are each hydrogen. In some embodiments, one of R^{X1} and R^{X2} is hydrogen and the other of R^{X1} and R² is C₁₋₆ alkyl. In some embodiments, one of R^{X1} and R² is hydrogen and the other of R^{X1} and R^{X2} is methyl.

In some embodiments, one R^{X} is a C₂-C₆ alkynyl group.

In some embodiments, X is substituted with two R^{X}; wherein each R^{X} is an independently selected unsubstituted C₁-C₆ alkyl group.

In some embodiments, X is substituted with one R^{X}; wherein R^{X} is an unsubstituted C₁-C₆ alkyl group.

In some embodiments, X is substituted with two R^{X}; wherein the two R^{X} are attached to the same or adjacent carbon atom(s) of X, and together with the carbon atom(s) to which they are attached form an unsubstituted 5-6 membered heterocyclyl. In some embodiments, X is substituted with two R^{X}; wherein the two R^{X} are attached to the same carbon atom of X, and together with the carbon atom to which they are attached form an unsubstituted 5-6 membered heterocyclyl. In some embodiments, X is substituted with two R^{X}; wherein the two R^{X} are attached to adjacent carbon atoms of X, and together with the carbon atoms to which they are attached form an unsubstituted 5-6 membered heterocyclyl. In some embodiments, the two R^{X}, together with the carbon atom(s) to which they are attached form an unsubstituted 5-6 membered heterocyclyl selected from the group consisting of pyrrolidine, imidazolidine, piperidine, piperazine, and morpholine. In some embodiments, the two R^{X}, together with the carbon atom(s) to which they are attached form an unsubstituted pyrrolidine.

In some embodiments, X is substituted with two R^{X}; wherein one R^{X} is a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; wherein each R^{X1} and R^{X2} are independently hydrogen or C₁₋₆ alkyl; and the other R^{X} is an unsubstituted C₁-C₆ alkyl group.

In some embodiments, X is substituted with one, two, or three methyl groups. In some embodiments, X is substituted with one or two (N)-methyl groups (i.e., X is substituted with methyl group on a nitrogen atom of X). In some embodiments, X is substituted with a geminal dimethyl group (two methyl groups attached to the same atom).

In some embodiments, X is —NH(C₂-C₆ alkylene)NH— optionally substituted with C₁-C₆ alkyl. In some embodiments, X is —NH(C₂-C₃ alkylene)NH— optionally substituted with C₁-C₆ alkyl. In some embodiments, X is —NH(C₂-C₆ alkylene)NH— optionally substituted with two independently selected C₁-C₆ alkyl groups. In some embodiments, X is —NH(C₂-C₃ alkylene)NH-optionally substituted with two independently selected C₁-C₆ alkyl groups. In some embodiments, X is ##—NH(C₂-C₆ alkylene)NH-(PEG2 to PEG4)-, wherein ## indicates attachment to D.

In some embodiments, X is ##—NH(C₂-C₆ alkylene)-, wherein ## indicates attachment to D. In some embodiments, X is ##—NH(C₂-C₆ alkylene)-(PEG2 to PEG4)-, wherein ## indicates attachment to D. In some embodiments, X is ##—NH(C₂-C₆ alkylene)NH—[(C(O)CH₂NH]₁₋₂— or ##—NH(C₂-C₆ alkylene)NH—[(C(O)CHR^{X}NH]1.3-, wherein R^{X} is C₁₋₃ alkyl optionally substituted with —OH and ## indicates attachment to D. In some embodiments, X is ##-[NHCH₂C(O)]₁₋₃—NH(C₂-C₆ alkylene)NH— or ##-[NHCHR^{X}C(O)]₁₋₃—NH(C₂-C₆ alkylene)NH—, wherein R^{X} is C₁₋₃ alkyl optionally substituted with —OH and ## indicates attachment to D. In some embodiments, X is ##—NR^{X}(C₂-C₆ alkylene)NR^{X}—, wherein R^{X} is C₁₋₃ alkyl and ## indicates attachment to D. In some embodiments, X is ##-[NHCH₂C(O)]₁₋₃- or ##-[NHCHR^{X}C(O)]1.3-, wherein R^{X} is C₁₋₃ alkyl optionally substituted with —OH and ## indicates attachment to D.

In some embodiments, X is an unsubstituted 4-16 membered heteroalkylene. In some embodiments, X is an unsubstituted 4-12 membered heteroalkylene. In some embodiments, X is an unsubstituted 4-8 membered heteroalkylene.

In some embodiments, X is selected from the group consisting of:

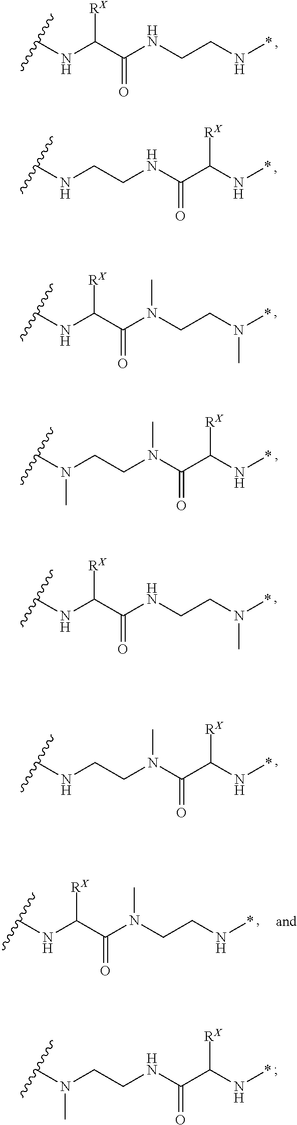

wherein the wavy line represents covalent attachment to Y, W, A, or M; and the * represents covalent attachment to D.

In some embodiments, X is selected from the group consisting of:

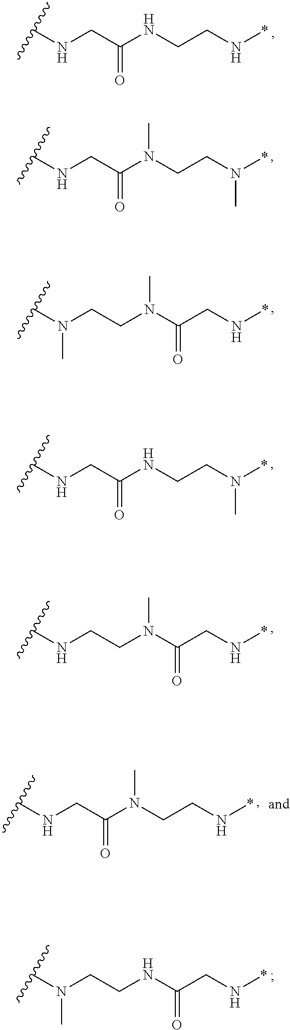

wherein the wavy line represents covalent attachment to Y, W, A, or M; and the * represents covalent attachment to D.

In some embodiments, X is not

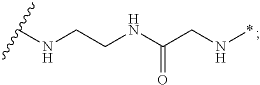 [SMILES: *NCCNC(=O)CN*]

wherein the wavy line represents covalent attachment to Y, W, A, or M; and the * represents covalent attachment to D.

In some embodiments, X is not PEG1-PEG8. In some embodiments, X is not PEG1. In some embodiments, X is not PEG2. In some embodiments, X is not PEG3. In some embodiments, X is not PEG4. In some embodiments, X is not PEG5. In some embodiments, X is not PEG6. In some embodiments, X is not PEG7. In some embodiments, X is not PEG8.

In some embodiments, each D-X is:

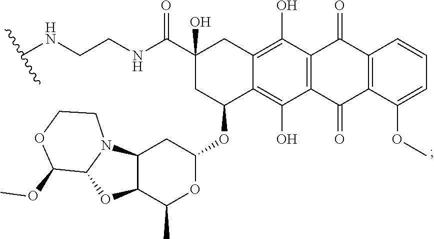

wherein [symbol] represents covalent attachment to Y, W, A, or M.

In some embodiments, each D-X is:

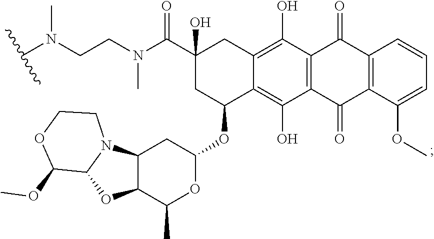

wherein [symbol] represents covalent attachment to Y, W, A, or M.

In some embodiments, each D-X is:

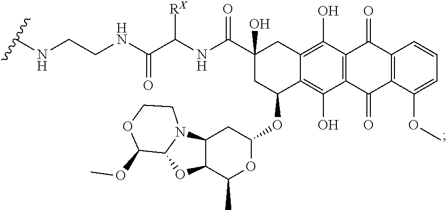

wherein [symbol] represents covalent attachment to Y, W, A, or M.

In some embodiments, each D-X is:

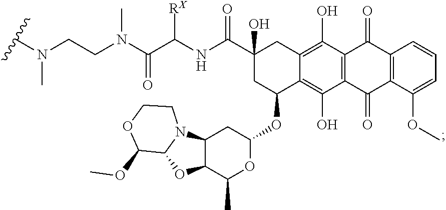

wherein [symbol] represents covalent attachment to Y, W, A, or M.

In some embodiments, each D-X is:

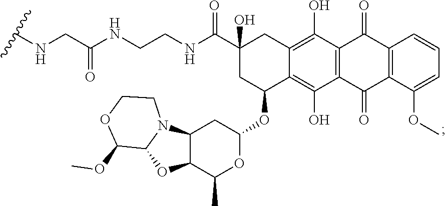

wherein [symbol] represents covalent attachment to Y, W, A, or M.

In some embodiments, each D-X is:

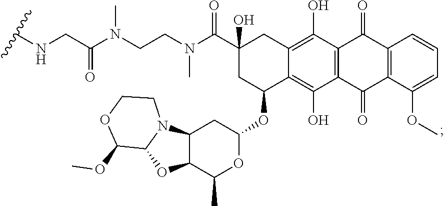

wherein [symbol] represents covalent attachment to Y, W, A, or M.

In some embodiments, each D-X is:

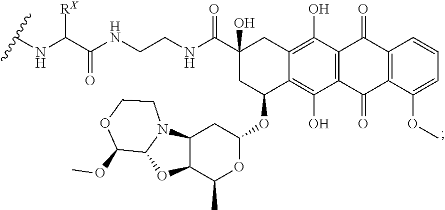

wherein [symbol] represents covalent attachment to Y, W, A, or M.

In some embodiments, each D-X is:

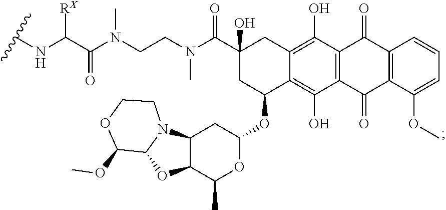

wherein [symbol] represents covalent attachment to Y, W, A, or M.

In some embodiments, subscript y is 0. In some embodiments subscript y is 1.

In some embodiments, Y is a self-immolative moiety, a non-self-immolative releasable moiety, or a non-cleavable moiety. In some embodiments, Y is a self-immolative moiety or a non-self-immolative releasable moiety. In some embodiments, Y is a self-immolative moiety. In some embodiments, Y is a non-self-immolative moiety.

A non-self-immolative moiety is one which requires enzymatic cleavage, and in which part or all of the group remains bound to the Drug Unit after cleavage from the ADC, thereby forming free drug. Examples of a non-self-immolative moiety include, but are not limited to: -glycine-; -glycine-glycine-; and a p-aminobenzyl alcohol (PAB) optionally substituted with 1-4 substituents independently selected from halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂. When an ADC having Y is -glycine- or -glycine-glycine-undergoes enzymatic cleavage (for example, via a cancer-cell-associated protease or a lymphocyte-associated protease), the Drug Unit is cleaved from the ADC such that the free drug includes the glycine or glycine-glycine group from Y. In some embodiments, an independent hydrolysis reaction takes place within, or in proximity to, the target cell, further cleaving the glycine or glycine-glycine group from the free drug. For example, an ADC with a non-self-immolative linker with a PAB optionally substituted with 1-4 substituents independently selected from halogen, cyano, and nitro, can undergo enzymatic cleavage of the linker (for example, via a cancer-cell-associated protease or a lymphocyte-associated protease), releasing a free drug which includes the optionally substituted PAB. This compound may further undergo 1,6-elimination of the PAB, removing any portion of Y from the free drug. See, e.g., Told et al., 2002, J. Org. Chem. 67:1866-1872. In some embodiments, enzymatic cleavage of the non-self-immolative moiety, as described herein, does not result in any further hydrolysis step(s).

Other examples of self-immolative groups include, but are not limited to, aromatic compounds that are electronically similar to the PAB group such as 2-aminoimidazol-5-methanol derivatives (see, e.g., Hay et al., 1999, Bioorg. Med. Chem. Lett. 9:2237), ortho or para-aminobenzylacetals, substituted and unsubstituted 4-aminobutyric acid amides (see, e.g., Rodrigues et al., 1995, Chemistry Biology 2:223), appropriately substituted bicyclo[2.2.1] and bicyclo[2.2.2] ring systems (see, e.g., Storm et al., 1972, J. Amer. Chem. Soc. 94:5815), 2-aminophenylpropionic acid amides (see, e.g., Amsberry et al., 1990, J. Org. Chem. 55:5867), and elimination of amine-containing drugs that are substituted at the α-position of glycine (see, e.g., Kingsbury et al., 1984, J. Med. Chem. 27:1447).

In some embodiments, Y is a p-aminobenzyl alcohol (PAB) optionally substituted with 1-4 substituents independently selected from halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂. In some embodiments, Y is an unsubstituted p-aminobenzyl alcohol (PAB).

In some embodiments, Y is a para-aminobenzyloxy-carbonyl (PABC) group optionally substituted with a sugar moiety. In some embodiments, Y is -glycine- or -glycine-glycine-. In some embodiments, Y is a branched bis(hydroxymethyl)styrene (BHMS) unit, which is capable of incorporating (and releasing) multiple Drug Units.

In some embodiments, Y is

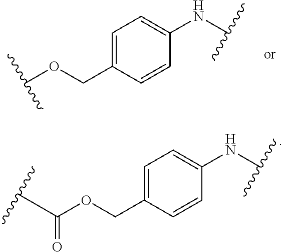 [SMILES: *Nc1ccc(CO*)cc1 ; *Nc1ccc(COC(*)=O)cc1]

In some embodiments, subscript w is 0. In some embodiments subscript w is 1.

In some embodiments, W is a single amino acid. In some embodiments, W is a single natural amino acid. In some embodiments, W is a peptide including from 2-6 amino acids, wherein each amino acid is independently a natural or non-natural amino acid. In some embodiments, each amino acid is independently a natural amino acid. In some embodiments, W is a peptide including from 2-6 amino acids, wherein each amino acid is independently selected from non-natural amino acids. In some embodiments, W is a peptide including from 2-6 amino acids, wherein each amino acid is independently selected from non-classical amino acids. In some embodiments, W is a peptide including from 2-6 amino acids, wherein each amino acid is independently selected from a combination of natural amino acids, non-natural amino acids, and/or non-classical amino acids. In some embodiments, W is a peptide including from 1-3 amino acids, wherein each amino acid is independently selected from a combination of natural amino acids, non-natural amino acids, and/or non-classical amino acids. In some embodiments, W is a dipeptide. In some embodiments, W is a tripeptide. In some embodiments, W is a tetrapeptide. In some embodiments, W is a pentapeptide. In some embodiments, W is a hexapeptide.

In some embodiments, each amino acid of W is independently selected from the group consisting of valine, alanine, β-alanine, glycine, lysine, leucine, phenylalanine, proline, aspartic acid, serine, glutamic acid, homoserine methyl ether, aspartate methyl ester, N,N-dimethyl lysine, arginine, citrulline, isoleucine, histidine, threonine, O-methylserine, O-methylaspartic acid, O-methylglutamic acid, N-methyllysine, O-methyltyrosine, O-methylhistidine, and O-methylthreonine. In some embodiments, W is an aspartic acid. In some embodiments, W is a lysine. In some embodiments, W is a glycine. In some embodiments, W is an alanine. In some embodiments, W is aspartate methyl ester. In some embodiments, W is a N,N-dimethyl lysine. In some embodiments, W is a homoserine methyl ether. In some embodiments, W is a serine.

In some embodiments, W is a dipeptide selected from the group consisting of valine-alanine, valine-citrulline, and phenylalanine-lysine. In some embodiments, W is valine-alanine. In some embodiments, W is valine-citrulline. In some embodiments, W is phenylalanine-lysine.

In some embodiments, when W is from 2-6 amino acids, each amino acid is independently selected from group consisting of valine, alanine, β-alanine, lysine, leucine, phenylalanine, proline, aspartic acid, serine, glutamic acid, homoserine methyl ether, aspartate methyl ester, N,N-dimethyl lysine, arginine, valine-alanine, valine-citrulline, phenylalanine-lysine, and citrulline.

In some embodiments, W is from 2-6 amino acids; and the bond between W and X or between W and Y is enzymatically cleavable by a tumor-associated protease. In some embodiments, the tumor-associated protease is a cathepsin. In some embodiments, the tumor-associated protease is cathepsin B, C, or D.

In some embodiments, W does not include any glycine residues. In some embodiments, W does not include any contiguous glycine residues, such as di-glycine, tri-glycine, tetra-glycine, penta-glycine, or hexa-glycine. In some embodiments, W includes one or more non-contiguous glycine residues.

In some embodiments, W is not a sortase enzyme recognition motif.

In some embodiments, W is not -Leu-Pro-*-Thr-Gly-, -Gly-Thr-*-Pro-Leu-, -Gly-Ser-*-Pro-Leu-, -Gly-Thr-*-Ala-Leu-, -Gly-Thr-*-Pro-Leu-, -Gly-Ser-*-Pro-Leu-, -Gly-Thr-*-Ala-Leu-, -Thr-*-Pro-Leu-, -Ser-*-Pro-Leu-, -Thr-*-Ala-Leu-, -Thr-*-Pro-Leu-, -Ser-*-Pro-Leu-, -Thr-*-Ala-Leu-, -Gln-Pro-Gln-Thr-Asp-; wherein * is any natural amino acid.

In some embodiments, W is not -Lys-Pro-Gly-Thr-Gly- or -Asp-Pro-Gln-Thr-Gln-.

In some embodiments, W has the structure of:

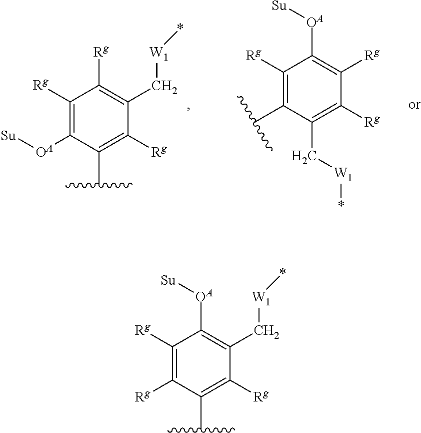

wherein Su is a Sugar moiety;
—O^{A}— represents the oxygen atom of a glycosidic bond;
each R⁹ is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W₁ is absent, *—C(═O)—O—, or *—O—C(═O)—;
[symbol] represents covalent attachment to A or M; and
the * represents covalent attachment to Y or X;

In some embodiments, —O^{A}— represents the oxygen atom of a glycosidic bond. In some embodiments, the glycosidic bond provides a β-glucuronidase or a α-mannosidase-cleavage site. In some embodiments, the β-glucuronidase or a α-mannosidase-cleavage site is cleavable by human lysosomal β-glucuronidase or by human lysosomal α-mannosidase.

In some embodiments, W is

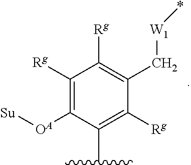

In some embodiments, W is

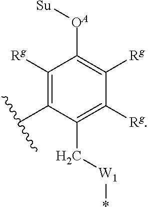

In some embodiments, W is

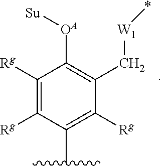

In some embodiments, each R⁹ is hydrogen. In some embodiments, one R⁹ is hydrogen, and the remaining R⁹ are independently halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂. In some embodiments, two R⁹ are hydrogen, and the remaining R⁹ is halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂.

In some embodiments, one R⁹ is halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂, and the other R⁹ are hydrogen.

In some embodiments, O^{A}—Su is charged neutral at physiological pH. In some embodiments, O^{A}—Su is mannose. In some embodiments, O^{A}—Su is

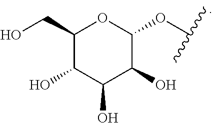 [SMILES: *O[C@H]1O[C@H](CO)[C@@H](O)[C@H](O)[C@@H]1O]

In some embodiments, O^{A}—Su comprises a carboxylate moiety. In some embodiments, O^{A}—Su is glucuronic acid. In some embodiments, O^{A}-Su is

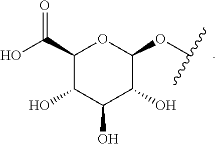 [SMILES: *O[C@@H]1O[C@H](C(=O)O)[C@@H](O)[C@H](O)[C@H]1O]

In some embodiments, W is

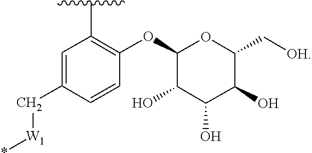

In some embodiments, W is

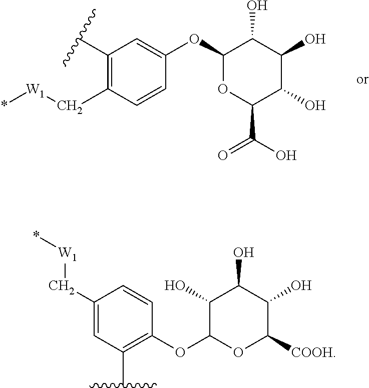

In some embodiments, W is

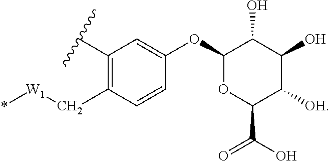

In some embodiments, W is

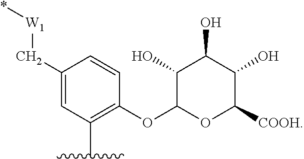

In some embodiments, W₁ is absent. In some embodiments, W₁ is *—C(═O)—O—. In some embodiments, W₁ is absent or *—O—C(═O)—. In some embodiments, W₁ is *—O—C(═O)—.

In some embodiments, W is a Cleavable Unit. In some embodiments, W is a Peptide Cleavable Unit. In some embodiments, W is a Glucuronide Unit.

In some embodiments,
A is a C₂₋₁₀ alkylene optionally substituted with 1-3 R^{a1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
W is from 2-6 amino acids, wherein:
   W is not a sortase enzyme recognition motif, and W does not include

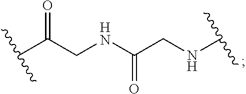 [SMILES: *NCC(=O)NCC(*)=O]

Y is a self-immolative moiety, a non-self-immolative releasable moiety, or a non-cleavable moiety; and
L is optionally substituted with a PEG Unit from PEG1 to PEG72.

In some embodiments,
A is a C₂₋₁₀ alkylene optionally substituted with 1-3 Ra;
each R^{a1} is independently selected from the group consisting of: ═O, —NR^{d1}R^{e1}, —(C₁₋₆ alkylene)-NR^{d1}R^{e1}, and —C(═O)NR^{d1}R^{e1};
each R^{d1} and R^{e1} are independently hydrogen or C₁₋₃ alkyl;
W is from 2-6 amino acids, wherein:
   W is not a sortase enzyme recognition motif, and W does not include

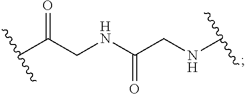 [SMILES: *NCC(=O)NCC(*)=O]

Y is a self-immolative moiety, a non-self-immolative releasable moiety, or a non-cleavable moiety; and
L is optionally substituted with a PEG Unit from PEG1 to PEG72.

In some embodiments:
subscript y is 0;
subscript w is 0;
subscript a is 1; and
each D-X is:

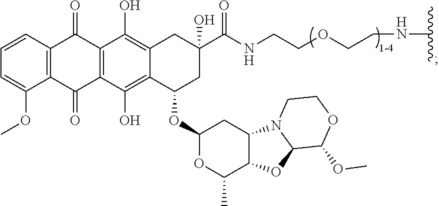

wherein [symbol] represents covalent attachment to A.

In some embodiments, subscript a is 0. In some embodiments subscript a is 1.

In some embodiments, A is a C₂₋₁₀ alkylene optionally substituted with 1-3 R^{a1}. In some embodiments, A is a C₄₋₁₀ alkylene optionally substituted with 1-3 R^{a1}. In some embodiments, A is a C₂₋₁₀ alkylene substituted with one R^{a1}. In some embodiments, A is a C₄₋₁₀ alkylene substituted with one R^{a1}. In some embodiments, A is a C₄₋₈ alkylene substituted with one R^{a1}.

In some embodiments, each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —C(═O)NR^{d1}R^{e1}, —C(═O)(C₁₋₆ alkyl), and —C(═O)O(C₁₋₆ alkyl). In some embodiments, each R^{a1} is C₁₋₆ alkyl. In some embodiments, each R^{a1} is C₁₋₆ haloalkyl. In some embodiments, each R^{a1} is C₁₋₆ alkoxy. In some embodiments, each R^{a1} is C₁₋₆ haloalkoxy. In some embodiments, each R^{a1} is halogen. In some embodiments, each R^{a1} is —OH. In some embodiments, each R^{a1} is ═O. In some embodiments, each R^{a1} is —NR^{d1}R^{e1}. In some embodiments, each R^{a1} is —(C₁₋₆ alkylene)-NR^{d1}R^{e1}. In some embodiments, each R^{a1} is —C(═O)NR^{d1}R^{e1}. In some embodiments, each R^{a1} is —C(═O)(C₁₋₆ alkyl). In some embodiments, each R^{a1} is —C(═O)O(C₁₋₆ alkyl). In some embodiments, one occurrence of R^{a1} is —NR^{d1}R^{e1}. In some embodiments, one occurrence of R^{a1} is —(C₁₋₆ alkylene)-NR^{d1}R^{e1}. In some embodiments, one occurrence of R^{a1} is —(C₁₋₂ alkylene)-NR^{d1}R^{e1}. In some embodiments, A is a C₂₋₂₀ alkylene substituted with 1 or 2 R^{a1}, each of which is ═O.

In some embodiments, R^{d1} and R^{e1} are independently hydrogen or C₁₋₃ alkyl. In some embodiments, one of R^{d1} and R^{e1} is hydrogen, and the other of R^{d1} and R^{e1} is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, A is an unsubstituted C₂₋₁₀ alkylene. In some embodiments, A is an unsubstituted C₂₋₆ alkylene. In some embodiments, A is an unsubstituted C₄₋₈ alkylene. In some embodiments, A is an unsubstituted C₄₋₁₀ alkylene.

In some embodiments, A is a 3 to 20 membered heteroalkylene optionally substituted with 1-3 R^{b1}. In some embodiments, A is a 3 to 12 membered heteroalkylene optionally substituted with 1-3 R^{b1}. In some embodiments, A is a 4 to 12 membered heteroalkylene optionally substituted with 1-3 R^{b1}. In some embodiments, A is a 4 to 8 membered heteroalkylene optionally substituted with 1-3 R^{b1}. In some embodiments, A is a 3 to 20 membered heteroalkylene substituted with one R^{b1}. In some embodiments, A is a 3 to 12 membered heteroalkylene substituted with one R^{b1}. In some embodiments, A is a 4 to 12 membered heteroalkylene substituted with one R^{b1}. In some embodiments, A is a 4 to 8 membered heteroalkylene substituted with one R^{b1}.

In some embodiments, each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl). In some embodiments, each R^{b1} is C₁₋₆ alkyl. In some embodiments, each R^{b1} is C₁₋₆ haloalkyl. In some embodiments, each R^{b1} is C₁₋₆ alkoxy. In some embodiments, each R^{b1} is C₁₋₆ haloalkoxy. In some embodiments, each R^{b1} is halogen. In some embodiments, each R^{b1} is —OH. In some embodiments, each R^{b1} is —NR^{d1}R^{e1}. In some embodiments, each R^{b1} is —(C₁₋₆ alkylene)-NR^{d1}R^{e1}. In some embodiments, each R^{b1} is C(═O)NR^{d1}R^{e1}. In some embodiments, each R^{b1} is —C(═O)(C₁₋₆ alkyl). In some embodiments, each R^{b1} is —C(═O)O(C₁₋₆ alkyl). In some embodiments, one occurrence of R^{b1} is —NR^{d1}R^{e1}. In some embodiments, one occurrence of R^{b1} is —(C₁₋₆ alkylene)-NR^{d1}R^{e1}. In some embodiments, one occurrence of R^{b1} is —(C₁-2 alkylene)-NR^{d1}R^{e1}.

In some embodiments, R^{d1} and R^{e1} are independently hydrogen or C₁₋₃ alkyl. In some embodiments, one of R^{d1} and R^{e1} is hydrogen, and the other of R^{d1} and R^{e1} is C₁₋₃ alkyl. In some embodiments, R^{d1} and R^{e1} are both hydrogen or C₁₋₃ alkyl. In some embodiments, R^{d1} and R^{e1} are both C₁₋₃ alkyl. In some embodiments, R^{d1} and R^{e1} are both methyl.

In some embodiments, A is a 3 to 20 membered heteroalkylene. In some embodiments, A is a 3 to 12 membered heteroalkylene. In some embodiments, A is a 4 to 12 membered heteroalkylene. In some embodiments, A is a 4 to 8 membered heteroalkylene.

In some embodiments, A is selected from the group consisting of:

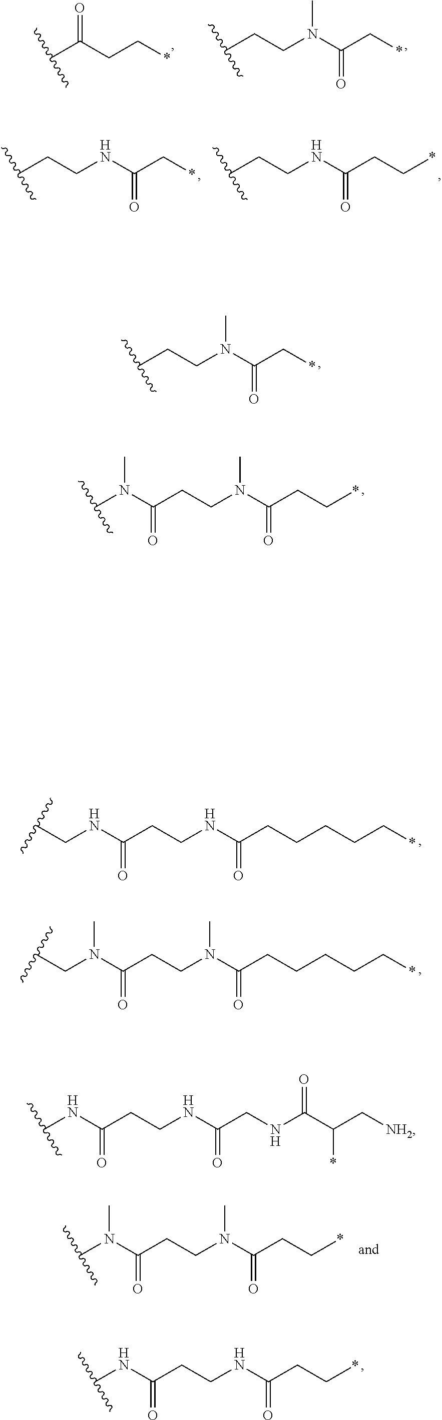

wherein the wavy line represents covalent attachment to W, Y, or X, and * represents covalent linkage to M.

In some embodiments, A is selected from —(CH₂)₁₋₆—, —C(O)(CH₂)₁₋₆-#, —[NHC(O)(CH₂)₁₋₄]₁₋₃-#, and —NH(CH₂)₁₋₆[NHC(O)(CH₂)₁₋₄]₁₋₂-#, wherein # indicates attachment to M.

In some embodiments, M is a succinimide. In some embodiments, M is a hydrolyzed succinimide. It will be understood that a hydrolyzed succinimide may exist in two regioisomeric form(s). Those forms are exemplified below for hydrolysis of M, wherein the structures representing the regioisomers from that hydrolysis are formula M′ and M″; wherein wavy line a indicates the point of covalent attachment to the antibody, and wavy line b indicates the point of covalent attachment to A.

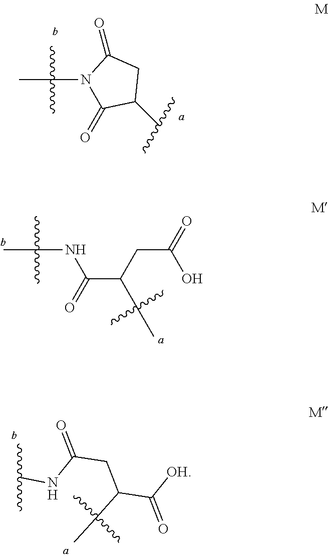 [SMILES: *C1CC(=O)N(*)C1=O ; *NC(=O)C(*)CC(=O)O ; *NC(=O)CC(*)C(=O)O]

In some embodiments, M′ is

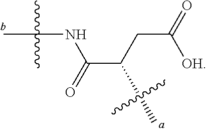

In some embodiments, M′ is

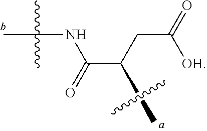

In some embodiments, M″ is

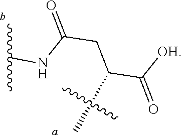

In some embodiments, M″ is

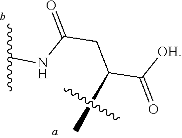

In some embodiments, -M-A- is selected from the group consisting of:

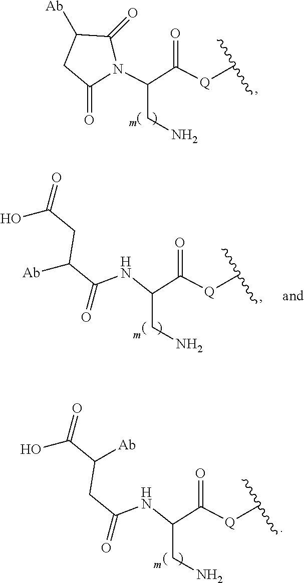

wherein subscript m is 1 or 2; Q is —NH—(C₁-C₅ alkylene)-C(═O)—, or is absent;
wherein Ab is the antibody and the wavy line represents covalent attachment to W, Y, or X.

In some embodiments, -M-A- is selected from the group consisting of:

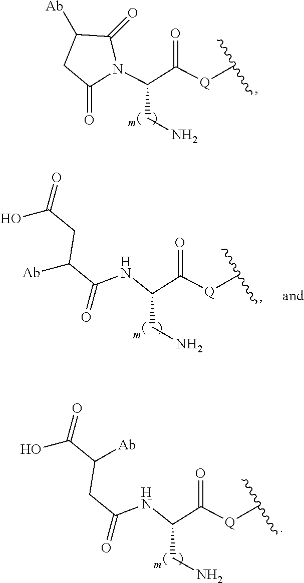

In some embodiments, -M-A- is selected from the group consisting of:

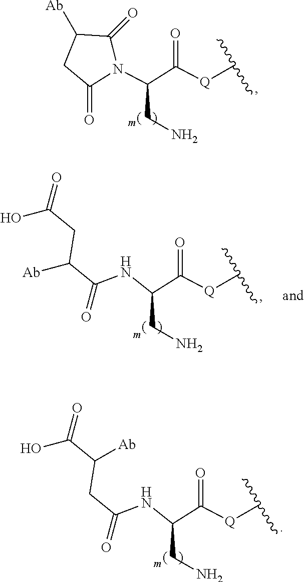

In some embodiments, Q is —NH—CH₂—C(═O)—. In some embodiments, Q is absent.

In some embodiments, -M-A- is

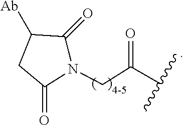

In some embodiments, -M-(A)ₐ-(W)_{w}—(Y)_{y}—(X)— is a non-self-immolative releasable linker, which provides release of the free drug once the ADC has been internalized into the target cell. In some embodiments, -M-(A)ₐ-(W)_{w}—(Y)_{y}—(X)— is a releasable linker, which provides release of the free drug with, or in the vicinity, of targeted cells. In some embodiments, releasable linkers possess a recognition site, such as a peptide cleavage site, sugar cleavage site, or disulfide cleavage site. In some embodiments, each releasable linker is a di-peptide. In some embodiments, each releasable linker is a disulfide. In some embodiments, each releasable linker is a hydrazone. In some embodiments, each releasable linker is independently selected from the group consisting of Val-Cit-, -Phe-Lys-, and -Val-Ala-. In some embodiments, each releasable linker, when bound to a succinimide or hydrolyzed succinimide, is independently selected from the group consisting of succinimido-caproyl (me), succinimido-caproyl-valine-citrulline (sc-vc), succinimido-caproyl-valine-citrulline-paraaminobenzyloxycarbonyl (sc-vc-PABC), and SDPr-vc (where “S” refers to succinimido).

In some embodiments, -M-(A)ₐ-(W)_{w}-(Y)_{y}-(X)— comprises a non-cleavable linker. Non-cleavable linkers are known in the art and can be adapted for use with the ADCs described herein as the “Y” group. A non-cleavable linker is capable of linking a Drug Unit to an antibody in a generally stable and covalent manner and is substantially resistant to cleavage, such as acid-induced cleavage, light-induced cleavage, peptidase- or esterase-induced cleavage, and disulfide bond cleavage. The free drug can be released from the ADCs containing non-cleavable linkers via alternative mechanisms, such as proteolytic antibody degradation. In some embodiments, the Drug Unit can exert a biological effect as a part of the ADC (i.e., while still conjugated to the antibody via a linker).

Reagents that form non-cleavable linker-maleimide and non-cleavable linker-succinimide compounds are known in the art and can adapted for use herein. Exemplary reagents comprise a maleimido or haloacetyl-based moiety, such as 6-maleimidocaproic acid N-hydroxy succinimide ester (MCC), N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), maleimidoundecanoic acid N-succinimidyl ester (KMUA), 7-maleimidobutyric acid N-succinimidyl ester (GMBS), c-maleimidocaproic acid N-hydroxysuccinimide ester (EMCS), m-maleimidobenzoyl-N-hydroxysuccinimide ester (MBS), N-(α-maleimidoacetoxy)-succinimide ester [AMAS], succinimidyl-6-(β-maleimidopropionamido)hexanoate (SMPH), N-succinimidyl-4-(p-maleimidophenyl)-butyrate (SMPB), and N-(p-maleimidophenyl)isocyanate (PMPI), N-succinimidyl-4-(iodoacetyl)-aminobenzoate (STAB), N-succinimidyl iodoacetate (SIA), N-succinimidyl bromoacetate (SBA) and N-succinimidyl 3-(bromoacetamido)propionate (SBAP). Additional “A-M” groups for use in the ADCs described herein can be found, for example, in U.S. Pat. No. 8,142,784, incorporated herein by reference in its entirety.

In some embodiments, Y is

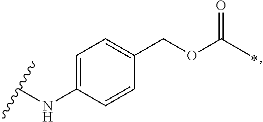 [SMILES: *Nc1ccc(COC(C)=O)cc1]

wherein the wavy line represents connection to W, A, or M; and the * represents connection to X in the ADCs described herein.

In some embodiments, -M-(A)ₐ-(W)_{w}—(Y)_{y}—(X)—, comprises a non-releasable linker, wherein the free drug is released after the ADC has been internalized into the target cell and degraded, liberating the free drug.

In some embodiments, subscript y is 0; subscript w is 1; subscript a is 1; and M is a succinimide or a hydrolyzed succinimide.

In some embodiments, subscript y is 0; subscript w is 1; subscript a is 1; M is a succinimide or a hydrolyzed succinimide; and W has the structure of:

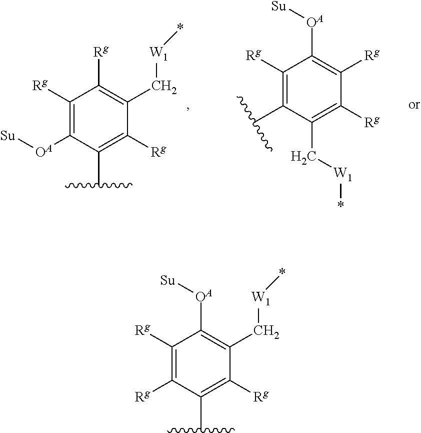

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W₁ is absent;
the wavy line represents covalent attachment to A; and
the * represents covalent attachment to X.

In some embodiments, subscript y is 0; subscript w is 1; subscript a is 1; M is a succinimide or a hydrolyzed succinimide; and W has the structure of:

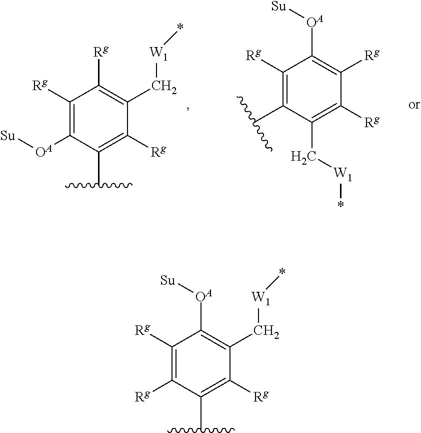

wherein Su is a Sugar moiety;
—O^{A}— represents the oxygen atom of a glycosidic bond;
each R^{g} is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W₁ is *—C(═O)—O—;
the wavy line represents covalent attachment to A; and
the * represents covalent attachment to X.

In some embodiments, subscript y is 1; subscript w is 1; subscript a is 1; and M is a succinimide or a hydrolyzed succinimide. In some embodiments, Y is a PAB group and W is a dipeptide.

In some embodiments, A is covalently attached to M; Y is attached to X; and M is attached to Ab.

In some embodiments, A comprises PEG2-PEG8. In some embodiments, X comprises PEG2-PEG6. In some embodiments, only one of X and A comprises PEG2-PEG8. In some embodiments, X does not include PEG2-PEG8. In some embodiments, A does not include PEG2-PEG8. In some embodiments, X and A do not include PEG2-PEG8 as part of the X or A groups, respectively (i.e., X and/or A can be optionally substituted with PEG, as described herein).

In some embodiments, the linker (L) is substituted with a polyethylene glycol moiety selected from the group consisting of PEG2 to PEG20. In some embodiments, L is substituted with a polyethylene glycol moiety selected from the group consisting of PEG2, PEG4, PEG6, PEG8, PEG10, PEG12, PEG16, and PEG20. In some embodiments, L is not substituted with a polyethylene glycol moiety selected from the group consisting of PEG2 to PEG20.

In some embodiments, A is substituted with a polyethylene glycol moiety selected from the group consisting of PEG2 to PEG20. In some embodiments, W is substituted with a polyethylene glycol moiety selected from the group consisting of PEG2 to PEG20. In some embodiments, Y is substituted with a polyethylene glycol moiety selected from the group consisting of PEG2 to PEG20. In some embodiments, X is substituted with a polyethylene glycol moiety selected from the group consisting of PEG2 to PEG20. In some embodiments, the linker (L) is substituted with one polyethylene glycol moiety. In some embodiments, the linker (L) is substituted with 2 or 3 independently selected polyethylene glycol moieties.

Polydisperse PEGs, monodisperse PEGs and discrete PEGs can be used to make the ADCs and intermediates thereof. Polydisperse PEGs are a heterogeneous mixture of sizes and molecular weights whereas monodisperse PEGs are typically purified from heterogeneous mixtures and therefore provide a single chain length and molecular weight. Discrete PEGs are synthesized in step-wise fashion and not via a polymerization process. Discrete PEGs provide a single molecule with defined and specified chain length. The number of —CH₂CH₂O— subunits of a PEG Unit ranges, for example, from 8 to 24 or from 12 to 24, referred to as PEG8 to PEG24 and PEG12 to PEG24, respectively.

The PEG moieties provided herein, which are also referred to as PEG Units, comprise one or multiple polyethylene glycol chains. The polyethylene glycol chains are linked together, for example, in a linear, branched or star shaped configuration. Typically, at least one of the polyethylene glycol chains of a PEG Unit is derivatized at one end for covalent attachment to an appropriate site on a component of the ADC (e.g., L). Exemplary attachments to ADCs are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some embodiments, attachment to ADC is by means of a non-conditionally cleavable linkage. In some embodiments, attachment to the ADC is not via an ester linkage, hydrazone linkage, oxime linkage, or disulfide linkage. In some embodiments, attachment to the ADC is not via a hydrazone linkage.

A conditionally cleavable linkage refers to a linkage that is not substantially sensitive to cleavage while circulating in plasma but is sensitive to cleavage in an intracellular or intratumoral environment. A non-conditionally cleavable linkage is one that is not substantially sensitive to cleavage in any biologically relevant environment in a subject that is administered the ADC. Chemical hydrolysis of a hydrazone, reduction of a disulfide bond, and enzymatic cleavage of a peptide bond or glycosidic bond of a Glucuronide Unit as described by WO 2007/011968 (which is incorporated by reference in its entirety) are examples of conditionally cleavable linkages.

In some embodiments, the PEG Unit is directly attached to the ADC (or an intermediate thereof) at L. In those embodiments, the other terminus (or termini) of the PEG Unit is free and untethered (i.e., not covalently attached), and in some embodiments, is a methoxy, carboxylic acid, alcohol or other suitable functional group. The methoxy, carboxylic acid, alcohol or other suitable functional group acts as a cap for the terminal polyethylene glycol subunit of the PEG Unit. By untethered, it is meant that the PEG Unit will not be covalently attached at that untethered site to a Drug Unit, to an antibody, or to a linking component to a Drug Unit and/or an antibody. Such an arrangement can allow a PEG Unit of sufficient length to assume a parallel orientation with respect to the drug in conjugated form, i.e., as a Drug Unit (D). For those embodiments in which the PEG Unit comprises more than one polyethylene glycol chain, the multiple polyethylene glycol chains are independently chosen, e.g., are the same or different chemical moieties (e.g., polyethylene glycol chains of different molecular weight or number of —CH₂CH₂O— subunits). A PEG Unit having multiple polyethylene glycol chains is attached to the ADC at a single attachment site. The skilled artisan will understand that the PEG Unit, in addition to comprising repeating polyethylene glycol subunits, may also contain non-PEG material (e.g., to facilitate coupling of multiple polyethylene glycol chains to each other or to facilitate coupling to the ADC). Non-PEG material refers to the atoms in the PEG Unit that are not part of the repeating —CH₂CH₂O— subunits. In some embodiments provided herein, the PEG Unit comprises two monomeric polyethylene glycol chains attached to each other via non-PEG elements. In other embodiments provided herein, the PEG Unit comprises two linear polyethylene glycol chains attached to a central core that is attached to the ADC (i.e., the PEG Unit itself is branched).

There are a number of PEG attachment methods available to those skilled in the art: see, for example: Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); ACT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal α-carbon of a peptide); Veronese et al., (1985) Appl. Biochem. Bioechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation).

For example, a PEG Unit may be covalently bound to an amino acid residue via reactive groups of a polyethylene glycol-containing compound and the amino acid residue. Reactive groups of the amino acid residue include those that are reactive to an activated PEG molecule (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) are also useful as a reactive group for forming a covalent attachment to a PEG. In addition, enzyme-assisted methods for introducing activated groups (e.g., hydrazide, aldehyde, and aromatic-amino groups) specifically at the C-terminus of a polypeptide have been described. See Schwarz, et al. (1990) Methods Enzymol. 184:160; Rose, et al. (1991) Bioconjugate Chem. 2:154; and Gaertner, et al. (1994) J. Biol. Chem. 269: 7224.

In some embodiments, a polyethylene glycol-containing compound forms a covalent attachment to an amino group using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG₂-succinimidyl succinate (mPEG₂-SS); mPEG-succinimidyl carbonate (mPEG-SC), mPEG₂-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG-SPA); mPEG-N-hydroxy-succinimide (mPEG-NHS); mPEG₂-N-hydroxy-succinimide (mPEG₂-NHS); mPEG-cyanuric chloride; mPEG₂-cyanuric chloride; mPEG₂-Lysinol-NPC, and mPEG₂-Lys-NHS.

Generally, at least one of the polyethylene glycol chains that make up the PEG is functionalized to provide covalent attachment to the ADC. Functionalization of the polyethylene glycol-containing compound that is the precursor to the PEG includes, for example, via an amine, thiol, NHS ester, maleimide, alkyne, azide, carbonyl, or other functional group. In some embodiments, the PEG further comprises non-PEG material (i.e., material not comprised of —CH₂CH₂O—) that provides coupling to the ADC or in constructing the polyethylene glycol-containing compound or PEG facilitates coupling of two or more polyethylene glycol chains.

In some embodiments, the presence of the PEG Unit in an ADC is capable of having two potential impacts upon the pharmacokinetics of the resulting ADC. One impact is a decrease in clearance (and consequent increase in exposure) that arises from the reduction in non-specific interactions induced by the exposed hydrophobic elements of the Drug Unit. The second impact is a decrease in volume and rate of distribution that sometimes arises from the increase in the molecular weight of the ADC. Increasing the number of polyethylene glycol subunits increases the hydrodynamic radius of a conjugate, typically resulting in decreased diffusivity. In turn, decreased diffusivity typically diminishes the ability of the ADC to penetrate into a tumor. See Schmidt and Wittrup, Mol Cancer Ther 2009; 8:2861-2871. Because of these two competing pharmacokinetic effects, it can be desirable to use a PEG Unit that is sufficiently large to decrease the ADC clearance thus increasing plasma exposure, but not so large as to greatly diminish its diffusivity to an extent that it interferes with the ability of the ADC to reach the intended target cell population. See, e.g., Examples 1, 18, and 21 of US 2016/0310612, which is incorporated by reference herein (e.g., for methodology for selecting an optimal size of a PEG Unit for a particular Drug Unit, Linker, and/or drug-linker compound).

In some embodiments, the PEG Unit comprises one or more linear polyethylene glycol chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some embodiments, the PEG comprises a combined total of at least 8 subunits, at least 10 subunits, or at least 12 subunits. In some such embodiments, the PEG comprises no more than a combined total of about 72 subunits. In some such embodiments, the PEG comprises no more than a combined total of about 36 subunits. In some embodiments, the PEG comprises about 8 to about 24 subunits (referred to as PEG8 to PEG24).

In some embodiments, the PEG Unit comprises a combined total of from 2 to 72, 2 to 60, 2 to 48, 2 to 36 or 2 to 24 subunits, from 3 to 72, 3 to 60, 3 to 48, 3 to 36 or 3 to 24 subunits, from 4 to 72, 8 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits. In some embodiments, the PEG Unit comprises a combined total of from 2 to 24 subunits, 2 to 16 subunits, 2 to 12 subunits, 2 to 8 subunits, or 2 to 6 subunits.

Illustrative linear PEGs that can be used in any of the embodiments provided herein are as follows:

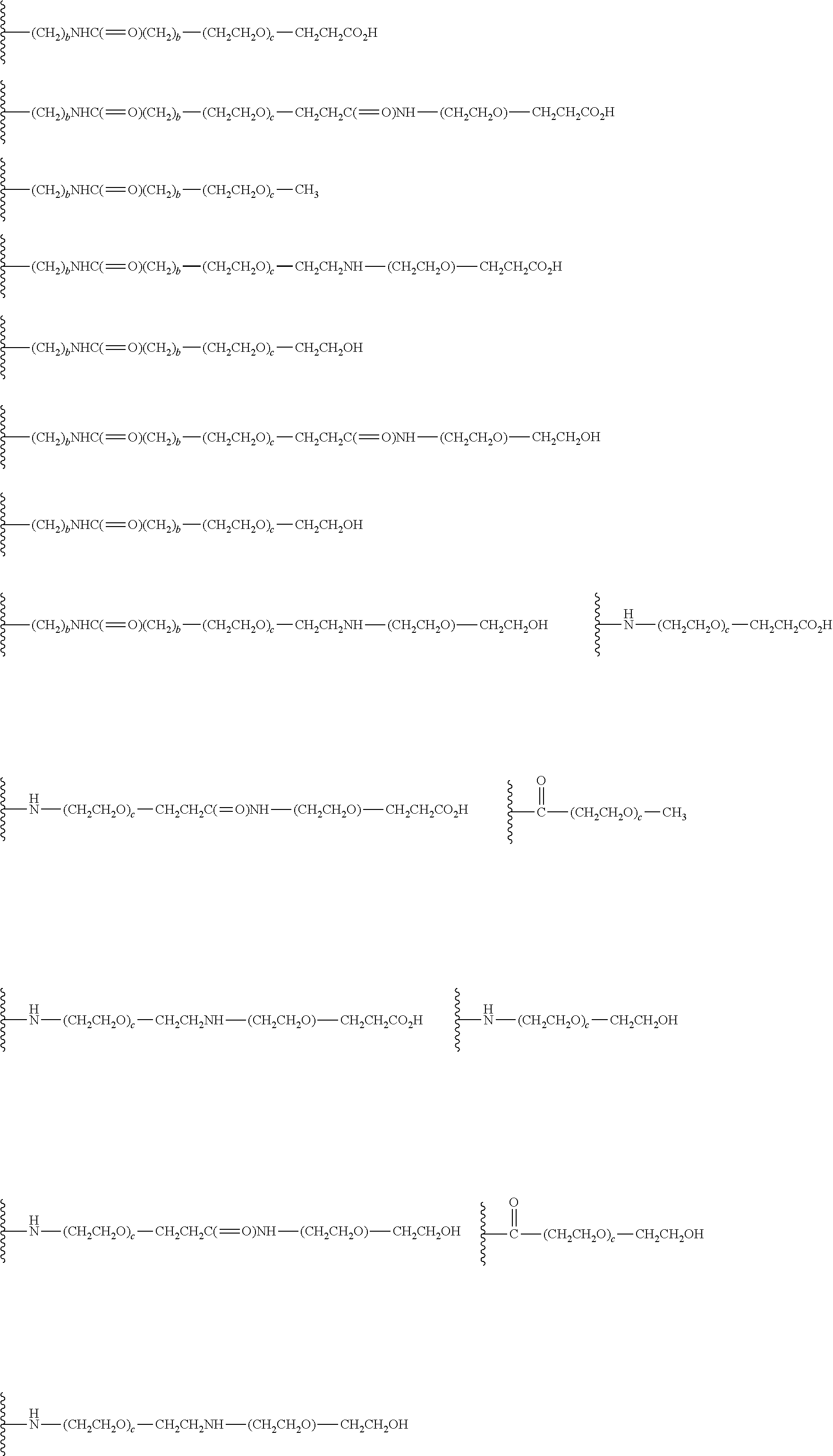

wherein the wavy line indicates the site of attachment to the ADC; each subscript b is independently selected from the group consisting of 2 to 12; and each subscript c is independently selected from the group consisting of 1 to 72, 8 to 72, 10 to 72, 12 to 72, 6 to 24, or 8 to 24. In some embodiments, each subscript b is 2 to 6. In some embodiments, each subscript c is about 2, about 4, about 8, about 12, or about 24.

In some embodiments, the sum of subscript b and subscript c (b+c) ranges from 6 to 72. In some embodiments, the sum of subscript b and subscript c (b+c) ranges from 8 to 72. In some embodiments, the sum of subscript b and subscript c (b+c) ranges from 10 to 72. In some embodiments, the sum of subscript b and subscript c (b+c) ranges from 12 to 72. In some embodiments, the sum of subscript b and subscript c (b+c) ranges from 6 to 24. In some embodiments, the sum of subscript b and subscript c (b+c) ranges from 8 to 24. In some embodiments, the sum of subscript b and subscript c (b+c) ranges from 12 to 36. In some embodiments, the sum of subscript b and subscript c (b+c) ranges from 24 to 48. In some embodiments, the sum of subscript b and subscript c (b+c) ranges from 36 to 72. In some embodiments, the sum of subscript b and subscript c (b+c) is about 8, about 12, or about 24.

As described herein, the PEG Unit can be selected such that it improves clearance of the resultant ADC but does not significantly impact the ability of the ADC to penetrate into the tumor.

In some embodiments, the PEG moiety is from about 300 Daltons to about 5,000 Daltons; from about 300 Daltons to about 4,000 Daltons; from about 300 Daltons to about 3,000 Daltons; from about 300 Daltons to about 2,000 Daltons; from about 300 Daltons to about 1,000 Daltons; or any value in between. In some embodiments, the PEG moiety has at least 8, 10 or 12 subunits. In some embodiments, the PEG Unit is PEG8 to PEG72, for example, PEG8, PEG10, PEG12, PEG16, PEG20, PEG24, PEG28, PEG32, PEG36, PEG48, or PEG72.

In some embodiments, apart from the PEGylation of the ADC, there are no other PEG subunits present in the ADC (i.e., no PEG subunits are present as part of any of the other components of the conjugates and linkers provided herein, such as A and X). In some embodiments, apart from the PEG, there are no more than 8, no more than 7, no more than 6, no more than 5, no more than 4, no more than 3, no more than 2 or no more than 1 other polyethylene glycol (—CH₂CH₂O—) subunits present in the ADC, or intermediate thereof (i.e., no more than 8, 7, 6, 5, 4, 3, 2, or 1 other polyethylene glycol subunits in other components of the ADCs (or intermediates thereof) provided herein).

It will be appreciated that when referring to polyethylene glycol subunits of a PEG Unit, and depending on context, the number of subunits can represent an average number, e.g., when referring to a population of ADCs or intermediates thereto and/or using polydisperse PEGs.

### Methods of Use of ADCs

In some embodiments, the ADCs described herein, or pharmaceutically acceptable salts thereof, are used to deliver the conjugated drug to a target cell. Without being bound by theory, in some embodiments, an ADC associates with an antigen on the surface of a target cell. The Drug Unit can then be released as free drug to induce its biological effect (such as a cytotoxic effect). The Drug Unit can also remain attached to the antibody, or a portion of the antibody and/or linker, and induce its biological effect.

Some embodiments provide a method of treating a viral or bacterial infection in a subject in need thereof, comprising administering to the subject a therapeutically effective amount of an ADC described herein, or a salt thereof.

Some embodiments provide a method of treating an autoimmune disorder in a subject in need thereof, comprising administering to the subject a therapeutically effective amount of an ADC described herein, or a salt thereof.

Some embodiments provide a method of treating cancer in a subject in need thereof, comprising administering to the subject a therapeutically effective amount of an ADC described herein, or a salt thereof.

Some embodiments provide a method of treating cancer in a subject in need thereof, comprising administering to the subject a therapeutically effective amount of a composition comprising an ADC described herein, or a salt thereof.

Some embodiments provide a method of treating cancer in a subject in need thereof, comprising administering to the subject a therapeutically effective amount of an ADC as described herein, or a salt thereof, in combination with another anticancer therapy (e.g., surgery and radiation therapy) and/or anticancer agent (e.g., an immunotherapy such as nivolumab or pembrolizumab). The ADCs described herein can be administered to the subject before, during, or after administration of the anticancer therapy and/or anticancer agent and/or surgery. In some embodiments, the ADCs described herein can be administered to the subject following treatment with radiation and/or after surgery.

Some embodiments provide a method for delaying or preventing acquired resistance to an anticancer agent, comprising administering to the subject a therapeutically effective amount of an ADC as described herein, or a salt thereof, to a patient at risk for developing or having acquired resistance to an anticancer agent. In some embodiments, the patient is administered a dose of the anticancer agent (e.g., at substantially the same time as a dose of an ADC as described herein, or a salt thereof is administered to the patient).

Some embodiments provide a method of delaying and/or preventing development of cancer resistant to an anticancer agent in a subject, comprising administering to the subject a therapeutically effective amount of an ADC as described herein, or a salt thereof, before, during, or after administration of a therapeutically effective amount of the anticancer agent.

The ADCs described herein are useful for inhibiting the multiplication of a cancer cell, causing apoptosis in a cancer cell, for increasing phagocytosis of a cancer cell, and/or for treating cancer in a subject in need thereof. The ADCs can be used accordingly in a variety of settings for the treatment of cancers. The ADCs can be used to deliver a drug to a cancer cell. Without being bound by theory, in some embodiments, the antibody of an ADC binds to or associates with a cancer-cell-associated antigen. The antigen can be attached to a cancer cell or can be an extracellular matrix protein associated with the cancer cell. In some embodiments, the Drug Unit is cleaved from the ADC outside the cancer cell. In some embodiments, the Drug Unit remains attached to the antibody bound to the antigen.

In some embodiments, the antibody binds to the cancer cell. In some embodiments, the antibody binds to a cancer cell antigen which is on the surface of the cancer cell. In some embodiments, the antibody binds to a cancer cell antigen which is an extracellular matrix protein associated with the tumor cell or cancer cell. In some embodiments, the antibody of an ADC binds to or associates with a cancer-associated cell or an antigen on a cancer-associated cell. In some embodiments, the cancer-associated cell is a stromal cell in a tumor, for example, a cancer-associated fibroblast (CAF).

In some embodiments, the antibody of an ADC binds to or associates with an immune cell or an immune-cell-associated antigen. The antigen can be attached to an immune cell or can be an extracellular matrix protein associated with the immune cell. The drug can be released in proximity to the immune cell, thus recruiting/activating the immune cell to attack a cancer cell. In some embodiments, the Drug Unit is cleaved from the ADC outside the immune cell. In some embodiments, the Drug Unit remains attached to the antibody bound to the antigen. In some embodiments, the immune cell is a lymphocyte, an antigen-presenting cell, a natural killer (NK) cell, a neutrophil, an eosinophil, a basophil, a mast cell, an innate lymphoid cell, or a combination of any of the foregoing. In some embodiments, the immune cell is selected from the group consisting of B cells, plasma cells, T cells, NKT cells, gamma delta T cells, monocytes, macrophages, dendritic cells, natural killer (NK) cells, neutrophils, eosinophils, basophils, mast cells, and a combination of any of the foregoing.

The specificity of the antibody for a particular cancer cell can be important for determining those tumors or cancers that are most effectively treated. For example, ADCs that target a cancer cell antigen present on hematopoietic cancer cells in some embodiments treat hematologic malignancies. In some embodiments, ADCs target a cancer cell antigen present on abnormal cells of solid tumors for treating such solid tumors. In some embodiments an ADC are directed against abnormal cells of hematopoietic cancers such as, for example, lymphomas (Hodgkin Lymphoma and Non-Hodgkin Lymphomas) and leukemias.

Cancers, including, but not limited to, a tumor, metastasis, or other disease or disorder characterized by abnormal cells that are characterized by uncontrolled cell growth in some embodiments are treated or inhibited by administration of an ADC.

In some embodiments, the subject has previously undergone treatment for the cancer. In some embodiments, the prior treatment is surgery, radiation therapy, administration of one or more anticancer agents, or a combination of any of the foregoing.

In any of the methods described herein, the cancer is selected from the group consisting of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, esophagus and stomach carcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, kidney carcinoma, leukemia lymphocytic, leukemia lymphocytic chronic, liver cholangiocarcinoma, liver hepatocellular carcinoma, liver carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, upper aerodigestive tract squamous cell carcinoma, upper aerodigestive tract carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkins, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), large intestine/colon carcinoma, large intestine adenocarcinoma, soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheliod cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, lymphoid neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

In some embodiments, the subject is concurrently administered one or more additional anticancer agents with the ADCs described herein, or a salt thereof. In some embodiments, the subject is concurrently receiving radiation therapy with the ADCs described herein, or a salt thereof. In some embodiments, the subject is administered one or more additional anticancer agents after administration of the ADCs described herein, or a salt thereof. In some embodiments, the subject receives radiation therapy after administration of the ADCs described herein, or a salt thereof.

In some embodiments, the subject has discontinued a prior therapy, for example, due to unacceptable or unbearable side effects, wherein the prior therapy was too toxic, or wherein the subject developed resistance to the prior therapy.

Some embodiments provide a method for delaying or preventing a disease or disorder, comprising administering to the subject a therapeutically effective amount of an ADC as described herein, or a salt thereof, and a vaccine against the disease or disorder, to a patient at risk for developing the disease or disorder. In some embodiments, the disease or disorder is cancer, as described herein. In some embodiments, the disease or disorder is a viral pathogen. In some embodiments, the vaccine is administered subcutaneously. In some embodiments, the vaccine is administered intramuscularly. In some embodiments, the ADC and the vaccine are administered via the same route (for example, the ADC and the vaccine are both administered subcutaneously). In some embodiments, the ADC, or a salt thereof, and the vaccine are administered via different routes. In some embodiments, the vaccine and the ADC, or a salt thereof, are provided in a single formulation. In some embodiments, the vaccine and the ADC, or a salt thereof, are provided in separate formulations.

In some embodiments, the ADCs described herein are present in the form of a salt. In some embodiments, the salt is a pharmaceutically acceptable salt.

### Compositions and Methods of Administration of ADCs

Some embodiments provide an ADC composition comprising a distribution of ADCs, as described herein. In some embodiments, the composition comprises a distribution of ADCs, as described herein and at least one pharmaceutically acceptable carrier. In some embodiments, the route of administration is parenteral. Parenteral administration includes subcutaneous injections, intravenous, intramuscular, intrasternal injection or infusion techniques. In some embodiments, the compositions are administered parenterally. In one of those embodiments, the ADCs are administered intravenously. Administration is typically through any convenient route, for example by infusion or bolus injection.

Compositions of an ADC are formulated so as to allow the ADC to be bioavailable upon administration of the composition to a subject. Compositions can be in the form of one or more injectable dosage units.

Materials used in preparing the compositions can be non-toxic in the amounts used. It will be evident to those of ordinary skill in the art that the optimal dosage of the active ingredient(s) in the composition will depend on a variety of factors. Relevant factors include, without limitation, the type of animal (e.g., human), the particular form of the compound, the manner of administration, and the composition employed.

In some embodiments, the ADC composition is a solid, for example, as a lyophilized powder, suitable for reconstitution into a liquid prior to administration. In some embodiments, the ADC composition is a liquid composition, such as a solution or a suspension. A liquid composition or suspension is useful for delivery by injection and a lyophilized solid is suitable for reconstitution as a liquid or suspension using a diluent suitable for injection. In a composition administered by injection, one or more of a surfactant, preservative, wetting agent, dispersing agent, suspending agent, buffer, stabilizer and isotonic agent is typically included.

In some embodiments, the liquid compositions, whether they are solutions, suspensions or other like form, can also include one or more of the following: sterile diluents such as water for injection, saline solution, physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is typically enclosed in ampoule, a disposable syringe or a multiple-dose vial made of glass, plastic or other material. In some embodiments, the sterile diluent comprises physiological saline. In some embodiments, the sterile diluent is physiological saline. In some embodiments, the composition described herein are liquid injectable compositions that are sterile.

The amount of the ADC that is effective in the treatment of a particular disorder or condition will depend on the nature of the disorder or condition, which is usually determined by standard clinical techniques. In addition, in vitro or in vivo assays are sometimes employed to help identify optimal dosage ranges. The precise dose to be employed in the compositions will also depend on the route of parenteral administration, and the seriousness of the disease or disorder, and should be decided according to the judgment of the practitioner and each subject's circumstances.

In some embodiments, the compositions comprise an effective amount of an ADC such that a suitable dosage will be obtained. Typically, this amount is at least about 0.01% of the ADC by weight of the composition.

In some embodiments, the compositions dosage of an ADC administered to a subject is from about 0.01 mg/kg to about 100 mg/kg, from about 1 to about 100 mg of a per kg or from about 0.1 to about 25 mg/kg of the subject's body weight. In some embodiments, the dosage administered to a subject is about 0.01 mg/kg to about 15 mg/kg of the subject's body weight. In some embodiments, the dosage administered to a subject is about 0.1 mg/kg to about 15 mg/kg of the subject's body weight. In some embodiments, the dosage administered to a subject is about 0.1 mg/kg to about 20 mg/kg of the subject's body weight. In some embodiments, the dosage administered is about 0.1 mg/kg to about 5 mg/kg or about 0.1 mg/kg to about 10 mg/kg of the subject's body weight. In some embodiments, the dosage administered is about 1 mg/kg to about 15 mg/kg of the subject's body weight. In some embodiments, the dosage administered is about 1 mg/kg to about 10 mg/kg of the subject's body weight. In some embodiments, the dosage administered is about 0.1 to about 4 mg/kg, about 0.1 to about 3.2 mg/kg, or about 0.1 to about 2.7 mg/kg of the subject's body weight over a treatment cycle.

The term “carrier” refers to a diluent, adjuvant or excipient, with which a compound is administered. Such pharmaceutical carriers are liquids. Water is an exemplary carrier when the compounds are administered intravenously. Saline solutions and aqueous dextrose and glycerol solutions are also useful as liquid carriers for injectable solutions. Suitable pharmaceutical carriers also include glycerol, propylene, glycol, or ethanol. The present compositions, if desired, will in some embodiments also contain minor amounts of wetting or emulsifying agents, and/or pH buffering agents.

In some embodiments, the ADCs are formulated in accordance with routine procedures as a composition adapted for intravenous administration to animals, particularly human beings. Typically, the carriers or vehicles for intravenous administration are sterile isotonic aqueous buffer solutions. In some embodiments, the composition further comprises a local anesthetic, such as lignocaine, to ease pain at the site of the injection. In some embodiments, the ADC and the remainder of the formulation are supplied either separately or mixed together in unit dosage form, for example, as a dry lyophilized powder or water free concentrate in a hermetically sealed container such as an ampoule or sachette indicating the quantity of active agent. Where an ADC is to be administered by infusion, it is sometimes dispensed, for example, with an infusion bottle containing sterile pharmaceutical grade water or saline. Where the ADCs are administered by injection, an ampoule of sterile water for injection or saline is typically provided so that the ingredients can be mixed prior to administration.

The compositions are generally formulated as sterile, substantially isotonic and in full compliance with all Good Manufacturing Practice (GMP) regulations of the U.S. Food and Drug Administration.

### Drug-Linker Intermediates (L¹-D)

Some embodiments provide a compound having the formula L¹-D, or a salt thereof, wherein:
L¹ is a linker intermediate having the formula M¹-(A)ₐ-(W)_{w}—(Y)_{y}—(X)—;
wherein A, W, Y, X, are as defined for the linker (L);
wherein subscripts a, w, and y, are each independently 0 or 1;
wherein the sum of subscripts a, w, and y, is 1, 2, or 3; and
wherein D is as defined for the ADCs described herein.

In some embodiments, M¹ comprises a functional group that will react with an antibody to form a covalent bond (the Ab-M bond). In some embodiments, M¹ is selected from the group consisting of maleimido, azido, C₂-C₆ alkynyl, cycloalkynyl optionally substituted with 1 or 2 fluoro (e.g., cyclooctynyl or DIFO), sulfhydryl, succinimidyl esters (e.g., N-hydroxysuccinimidyl (NHS) or sulfo-NHS esters), 4-nitrophenyl esters, pentafluorophenyl esters, tetrafluorophenyl esters, anhydrides, acid chlorides, sulfonyl chlorides, isocyanates, isothiocyanates, alpha-haloketones, alpha-O-sulfonate (e.g., mesyl or tosyl) ketones, alkyl hydrazines, hydrazides, and hydroxylamines. In some embodiments, M¹ is selected from the group consisting of maleimido, azido, C₂-C₆ alkynyl, cycloalkynyl optionally substituted with 1 or 2 fluoro (e.g., cyclooctynyl or DIFO), sulfhydryl, succinimidyl esters. Additional examples of functional groups that will react with an antibody to form a covalent bond are described in PCT Publication No. WO2016/040684, which is hereby incorporated by reference in its entirety.

In some embodiments, M¹ is

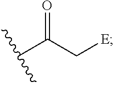

wherein the wavy line indicates the covalent bond to the remainder of L¹ (e.g., A, W, Y, or X); and wherein E is halogen or —O(SO₂)-E′; wherein E′ is alkyl, aryl, or aryl substituted with alkyl, as described herein (e.g., tosyl or mesyl).

In some embodiments, M¹ is

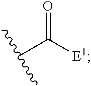

wherein the wavy line indicates the covalent bond to the remainder of L¹ (e.g., A, W, Y, or X); wherein E¹ is halogen, —O—N-succinimide, —O-(aryl), wherein the aryl is substituted with nitro, 4 or 5 fluoro, —OC(═O)—O(C₁-C₆ alkyl), or —OC(═O)—O(aryl).

In some embodiments, M¹ is

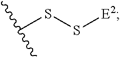

wherein the wavy line indicates the covalent bond to the remainder of L¹ (e.g., A, W, Y, or X); wherein E² is aryl or heteroaryl, as described herein.

In some embodiments, M¹ is

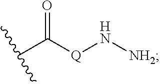

wherein the wavy line indicates the covalent bond to the remainder of L¹ (e.g., A, W, Y, or X); and wherein Q is a bond or C₁-C₁₀ alkylene.

In some embodiments, M¹ is

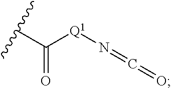

wherein the wavy line indicates the covalent bond to the remainder of L¹ (e.g., A, W, Y, or X); and wherein Q¹ is C₁-C₁₀ alkylene.

In some embodiments, M¹ is

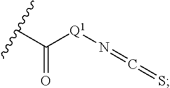

wherein the wavy line indicates the covalent bond to the remainder of L¹ (e.g., A, W, Y, or X); and wherein Q¹ is C₁-C₁₀ alkylene.

In some embodiments, M¹ is

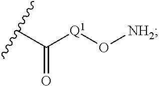

wherein the wavy line indicates the covalent bond to the remainder of L¹ (e.g., A, W, Y, or X); and wherein Q¹ is C₁-C₁₀ alkylene.

In some embodiments, M¹ is

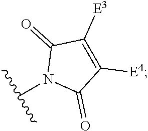

wherein the wavy line indicates the covalent bond to the remainder of L¹ (e.g., A, W, Y, or X); and wherein E³ and E⁴ are independently selected from the group consisting of hydrogen, halogen, C₁-C₆ alkyl, and —O(SO₂)-E⁵; wherein E⁵ is alkyl, aryl, or aryl substituted with alkyl, as described herein (e.g., tosyl or mesyl).

In some embodiments, M¹ is

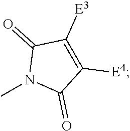

and E³ and E⁴ are both hydrogen. As such, in some embodiments M¹ is

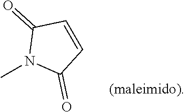

In some embodiments, -M¹-A- is

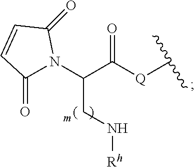

wherein subscript m is 1 or 2; Q is —NH—(C₁-C₅ alkylene)-C(═O)— or is absent; R^{h} is hydrogen or a nitrogen protecting group;
wherein the wavy line represents covalent attachment to W, Y, or X.

In some embodiments, Q is —NH—CH₂—C(═O)—. In some embodiments, Q is absent.

In some embodiments, the nitrogen protecting group is an acid-labile protecting group. In some embodiments, the nitrogen protecting group is a carbamate protecting group. In some embodiments, the nitrogen protecting group is t-butyloxycarbonyl (Boc) or carboxybenzyl (Cbz).

In some embodiments, -M¹-A- is selected from the group consisting of:

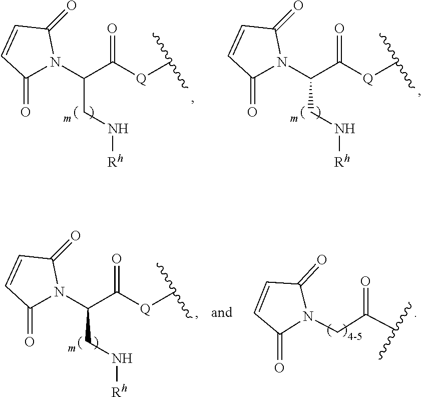

In some embodiments, the compound of L¹-D is selected from the compounds shown in Table 1, or a salt thereof.

TABLE 1

| Cmpd | |
|---|---|
| No. | Structure |
| 14 | 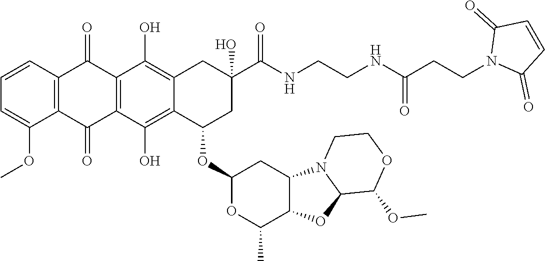 [SMILES: COc1cccc2c1C(=O)c1c(O)c3c(c(O)c1C2=O)C[C@@](O)(C(=O)NCCNC(=O)CCN1C(=O)C=CC1=O)C[C@@H]3O[C@H]1C[C@H]2[C@H](O[C@@H]3[C@@H](OC)OCCN32)[C@H](C)O1] |
| 18 | 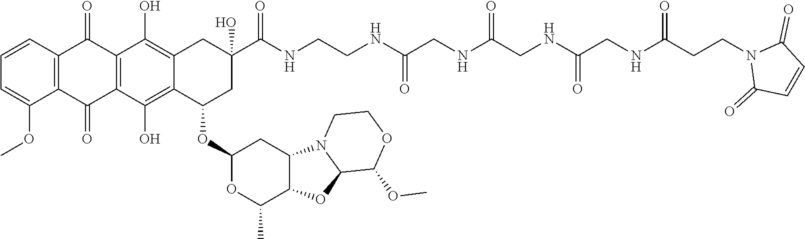 [SMILES: COc1cccc2c1C(=O)c1c(O)c3c(c(O)c1C2=O)C[C@@](O)(C(=O)NCCNC(=O)CNC(=O)CNC(=O)CNC(=O)CCN1C(=O)C=CC1=O)C[C@@H]3O[C@H]1C[C@H]2[C@H](O[C@@H]3[C@@H](OC)OCCN32)[C@H](C)O1] |
| 21 | 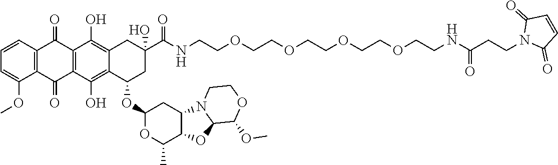 [SMILES: COc1cccc2c1C(=O)c1c(O)c3c(c(O)c1C2=O)C[C@@](O)(C(=O)NCCOCCOCCOCCOCCNC(=O)CCN1C(=O)C=CC1=O)C[C@@H]3O[C@H]1C[C@H]2[C@H](O[C@@H]3[C@@H](OC)OCCN32)[C@H](C)O1] |
| 24 | 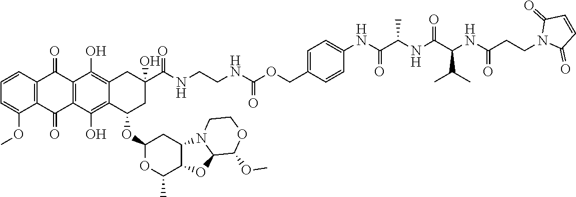 [SMILES: COc1cccc2c1C(=O)c1c(O)c3c(c(O)c1C2=O)C[C@@](O)(C(=O)NCCNC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1] |
| 29 | 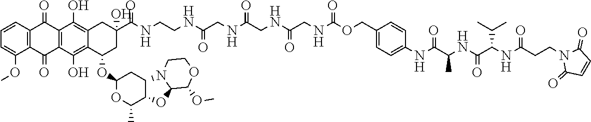 [SMILES: COc1cccc2c1C(=O)c1c(O)c3c(c(O)c1C2=O)C[C@@](O)(C(=O)NCCNC(=O)CNC(=O)CNC(=O)CNC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1] |
| 32 | 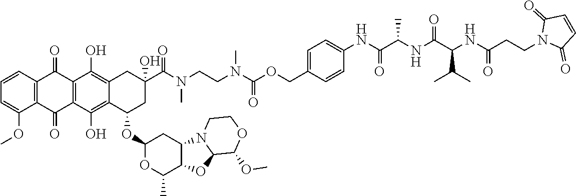 [SMILES: COc1cccc2c1C(=O)c1c(O)c3c(c(O)c1C2=O)C[C@@](O)(C(=O)N(C)CCN(C)C(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1] |
| 33 | 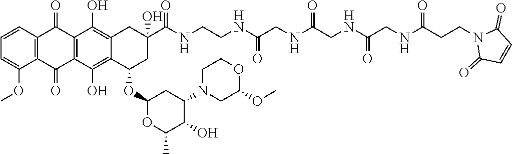 [SMILES: COc1cccc2c1C(=O)c1c(O)c3c(c(O)c1C2=O)C[C@@](O)(C(=O)NCCNC(=O)CNC(=O)CNC(=O)CNC(=O)CCN1C(=O)C=CC1=O)C[C@@H]3O[C@H]1C[C@H](N2CCO[C@H](OC)C2)[C@H](O)[C@H](C)O1] |
| 34 | 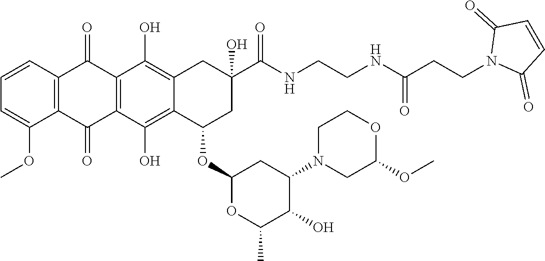 [SMILES: COc1cccc2c1C(=O)c1c(O)c3c(c(O)c1C2=O)C[C@@](O)(C(=O)NCCNC(=O)CCN1C(=O)C=CC1=O)C[C@@H]3O[C@H]1C[C@H](N2CCO[C@H](OC)C2)[C@H](O)[C@H](C)O1] |
| 36 | 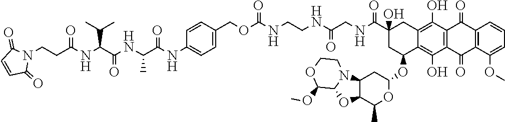 [SMILES: COc1cccc2c1C(=O)c1c(O)c3c(c(O)c1C2=O)C[C@@](O)(C(=O)NCC(=O)NCCNC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1] |
| 38 | 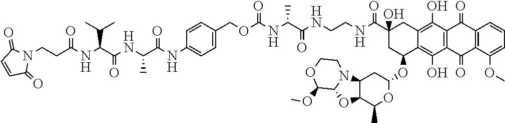 [SMILES: COc1cccc2c1C(=O)c1c(O)c3c(c(O)c1C2=O)C[C@@](O)(C(=O)NCCNC(=O)[C@@H](C)NC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1] |
| 40 | 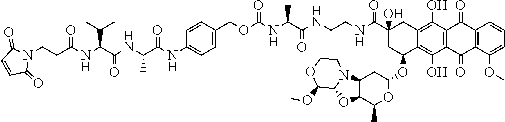 [SMILES: COc1cccc2c1C(=O)c1c(O)c3c(c(O)c1C2=O)C[C@@](O)(C(=O)NCCNC(=O)[C@H](C)NC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1] |
| 42 | 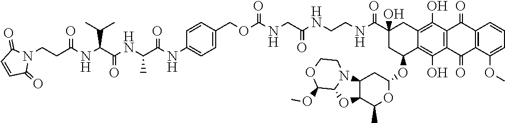 [SMILES: COc1cccc2c1C(=O)c1c(O)c3c(c(O)c1C2=O)C[C@@](O)(C(=O)NCCNC(=O)CNC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1] |
| 44 | 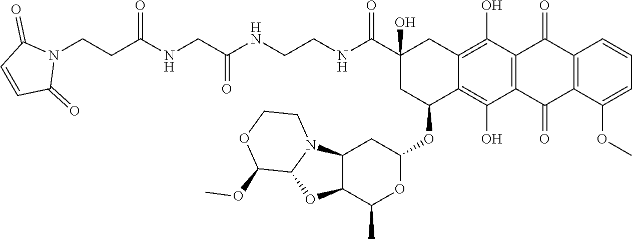 [SMILES: COc1cccc2c1C(=O)c1c(O)c3c(c(O)c1C2=O)C[C@@](O)(C(=O)NCCNC(=O)CNC(=O)CCN1C(=O)C=CC1=O)C[C@@H]3O[C@H]1C[C@H]2[C@H](O[C@@H]3[C@@H](OC)OCCN32)[C@H](C)O1] |
| 46 | 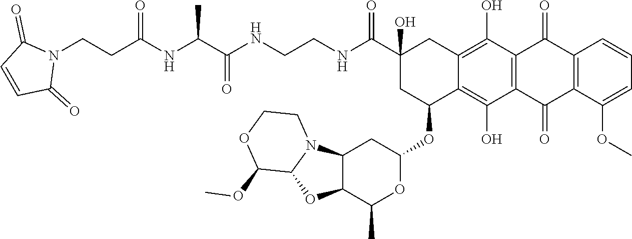 [SMILES: COc1cccc2c1C(=O)c1c(O)c3c(c(O)c1C2=O)C[C@@](O)(C(=O)NCCNC(=O)[C@H](C)NC(=O)CCN1C(=O)C=CC1=O)C[C@@H]3O[C@H]1C[C@H]2[C@H](O[C@@H]3[C@@H](OC)OCCN32)[C@H](C)O1] |
| 48 | 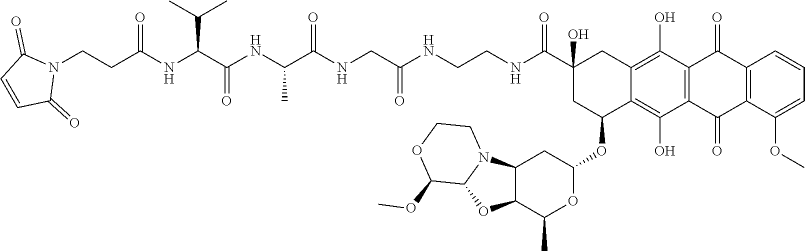 [SMILES: COc1cccc2c1C(=O)c1c(O)c3c(c(O)c1C2=O)C[C@@](O)(C(=O)NCCNC(=O)CNC(=O)[C@H](C)NC(=O)[C@@H](NC(=O)CCN1C(=O)C=CC1=O)C(C)C)C[C@@H]3O[C@H]1C[C@H]2[C@H](O[C@@H]3[C@@H](OC)OCCN32)[C@H](C)O1] |

In some embodiments, the compounds of Formula L¹-D described herein are present in the form of a salt. In some embodiments, the salt is a pharmaceutically acceptable salt.

### EXAMPLES

Synthetic Procedures—General All commercially available anhydrous solvents were used without further purification. Flash column chromatography was performed on a Biotage Isolera One flash purification system (Charlotte, NC). UPLC-MS systems consisted of either a Waters SQD2 mass detector interfaced to an Acquity Ultra Performance LC or a Waters Xevo G2 ToF interfaced to an Acquity H-class Ultra Performance LC. The HPLC column used for analysis was a CORTECS C18 2.1×50 mm, 1.6 m reversed-phase column. Analytes were eluted by running a gradient of 3% acetonitrile/97% water to 100% acetonitrile, at a flow rate of 0.5 mL/min. The organic acetonitrile (MeCN) and aqueous mobile phases were modified with either 0.1% (v/v) formic acid (gradient 1) or 10 mM ammonium chloride (NH₄Cl) in MeCN/water, pH 4.5 (gradient 2). Preparative HPLC was carried out using a Waters Prep 150 LC system paired with a 2998 photodiode array detector, using a C12 Phenomenex Synergi 10.0×250 mm, 4 m, 80 A reversed-phase column, and eluting with 0.1% (v/v) trifluoroacetic acid (TFA) or 10 mM NH₄Cl in water (solvent A) and 0.1% (v/v) TFA in MeCN or MeCN (solvent B). The purification methods generally consisted of linear gradients of solvent A to solvent B, ramping from 90% aqueous solvent A to 10% solvent A over 1 hour. The flow rate was 4.6 mL/min with monitoring by ultraviolet (UV) at 254 nm.

### Example 1: (2S,4S)-4-(((2R,4S,5S,6S)-4-amino-5-hydroxy-6-methyltetrahydro-2H-pyran-2-yl)oxy)-2,5,12-trihydroxy-7-methoxy-6,11-dioxo-1,2,3,4,6,11-hexahydrotetracene-2-carboxylic acid (2)

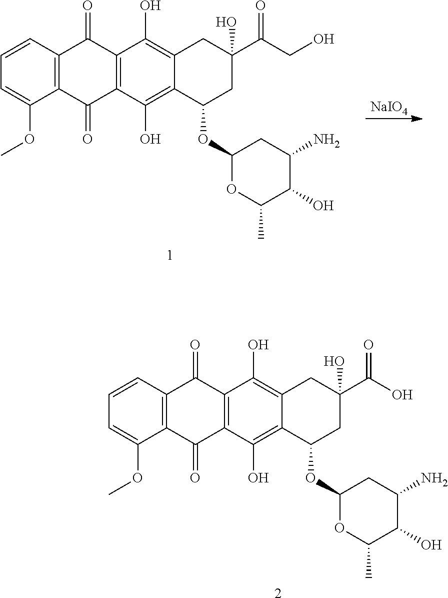

Doxorubicin (1, 14 mg, 26 μmol) was dissolved in a mixture of MeOH (3 mL) and H₂O (2 mL). A solution of sodium periodate (NaIO₄, 7 mg, 31 μmol) in H₂O (1 mL) was added to the above solution with stirring. After 1 hour, the solvents were removed under reduced pressure and the material was used in subsequent steps without further purification. Analytical UPLC-MS (gradient 1): HPLC retention time=1.19 min; m/z (ESI+) calculated 530.17 [M+H]⁺; found 530.54.

### Example 2: (2S,4S)-2,5,12-trihydroxy-4-(((2R,4S,5S,6S)-5-hydroxy-4-((S)-2-methoxymorpholino)-6-methyltetrahydro-2H-pyran-2-yl)oxy)-7-methoxy-6,11-dioxo-1,2,3,4,6,11-hexahydrotetracene-2-carboxylic acid (4)

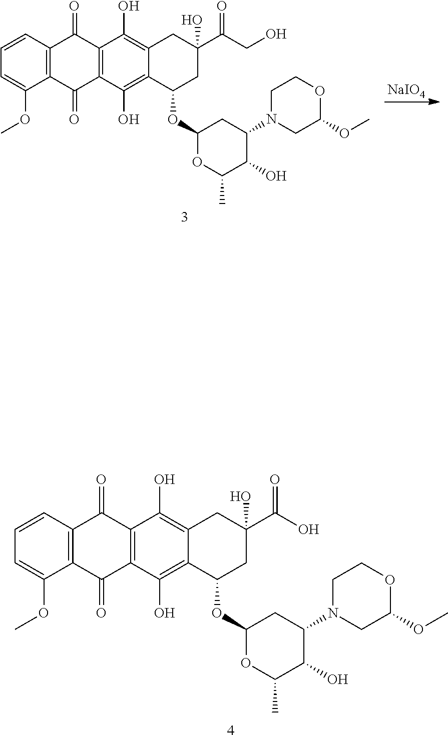

Nemorubicin (3, MedChemExpress, 10 mg, 16 μmol) was dissolved in a mixture of MeOH (0.6 mL) and H₂O (0.4 mL). A solution of NaIO₄ (25 mg, 117 μmol) in H₂O (0.2 mL) was added to the above solution with stirring. After 1 hour, the solvents were removed under reduced pressure and the material was used in subsequent steps without further purification. Analytical UPLC-MS (gradient 1): HPLC retention time=1.30 min; m/z (ESI+) calculated 630.22 [M+H]⁺; found 630.46.

### Example 3: (2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxylic acid (6)

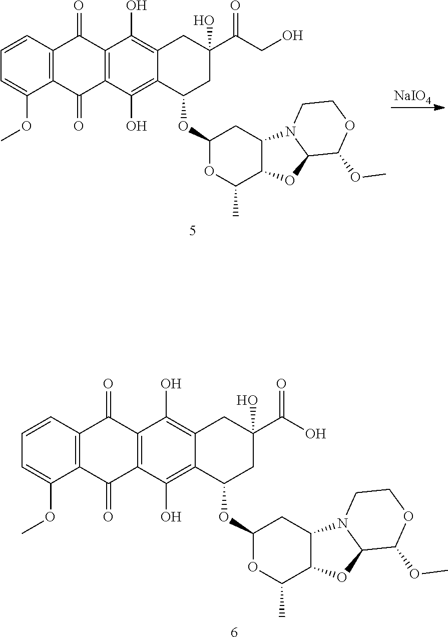 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC)OCCN32)[C@H](C)O1 ; "NaIO4" ; "5" ; COc1cccc2c1C(=O)c1c(O)c3c(c(O)c1C2=O)C[C@@](O)(C(=O)O)C[C@@H]3O[C@H]1C[C@H]2[C@H](O[C@@H]3[C@@H](OC)OCCN32)[C@H](C)O1 ; "6"]

PNU-159682 (5, Levena Biopharma cat #, 50 mg, 78 μmol) was dissolved in a mixture of MeOH (3 mL) and H₂O (2 mL). A solution of NaIO₄ (25 mg, 117 μmol) in H₂O (1 mL) was added to the above solution with stirring. After 1 hour, the solvents were removed under reduced pressure and the material used in subsequent steps without further purification. Analytical UPLC-MS (gradient 1): HPLC retention time=1.59 min; m/z (ESI+) calculated 628.20 [M+H]⁺; found 628.60.

### Example 4: (2S,4S)—N-(2-aminoethyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (9)

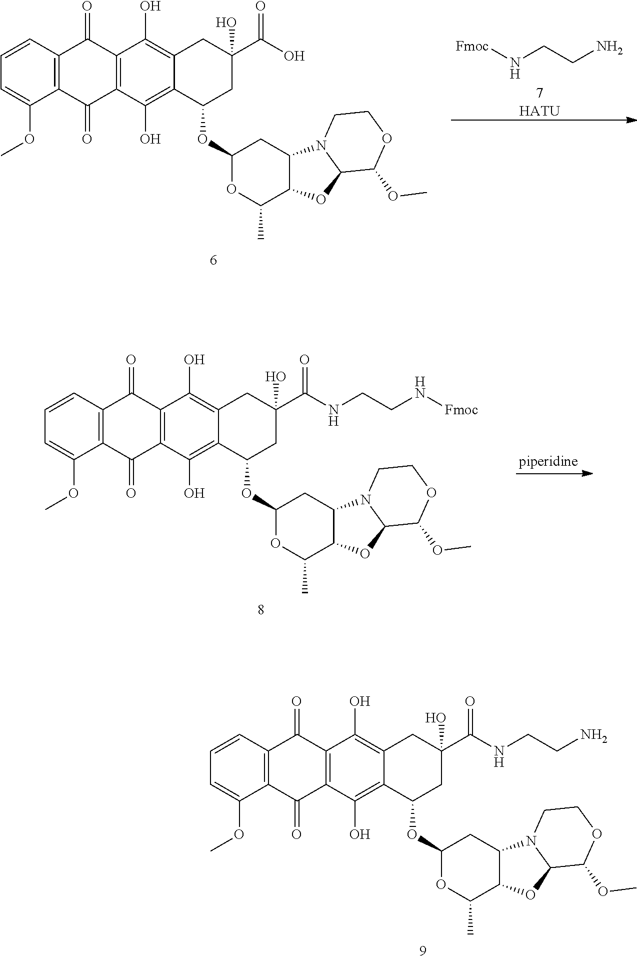

### (9H-fluoren-9-yl)methyl (2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)ethyl)carbamate (8)

PNU—OH (6, 28 mg, 45 μmol) and 1-[bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide (HATU, 34 mg, 90 μmol) were dissolved in anhydrous N,N-dimethylformamide (DMF, 0.90 mL). N-Fmoc-ethylenediamine hydrochloride (7, Santa Cruz Biotech, 25 mg, 90 μmol) was added to the above solution as a solid, followed by the addition of N,N-diisopropylethylamine (DIPEA, 31 μL, 179 μmol). After 15 minutes, the reaction mixture was diluted with 0.5 mL MeCN and 0.5 mL 0.05% (v/v) aqueous TFA, and purified by preparative LC using TFA as mobile phase modifier to provide 8 (13 mg, 33%). Analytical UPLC-MS (gradient 1): HPLC retention time=2.26 min; m/z (ESI+) calculated 892.33 [M+H]⁺; found 892.48.

### (2S,4S)—N-(2-aminoethyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (9)

Fmoc-EDA-PNU (8, 5 mg, 6 μmol) was dissolved in a mixture of DMF (0.18 mL) and piperidine (45 μL). After 15 minutes, the reaction mixture was diluted with 0.5 mL MeCN and 0.5 mL 0.05% (v/v) aqueous TFA, and purified by preparative LC using TFA as mobile phase modifier to provide 9 (2.4 mg, 60%). Analytical UPLC-MS (gradient 1): HPLC retention time=1.24 min; m/z (ESI+) calculated 670.26 [M+H]⁺; found 670.41.

### Example 5: N-(2-aminoethyl)-3-(2,5-dioxo-2,5-dihydro-1H-pyrrol-1-yl)propenamide (13)

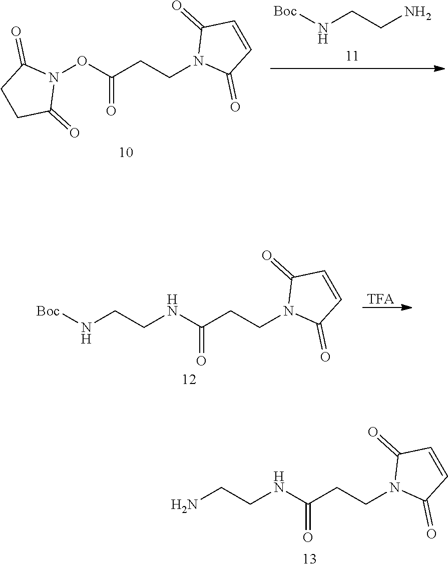 [SMILES: "TFA" ; O=C(CCN1C(=O)C=CC1=O)ON1C(=O)CCC1=O ; CC(C)(C)OC(=O)NCCN ; CC(C)(C)OC(=O)NCCNC(=O)CCN1C(=O)C=CC1=O ; NCCNC(=O)CCN1C(=O)C=CC1=O]

### tert-butyl (2-(3-(2,5-dioxo-2,5-dihydro-1H-pyrrol-1-yl)propanamido)ethyl)carbamate (12)

A flame dried flask was charged with 3-(maleimido)propionic acid N-hydroxysuccinimide ester (mp-OSu 10, 50 mg, 188 μmol), anhydrous DMF (0.75 mL), and DIPEA (101 μL, 564 μmol). N-Boc-ethylenediamine (11, 169 μmol, 27 μL) was diluted in minimal volume of DMF and added dropwise to the mp-OSu solution with stirring. The reaction was quenched after 30 minutes by adding 101 μL glacial acetic acid (AcOH), and the reaction mixture was diluted with minimal volume of 1:1 MeCN:0.05% (v/v) aqueous TFA, and purified by preparative LC using TFA as mobile phase modifier to provide 12 (34 mg, 58%). Analytical UPLC-MS (gradient 1): HPLC retention time=1.28 min; m/z (ESI+) calculated 334.14 [M+Na]⁺; found 334.39.

### N-(2-aminoethyl)-3-(2,5-dioxo-2,5-dihydro-1H-pyrrol-1-yl)propenamide (13)

mp-EDA-Boc (12, 34 mg, 108 μmol) was dissolved in anhydrous dichloromethane (DCM, 0.8 mL) and cooled to 0° C. using an ice bath. TFA (0.2 mL) was added dropwise to the above solution with stirring. After 1 hour, the solvents were removed under reduced pressure and the crude product was used in subsequent steps without further purification. Analytical UPLC-MS (gradient 1): HPLC retention time=0.42 min; m/z (ESI+) calculated 234.19 [M+Na]⁺; found 234.08.

### Example 6: (2S,4S)—N-(2-(3-(2,5-dioxo-2,5-dihydro-1H-pyrrol-1-yl)propanamido)ethyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (14)

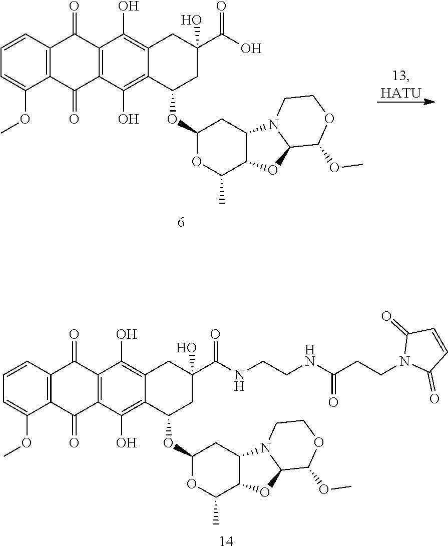

PNU-OH (6, 10 mg, 16 μmol) and HATU (6 mg, 16 μmol) were dissolved in anhydrous DMF (0.17 mL). mp-EDA (13, 3 mg, 16 μmol), as a solution in DMF (0.17 mL) was added to the above solution with stirring, followed by the addition of DIPEA (8 μL, 48 μmol). After 15 minutes, the reaction mixture was diluted with 0.5 mL MeCN and 0.5 mL 10 mM aqueous NH₄Cl, and purified by preparative LC using NH₄Cl as mobile phase modifier to provide 14 (4 mg, 30%). Analytical UPLC-MS (gradient 2): HPLC retention time=1.72 min; m/z (ESI+) calculated 821.29 [M+H]⁺; found 821.65.

### Example 7: N-(2-((2-((2-((2-aminoethyl)amino)-2-oxoethyl)amino)-2-oxoethyl)amino)-2-oxoethyl)-3-(2,5-dioxo-2,5-dihydro-1H-pyrrol-1-yl)propenamide (17)

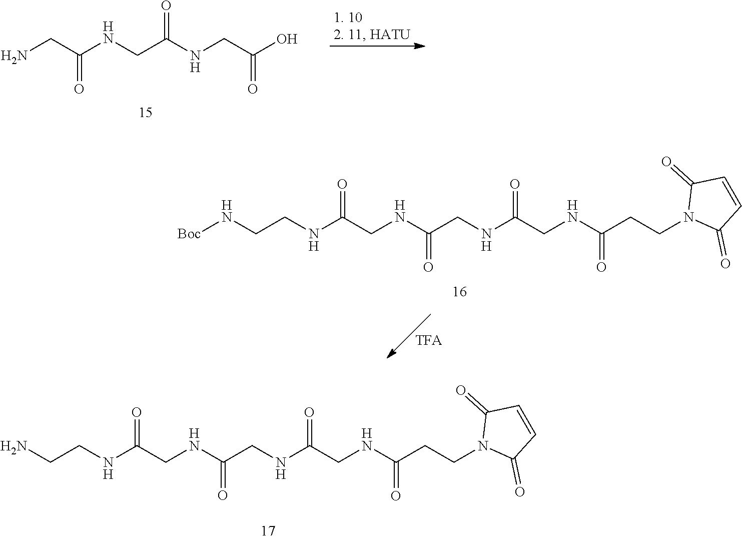 [SMILES: "TFA" ; NCC(=O)NCC(=O)NCC(=O)O ; CC(C)(C)OC(=O)NCCNC(=O)CNC(=O)CNC(=O)CNC(=O)CCN1C(=O)C=CC1=O ; NCCNC(=O)CNC(=O)CNC(=O)CNC(=O)CCN1C(=O)C=CC1=O]

### tert-butyl (15-(2,5-dioxo-2,5-dihydro-1H-pyrrol-1-yl)-4,7,10,13-tetraoxo-3,6,9,12-tetraazapentadecyl)carbamate (16)

A flame-dried flask was charged with H-Gly₃-OH (15, 71 mg, 376 μmol), mp-OSu (10, 100 mg, 376 μmol), and anhydrous dimethylsulfoxide (DMSO, 3.76 mL). DIPEA (262 L, 1.5 mmol) was added to the above solution, and the reaction mixture was stirred overnight under N₂. HATU (157 mg, 414 μmol) was then added to the reaction mixture followed by Boc-EDA (11, 60 μL, 376 μmol). After 30 minutes, the reaction mixture was directly loaded onto a preparative LC and the desired product purified, using TFA as the mobile phase modifier to provide 16 (91 mg, 50%). Analytical UPLC-MS (gradient 1): HPLC retention time=1.06 min; m/z (ESI+) calculated 483.22 [M+H]⁺; found 483.47.

### N-(2-((2-((2-((2-aminoethyl)amino)-2-oxoethyl)amino)-2-oxoethyl)amino)-2-oxoethyl)-3-(2,5-dioxo-2,5-dihydro-1H-pyrrol-1-yl)propenamide (17)

mp-Gly₃-EDA-Boc (16, 91 mg, 188 μmol) was dissolved in anhydrous DCM (1.6 mL) and cooled to 0° C. TFA (0.4 mL) was added dropwise to this solution with stirring. After 1 hour, the solvents were removed under reduced pressure and the crude product was used in subsequent steps without further purification. Analytical UPLC-MS (gradient 1): HPLC retention time=0.43 min; m/z (ESI+) calculated 383.17 [M+H]⁺; found 383.36.

### Example 8: (2S,4S)—N-(15-(2,5-dioxo-2,5-dihydro-1H-pyrrol-1-yl)-4,7,10,13-tetraoxo-3,6,9,12-tetraazapentadecyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (18)

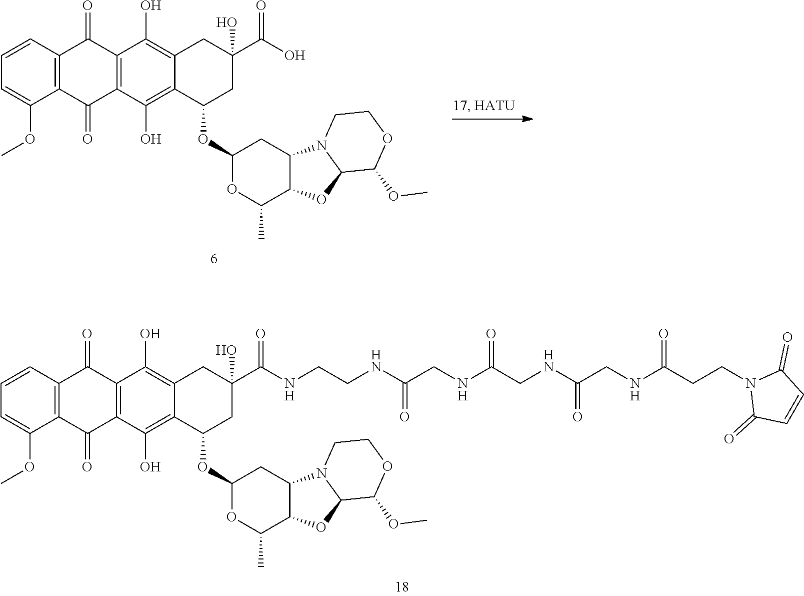 [SMILES: COc1cccc2c1C(=O)c1c(O)c3c(c(O)c1C2=O)C[C@@](O)(C(=O)O)C[C@@H]3O[C@H]1C[C@H]2[C@H](O[C@@H]3[C@@H](OC)OCCN32)[C@H](C)O1 ; "17, HATU" ; "6" ; COc1cccc2c1C(=O)c1c(O)c3c(c(O)c1C2=O)C[C@@](O)(C(=O)NCCNC(=O)CNC(=O)CNC(=O)CNC(=O)CCN1C(=O)C=CC1=O)C[C@@H]3O[C@H]1C[C@H]2[C@H](O[C@@H]3[C@@H](OC)OCCN32)[C@H](C)O1]

PNU-OH (6, 5 mg, 8 μmol) and HATU (3 mg, 7 μmol) were dissolved in anhydrous DMF (0.2 mL). mp-Gly₃-EDA (17, 3 mg, 6 μmol), as solution in DMF (0.2 mL), was added to the above solution with stirring, followed by the addition of DIPEA (4 μL, 25 μmol). After 15 minutes, the reaction mixture was diluted with 0.5 mL MeCN and 0.5 mL 10 mM aqueous NH₄C₁, and purified by preparative LC using NH₄Cl as mobile phase modifier to provide 18 (4 mg, 30%). Analytical UPLC-MS (gradient 2): HPLC retention time=1.48 min; m/z (ES+) calculated 992.35 [M+H]⁺; found 992.87.

### Example 9: N-(14-amino-3,6,9,12-tetraoxatetradecyl)-3-(2,5-dioxo-2,5-dihydro-1H-pyrrol-1-yl)propenamide (20)

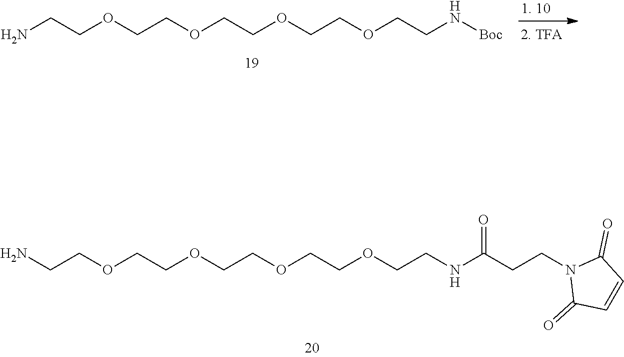 [SMILES: CC(C)(C)OC(=O)NCCOCCOCCOCCOCCN ; NCCOCCOCCOCCOCCNC(=O)CCN1C(=O)C=CC1=O]

A flame-dried flask was charged with mp-OSu (10, 25 mg, 94 μmol), anhydrous DMF (0.94 mL), and DIPEA (65 μL, 376 μmol). Boc-PEG₄-NH₂ (19, 30 μL, 94 μmol) was dissolved in minimal volume of DMF and added dropwise to the mp-OSu solution with stirring. The solvents were removed under reduced pressure, and the resulting residue was then re-dissolved in 0.8 mL DCM and 0.2 mL TFA. After 30 minutes, the volatiles were removed under reduced pressure, the resulting residue was re-solubilized in minimal volume of 1:1 MeCN: 0.05% (v/v) aqueous TFA, and purified by preparative LC using TFA as mobile phase modifier to provide 20 (40 mg, 87%). Analytical UPLC-MS (gradient 1): HPLC retention time=0.71 min; m/z (ESI+) calculated 388.21 [M+H]⁺; found 388.39.

### Example 10: (2S,4S)—N-(18-(2,5-dioxo-2,5-dihydro-1H-pyrrol-1-yl)-16-oxo-3,6,9,12-tetraoxa-15-azaoctadecyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (21)

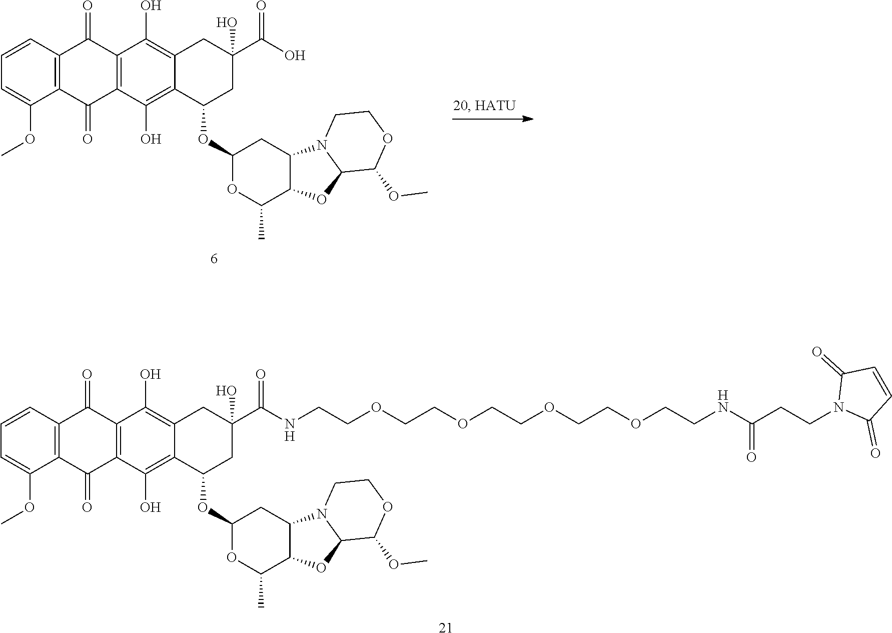 [SMILES: COc1cccc2c1C(=O)c1c(O)c3c(c(O)c1C2=O)C[C@@](O)(C(=O)O)C[C@@H]3O[C@H]1C[C@H]2[C@H](O[C@@H]3[C@@H](OC)OCCN32)[C@H](C)O1 ; "20, HATU" ; "6" ; COc1cccc2c1C(=O)c1c(O)c3c(c(O)c1C2=O)C[C@@](O)(C(=O)NCCOCCOCCOCCOCCNC(=O)CCN1C(=O)C=CC1=O)C[C@@H]3O[C@H]1C[C@H]2[C@H](O[C@@H]3[C@@H](OC)OCCN32)[C@H](C)O1 ; "21"]

PNU-OH (6, 5 mg, 8 μmol) and HATU (1.7 mg, 4.3 μmol) were dissolved in anhydrous DMF (0.13 mL). mp-PEG₄-NH₂ (20, 1.7 mg, 4.3 μmol) was added as a solution in DMF (0.13 mL), followed by the addition of DIPEA (3 μL, 17 μmol). After 15 minutes, the reaction mixture was diluted with 0.5 mL MeCN and 0.5 mL 0.05% (v/v) aqueous TFA, and purified by preparative LC using TFA as mobile phase modifier to provide 21 (2.2 mg, 51%). Analytical UPLC-MS (gradient 1): HPLC retention time=1.65 min; m/z (ESI+) calculated 997.40 [M+H]⁺; found 997.92.

### Example 11: 4-((S)-2-((S)-2-(3-(2,5-dioxo-2,5-dihydro-1H-pyrrol-1-yl)propanamido)-3-methylbutanamido)propanamido)benzyl (2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)ethyl)carbamate (24)

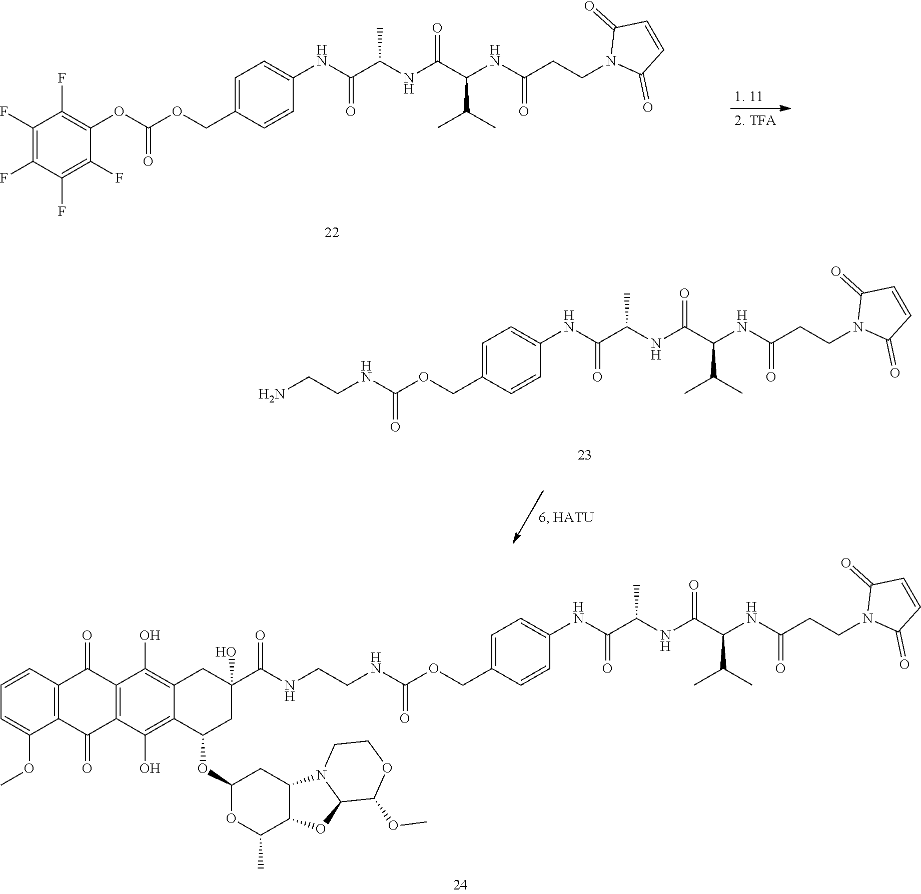 [SMILES: CC(C)[C@H](NC(=O)CCN1C(=O)C=CC1=O)C(=O)N[C@@H](C)C(=O)Nc1ccc(COC(=O)Oc2c(F)c(F)c(F)c(F)c2F)cc1 ; CC(C)[C@H](NC(=O)CCN1C(=O)C=CC1=O)C(=O)N[C@@H](C)C(=O)Nc1ccc(COC(=O)NCCN)cc1 ; COc1cccc2c1C(=O)c1c(O)c3c(c(O)c1C2=O)C[C@@](O)(C(=O)NCCNC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1]

### 4-((S)-2-((S)-2-(3-(2,5-dioxo-2,5-dihydro-1H-pyrrol-1-yl)propanamido)-3-methylbutanamido)propanamido)benzyl (2-aminoethyl)carbamate (23)

A flame-dried flask was charged with mp-ValAlaPAB-OPFP (22, 25 mg, 38 μmol) and Boc-EDA (11, 7 μL, 42 μmol) in anhydrous DMF (0.38 mL). DIPEA (27 μL, 153 μmol) was added to the above solution with stirring. After 30 minutes, solvents were removed under reduced pressure and the resulting residue was resolubilized in DCM (0.3 mL) and TFA (76 μL) for an additional 30 minutes. The volatiles were removed under reduced pressure, and the resulting residue was re-dissolved in minimal volume of 1:1 MeCN: 0.05% (v/v) aqueous TFA, and purified by preparative LC using TFA as mobile phase modifier to provide 23 (8 mg, 33%). Analytical UPLC-MS (gradient 1): HPLC retention time=0.93 min; m/z (ESI+) calculated 531.26 [M+H]⁺; found 531.58.

### 4-((S)-2-((S)-2-(3-(2,5-dioxo-2,5-dihydro-1H-pyrrol-1-yl)propanamido)-3-methylbutanamido)propanamido)benzyl (2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)ethyl)carbamate (24)

PNU-OH (6, 5.4 mg, 8.6 μmol) and HATU (3.6 mg, 9.5 μmol) were dissolved in anhydrous DMF (0.34 mL). mp-ValAlaPAB-EDA (23, 4.6 mg, 8.6 μmol) was added as a solution in DMF (0.34 mL) followed by the addition of DIPEA (6.0 μL, 34 μmol). After 15 minutes, the reaction mixture was diluted with 0.5 mL MeCN and 0.5 mL 10 mM aqueous NH₄Cl, and purified by preparative LC using NH₄Cl as mobile phase modifier to provide 24 (0.8 mg, 8%). Analytical UPLC-MS (gradient 2): HPLC retention time=1.83 min; m/z (ESI+) calculated 1140.44 [M+H]⁺; found 1140.75.

### Example 12: 4-((S)-2-((S)-2-(3-(2,5-dioxo-2,5-dihydro-1H-pyrrol-1-yl)propanamido)-3-methylbutanamido)propanamido)benzyl (1,6,9,12-tetraoxo-1-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)-2,5,8,11-tetraazatridecan-13-yl)carbamate (29)

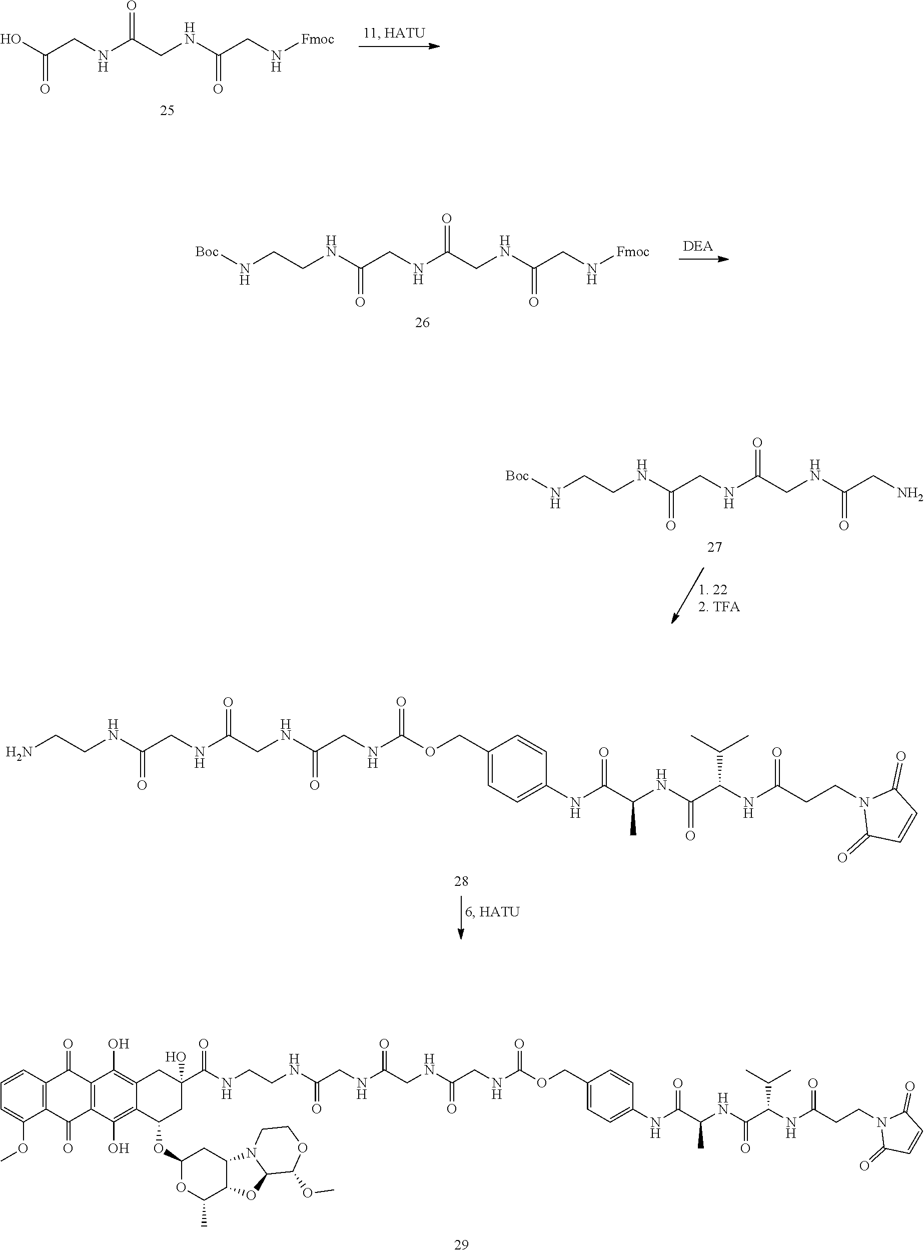

### (9H-fluoren-9-yl)methyl (2,2-dimethyl-4,9,12,15-tetraoxo-3-oxa-5,8,11,14-tetraazahexadecan-16-yl)carbamate (26)

Fmoc-Gly₃-OH (25, 50 mg, 122 μmol) and HATU (46 mg, 122 μmol) were dissolved in anhydrous DMF (1.2 mL). Boc-EDA (11, 19 μL, 122 μmol) was added to this solution with stirring, followed by the addition of DIPEA (85 μL, 486 μmol). After 15 minutes, the volatiles were removed under reduced pressure and the crude product was purified by flash chromatography over silica gel (2%-20% MeOH in DCM linear gradient) to provide 26 (66 mg, 99%). Analytical UPLC-MS (gradient 1): HPLC retention time=1.79 min; m/z (ESI+) calculated 554.26 [M+H]⁺; found 554.45.

### tert-butyl (2-(2-(2-(2-aminoacetamido)acetamido)acetamido)ethyl)carbamate (27)

Boc-EDA-Gly₃-Fmoc (26, 66 mg, 119 μmol) was dissolved in a premixed solution of 1:1 (v/v) DCM and diethylamine (1.21 mL each). After 30 minutes, MeOH was added dropwise to the reaction mixture until all precipitate dissolved. MTBE was subsequently added dropwise until significant precipitation was observed, then the reaction was cooled to −20° C. and stirred for 1 hour. The resulting solid was collected using a fritted funnel, resolubilized in MeOH, and dried to provide 27 (28 mg, 71%). This crude product was used in subsequent steps without further purification. Analytical UPLC-MS (gradient 1): HPLC retention time=0.78 min; m/z (ESI+) calculated 332.20 [M+H]⁺; found 332.31.

### 4-((S)-2-((S)-2-(3-(2,5-dioxo-2,5-dihydro-1H-pyrrol-1-yl)propanamido)-3-methylbutanamido)propanamido)benzyl (2-((2-((2-((2-aminoethyl)amino)-2-oxoethyl)amino)-2-oxoethyl)amino)-2-oxoethyl)carbamate (28)

A flame-dried flask was charged with mp-ValAlaPAB-OPFP (22, 55 mg, 84 μmol) and Boc-EDA-Gly₃ (27, 28 mg, 84 μmol) in anhydrous DMF (0.85 mL). DIPEA (59 μL, 338 μmol) was added to the above solution with stirring. After 30 minutes, solvents were removed under reduced pressure and the resulting residue was resolubilized in DCM (0.68 mL) and TFA (0.17 μL), and stirred for an additional 30 minutes. The volatiles were removed under reduced pressure, and the resulting residue was re-dissolved in minimal volume of 1:1 MeCN: 0.05% (v/v) aqueous TFA, and purified by preparative LC using TFA as mobile phase modifier to provide 28 (38 mg, 64%). Analytical UPLC-MS (gradient 1): HPLC retention time=0.92 min; m/z (ESI+) calculated 702.32 [M+H]⁺; found 702.30.

### 4-((S)-2-((S)-2-(3-(2,5-dioxo-2,5-dihydro-1H-pyrrol-1-yl)propanamido)-3-methylbutanamido)propanamido)benzyl (1,6,9,12-tetraoxo-1-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracen-2-yl)-2,5,8,11-tetraazatridecan-13-yl)carbamate (29)

PNU-OH (6, 10 mg, 16 μmol) and HATU (6 mg, 16 μmol) were dissolved in anhydrous DMF (0.32 mL). mp-ValAlaPAB-EDA-Gly₃ (28, 11 mg, 16 μmol) was added as a solution in DMF (0.2 mL) to the above solution, followed by the addition of DIPEA (11 μL, 64 mol). After 15 minutes, the reaction mixture was diluted with 0.5 mL MeCN and 0.5 mL 10 mM aqueous NH₄Cl and purified by preparative LC using NH₄Cl as mobile phase modifier to provide 29 (11 mg, 53%). Analytical UPLC-MS (gradient 2): HPLC retention time=1.58 min; m/z (ESI+) calculated 1311.51 [M+H]⁺; found 1311.87.

### Example 13: 4-((S)-2-((S)-2-(3-(2,5-dioxo-2,5-dihydro-1H-pyrrol-1-yl)propanamido)-3-methylbutanamido)propanamido)benzyl methyl(2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-N-methyl-6,11-dioxo-1,2,3,4,6,11-hexahydrotetracene-2-carboxamido)ethyl)carbamate (32)

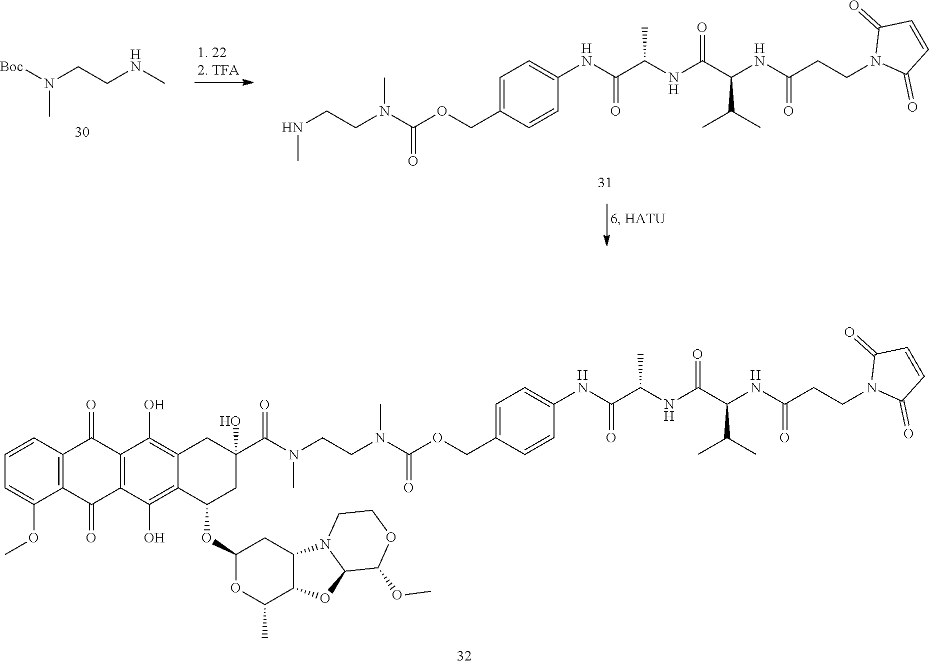 [SMILES: CNCCN(C)C(=O)OC(C)(C)C ; CNCCN(C)C(=O)OCc1ccc(NC(=O)[C@H](C)NC(=O)[C@@H](NC(=O)CCN2C(=O)C=CC2=O)C(C)C)cc1 ; COc1cccc2c1C(=O)c1c(O)c3c(c(O)c1C2=O)C[C@@](O)(C(=O)N(C)CCN(C)C(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1]

### 4-((S)-2-((S)-2-(3-(2,5-dioxo-2,5-dihydro-1H-pyrrol-1-yl)propanamido)-3-methylbutanamido)propanamido)benzyl methyl(2-(methylamino)ethyl)carbamate (31)

A flame-dried flask was charged with mp-ValAlaPAB-OPFP (22, 20 mg, 31 mol) and Boc-N,N-DiMeEDA (30, 13 μL, 61 μmol) in anhydrous DMF (0.23 mL). DIPEA (21 L, 122 μmol) was added to the above solution with stirring. After 30 minutes, the solvents were removed under reduced pressure and the resulting residue was resolubilized in DCM (0.18 mL) and TFA (46 μL), and stirred for an additional 30 minutes. The volatiles were removed under reduced pressure, and the resulting residue was dissolved in minimal volume of 1:1 MeCN: 0.05% (v/v) aqueous TFA, and purified by preparative LC using TFA as mobile phase modifier to provide 31 (3.1 mg, 18%). Analytical UPLC-MS (gradient 1): HPLC retention time=0.96 min; m/z (ESI+) calculated 559.29 [M+H]⁺; found 559.10.

### 4-((S)-2-((S)-2-(3-(2,5-dioxo-2,5-dihydro-1H-pyrrol-1-yl)propanamido)-3-methylbutanamido)propanamido)benzyl methyl(2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-N-methyl-6,11-dioxo-1,2,3,4,6,11-hexahydrotetracene-2-carboxamido)ethyl)carbamate (32)

PNU-OH (6, 5.2 mg, 8.3 μmol) and HATU (3.2 mg, 8.3 μmol) were dissolved in anhydrous DMF (0.14 mL). mp-ValAlaPAB-N,N-DiMeEDA (31, 3.1 mg, 5.5 μmol) was added as a solution in DMF (0.14 mL), followed by the addition of DIPEA (3.9 μL, 22 μmol). After 15 minutes, the reaction mixture was diluted with 0.5 mL MeCN and 0.5 mL 0.05% (v/v) aqueous TFA, and purified by preparative LC using TFA as mobile phase modifier to provide 32 (2.1 mg, 33%). Analytical UPLC-MS (gradient 1): HPLC retention time=1.83 min; m/z (ESI+) calculated 1168.47 [M+H]⁺; found 1168.65.

### Example 14: (2S,4S)—N-(15-(2,5-dioxo-2,5-dihydro-1H-pyrrol-1-yl)-4,7,10,13-tetraoxo-3,6,9,12-tetraazapentadecyl)-2,5,12-trihydroxy-4-(((2R,4S,5S,6S)-5-hydroxy-4-((S)-2-methoxymorpholino)-6-methyltetrahydro-2H-pyran-2-yl)oxy)-7-methoxy-6,11-dioxo-1,2,3,4,6,11-hexahydrotetracene-2-carboxamide (33)

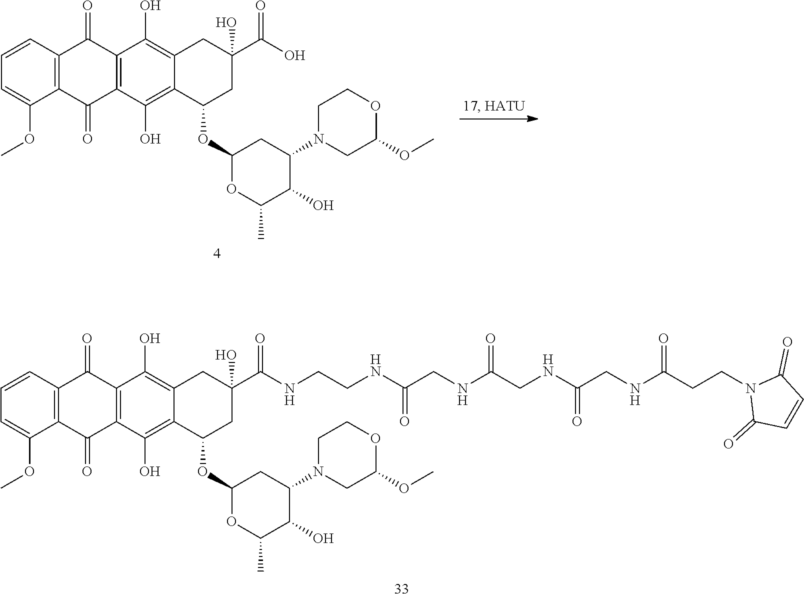

Nemorubicin-OH (4, 3.7 mg, 5.9 μmol) and HATU (2.5 mg, 6.5 μmol) were dissolved in anhydrous DMF (0.15 mL). mp-Gly₃-EDA (17, 2.9 mg, 5.9 μmol) as a solution in DMF (0.15 mL) was added to this solution with stirring, followed by the addition of DIPEA (4 L, 24 μmol). After 15 minutes, the reaction mixture was diluted with minimal volume of 1:1 MeCN: 0.05% (v/v) aqueous TFA, and purified by preparative LC using TFA as mobile phase modifier to provide 33 (1.0 mg, 17%). Analytical UPLC-MS (gradient 1): HPLC retention time=1.19 min; m/z (ESI+) calculated 994.37 [M+H]⁺; found 995.07.

### Example 15: (2S,4S)—N-(2-(3-(2,5-dioxo-2,5-dihydro-1H-pyrrol-1-yl)propanamido)ethyl)-2,5,12-trihydroxy-4-(((2R,4S,5S,6S)-5-hydroxy-4-((S)-2-methoxymorpholino)-6-methyltetrahydro-2H-pyran-2-yl)oxy)-7-methoxy-6,11-dioxo-1,2,3,4,6,11-hexahydrotetracene-2-carboxamide (34)

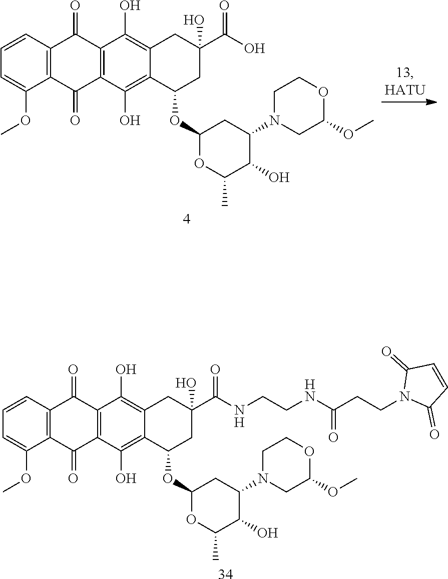

Nemorubicin-OH (4, 23 mg, 37 μmol) and HATU (21 mg, 55 μmol) were dissolved in anhydrous DMF (0.73 mL). mp-EDA (13, 12 mg, 37 μmol) as a solution in DMF (0.12 mL) was added to the above solution with stirring, followed by the addition of DIPEA (25 L, 148 μmol). After 15 minutes, the reaction mixture was diluted with 0.5 mL MeCN and 0.5 mL 0.05% (v/v) aqueous TFA, and purified by preparative LC using TFA as mobile phase modifier to provide 34 (7.0 mg, 23%). Analytical UPLC-MS (gradient 1): HPLC retention time=1.54 min; m/z (ESI+) calculated 823.30 [M+H]⁺; found 823.37.

### Example 16: 4-((S)-2-((S)-2-(3-(2,5-dioxo-2,5-dihydro-1H-pyrrol-1-yl)propanamido)-3-methylbutanamido)propanamido)benzyl (2-(2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)acetamido)ethyl)carbamate (36)

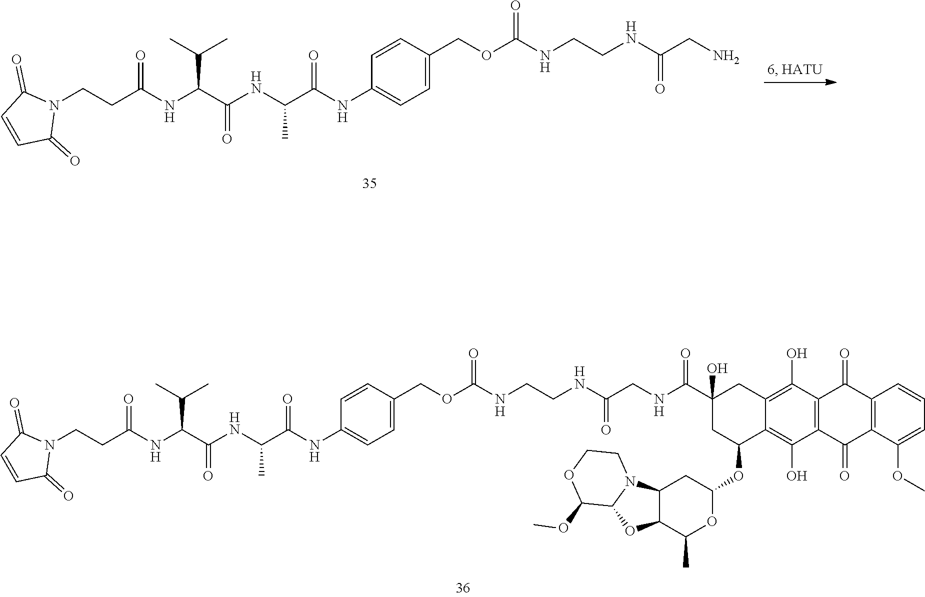 [SMILES: CC(C)[C@H](NC(=O)CCN1C(=O)C=CC1=O)C(=O)N[C@@H](C)C(=O)Nc1ccc(COC(=O)NCCNC(=O)CN)cc1 ; COc1cccc2c1C(=O)c1c(O)c3c(c(O)c1C2=O)C[C@@](O)(C(=O)NCC(=O)NCCNC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1]

mp-ValAlaPAB-EDA-Gly (35, 9 mg, 15 μmol), PNU-OH (6, 9 mg, 15 μmol) and HATU (6 mg, 15 μmol) were dissolved in anhydrous DMF (0.30 mL). The reaction was cooled to 0° C. followed by the addition of DIPEA (8 μL, 44 μmol). After 15 minutes, the reaction mixture was quenched with 8 μL of AcOH, diluted with 0.5 mL MeCN and 0.5 mL 10 mM aqueous NH₄OAc, then purified by preparative LC using NH₄OAc as mobile phase modifier to provide 36 (2 mg, 13%). Analytical UPLC-MS (gradient 2): HPLC retention time=1.72 min; m/z (ESI+) calculated 1197.46 [M+H]⁺; found 1197.80.

### Example 17: 4-((S)-2-((S)-2-(3-(2,5-dioxo-2,5-dihydro-1H-pyrrol-1-yl)propanamido)-3-methylbutanamido)propanamido)benzyl ((R)-1-oxo-1-((2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)ethyl)amino)propan-2-yl)carbamate (38)

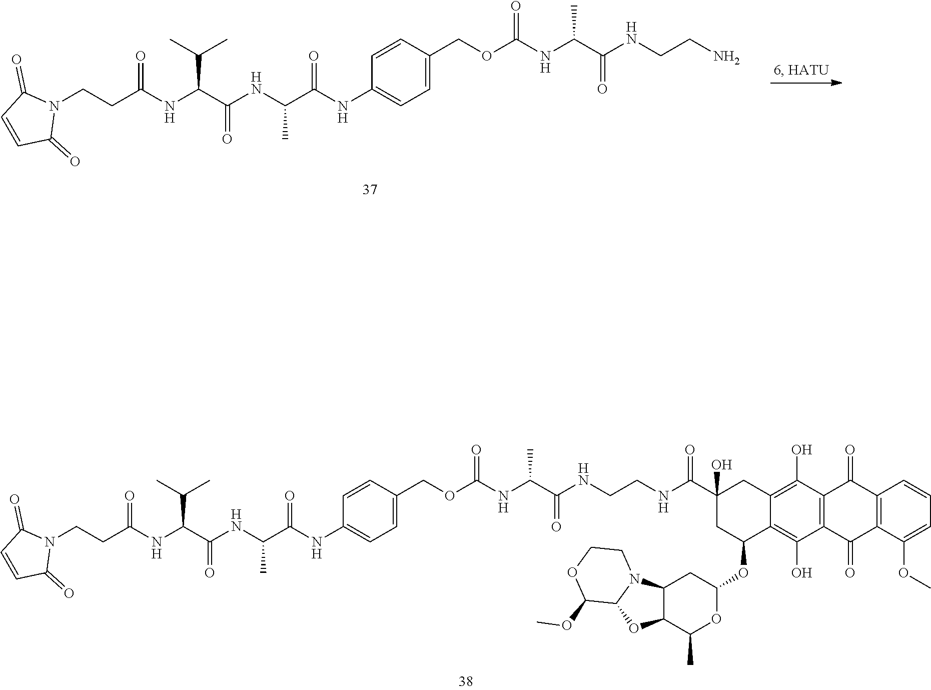 [SMILES: CC(C)[C@H](NC(=O)CCN1C(=O)C=CC1=O)C(=O)N[C@@H](C)C(=O)Nc1ccc(COC(=O)N[C@H](C)C(=O)NCCN)cc1 ; COc1cccc2c1C(=O)c1c(O)c3c(c(O)c1C2=O)C[C@@](O)(C(=O)NCCNC(=O)[C@@H](C)NC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1]

mp-ValAlaPAB-Ala(D)-EDA (37, 9 mg, 15 μmol), PNU-OH (6, 9 mg, 15 mol) and HATU (6 mg, 15 μmol) were dissolved in anhydrous DMF (0.30 mL). The reaction was cooled to 0° C. followed by the addition of DIPEA (8 μL, 44 μmol). After 15 minutes, the reaction mixture was quenched with 8 μL of AcOH, diluted with 0.5 mL MeCN and 0.5 mL 10 mM aqueous NH₄OAc, then purified by preparative LC using NH₄OAc as mobile phase modifier to provide 38 (4 mg, 24%). Analytical UPLC-MS (gradient 2): HPLC retention time=1.73 min; m/z (ESI+) calculated 1211.48 [M+H]⁺; found 1211.85.

### Example 18: 4-((S)-2-((S)-2-(3-(2,5-dioxo-2,5-dihydro-1H-pyrrol-1-yl)propanamido)-3-methylbutanamido)propanamido)benzyl ((S)-1-oxo-1-((2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)ethyl)amino)propan-2-yl)carbamate (40)

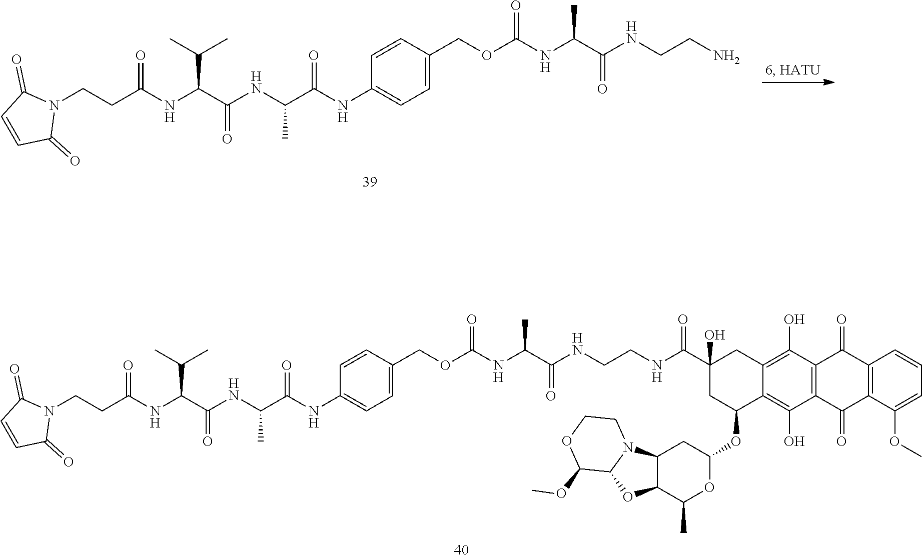 [SMILES: CC(C)[C@H](NC(=O)CCN1C(=O)C=CC1=O)C(=O)N[C@@H](C)C(=O)Nc1ccc(COC(=O)N[C@@H](C)C(=O)NCCN)cc1 ; COc1cccc2c1C(=O)c1c(O)c3c(c(O)c1C2=O)C[C@@](O)(C(=O)NCCNC(=O)[C@H](C)NC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1]

mp-ValAlaPAB-Ala-EDA (39, 9 mg, 15 μmol), PNU-OH (6, 9 mg, 15 μmol) and HATU (6 mg, 15 μmol) were dissolved in anhydrous DMF (0.30 mL). The reaction was cooled to 0° C. followed by the addition of DIPEA (8 μL, 44 μmol). After 15 minutes, the reaction mixture was quenched with 8 μL of AcOH, diluted with 0.5 mL MeCN and 0.5 mL 10 mM aqueous NH₄OAc, then purified by preparative LC using NH₄OAc as mobile phase modifier to provide 40 (4 mg, 24%). Analytical UPLC-MS (gradient 2): HPLC retention time=1.76 min; m/z (ESI+) calculated 1211.48 [M+H]⁺; found 1211.85.

### Example 19: 4-((S)-2-((S)-2-(3-(2,5-dioxo-2,5-dihydro-1H-pyrrol-1-yl)propanamido)-3-methylbutanamido)propanamido)benzyl (2-oxo-2-((2-((2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamido)ethyl)amino)ethyl)carbamate (42)

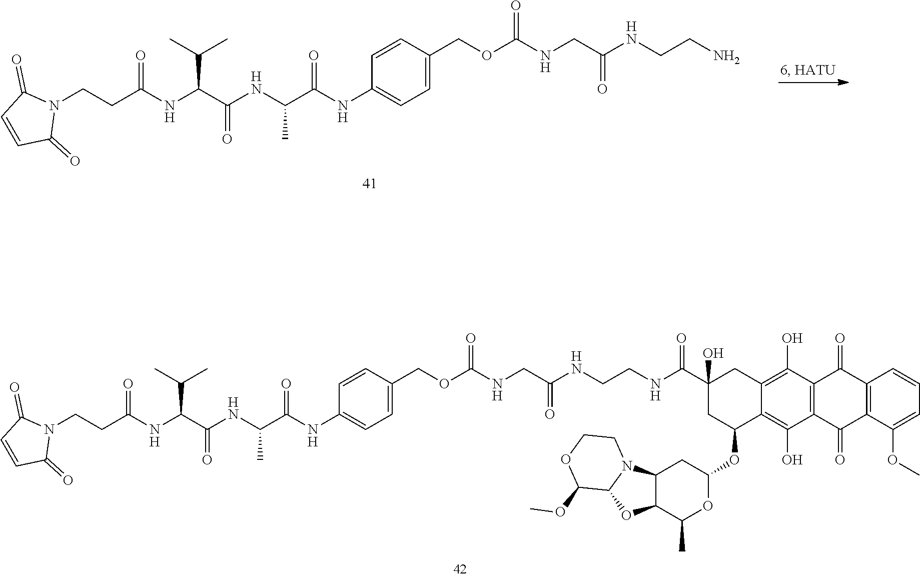 [SMILES: CC(C)[C@H](NC(=O)CCN1C(=O)C=CC1=O)C(=O)N[C@@H](C)C(=O)Nc1ccc(COC(=O)NCC(=O)NCCN)cc1 ; COc1cccc2c1C(=O)c1c(O)c3c(c(O)c1C2=O)C[C@@](O)(C(=O)NCCNC(=O)CNC(=O)OCc1ccc(NC(=O)[C@H](C)NC(=O)[C@@H](NC(=O)CCN2C(=O)C=CC2=O)C(C)C)cc1)C[C@@H]3O[C@H]1C[C@H]2[C@H](O[C@@H]3[C@@H](OC)OCCN32)[C@H](C)O1]

mp-ValAlaPAB-Gly-EDA (41, 9 mg, 15 μmol), PNU-OH (6, 9 mg, 15 μmol) and HATU (6 mg, 15 μmol) were dissolved in anhydrous DMF (0.30 mL). The reaction was cooled to 0° C. followed by the addition of DIPEA (8 μL, 44 μmol). After 15 minutes, the reaction mixture was quenched with 8 μL of AcOH, diluted with 0.5 mL MeCN and 0.5 mL 10 mM aqueous NH₄OAc, then purified by preparative LC using NH₄OAc as mobile phase modifier to provide 42 (4 mg, 24%). Analytical UPLC-MS (gradient 2): HPLC retention time=1.71 min; m/z (ESI+) calculated 1197.46 [M+H]⁺; found 1197.80.

### Example 20: (2S,4S)—N-(2-(2-(3-(2,5-dioxo-2,5-dihydro-1H-pyrrol-1-yl)propanamido)acetamido)ethyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (44)

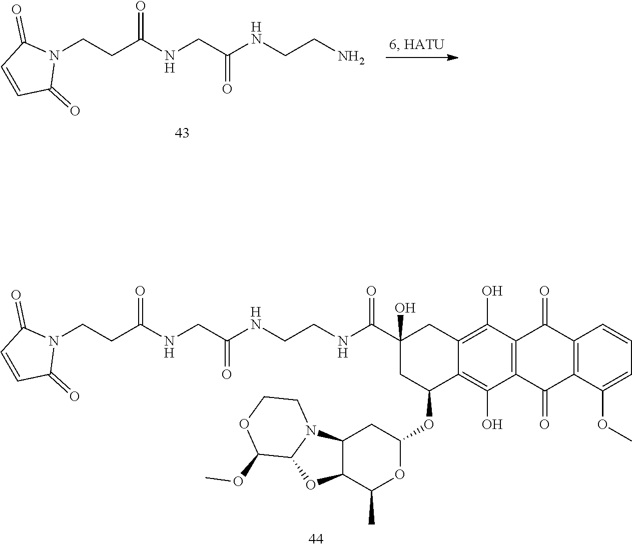 [SMILES: NCCNC(=O)CNC(=O)CCN1C(=O)C=CC1=O ; COc1cccc2c1C(=O)c1c(O)c3c(c(O)c1C2=O)C[C@@](O)(C(=O)NCCNC(=O)CNC(=O)CCN1C(=O)C=CC1=O)C[C@@H]3O[C@H]1C[C@H]2[C@H](O[C@@H]3[C@@H](OC)OCCN32)[C@H](C)O1]

PNU-OH (6, 17 mg, 27 μmol) and HATU (10 mg, 27 μmol) were dissolved in anhydrous DMF (0.30 mL) and cooled to 0° C. DIPEA (14 μL, 81 μmol) was added the reaction was allowed to preactivate for 5 minutes. mp-Gly-EDA (43, 7 mg, 27 μmol) was added with minimal DMF. After 15 minutes, the reaction mixture was quenched with 14 μL of AcOH, diluted with 0.5 mL MeCN and 0.5 mL 10 mM aqueous NH₄OAc, then purified by preparative LC using NH₄OAc as mobile phase modifier to provide 44 (1 mg, 4%). Analytical UPLC-MS (gradient 2): HPLC retention time=1.57 min; m/z (ESI+) calculated 878.31 [M+H]⁺; found 878.59.

### Example 21: (2S,4S)—N-(2-((S)-2-(3-(2,5-dioxo-2,5-dihydro-1H-pyrrol-1-yl)propanamido)propanamido)ethyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (46)

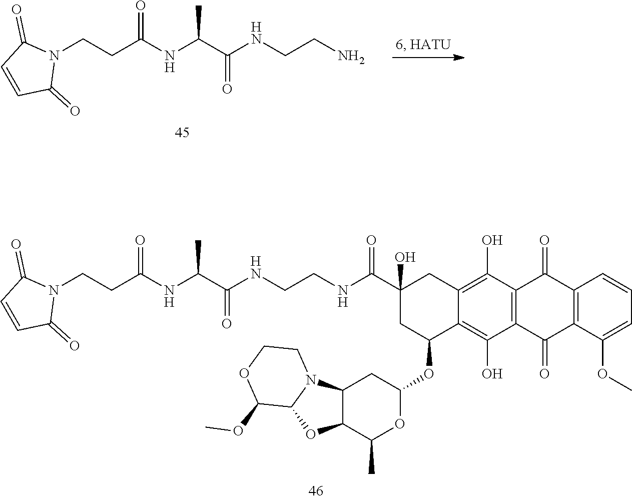 [SMILES: C[C@H](NC(=O)CCN1C(=O)C=CC1=O)C(=O)NCCN ; COc1cccc2c1C(=O)c1c(O)c3c(c(O)c1C2=O)C[C@@](O)(C(=O)NCCNC(=O)[C@H](C)NC(=O)CCN1C(=O)C=CC1=O)C[C@@H]3O[C@H]1C[C@H]2[C@H](O[C@@H]3[C@@H](OC)OCCN32)[C@H](C)O1]

mp-Ala-EDA (45, 4 mg, 13 μmol), PNU-OH (6, 7 mg, 12 μmol) and HATU (5 mg, 12 μmol) were dissolved in anhydrous DMF (0.26 mL). The reaction was cooled to 0° C. followed by the addition of DIPEA (7 μL, 39 μmol). After 15 minutes, the reaction mixture was quenched with 7 μL of AcOH, diluted with 0.5 mL MeCN and 0.5 mL 10 mM aqueous NH₄OAc, then purified by preparative LC using NH₄OAc as mobile phase modifier to provide 46 (3 mg, 24%). Analytical UPLC-MS (gradient 2): HPLC retention time=1.61 min; m/z (ESI+) calculated 892.33 [M+H]⁺; found 892.55.

### Example 22: (2S,4S)—N-((8S,11S)-15-(2,5-dioxo-2,5-dihydro-1H-pyrrol-1-yl)-11-isopropyl-8-methyl-4,7,10,13-tetraoxo-3,6,9,12-tetraazapentadecyl)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-1,2,3,4,6,11-hexahydrotetracene-2-carboxamide (48)

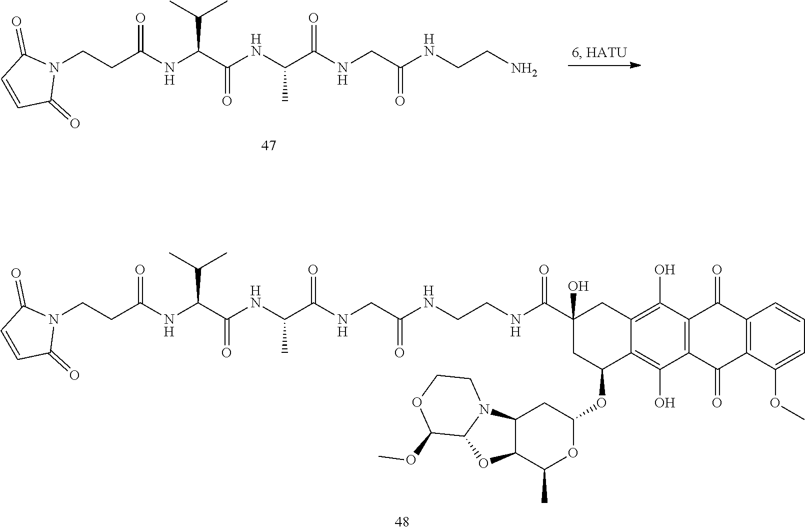 [SMILES: CC(C)[C@H](NC(=O)CCN1C(=O)C=CC1=O)C(=O)N[C@@H](C)C(=O)NCC(=O)NCCN ; COc1cccc2c1C(=O)c1c(O)c3c(c(O)c1C2=O)C[C@@](O)(C(=O)NCCNC(=O)CNC(=O)[C@H](C)NC(=O)[C@@H](NC(=O)CCN1C(=O)C=CC1=O)C(C)C)C[C@@H]3O[C@H]1C[C@H]2[C@H](O[C@@H]3[C@@H](OC)OCCN32)[C@H](C)O1]

mp-ValAlaGly-EDA (47, 6 mg, 14 μmol), PNU-OH (6, 9 mg, 114 μmol) and HATU (5 mg, 14 μmol) were dissolved in anhydrous DMF (0.28 mL). The reaction was cooled to 0° C. followed by the addition of DIPEA (7 μL, 42 μmol). After 15 minutes, the reaction mixture was quenched with 7 μL of AcOH, diluted with 0.5 mL MeCN and 0.5 mL 10 mM aqueous NH₄OAc, then purified by preparative LC using NH₄OAc as mobile phase modifier to provide 48 (1.4 mg, 10%). Analytical UPLC-MS (gradient 2): HPLC retention time=1.61 min; m/z (ESI+) calculated 1048.41 [M+H]⁺; found 1048.68.

### In Vitro Assays

Cell culture of cell lines as listed in Table 2-4 in log-phase growth were seeded in 96-well plates and grown for 24 h in cell culture media containing 150 μL RPMI 1640 supplemented with 20% FBS (see Mol. Cancer Ther., 2016, 15(5), 938-945 and Mol. Cancer Ther., 2018, 17(8), 1752-1760). Serial dilutions of free drugs or antibody-drug conjugates (final concentration between 0.004 nM and 1 μM) in cell culture media were prepared at 4× working concentrations; 50 μL of each dilution was added to the 96-well plates. Following addition of the drug or ADC, the cells were incubated with test articles (free drugs or ADCs) for 4 d at 37° C. After 96 h, growth inhibition was assessed by CellTiter-Glo® (Promega, Madison, WI) and luminescence was measured on a plate reader (brand, model). The IC₅₀ values, determined in triplicate, are defined herein as the concentration that results in a 50% reduction in cell growth relative to untreated controls.

### (A) Anthracycline Free Drugs

Cells were treated for 96 hours with anthracycline analogues listed in Table 2, and then assessed for viability as described in the procedures as described above. The IC₅₀ ranges are as follows: A denotes IC₅₀<10 nM; B denotes 10 nM≤IC₅₀<100 nM; C denotes 100 nM≤IC₅₀<1000 nM; D denotes IC₅₀≥1000 nM. ND denotes value not determined with that assay for the specified compound.

TABLE 2

| | | IC₅₀ (nM) | | | | |
|---|---|---|---|---|---|---|
| Compound | | L540cy, | 786-O, | BxPC3, | HL60, | HL60/RV, |
| No. | Drug | HL | MDR + RCC | PancCa | AML | MDR + AML |
| 1 | DOX | B | B | C | B | C |
| 2 | DOX-COOH | D | D | D | D | D |
| 3 | Nemorubicin | A | A | A | ND | ND |
| 4 | Nemorubicin- | B | B | B | ND | ND |
| | COOH | | | | | |
| 5 | PNU-159682 | A | A | A | A | A |
| 6 | PNU-COOH | A | A | B | B | B |
| 9 | PNU-EDA | A | A | A | A | C |
| — | PNU-EDA-Gly₃ | B | B | B | B | C |

Doxorubicin (1) displayed cytotoxic activity on the cancer lines tested with IC₅₀ values in the range of 13-390 nM. Oxidation of doxorubicin to form the carboxylic acid derivative (DOX-COOH, 2) resulted in complete loss of cytotoxic activity wherein the IC₅₀s are >1000 nM. Nemorubicin (3), which displayed 2-3 nM potency, lost roughly one order of magnitude in potency upon oxidation to the corresponding nemorubicin-COOH derivative (4). PNU-159682 (5) was the most potent analogue tested with IC₅₀s around 0.01 nM for most of the cell lines tested, and the lowest in the case of 786-0 with an IC₅₀ value of <0.004 nM. Oxidation of PNU-159682 to the carboxylic acid derivative (PNU-COOH, 6) reduced potency slightly to the range of 5-19 nM. Elaboration of the PNU-COOH derivative by amide coupling to an ethylene diamine linker yielded the PNU-EDA derivative (9), which has an increased potency in 4 of 5 cell lines relative to PNU-159682. Further elaboration of Compound 9 with a glycine tripeptide (PNU-EDA-Gly₃, commercially available), however, led to a reduced potency relative to 9, with IC₅₀s ranging from 13-890 nM.

### (B) In Vitro Assays—Anthracycline ADCs.

ADCs were prepared by full reduction of interchain disulfides to reveal 8-conjugatable cysteines/antibody, and subsequent alkylation with the maleimide-containing drug-linkers (Compounds 14, 18, 21, 24, 29, 32, 33, 34, and 48) according to the procedures described in Mol. Cancer Ther. 2018, 17(8). 1752-1760. Conjugates of antibody cOKT9 were prepared with anthracycline linkers. Cancer cell lines were treated with cOKT9 ADCs (average DAR of 8:1) for 96 h and then assessed for viability. The IC₅₀s are shown in Table 3. The IC₅₀ ranges are as follows: A denotes IC₅₀<10 ng/mL; B denotes 10 ng/mL≤IC₅₀<100 ng/mL; C denotes 100 ng/mL≤IC₅₀<1000 ng/mL; D denotes IC₅₀≥1000 ng/mL. ND denotes value not determined with that assay for the specified compound.

TABLE 3

| | | IC₅₀ (ng/mL) | | | | |
|---|---|---|---|---|---|---|
| | | | 786-O, | | | |
| cOKT9 ADCs | | L540cy, | MDR + | BxPC3, | HL60, | HL60/RV, |
| No. | Drug linker | HL | RCC | PancCa | AML | MDR + AML |
| 24 | VA-EDA-PNU | A | B | A | B | B |
| 32 | VA-DMED-PNU | B | C | B | B | C |
| 29 | VA-Gly₃-EDA-PNU | A | B | A | B | D |
| 18 | mp-Gly₃-EDA-PNU | A | A | A | B | D |
| 14 | mp-EDA-PNU | A | A | A | C | D |
| 21 | mp-PEG₄-PNU | A | A | A | C | D |
| 33 | mp-Gly₃-EDA-Nemo | D | D | D | D | D |
| 34 | mp-EDA-Nemo | D | D | D | ND | ND |
| 48 | mp-VAG-EDA-PNU | A | B | A | ND | ND |

The results summarized in Table 3 show that, the conjugates bearing nemorubicin linkers 33 & 34 were inactive across the panel of cancer cell lines tested. In contrast, conjugates bearing PNU drug-linkers 24, 32, 29, 18, 14, and 21 were active on L540cy, 786-0, BxPC3, and HL60 cell lines, and conjugate 48 was active on L540cy, 786-0, and BxPC3, with IC₅₀s ranging from 1-150 ng/ml. On the multidrug resistant HL60/RV cell line, only the conjugates bearing PNU drug-linkers 24 and 32 were active.

Anti-CD30 ADCs comprising cAC10 antibody conjugated to drug-linkers 24, 32, 29, 18, 14, and 48 with an average DAR of 4 were prepared for evaluation on a CD30-expressing lymphoma cell line panel. As shown in the data summarized in Table 4, the conjugate bearing doxorubicin linker 33 was inactive with IC₅₀ values >20,000 ng/ml, the highest dose tested. Anti-CD30 conjugates bearing the PNU linkers 24, 32, 29, 18, and 14 were active on L540cy, DEL, and Karpas299 lymphoma cell lines, and conjugate 48 was active on L540cy and DEL cell lines, with IC₅₀s ranging from 0.2-4 ng/mL. On the multidrug resistant L428 lymphoma cell line, anti-CD30 conjugates containing PNU linkers 32, 18, and 14 displayed activity with IC₅₀s ranging from 3-610 ng/ml. All anti-CD30 PNU conjugates appeared to possess immunological specificity, wherein the IC₅₀s are generally 1-2 log units lower on CD30-negative Ramos NHL cell line as compared to the CD30 positive cell lines. The IC₅₀ ranges are as follows: A denotes IC₅₀<1 ng/mL; B denotes 1 ng/mL≤IC₅₀<10 ng/mL; C denotes 10 ng/mL≤IC₅₀<100 ng/mL; D denotes 100 ng/mL≤IC₅₀<500 ng/mL; E denotes IC₅₀≥500 ng/mL.

TABLE 4

| | | IC₅₀ (ng/mL) | | | | |
|---|---|---|---|---|---|---|
| cAC10 | | L540cy, | L428, | DEL, | Karpas299, | Ramos, |
| ADC No. | Drug linker | HL | MDR + HL | ALCL | ALCL | CD30 − NHL |
| 24 | VA-EDA-PNU | B | E | A | B | D |
| 32 | VA-DMED-PNU | C | E | B | B | D |
| 29 | VA-Gly₃-EDA- | B | E | A | A | E |
| | PNU | | | | | |
| 18 | mp-Gly₃-EDA- | B | D | A | A | D |
| | PNU | | | | | |
| 14 | mp-EDA-PNU | B | B | B | A | E |
| 48 | mp-VAG-EDA- | A | ND | A | ND | ND |
| | PNU | | | | | |

### In Vivo Xenograft Models

All experiments were conducted in concordance with the Animal Care and Use Committee in a facility fully accredited by the Association for Assessment and Accreditation of Laboratory Animal Care. Efficacy experiments were conducted using the L540cy Hodgkin's lymphoma and DEL/BVR anaplastic large cell lymphoma xenograft models (Mol. Cancer Ther., 2018, 17(8), 1752-1760). Tumor cells, as a cell suspension, were implanted subcutaneously in immune-compromised SCID female mice. Upon tumor engraftment, the mice were randomized into study groups when the average tumor volume reached about 100 mm³. The ADCs were dosed once via intraperitoneal injection. Tumor volume as a function of time was determined using the formula (L×W2)/2. Animals were euthanized when tumor volumes reached 1000 mm³.

### (A) L540cy CD30+ Hodgkin Lymphoma Xenograft Model

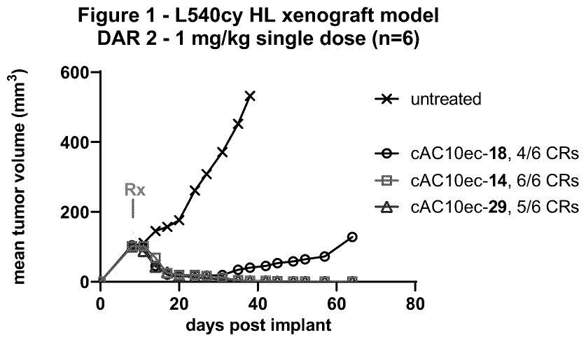

Anti-CD30 conjugates bearing PNU drug-linkers 29, 18, and 14 were evaluated in a CD30-expressing xenograft model as described above. cAC10 antibody with the S239C mutation is conjugated to the PNU drug linkers with an average DAR of 2 to minimize the effects of ADC pharmacokinetics. Tumor-bearing mice were administered a single dose intraperitoneally (i.p.) of test article once the average tumor volume reached 100 mm³ (typically on day 8). All three test articles were active with a majority of animals in a complete regression as shown in FIG. 1. Untreated mice were used as negative controls.

### (B) DEL/BVR MDR+, CD30+ Anaplastic Large Cell Lymphoma

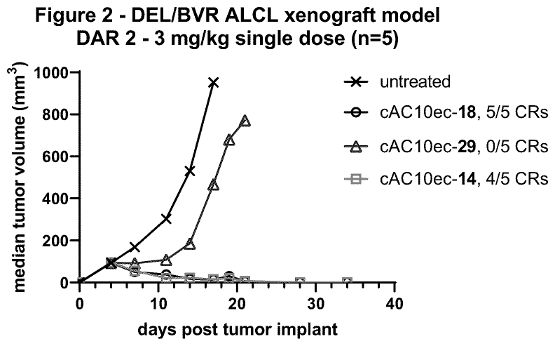

Anti-CD30 conjugates bearing PNU drug-linkers 29, 18, and 14 were further evaluated in a DEL/BVR MDR+, CD30+ anaplastic large cell lymphoma xenograft model. cAC10 antibody with the S239C mutation is conjugated to the PNU drug linkers with an average DAR of 2 to minimize the effects of ADC pharmacokinetics. Tumor-bearing mice were administered a 3 mg/kg single dose intraperitoneally (i.p.) of test article once the average tumor volume reached 100 mm³ (typically on day 4). Conjugates bearing PNU linkers 18 and 14 were shown to be efficacious in suppressing tumor growth, wherein almost all animals were in durable, complete regression at 18 days post treatment. However, the conjugate comprising PNU linker 29 appeared to be non-efficacious, and only provided minimal tumor growth delay (FIG. 2). Untreated mice were used as negative controls.

The contents of each of the references cited in the present disclosure are hereby incorporated by reference in their entirety.

A number of embodiments of the present disclosure have been described. Nevertheless, it will be understood that various modifications may be made without departing from the spirit and scope of the disclosure. Accordingly, other embodiments are within the scope of the following claims.

1. An antibody drug conjugate (ADC) having the structure: 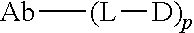
or a salt thereof;
wherein:
Ab is an antibody;
wherein each L is covalently attached to Ab via a sulfur atom of a cysteine residue or an ϵ-amino group of a lysine residue in Ab;
subscript p is an integer from 1 to 16;
each D is: 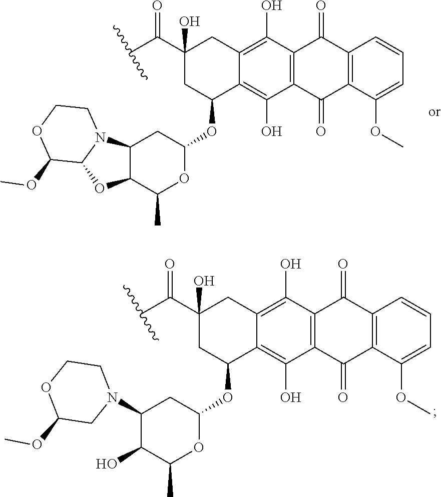
wherein [symbol] represents covalent attachment to L;
each L has the formula -M-(A)ₐ-(W)_{w}—(Y)_{y}—(X)—, wherein:
M is a succinimide, a hydrolyzed succinimide, an amide, or a triazole, wherein M is covalently attached to Ab;
subscript a is 0 or 1;
subscript y is 0 or 1;
subscript w is 0 or 1;
A is a C₂₋₁₀ alkylene optionally substituted with 1-3 R^{a1}; or a 3 to 20 membered heteroalkylene optionally substituted with 1-3 R^{b1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{b1} is independently selected from the group consisting of: C₁₋₆ alkyl, C₁₋₆ haloalkyl, C₁₋₆ alkoxy, C₁₋₆ haloalkoxy, halogen, —OH, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
W is from 1-6 amino acids; or
W has the structure 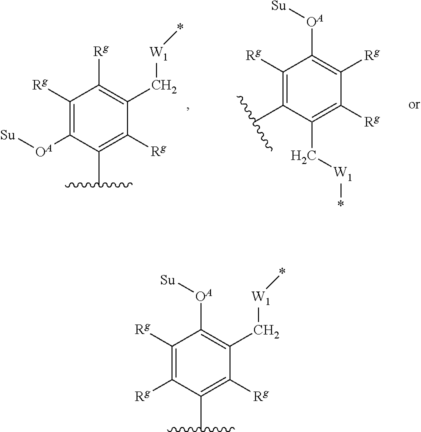
wherein Su is a Sugar moiety;
—O^{A}— represents the oxygen atom of a glycosidic bond;
each R⁹ is independently hydrogen, halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂;
W¹ is absent, *—C(═O)—O—, or *—O—C(═O)—; [symbol]
represents covalent attachment to A or M;
* represents covalent attachment to Y or X;
Y is a self-immolative moiety, a non-self-immolative releasable moiety, or a non-cleavable moiety;
X is a 4-16 membered heteroalkylene, wherein X is optionally substituted with 1-3 independently selected R^{X};
each R^{X} is independently a C₂-C₆ alkynyl group, —NR^{X1}R^{X2}, or a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl; or
two R^{X} attached to the same or adjacent carbon atom(s) of X, together with the carbon atom(s) to which they are attached form an unsubstituted 5-6 membered heterocyclyl;
each R^{X1} and R^{X2} are independently hydrogen or C₁₋₆ alkyl; and
L is optionally substituted with a PEG Unit from PEG1 to PEG72.

2. The ADC of claim 1, wherein M is a succinimide or a hydrolyzed succinimide.

3. (canceled)

4. (canceled)

5. (canceled)

6. The ADC of claim 1, wherein subscript a is 1.

7. The ADC of claim 1, wherein A is C₂₋₁₀ alkylene optionally substituted with 1-3 R^{a1}.

8. (canceled)

9. (canceled)

10. (canceled)

11. (canceled)

12. The ADC of claim 1, wherein A is a 3 to 20 membered heteroalkylene optionally substituted with 1-3 R^{b1}.

13. (canceled)

14. (canceled)

15. (canceled)

16. (canceled)

17. The ADC of claim 1, wherein A is selected from —(CH₂)₁₋₆—, —C(O)(CH₂)₁₋₆-#, —[NHC(O)(CH₂)₁₋₄]₁₋₃-#, and —NH(CH₂)₁₋₆[NHC(O)(CH₂)₁₋₄]₁₋₂-#, wherein # indicates attachment to M.

18. (canceled)

19. (canceled)

20. (canceled)

21. (canceled)

22. (canceled)

23. (canceled)

24. The ADC of claim 1, wherein one R^{g} is halogen, C₁-C₆ alkoxy, —N(C₁-C₆ alkyl)₂, —NHC(═O)(C₁-C₆ alkyl), —CN, —CF₃, acyl, carboxamido, C₁-C₆ alkyl, or —NO₂, and the remaining R^{g} are hydrogen.

25. The ADC of claim 1, wherein each R⁹ is hydrogen.

26. The ADC of claim 1, wherein W is from 2-6 amino acids.

27. (canceled)

28. The ADC of claim 1, wherein each amino acid in W is independently selected from the group consisting of alanine, glycine, lysine, serine, aspartic acid, aspartate methyl ester, N,N-dimethyl-lysine, phenylalanine, citrulline, valine, asparagine, homoserine methyl ether, isoleucine, leucine, glutamic acid, histidine, arginine, threonine, O-methylserine, O-methylaspartic acid, O-methylglutamic acid, N-methyllysine, O-methyltyrosine, O-methylhistidine, and O-methylthreonine.

29. The ADC of claim 1, wherein W is a dipeptide.

30. (canceled)

31. The ADC of claim 1, wherein W is a tripeptide.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The ADC of claim 1, wherein Y is

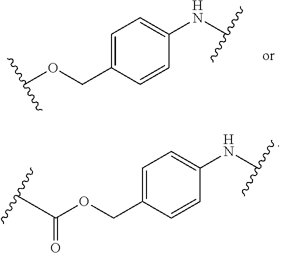 [SMILES: *Nc1ccc(CO*)cc1 ; *Nc1ccc(COC(*)=O)cc1]

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. The ADC of claim 1, wherein one R^{X} is a C₁-C₆ alkyl group optionally substituted with hydroxyl, —NR^{X1}R^{X2}, guanidino, 1 or 2 —CO₂H groups, —C(═O)NR^{X1}R^{X2}, urea, phenyl, naphthyl, indolyl, imidazolyl, —SH, —SCH₃, —SeCH₃, or 4-hydroxyphenyl optionally substituted with C₂-C₆ alkenyl.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. The ADC of claim 1, wherein X is —NH(C₂-C₆ alkylene)NH— optionally substituted with C₁-C₆ alkyl.

91. (canceled)

92. (canceled)

93. (canceled)

94. (canceled)

95. (canceled)

96. The ADC of claim 1, wherein X is ##—NH(C₂-C₆ alkylene)-(PEG2 to PEG4)-, wherein ## indicates attachment to D.

97. (canceled)

98. (canceled)

99. (canceled)

100. (canceled)

101. The ADC of claim 1, wherein X is an unsubstituted 4-16 membered heteroalkylene.

102. (canceled)

103. (canceled)

104. The ADC of claim 1, wherein X is selected from the group consisting of: 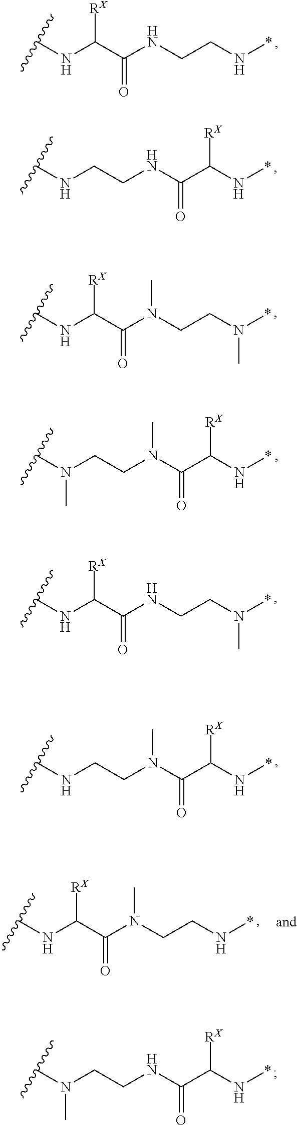
wherein the wavy line in X represents covalent attachment to Y, W, A, or M; and the * in X represents covalent attachment to D.

105. The ADC of claim 1, wherein X is selected from the group consisting of: 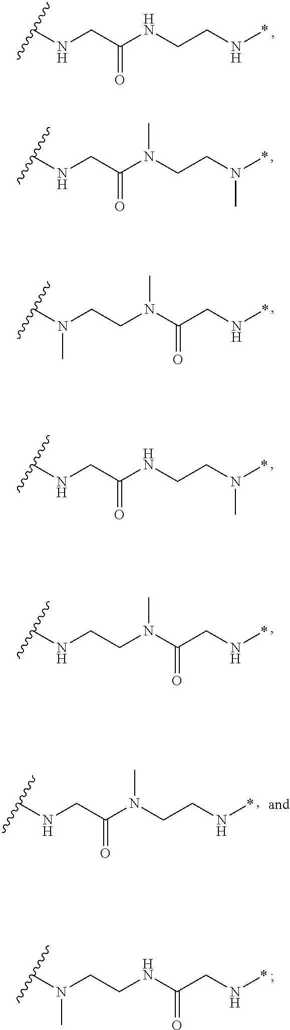
wherein the wavy line in X represents covalent attachment to Y, W, A, or M; and the * in X represents covalent attachment to D.

106. (canceled)

107. The ADC of claim 1, wherein each D-X is: 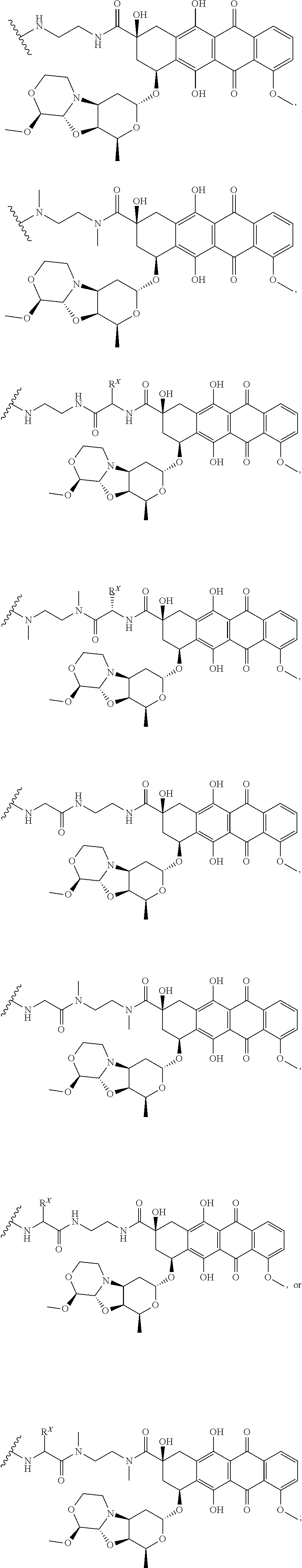
wherein [symbol] represents covalent attachment to Y, W, A, or M.

108. The ADC of claim 1, wherein each D-X is: 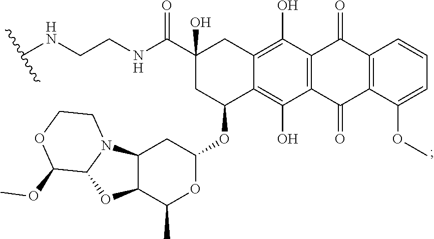
wherein [symbol] represents covalent attachment to Y, W, A, or M.

109. (canceled)

110. (canceled)

111. (canceled)

112. (canceled)

113. (canceled)

114. (canceled)

115. The ADC of claim 1, wherein:
A is a C₂₋₁₀ alkylene optionally substituted with 1-3 R^{a1};
each R^{a1} is independently selected from the group consisting of: C₁₋₆ alkyl, C₁₋₆ haloalkyl, C₁₋₆ alkoxy, C₁₋₆ haloalkoxy, halogen, —OH, ═O, —NR^{d1}R^{e1}, —(C₁₋₆ alkylene)-NR^{d1}R^{e1}, —C(═O)NR^{d1}R^{e1}, —C(═O)(C₁₋₆ alkyl), and —C(═O)O(C₁₋₆ alkyl);
each R^{d1} and R^{e1} are independently hydrogen or C₁₋₃ alkyl;
W is from 2-6 amino acids, wherein:
W is not a sortase enzyme recognition motif, and W does not include 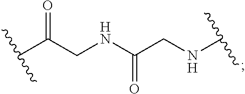 [SMILES: *NCC(=O)NCC(*)=O]
Y is a self-immolative moiety, a non-self-immolative releasable moiety, or a non-cleavable moiety; and
L is optionally substituted with a PEG Unit from PEG1 to PEG72.

116. (canceled)

117. (canceled)

118. (canceled)

119. The ADC of claim 1, wherein:
subscript y is 0;
subscript w is 0;
subscript a is 1; and
each D-X is: 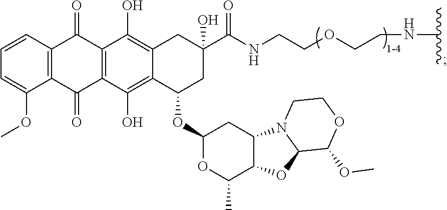
wherein [symbol] represents covalent attachment to Y, W, A, or M.

120. An ADC having the structure: 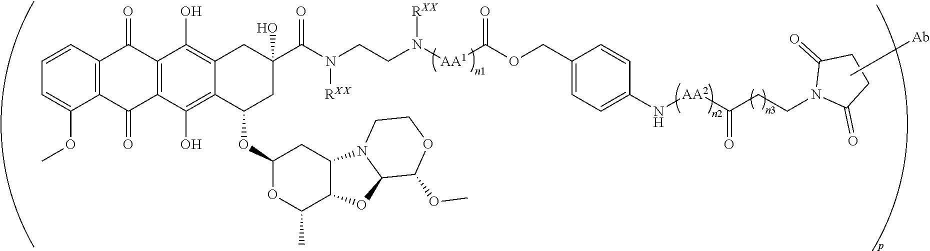
wherein:
each R^{XX} is independently hydrogen or C₁₋₃ alkyl;
n1 is an integer from 0 to 4;
n2 is an integer from 1 to 4;
n3 is an integer from 1 to 4;
each AA¹ is independently selected from the group consisting of alanine, glycine, lysine, serine, aspartic acid, aspartate methyl ester, N,N-dimethyl-lysine, phenylalanine, citrulline, valine, asparagine, homoserine methyl ether, isoleucine, leucine, glutamic acid, histidine, arginine, threonine, O-methylserine, O-methylaspartic acid, O-methylglutamic acid, N-methyllysine, O-methyltyrosine, O-methylhistidine, and O-methylthreonine;
each AA² is independently selected from from the group consisting of alanine, glycine, lysine, serine, aspartic acid, aspartate methyl ester, N,N-dimethyl-lysine, phenylalanine, citrulline, valine, asparagine, homoserine methyl ether, isoleucine, leucine, glutamic acid, histidine, arginine, threonine, O-methylserine, O-methylaspartic acid, O-methylglutamic acid, N-methyllysine, O-methyltyrosine, O-methylhistidine, and O-methylthreonine;
Ab is an antibody; and
p is an integer from 1 to 16.

121. The ADC of claim 121, wherein each AA¹ is independently selected from the group consisting of alanine, glycine, valine, and serine.

122. (canceled)

123. (canceled)

124. (canceled)

125. (canceled)

126. The ADC of claim 120, wherein each AA² is independently selected from the group consisting of alanine, glycine, valine, serine, leucine, and aspartic acid. In some embodiments, each AA² is independently selected from the group consisting of alanine and valine.

127. (canceled)

128. The ADC of claim 126, wherein (AA²)ₙ₂ is -Ala-Val-.

129. An ADC having the structure: 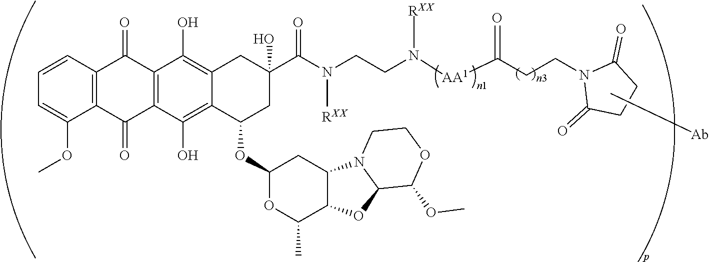
wherein:
each R^{XX} is independently hydrogen or C₁₋₃ alkyl;
n1 is an integer from 0 to 4;
each AA¹ is independently selected from the group consisting of alanine, glycine, lysine, serine, aspartic acid, aspartate methyl ester, N,N-dimethyl-lysine, phenylalanine, citrulline, valine, asparagine, homoserine methyl ether, isoleucine, leucine, glutamic acid, histidine, arginine, threonine, O-methylserine, O-methylaspartic acid, O-methylglutamic acid, N-methyllysine, O-methyltyrosine, O-methylhistidine, and O-methylthreonine;
n3 is an integer from 1 to 4;
Ab is an antibody; and
p is an integer from 1 to 16.

130. (canceled)

131. (canceled)

132. The ADC of claim 129, n1 is 2.

133. (canceled)

134. (canceled)

135. The ADC of claim 129, wherein each AA¹ is independently selected from the group consisting of alanine, glycine, valine, serine, leucine, arginine, and aspartic acid. In some embodiments, each AA¹ is independently selected from the group consisting of alanine, glycine, valine, and serine.

136. The ADC of claim 129, wherein
n1 is 3; and
each AA¹ is independently selected from the group consisting of alanine, glycine, valine, serine, leucine, arginine, and aspartic acid; and wherein at least one AA¹ is not glycine.

137. An ADC having the structure: 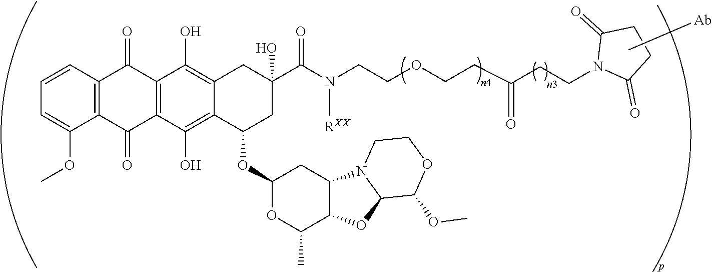
wherein:
R^{XX} is hydrogen or C₁₋₃ alkyl;
n4 is an integer from 2 to 8;
n3 is an integer from 1 to 4;
Ab is an antibody; and
p is an integer from 1 to 16.

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. A composition comprising a distribution of the ADCs of claim 1, or a salt thereof.

151. The composition of claim 150, further comprising at least one pharmaceutically acceptable carrier.

152. A method of treating cancer in a subject in need thereof, comprising administering to the subject a therapeutically effective amount of the ADC of claim 1, or a salt thereof.

153. A method of treating cancer in a subject in need thereof, comprising administering to the subject a therapeutically effective amount of the composition of claim 150.

154. A method of treating an autoimmune disorder in a subject in need thereof, comprising administering to the subject a therapeutically effective amount of the ADC of claim 1, or a salt thereof.

155. A method of treating an autoimmune disorder in a subject in need thereof, comprising administering to the subject a therapeutically effective amount of the composition of claim 150.


SEQUENCE LISTING (889 sequences)

SEQ ID NO: 1 - protein, 5 aa, synthetic construct
  REGION 1..5: note = Synthetic
DYYIT

SEQ ID NO: 2 - protein, 17 aa, synthetic construct
  REGION 1..17: note = Synthetic
WIYPGSGNTKYNEKFKG

SEQ ID NO: 3 - protein, 8 aa, synthetic construct
  REGION 1..8: note = Synthetic
YGNYWFAY

SEQ ID NO: 4 - protein, 15 aa, synthetic construct
  REGION 1..15: note = Synthetic
KASQSVDFDGDSYMN

SEQ ID NO: 5 - protein, 7 aa, synthetic construct
  REGION 1..7: note = Synthetic
AASNLES

SEQ ID NO: 6 - protein, 9 aa, synthetic construct
  REGION 1..9: note = Synthetic
QQSNEDPWT

SEQ ID NO: 7 - protein, 117 aa, synthetic construct
  REGION 1..117: note = Synthetic
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, synthetic construct
  REGION 1..111: note = Synthetic
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 447 aa, synthetic construct
  REGION 1..447: note = Synthetic
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, synthetic construct
  REGION 1..446: note = Synthetic
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 218 aa, synthetic construct
  REGION 1..218: note = Synthetic
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 118 aa, synthetic construct
  REGION 1..118: note = Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 13 - protein, 111 aa, synthetic construct
  REGION 1..111: note = Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 14 - protein, 448 aa, synthetic construct
  REGION 1..448: note = Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 218 aa, synthetic construct
  REGION 1..218: note = Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
  REGION 1..5: note = Synthetic
NYGMN

SEQ ID NO: 17 - protein, 17 aa, synthetic construct
  REGION 1..17: note = Synthetic
WINTYTGEPTYTDDFKG

SEQ ID NO: 18 - protein, 12 aa, synthetic construct
  REGION 1..12: note = Synthetic
GGFGSSYWYFDV

SEQ ID NO: 19 - protein, 11 aa, synthetic construct
  REGION 1..11: note = Synthetic
KASQDVSIAVA

SEQ ID NO: 20 - protein, 7 aa, synthetic construct
  REGION 1..7: note = Synthetic
SASYRYT

SEQ ID NO: 21 - protein, 9 aa, synthetic construct
  REGION 1..9: note = Synthetic
QQHYITPLT

SEQ ID NO: 22 - protein, 121 aa, synthetic construct
  REGION 1..121: note = Synthetic
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 23 - protein, 107 aa, synthetic construct
  REGION 1..107: note = Synthetic
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 24 - protein, 5 aa, synthetic construct
  REGION 1..5: note = Synthetic
TAGMQ

SEQ ID NO: 25 - protein, 17 aa, synthetic construct
  REGION 1..17: note = Synthetic
WINTHSGVPKYAEDFKG

SEQ ID NO: 26 - protein, 12 aa, synthetic construct
  REGION 1..12: note = Synthetic
SGFGSSYWYFDV

SEQ ID NO: 27 - protein, 11 aa, synthetic construct
  REGION 1..11: note = Synthetic
KASQDVSTAVA

SEQ ID NO: 28 - protein, 7 aa, synthetic construct
  REGION 1..7: note = Synthetic
SASYRYT

SEQ ID NO: 29 - protein, 9 aa, synthetic construct
  REGION 1..9: note = Synthetic
QQHYITPLT

SEQ ID NO: 30 - protein, 121 aa, synthetic construct
  REGION 1..121: note = Synthetic
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 31 - protein, 107 aa, synthetic construct
  REGION 1..107: note = Synthetic
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 32 - protein, 5 aa, synthetic construct
  REGION 1..5: note = Synthetic
SQNIY

SEQ ID NO: 33 - protein, 17 aa, synthetic construct
  REGION 1..17: note = Synthetic
YIEPYNVVPMYNPKFKG

SEQ ID NO: 34 - protein, 8 aa, synthetic construct
  REGION 1..8: note = Synthetic
SGSSNFDY

SEQ ID NO: 35 - protein, 12 aa, synthetic construct
  REGION 1..12: note = Synthetic
SASSSISSHYLH

SEQ ID NO: 36 - protein, 7 aa, synthetic construct
  REGION 1..7: note = Synthetic
RTSNLAS

SEQ ID NO: 37 - protein, 9 aa, synthetic construct
  REGION 1..9: note = Synthetic
QQGSSLPLT

SEQ ID NO: 38 - protein, 117 aa, synthetic construct
  REGION 1..117: note = Synthetic
EIQLVQSGAEVKKPGASVKVSCKASGYAFTSQNIYWVRQAPGQGLEWIGYIEPYNVVPMYNPKFKGRATLTVDKSTSTAYLELSSLRSEDTAVYYCARSGSSNFDYWGQGTLVTVSS

SEQ ID NO: 39 - protein, 108 aa, synthetic construct
  REGION 1..108: note = Synthetic
DIQLTQSPSSLSASVGDRVTITCSASSSISSHYLHWYQQKPGKSPKLLIYRTSNLASGVPSRFSGSGSGTDYTLTISSLQPEDFATYYCQQGSSLPLTFGQGTKVEIK

SEQ ID NO: 40 - protein, 5 aa, synthetic construct
  REGION 1..5: note = Synthetic
NYAMH

SEQ ID NO: 41 - protein, 17 aa, synthetic construct
  REGION 1..17: note = Synthetic
LIWYDGSNKFYGDSVKG

SEQ ID NO: 42 - protein, 6 aa, synthetic construct
  REGION 1..6: note = Synthetic
EGSGHY

SEQ ID NO: 43 - protein, 11 aa, synthetic construct
  REGION 1..11: note = Synthetic
RASQGISSALA

SEQ ID NO: 44 - protein, 7 aa, synthetic construct
  REGION 1..7: note = Synthetic
DASSLES

SEQ ID NO: 45 - protein, 9 aa, synthetic construct
  REGION 1..9: note = Synthetic
QQFNSYPIT

SEQ ID NO: 46 - protein, 115 aa, synthetic construct
  REGION 1..115: note = Synthetic
QVQLVESGGGVVQPGRSLRLSCAASGFTFSNYAMHWVRQAPGEGLEWVALIWYDGSNKFYGDSVKGRFTISRDNSKNTLYLQMNSLSAEDTAVYYCAREGSGHYWGQGTLVTVSS

SEQ ID NO: 47 - protein, 107 aa, synthetic construct
  REGION 1..107: note = Synthetic
AIQLTQSPSSLSASVGDRVTITCRASQGISSALAWYQQKPGKVPKSLIYDASSLESGVPSRFSGSGSGTDFTLTISSLQPEDFATYYCQQFNSYPITFGQGTRLEIK

SEQ ID NO: 48 - protein, 5 aa, synthetic construct
  REGION 1..5: note = Synthetic
NYAMS

SEQ ID NO: 49 - protein, 17 aa, synthetic construct
  REGION 1..17: note = Synthetic
YISPGGDYIYYADSVKG

SEQ ID NO: 50 - protein, 12 aa, synthetic construct
  REGION 1..12: note = Synthetic
DRRHYGSYAMDY

SEQ ID NO: 51 - protein, 16 aa, synthetic construct
  REGION 1..16: note = Synthetic
RSSKSLLHSNLNTYLY

SEQ ID NO: 52 - protein, 7 aa, synthetic construct
  REGION 1..7: note = Synthetic
RMSNLAS

SEQ ID NO: 53 - protein, 9 aa, synthetic construct
  REGION 1..9: note = Synthetic
MQHLEYPFT

SEQ ID NO: 54 - protein, 121 aa, synthetic construct
  REGION 1..121: note = Synthetic
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSS

SEQ ID NO: 55 - protein, 112 aa, synthetic construct
  REGION 1..112: note = Synthetic
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIK

SEQ ID NO: 56 - protein, 5 aa, synthetic construct
  REGION 1..5: note = Synthetic
TYAFH

SEQ ID NO: 57 - protein, 17 aa, synthetic construct
  REGION 1..17: note = Synthetic
GIVPIFGTLKYAQKFQD

SEQ ID NO: 58 - protein, 11 aa, synthetic construct
  REGION 1..11: note = Synthetic
AIQLEGRPFDH

SEQ ID NO: 59 - protein, 11 aa, synthetic construct
  REGION 1..11: note = Synthetic
RASQGITSYLA

SEQ ID NO: 60 - protein, 7 aa, synthetic construct
  REGION 1..7: note = Synthetic
AASALQS

SEQ ID NO: 61 - protein, 10 aa, synthetic construct
  REGION 1..10: note = Synthetic
QQVNRGAAIT

SEQ ID NO: 62 - protein, 120 aa, synthetic construct
  REGION 1..120: note = Synthetic
QVQLVQSGAEVKKPGSSVRVSCRASGGSSTTYAFHWVRQAPGQGLEWMGGIVPIFGTLKYAQKFQDRVTLTADKSTGTAYMELNSLRLDDTAVYYCARAIQLEGRPFDHWGQGTQVTVSA

SEQ ID NO: 63 - protein, 108 aa, synthetic construct
  REGION 1..108: note = Synthetic
DIQLTQSPSFLSASVGDRVTITCRASQGITSYLAWYQQKPGKAPKLLIYAASALQSGVPSRFSGRGSGTEFTLTISSLQPEDFATYYCQQVNRGAAITFGHGTRLDIK

SEQ ID NO: 64 - protein, 5 aa, synthetic construct
  REGION 1..5: note = Synthetic
TYAFH

SEQ ID NO: 65 - protein, 17 aa, synthetic construct
  REGION 1..17: note = Synthetic
GIVPIFGTLKYAQKFQD

SEQ ID NO: 66 - protein, 11 aa, synthetic construct
  REGION 1..11: note = Synthetic
AIQLEGRPFDH

SEQ ID NO: 67 - protein, 11 aa, synthetic construct
  REGION 1..11: note = Synthetic
RASQGITSYLA

SEQ ID NO: 68 - protein, 7 aa, synthetic construct
  REGION 1..7: note = Synthetic
AASALQS

SEQ ID NO: 69 - protein, 10 aa, synthetic construct
  REGION 1..10: note = Synthetic
QQVNRGAAIT

SEQ ID NO: 70 - protein, 120 aa, synthetic construct; identical to SEQ ID NO: 62
  REGION 1..120: note = Synthetic

SEQ ID NO: 71 - protein, 108 aa, synthetic construct; identical to SEQ ID NO: 63
  REGION 1..108: note = Synthetic

SEQ ID NO: 72 - protein, 5 aa, synthetic construct
  REGION 1..5: note = Synthetic
RYTMH

SEQ ID NO: 73 - protein, 17 aa, synthetic construct
  REGION 1..17: note = Synthetic
VISFDGSNKYYVDSVKG

SEQ ID NO: 74 - protein, 10 aa, synthetic construct
  REGION 1..10: note = Synthetic
EARGSYAFDI

SEQ ID NO: 75 - protein, 11 aa, synthetic construct
  REGION 1..11: note = Synthetic
RASQSVSSYLA

SEQ ID NO: 76 - protein, 7 aa, synthetic construct
  REGION 1..7: note = Synthetic
DASNRAT

SEQ ID NO: 77 - protein, 10 aa, synthetic construct
  REGION 1..10: note = Synthetic
QQRSNWPPFT

SEQ ID NO: 78 - protein, 119 aa, synthetic construct
  REGION 1..119: note = Synthetic
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 79 - protein, 108 aa, synthetic construct
  REGION 1..108: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 80 - protein, 5 aa, synthetic construct
  REGION 1..5: note = Synthetic
SFWMH

SEQ ID NO: 81 - protein, 17 aa, synthetic construct
  REGION 1..17: note = Synthetic
YINPRSGYTEYNEIFRD

SEQ ID NO: 82 - protein, 9 aa, synthetic construct
  REGION 1..9: note = Synthetic
FLGRGAMDY

SEQ ID NO: 83 - protein, 11 aa, synthetic construct
  REGION 1..11: note = Synthetic
RASQDISNYLA

SEQ ID NO: 84 - protein, 7 aa, synthetic construct
  REGION 1..7: note = Synthetic
YTSKIHS

SEQ ID NO: 85 - protein, 9 aa, synthetic construct
  REGION 1..9: note = Synthetic
QQGNTFPYT

SEQ ID NO: 86 - protein, 118 aa, synthetic construct [sequence omitted]

SEQ ID NO: 87 - protein, 107 aa, synthetic construct [sequence omitted]

SEQ ID NO: 88 - protein, 7 aa, synthetic construct
  REGION 1..7: note = Synthetic
TSSYYWG

SEQ ID NO: 89 - protein, 16 aa, synthetic construct
  REGION 1..16: note = Synthetic
TIYYNGSTYYSPSLKS

SEQ ID NO: 90 - protein, 11 aa, synthetic construct
  REGION 1..11: note = Synthetic
QGYDIKINIDV

SEQ ID NO: 91 - protein, 11 aa, synthetic construct
  REGION 1..11: note = Synthetic
RASQSVSSYLA

SEQ ID NO: 92 - protein, 7 aa, synthetic construct
  REGION 1..7: note = Synthetic
VASNRAT

SEQ ID NO: 93 - protein, 9 aa, synthetic construct
  REGION 1..9: note = Synthetic
QQRSNWPLT

SEQ ID NO: 94 - protein, 121 aa, synthetic construct
  REGION 1..121: note = Synthetic
QLQLQESGPGLVKPSETLSLTCTVSGGSISTSSYYWGWIRQPPGKGLEWIGTIYYNGSTYYSPSLKSRVSISVDTSKNQFSLKLSSVTAADTSVYYCARQGYDIKINIDVWGQGTTVTVSS

SEQ ID NO: 95 - protein, 107 aa, synthetic construct
  REGION 1..107: note = Synthetic
EIVLTQSPATLSLSPGERATLSCRASQSVSSYLAWYQQKPGQAPRLLIYVASNRATGIPARFSGSGSGTDFTLTISSLEPEDFAVYYCQQRSNWPLTFGGGTKVEIK

SEQ ID NO: 96 - protein, 5 aa, synthetic construct
  REGION 1..5: note = Synthetic
SSWMN

SEQ ID NO: 97 - protein, 17 aa, synthetic construct
  REGION 1..17: note = Synthetic
RIYPGDGNTHYAQKFQG

SEQ ID NO: 98 - protein, 8 aa, synthetic construct
  REGION 1..8: note = Synthetic
GYLDPMDY

SEQ ID NO: 99 - protein, 11 aa, synthetic construct
  REGION 1..11: note = Synthetic
QASQGINNYLN

SEQ ID NO: 100 - protein, 7 aa, synthetic construct
  REGION 1..7: note = Synthetic
YTSGLHA

SEQ ID NO: 101 - protein, 9 aa, synthetic construct
  REGION 1..9: note = Synthetic
QQYSILPWT

SEQ ID NO: 102 - protein, 117 aa, synthetic construct
  REGION 1..117: note = Synthetic
QVQLVQSGAEVKKPGSSVKVSCKASGYAFTSSWMNWVRQAPGQGLEWMGRIYPGDGNTHYAQKFQGRVTLTADKSTSTAYMELSSLRSEDTAVYYCGEGYLDPMDYWGQGTLVTVSS

SEQ ID NO: 103 - protein, 107 aa, synthetic construct
  REGION 1..107: note = Synthetic
DIQMTQSPSSLSASVGDRVTITCQASQGINNYLNWYQQKPGKAPKLLIHYTSGLHAGVPSRFSGSGSGTDYTLTISSLEPEDVATYYCQQYSILPWTFGGGTKVEIK

SEQ ID NO: 104 - protein, 5 aa, synthetic construct
  REGION 1..5: note = Synthetic
SYGMH

SEQ ID NO: 105 - protein, 17 aa, synthetic construct
  REGION 1..17: note = Synthetic
VISYDGSNKYYADSVKG

SEQ ID NO: 106 - skipped

SEQ ID NO: 107 - protein, 11 aa, synthetic construct
  REGION 1..11: note = Synthetic
RTSQSISSYLN

SEQ ID NO: 108 - protein, 7 aa, synthetic construct
  REGION 1..7: note = Synthetic
WASTRES

SEQ ID NO: 109 - protein, 9 aa, synthetic construct
  REGION 1..9: note = Synthetic
QQSYDIPYT

SEQ ID NO: 110 - protein, 127 aa, synthetic construct
  REGION 1..127: note = Synthetic
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 111 - protein, 107 aa, synthetic construct
  REGION 1..107: note = Synthetic
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 112 - protein, 5 aa, synthetic construct
  REGION 1..5: note = Synthetic
NYWMS

SEQ ID NO: 113 - protein, 17 aa, synthetic construct
  REGION 1..17: note = Synthetic
NIKQDGSEKFYADSVKG

SEQ ID NO: 114 - protein, 9 aa, synthetic construct
  REGION 1..9: note = Synthetic
VGPSWEQDY

SEQ ID NO: 115 - protein, 14 aa, synthetic construct
  REGION 1..14: note = Synthetic
TGSSSNIGSYYGVH

SEQ ID NO: 116 - protein, 7 aa, synthetic construct
  REGION 1..7: note = Synthetic
SDTNRPS

SEQ ID NO: 117 - protein, 11 aa, synthetic construct
  REGION 1..11: note = Synthetic
QSYDKGFGHRV

SEQ ID NO: 118 - protein, 118 aa, synthetic construct
  REGION 1..118: note = Synthetic
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSA

SEQ ID NO: 119 - protein, 94 aa, synthetic construct
  REGION 1..94: note = Synthetic
QSVLTQPPSVSGAPGQRVTISCTGSSSNIGSYYGVHWYQQLPGTAPKLLIYSDTNRPSGVPDRFSGSKSGTSASLAITGLQAEDEADYYCQSYD

SEQ ID NO: 120 - protein, 5 aa, synthetic construct
  REGION 1..5: note = Synthetic
SYAIS

SEQ ID NO: 121 - protein, 17 aa, synthetic construct
  REGION 1..17: note = Synthetic
GIIPIFGTANYAQKFQG

SEQ ID NO: 122 - protein, 6 aa, synthetic construct
  REGION 1..6: note = Synthetic
GLLWNY

SEQ ID NO: 123 - protein, 11 aa, synthetic construct
  REGION 1..11: note = Synthetic
RASQSVSSNLA

SEQ ID NO: 124 - protein, 7 aa, synthetic construct
  REGION 1..7: note = Synthetic
GASTTAS

SEQ ID NO: 125 - protein, 11 aa, synthetic construct
  REGION 1..11: note = Synthetic
QQYNNWPPAYT

SEQ ID NO: 126 - protein, 115 aa, synthetic construct
  REGION 1..115: note = Synthetic
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 127 - protein, 109 aa, synthetic construct
  REGION 1..109: note = Synthetic
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 128 - protein, 5 aa, synthetic construct
  REGION 1..5: note = Synthetic
NYGMN

SEQ ID NO: 129 - protein, 17 aa, synthetic construct
  REGION 1..17: note = Synthetic
WINTYTGEPTYGEDFKG

SEQ ID NO: 130 - protein, 7 aa, synthetic construct
  REGION 1..7: note = Synthetic
FGNYVDY

SEQ ID NO: 131 - protein, 16 aa, synthetic construct
  REGION 1..16: note = Synthetic
RSSKNLLHSNGITYLY

SEQ ID NO: 132 - protein, 7 aa, synthetic construct
  REGION 1..7: note = Synthetic
QMSNLAS

SEQ ID NO: 133 - protein, 9 aa, synthetic construct
  REGION 1..9: note = Synthetic
AQNLEIPRT

SEQ ID NO: 134 - protein, 116 aa, synthetic construct
  REGION 1..116: note = Synthetic
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSS

SEQ ID NO: 135 - protein, 112 aa, synthetic construct
  REGION 1..112: note = Synthetic
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 136 - protein, 5 aa, synthetic construct
  REGION 1..5: note = Synthetic
KYGMN

SEQ ID NO: 137 - protein, 17 aa, synthetic construct
  REGION 1..17: note = Synthetic
WINTYTEEPTYGDDFKG

SEQ ID NO: 138 - protein, 7 aa, synthetic construct
  REGION 1..7: note = Synthetic
FGSAVDY

SEQ ID NO: 139 - protein, 16 aa, synthetic construct
  REGION 1..16: note = Synthetic
RSSKSLLHSNGITYLY

SEQ ID NO: 140 - protein, 7 aa, synthetic construct
  REGION 1..7: note = Synthetic
QMSNRAS

SEQ ID NO: 141 - protein, 9 aa, synthetic construct
  REGION 1..9: note = Synthetic
AQNLELPRT

SEQ ID NO: 142 - protein, 116 aa, synthetic construct
  REGION 1..116: note = Synthetic
QIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSS

SEQ ID NO: 143 - protein, 113 aa, synthetic construct
  REGION 1..113: note = Synthetic
DIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKR

SEQ ID NO: 144 - protein, 5 aa, synthetic construct
  REGION 1..5: note = Synthetic
DYSMH

SEQ ID NO: 145 - protein, 17 aa, synthetic construct
  REGION 1..17: note = Synthetic
WINTETGEPTYADDFKG

SEQ ID NO: 146 - protein, 4 aa, synthetic construct
  REGION 1..4: note = Synthetic
TAVY

SEQ ID NO: 147 - protein, 11 aa, synthetic construct
  REGION 1..11: note = Synthetic
RASQEISVSLS

SEQ ID NO: 148 - protein, 7 aa, synthetic construct
  REGION 1..7: note = Synthetic
ATSTLDS

SEQ ID NO: 149 - protein, 9 aa, synthetic construct
  REGION 1..9: note = Synthetic
LQYASYPWT

SEQ ID NO: 150 - protein, 113 aa, synthetic construct
  REGION 1..113: note = Synthetic
QVKLQESGPELKKPGETVKISCKASGYTFTDYSMHWVKQAPGKGLKWMGWINTETGEPTYADDFKGRFAFSLETSASTAYLQINNLKNEDTATYFCARTAVYWGQGTTVTVSS

SEQ ID NO: 151 - protein, 108 aa, synthetic construct
  REGION 1..108: note = Synthetic
DIQMTQSPSSLSASLGERVSLTCRASQEISVSLSWLQQEPDGTIKRLIYATSTLDSGVPKRFSGSRSGSDYSLTISSLESEDFVDYYCLQYASYPWTFGGGTKLEIKR

SEQ ID NO: 152 - protein, 5 aa, synthetic construct
  REGION 1..5: note = Synthetic
NYGMN

SEQ ID NO: 153 - protein, 17 aa, synthetic construct
  REGION 1..17: note = Synthetic
WINTYSGEPRYADDFKG

SEQ ID NO: 154 - protein, 9 aa, synthetic construct
  REGION 1..9: note = Synthetic
DYGRWYFDV

SEQ ID NO: 155 - protein, 10 aa, synthetic construct
  REGION 1..10: note = Synthetic
RASSSVSHMH

SEQ ID NO: 156 - protein, 7 aa, synthetic construct
  REGION 1..7: note = Synthetic
ATSNLAS

SEQ ID NO: 157 - protein, 9 aa, synthetic construct
  REGION 1..9: note = Synthetic
QQWSSTPRT

SEQ ID NO: 158 - protein, 118 aa, synthetic construct
  REGION 1..118: note = Synthetic
QIQLVQSGSELKKPGASVKVSCKASGYTFTNYGMNWVRQAPGQDLKWMGWINTYSGEPRYADDFKGRFVFSLDKSVNTAYLQISSLKAEDTAVYYCARDYGRWYFDVWGQGTTVTVSS

SEQ ID NO: 159 - protein, 107 aa, synthetic construct
  REGION 1..107: note = Synthetic
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIKR

SEQ ID NO: 160 - protein, 5 aa, synthetic construct
  REGION 1..5: note = Synthetic
RYWMS

SEQ ID NO: 161 - protein, 17 aa, synthetic construct
  REGION 1..17: note = Synthetic
EINPDSSTINYAPSLKD

SEQ ID NO: 162 - protein, 10 aa, synthetic construct
  REGION 1..10: note = Synthetic
PDGNYWYFDV

SEQ ID NO: 163 - protein, 11 aa, synthetic construct
  REGION 1..11: note = Synthetic
KASQDVGIAVA

SEQ ID NO: 164 - protein, 7 aa, synthetic construct
  REGION 1..7: note = Synthetic
WASTRHT

SEQ ID NO: 165 - protein, 9 aa, synthetic construct
  REGION 1..9: note = Synthetic
QQYSSYPYT

SEQ ID NO: 166 - protein, 119 aa, synthetic construct
  REGION 1..119: note = Synthetic
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 167 - protein, 108 aa, synthetic construct
  REGION 1..108: note = Synthetic
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKR

SEQ ID NO: 168 - protein, 5 aa, synthetic construct
  REGION 1..5: note = Synthetic
SFAMS

SEQ ID NO: 169 - protein, 17 aa, synthetic construct
  REGION 1..17: note = Synthetic
AISGSGGGTYYADSVKG

SEQ ID NO: 170 - protein, 13 aa, synthetic construct
  REGION 1..13: note = Synthetic
DKILWFGEPVFDY

SEQ ID NO: 171 - protein, 11 aa, synthetic construct
  REGION 1..11: note = Synthetic
RASQSVSSYLA

SEQ ID NO: 172 - protein, 7 aa, synthetic construct
  REGION 1..7: note = Synthetic
DASNRAT

SEQ ID NO: 173 - protein, 9 aa, synthetic construct
  REGION 1..9: note = Synthetic
QQRSNWPPT

SEQ ID NO: 174 - protein, 122 aa, synthetic construct [sequence omitted]

SEQ ID NO: 175 - protein, 108 aa, synthetic construct
  REGION 1..108: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 176 - protein, 5 aa, synthetic construct
  REGION 1..5: note = Synthetic
SYRMH

SEQ ID NO: 177 - protein, 17 aa, synthetic construct
  REGION 1..17: note = Synthetic
YINPSTGYTEYNQKFKD

SEQ ID NO: 178 - protein, 7 aa, synthetic construct
  REGION 1..7: note = Synthetic
GGGVFDY

SEQ ID NO: 179 - protein, 10 aa, synthetic construct
  REGION 1..10: note = Synthetic
SASSSISYMH

SEQ ID NO: 180 - protein, 7 aa, synthetic construct
  REGION 1..7: note = Synthetic
TTSNLAS

SEQ ID NO: 181 - protein, 9 aa, synthetic construct
  REGION 1..9: note = Synthetic
HQRSTYPLT

SEQ ID NO: 182 - protein, 116 aa, synthetic construct
  REGION 1..116: note = Synthetic
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 183 - protein, 106 aa, synthetic construct
  REGION 1..106: note = Synthetic
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 184 - protein, 4 aa, synthetic construct
  REGION 1..4: note = Synthetic
SYWM

SEQ ID NO: 185 - protein, 17 aa, synthetic construct
  REGION 1..17: note = Synthetic
EIIPINGHTNYNEKFKS

SEQ ID NO: 186 - protein, 14 aa, synthetic construct
  REGION 1..14: note = Synthetic
GGYYYYGSRDYFDY

SEQ ID NO: 187 - protein, 15 aa, synthetic construct
  REGION 1..15: note = Synthetic
KASQSVDYDGDSYMN

SEQ ID NO: 188 - protein, 7 aa, synthetic construct
  REGION 1..7: note = Synthetic
AASDLES

SEQ ID NO: 189 - protein, 9 aa, synthetic construct
  REGION 1..9: note = Synthetic
QQSHEDPFT

SEQ ID NO: 190 - protein, 123 aa, synthetic construct
  REGION 1..123: note = Synthetic
QVQLQQPGAELVKPGASVKLSCKASGYTFTSYWMHWVKQRPGQGLEWIGEIIPINGHTNYNEKFKSKATLTLDKSSSTAYMQLSSLASEDSAVYYCARGGYYYYGSRDYFDYWGQGTTLTVSS

SEQ ID NO: 191 - protein, 111 aa, synthetic construct
  REGION 1..111: note = Synthetic
DIVLTQSPASLAVSLGQRATISCKASQSVDYDGDSYMNWYQQIPGQPPKLLIYAASDLESGIPARFSGSGSGTDFTLNIHPVEEEDAATYYCQQSHEDPFTFGGGTKLEIK

SEQ ID NO: 192 - protein, 4 aa, synthetic construct
  REGION 1..4: note = Synthetic
SYWM

SEQ ID NO: 193 - protein, 17 aa, synthetic construct
  REGION 1..17: note = Synthetic
EIIPIFGHTNYNEKFKS

SEQ ID NO: 194 - protein, 14 aa, synthetic construct
  REGION 1..14: note = Synthetic
GGYYYYPRQGFLDY

SEQ ID NO: 195 - protein, 16 aa, synthetic construct
  REGION 1..16: note = Synthetic
KASQSVDYDSGDSYMN

SEQ ID NO: 196 - protein, 7 aa, synthetic construct
  REGION 1..7: note = Synthetic
AASDLES

SEQ ID NO: 197 - protein, 9 aa, synthetic construct
  REGION 1..9: note = Synthetic
QQSHEDPFT

SEQ ID NO: 198 - protein, 123 aa, synthetic construct
  REGION 1..123: note = Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 199 - protein, 111 aa, synthetic construct
  REGION 1..111: note = Synthetic
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 200 - protein, 4 aa, synthetic construct
  REGION 1..4: note = Synthetic
YGMN

SEQ ID NO: 201 - protein, 16 aa, synthetic construct
  REGION 1..16: note = Synthetic
YISSSSSTIYADSVKG

SEQ ID NO: 202 - protein, 6 aa, synthetic construct
  REGION 1..6: note = Synthetic
GPGMDV

SEQ ID NO: 203 - protein, 17 aa, synthetic construct
  REGION 1..17: note = Synthetic
KSSQSVLYSSNNKNYLA

SEQ ID NO: 204 - protein, 7 aa, synthetic construct
  REGION 1..7: note = Synthetic
WASTRES

SEQ ID NO: 205 - protein, 10 aa, synthetic construct
  REGION 1..10: note = Synthetic
QQYYSTPQLT

SEQ ID NO: 206 - protein, 112 aa, synthetic construct
  REGION 1..112: note = Synthetic
QVQLQQSGGGVVQPGRSLGLSCAASFTFSSYGMNWVRQAPGKGLEWVSYISSSSSTIYADSVKGRFTISRDNSKNTLYLQMNSLRAEDTAVYYCARGPGMDVWGQGTTVTVS

SEQ ID NO: 207 - protein, 114 aa, synthetic construct
  REGION 1..114: note = Synthetic
DIVLTQSPDSLAVSLGERATINCKSSQSVLYSSNNKNYLAWYQQKPGQPPKLLIYWASTRESGVPDRFSGSGSGTDFTPAISSLQAEDVAVYYCQQYYSTPQLTFGGGTKVDIK

SEQ ID NO: 208 - protein, 7 aa, synthetic construct
  REGION 1..7: note = Synthetic
TSGMGVG

SEQ ID NO: 209 - protein, 16 aa, synthetic construct
  REGION 1..16: note = Synthetic
HIWWDDDKRYNPALKS

SEQ ID NO: 210 - protein, 10 aa, synthetic construct
  REGION 1..10: note = Synthetic
MELWSYYFDY

SEQ ID NO: 211 - protein, 10 aa, synthetic construct
  REGION 1..10: note = Synthetic
SASSSVSYMH

SEQ ID NO: 212 - protein, 7 aa, synthetic construct
  REGION 1..7: note = Synthetic
DTSKLAS

SEQ ID NO: 213 - protein, 9 aa, synthetic construct
  REGION 1..9: note = Synthetic
FQGSVYPFT

SEQ ID NO: 214 - protein, 120 aa, synthetic construct
  REGION 1..120: note = Synthetic
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 215 - protein, 107 aa, synthetic construct
  REGION 1..107: note = Synthetic
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKR

SEQ ID NO: 216 - protein, 5 aa, synthetic construct
  REGION 1..5: note = Synthetic
NYGMN

SEQ ID NO: 217 - protein, 17 aa, synthetic construct
  REGION 1..17: note = Synthetic
WINTYTGEPTYADAFKG

SEQ ID NO: 218 - protein, 9 aa, synthetic construct
  REGION 1..9: note = Synthetic
DYGDYGMDY

SEQ ID NO: 219 - protein, 15 aa, synthetic construct
  REGION 1..15: note = Synthetic
RASKSVSTSGYSFMH

SEQ ID NO: 220 - protein, 7 aa, synthetic construct
  REGION 1..7: note = Synthetic
LASNLES

SEQ ID NO: 221 - protein, 9 aa, synthetic construct
  REGION 1..9: note = Synthetic
QHSREVPWT

SEQ ID NO: 222 - protein, 118 aa, synthetic construct; identical to SEQ ID NO: 12
  REGION 1..118: note = Synthetic

SEQ ID NO: 223 - protein, 111 aa, synthetic construct; identical to SEQ ID NO: 13
  REGION 1..111: note = Synthetic

SEQ ID NO: 224 - protein, 6 aa, synthetic construct
  REGION 1..6: note = Synthetic
SGYSWH

SEQ ID NO: 225 - protein, 16 aa, synthetic construct
  REGION 1..16: note = Synthetic
YIHSSGSTNYNPSLKS

SEQ ID NO: 226 - protein, 7 aa, synthetic construct
  REGION 1..7: note = Synthetic
YDDYFEY

SEQ ID NO: 227 - protein, 11 aa, synthetic construct
  REGION 1..11: note = Synthetic
KASQNVGFNVA

SEQ ID NO: 228 - protein, 7 aa, synthetic construct
  REGION 1..7: note = Synthetic
SASYRYS

SEQ ID NO: 229 - protein, 9 aa, synthetic construct
  REGION 1..9: note = Synthetic
QQYNWYPFT

SEQ ID NO: 230 - protein, 116 aa, synthetic construct
  REGION 1..116: note = Synthetic
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 231 - protein, 107 aa, synthetic construct
  REGION 1..107: note = Synthetic
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 232 - protein, 5 aa, synthetic construct
  REGION 1..5: note = Synthetic
NYWIE

SEQ ID NO: 233 - protein, 17 aa, synthetic construct
  REGION 1..17: note = Synthetic
EILPGTGRTIYNEKFKG

SEQ ID NO: 234 - protein, 13 aa, synthetic construct
  REGION 1..13: note = Synthetic
RDYYGNFYYAMDY

SEQ ID NO: 235 - protein, 11 aa, synthetic construct
  REGION 1..11: note = Synthetic
SASQGINNYLN

SEQ ID NO: 236 - protein, 7 aa, synthetic construct
  REGION 1..7: note = Synthetic
YTSTLQS

SEQ ID NO: 237 - protein, 9 aa, synthetic construct
  REGION 1..9: note = Synthetic
QQYSKLPRT

SEQ ID NO: 238 - protein, 122 aa, synthetic construct
  REGION 1..122: note = Synthetic
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 239 - protein, 107 aa, synthetic construct
  REGION 1..107: note = Synthetic
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 240 - protein, 7 aa, synthetic construct
  REGION 1..7: note = Synthetic
TYGMGVG

SEQ ID NO: 241 - protein, 16 aa, synthetic construct
  REGION 1..16: note = Synthetic
NIWWSEDKHYSPSLKS

SEQ ID NO: 242 - protein, 11 aa, synthetic construct
  REGION 1..11: note = Synthetic
IDYGNDYAFTY

SEQ ID NO: 243 - protein, 16 aa, synthetic construct
  REGION 1..16: note = Synthetic
RSSKSLLHSNGITYLY

SEQ ID NO: 244 - protein, 7 aa, synthetic construct
  REGION 1..7: note = Synthetic
QMSNLAS

SEQ ID NO: 245 - protein, 9 aa, synthetic construct
  REGION 1..9: note = Synthetic
AQNLELPYT

SEQ ID NO: 246 - protein, 121 aa, synthetic construct
  REGION 1..121: note = Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 247 - protein, 112 aa, synthetic construct
  REGION 1..112: note = Synthetic
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 248 - protein, 5 aa, synthetic construct
  REGION 1..5: note = Synthetic
RYTMH

SEQ ID NO: 249 - protein, 17 aa, synthetic construct
  REGION 1..17: note = Synthetic
VISFDGSNKYYVDSVKG

SEQ ID NO: 250 - protein, 10 aa, synthetic construct
  REGION 1..10: note = Synthetic
EARGSYAFDI

SEQ ID NO: 251 - protein, 11 aa, synthetic construct
  REGION 1..11: note = Synthetic
RASQSVSSYLA

SEQ ID NO: 252 - protein, 7 aa, synthetic construct
  REGION 1..7: note = Synthetic
DASNRAT

SEQ ID NO: 253 - protein, 10 aa, synthetic construct
  REGION 1..10: note = Synthetic
QQRSNWPPFT

SEQ ID NO: 254 - protein, 119 aa, synthetic construct; identical to SEQ ID NO: 78
  REGION 1..119: note = Synthetic

SEQ ID NO: 255 - protein, 108 aa, synthetic construct; identical to SEQ ID NO: 79
  REGION 1..108: note = Synthetic

SEQ ID NO: 256 - protein, 5 aa, synthetic construct
  REGION 1..5: note = Synthetic
SFGMH

SEQ ID NO: 257 - protein, 17 aa, synthetic construct
  REGION 1..17: note = Synthetic
YISSGSFTIYYADSVKG

SEQ ID NO: 258 - protein, 9 aa, synthetic construct
  REGION 1..9: note = Synthetic
MRKGYAMDY

SEQ ID NO: 259 - protein, 16 aa, synthetic construct
  REGION 1..16: note = Synthetic
RSSQIIIHSDGNTYLE

SEQ ID NO: 260 - protein, 7 aa, synthetic construct
  REGION 1..7: note = Synthetic
KVSNRFS

SEQ ID NO: 261 - protein, 9 aa, synthetic construct
  REGION 1..9: note = Synthetic
FQGSHVPHT

SEQ ID NO: 262 - protein, 118 aa, synthetic construct
  REGION 1..118: note = Synthetic
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 263 - protein, 112 aa, synthetic construct
  REGION 1..112: note = Synthetic
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 264 - protein, 5 aa, synthetic construct
  REGION 1..5: note = Synthetic
NYGVN

SEQ ID NO: 265 - protein, 17 aa, synthetic construct
  REGION 1..17: note = Synthetic
WINPNTGEPTFDDDFKG

SEQ ID NO: 266 - protein, 11 aa, synthetic construct
  REGION 1..11: note = Synthetic
SRGKNEAWFAY

SEQ ID NO: 267 - protein, 16 aa, synthetic construct
  REGION 1..16: note = Synthetic
RSSQSLVHRNGNTYLH

SEQ ID NO: 268 - protein, 7 aa, synthetic construct
  REGION 1..7: note = Synthetic
TVSNRFS

SEQ ID NO: 269 - protein, 9 aa, synthetic construct
  REGION 1..9: note = Synthetic
SQSSHVPPT

SEQ ID NO: 270 - protein, 120 aa, synthetic construct
  REGION 1..120: note = Synthetic
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 271 - protein, 112 aa, synthetic construct
  REGION 1..112: note = Synthetic
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 272 - protein, 5 aa, synthetic construct
  REGION 1..5: note = Synthetic
TYWMS

SEQ ID NO: 273 - protein, 17 aa, synthetic construct
  REGION 1..17: note = Synthetic
EIHPDSSTINYAPSLKD

SEQ ID NO: 274 - protein, 10 aa, synthetic construct
  REGION 1..10: note = Synthetic
LYFGFPWFAY

SEQ ID NO: 275 - protein, 11 aa, synthetic construct
  REGION 1..11: note = Synthetic
KASQDVGTSVA

SEQ ID NO: 276 - protein, 7 aa, synthetic construct
  REGION 1..7: note = Synthetic
WTSTRHT

SEQ ID NO: 277 - protein, 8 aa, synthetic construct
  REGION 1..8: note = Synthetic
QQYSLYRS

SEQ ID NO: 278 - protein, 119 aa, synthetic construct
  REGION 1..119: note = Synthetic
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 279 - protein, 106 aa, synthetic construct
  REGION 1..106: note = Synthetic
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 280 - protein, 5 aa, synthetic construct
  REGION 1..5: note = Synthetic
YYGMN

SEQ ID NO: 281 - protein, 17 aa, synthetic construct
  REGION 1..17: note = Synthetic
WIDTTTGEPTYAQKFQG

SEQ ID NO: 282 - protein, 10 aa, synthetic construct
  REGION 1..10: note = Synthetic
RGPYNWYFDV

SEQ ID NO: 283 - protein, 16 aa, synthetic construct
  REGION 1..16: note = Synthetic
RSSKSLLHSNGNTYLY

SEQ ID NO: 284 - protein, 7 aa, synthetic construct
  REGION 1..7: note = Synthetic
RMSNLVS

SEQ ID NO: 285 - protein, 9 aa, synthetic construct
  REGION 1..9: note = Synthetic
LQHLEYPFT

SEQ ID NO: 286 - protein, 119 aa, synthetic construct
  REGION 1..119: note = Synthetic
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSS

SEQ ID NO: 287 - protein, 112 aa, synthetic construct
  REGION 1..112: note = Synthetic
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELK

SEQ ID NO: 288 - protein, 5 aa, synthetic construct
  REGION 1..5: note = Synthetic
NYGMN

SEQ ID NO: 289 - protein, 17 aa, synthetic construct
  REGION 1..17: note = Synthetic
WINTYTGEPTYADDFKG

SEQ ID NO: 290 - protein, 9 aa, synthetic construct
  REGION 1..9: note = Synthetic
IGDSSPSDY

SEQ ID NO: 291 - protein, 11 aa, synthetic construct
  REGION 1..11: note = Synthetic
KASQSVSNDVV

SEQ ID NO: 292 - protein, 7 aa, synthetic construct
  REGION 1..7: note = Synthetic
YASNRYT

SEQ ID NO: 293 - protein, 9 aa, synthetic construct
  REGION 1..9: note = Synthetic
QQDYTSPWT

SEQ ID NO: 294 - protein, 118 aa, synthetic construct
  REGION 1..118: note = Synthetic
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 295 - protein, 107 aa, synthetic construct
  REGION 1..107: note = Synthetic
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 296 - protein, 5 aa, synthetic construct
  REGION 1..5: note = Synthetic
SYWIE

SEQ ID NO: 297 - protein, 17 aa, synthetic construct
  REGION 1..17: note = Synthetic
EILPGSGNTYYNERFKD

SEQ ID NO: 298 - protein, 14 aa, synthetic construct
  REGION 1..14: note = Synthetic
RAAAYYSNPEWFAY

SEQ ID NO: 299 - protein, 12 aa, synthetic construct
  REGION 1..12: note = Synthetic
TASSSVNSFYLH

SEQ ID NO: 300 - protein, 7 aa, synthetic construct
  REGION 1..7: note = Synthetic
STSNLAS

SEQ ID NO: 301 - protein, 9 aa, synthetic construct
  REGION 1..9: note = Synthetic
HQYHRSPYT

SEQ ID NO: 302 - protein, 123 aa, synthetic construct
  REGION 1..123: note = Synthetic
QVQLVQSGAEVKKPGSSVKVSCKASGGTFSSYWIEWVRQAPGQGLEWMGEILPGSGNTYYNERFKDRVTITADESTSTAYMELSSLRSEDTAVYYCARRAAAYYSNPEWFAYWGQGTLVTVSS

SEQ ID NO: 303 - protein, 108 aa, synthetic construct
  REGION 1..108: note = Synthetic
EIVLTQSPATLSLSPGERATLSCTASSSVNSFYLHWYQQKPGLAPRLLIYSTSNLASGIPDRFSGSGSGTDFTLTISRLEPEDFAVYYCHQYHRSPYTFGQGTKLEIK

SEQ ID NO: 304 - protein, 5 aa, synthetic construct
  REGION 1..5: note = Synthetic
SYWMQ

SEQ ID NO: 305 - protein, 17 aa, synthetic construct
  REGION 1..17: note = Synthetic
TIYPGDGDTTYTQKFQG

SEQ ID NO: 306 - protein, 10 aa, synthetic construct
  REGION 1..10: note = Synthetic
YDAPGYAMDY

SEQ ID NO: 307 - protein, 11 aa, synthetic construct
  REGION 1..11: note = Synthetic
RASQDINNYLA

SEQ ID NO: 308 - protein, 7 aa, synthetic construct
  REGION 1..7: note = Synthetic
YTSTLHP

SEQ ID NO: 309 - protein, 9 aa, synthetic construct
  REGION 1..9: note = Synthetic
LQYDNLLYT

SEQ ID NO: 310 - protein, 119 aa, synthetic construct
  REGION 1..119: note = Synthetic
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSS

SEQ ID NO: 311 - protein, 107 aa, synthetic construct
  REGION 1..107: note = Synthetic
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIK

SEQ ID NO: 312 - protein, 6 aa, synthetic construct
  REGION 1..6: note = Synthetic
RDFAWN

SEQ ID NO: 313 - protein, 16 aa, synthetic construct
  REGION 1..16: note = Synthetic
YISYNGNTRYQPSLKS

SEQ ID NO: 314 - protein, 7 aa, synthetic construct
  REGION 1..7: note = Synthetic
ASRGFPY

SEQ ID NO: 315 - protein, 11 aa, synthetic construct
  REGION 1..11: note = Synthetic
HSSQDINSNIG

SEQ ID NO: 316 - protein, 7 aa, synthetic construct
  REGION 1..7: note = Synthetic
HGTNLDD

SEQ ID NO: 317 - protein, 9 aa, synthetic construct
  REGION 1..9: note = Synthetic
VQYAQFPWT

SEQ ID NO: 318 - protein, 116 aa, synthetic construct
  REGION 1..116: note = Synthetic
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 319 - protein, 107 aa, synthetic construct
  REGION 1..107: note = Synthetic
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 320 - protein, 6 aa, synthetic construct
  REGION 1..6: note = Synthetic
RDFAWN

SEQ ID NO: 321 - protein, 16 aa, synthetic construct
  REGION 1..16: note = Synthetic
YISYNGNTRYQPSLKS

SEQ ID NO: 322 - protein, 7 aa, synthetic construct
  REGION 1..7: note = Synthetic
ASRGFPY

SEQ ID NO: 323 - protein, 11 aa, synthetic construct
  REGION 1..11: note = Synthetic
HSSQDINSNIG

SEQ ID NO: 324 - protein, 7 aa, synthetic construct
  REGION 1..7: note = Synthetic
HGTNLDD

SEQ ID NO: 325 - protein, 9 aa, synthetic construct
  REGION 1..9: note = Synthetic
VQYAQFPWT

SEQ ID NO: 326 - protein, 116 aa, synthetic construct; identical to SEQ ID NO: 318
  REGION 1..116: note = Synthetic

SEQ ID NO: 327 - protein, 107 aa, synthetic construct; identical to SEQ ID NO: 319
  REGION 1..107: note = Synthetic

SEQ ID NO: 328 - protein, 5 aa, synthetic construct
  REGION 1..5: note = Synthetic
NYGVH

SEQ ID NO: 329 - protein, 16 aa, synthetic construct
  REGION 1..16: note = Synthetic
VIWSGGNTDYNTPFTS

SEQ ID NO: 330 - protein, 11 aa, synthetic construct
  REGION 1..11: note = Synthetic
ALTYYDYEFAY

SEQ ID NO: 331 - protein, 11 aa, synthetic construct
  REGION 1..11: note = Synthetic
RASQSIGTNIH

SEQ ID NO: 332 - protein, 7 aa, synthetic construct
  REGION 1..7: note = Synthetic
YASESIS

SEQ ID NO: 333 - protein, 9 aa, synthetic construct
  REGION 1..9: note = Synthetic
QQNNNWPTT

SEQ ID NO: 334 - protein, 119 aa, synthetic construct
  REGION 1..119: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 335 - protein, 107 aa, synthetic construct
  REGION 1..107: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 336 - protein, 5 aa, synthetic construct
  REGION 1..5: note = Synthetic
GYFMN

SEQ ID NO: 337 - protein, 17 aa, synthetic construct
  REGION 1..17: note = Synthetic
RIHPYDGDTFYNQKFQG

SEQ ID NO: 338 - protein, 9 aa, synthetic construct
  REGION 1..9: note = Synthetic
YDGSRAMDY

SEQ ID NO: 339 - protein, 15 aa, synthetic construct
  REGION 1..15: note = Synthetic
KASQSVSFAGTSLMH

SEQ ID NO: 340 - protein, 7 aa, synthetic construct
  REGION 1..7: note = Synthetic
RASNLEA

SEQ ID NO: 341 - protein, 9 aa, synthetic construct
  REGION 1..9: note = Synthetic
QQSREYPYT

SEQ ID NO: 342 - protein, 118 aa, synthetic construct
  REGION 1..118: note = Synthetic
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 343 - protein, 111 aa, synthetic construct
  REGION 1..111: note = Synthetic
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 344 - protein, 5 aa, synthetic construct
  REGION 1..5: note = Synthetic
GYGLS

SEQ ID NO: 345 - protein, 17 aa, synthetic construct
  REGION 1..17: note = Synthetic
MISSGGSYTYYADSVKG

SEQ ID NO: 346 - protein, 10 aa, synthetic construct
  REGION 1..10: note = Synthetic
HGDDPAWFAY

SEQ ID NO: 347 - protein, 12 aa, synthetic construct
  REGION 1..12: note = Synthetic
SVSSSISSNNLH

SEQ ID NO: 348 - protein, 7 aa, synthetic construct
  REGION 1..7: note = Synthetic
GTSNLAS

SEQ ID NO: 349 - protein, 11 aa, synthetic construct
  REGION 1..11: note = Synthetic
QQWSSYPYMYT

SEQ ID NO: 350 - protein, 119 aa, synthetic construct
  REGION 1..119: note = Synthetic
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 351 - protein, 110 aa, synthetic construct
  REGION 1..110: note = Synthetic
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 352 - protein, 5 aa, synthetic construct
  REGION 1..5: note = Synthetic
NYWMN

SEQ ID NO: 353 - protein, 19 aa, synthetic construct
  REGION 1..19: note = Synthetic
EIRLKSNNYTTHYAESVKG

SEQ ID NO: 354 - protein, 6 aa, synthetic construct
  REGION 1..6: note = Synthetic
HYYFDY

SEQ ID NO: 355 - protein, 16 aa, synthetic construct
  REGION 1..16: note = Synthetic
RSSKSLLHSNGITYFF

SEQ ID NO: 356 - protein, 7 aa, synthetic construct
  REGION 1..7: note = Synthetic
QMSNLAS

SEQ ID NO: 357 - protein, 9 aa, synthetic construct
  REGION 1..9: note = Synthetic
AQNLELPPT

SEQ ID NO: 358 - protein, 117 aa, synthetic construct
  REGION 1..117: note = Synthetic
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 359 - protein, 112 aa, synthetic construct
  REGION 1..112: note = Synthetic
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 360 - protein, 5 aa, synthetic construct
  REGION 1..5: note = Synthetic
SYWIG

SEQ ID NO: 361 - protein, 17 aa, synthetic construct
  REGION 1..17: note = Synthetic
IIDPGDSRTRYSPSFQG

SEQ ID NO: 362 - protein, 11 aa, synthetic construct
  REGION 1..11: note = Synthetic
GQLYGGTYMDG

SEQ ID NO: 363 - protein, 14 aa, synthetic construct
  REGION 1..14: note = Synthetic
TGTSSDIGGYNSVS

SEQ ID NO: 364 - protein, 7 aa, synthetic construct
  REGION 1..7: note = Synthetic
GVNNRPS

SEQ ID NO: 365 - protein, 11 aa, synthetic construct
  REGION 1..11: note = Synthetic
SSYDIESATPV

SEQ ID NO: 366 - protein, 120 aa, synthetic construct
  REGION 1..120: note = Synthetic
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 367 - protein, 111 aa, synthetic construct
  REGION 1..111: note = Synthetic
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 368 - protein, 5 aa, synthetic construct
  REGION 1..5: note = Synthetic
AYNIH

SEQ ID NO: 369 - protein, 17 aa, synthetic construct
  REGION 1..17: note = Synthetic
SFDPYDGGSSYNQKFKD

SEQ ID NO: 370 - protein, 7 aa, synthetic construct
  REGION 1..7: note = Synthetic
GWYYFDY

SEQ ID NO: 371 - protein, 11 aa, synthetic construct
  REGION 1..11: note = Synthetic
RASKSISKYLA

SEQ ID NO: 372 - protein, 7 aa, synthetic construct
  REGION 1..7: note = Synthetic
SGSTLQS

SEQ ID NO: 373 - protein, 9 aa, synthetic construct
  REGION 1..9: note = Synthetic
QQHDESPYT

SEQ ID NO: 374 - protein, 116 aa, synthetic construct
  REGION 1..116: note = Synthetic
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 375 - protein, 107 aa, synthetic construct
  REGION 1..107: note = Synthetic
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 376 - protein, 11 aa, synthetic construct
  REGION 1..11: note = Synthetic
GSIKSGSYYWG

SEQ ID NO: 377 - protein, 16 aa, synthetic construct
  REGION 1..16: note = Synthetic
NIYYSGSTYYNPSLRS

SEQ ID NO: 378 - protein, 12 aa, synthetic construct
  REGION 1..12: note = Synthetic
AREGSYPNQFDP

SEQ ID NO: 379 - protein, 11 aa, synthetic construct
  REGION 1..11: note = Synthetic
RASQSVSSNLA

SEQ ID NO: 380 - protein, 7 aa, synthetic construct
  REGION 1..7: note = Synthetic
GASTRAT

SEQ ID NO: 381 - protein, 9 aa, synthetic construct
  REGION 1..9: note = Synthetic
QQYHSFPFT

SEQ ID NO: 382 - protein, 120 aa, synthetic construct
  REGION 1..120: note = Synthetic
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 383 - protein, 107 aa, synthetic construct
  REGION 1..107: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 384 - protein, 5 aa, synthetic construct
  REGION 1..5: note = Synthetic
SFGMH

SEQ ID NO: 385 - protein, 11 aa, synthetic construct
  REGION 1..11: note = Synthetic
YISSDSSAIYY

SEQ ID NO: 386 - protein, 12 aa, synthetic construct
  REGION 1..12: note = Synthetic
GRENIYYGSRLD

SEQ ID NO: 387 - protein, 7 aa, synthetic construct
  REGION 1..7: note = Synthetic
KASQNVD

SEQ ID NO: 388 - protein, 11 aa, synthetic construct
  REGION 1..11: note = Synthetic
SASYRYSGVPD

SEQ ID NO: 389 - protein, 12 aa, synthetic construct
  REGION 1..12: note = Synthetic
QQYNNYPFTFGS

SEQ ID NO: 390 - protein, 122 aa, synthetic construct
  REGION 1..122: note = Synthetic
DVQLVESGGGLVQPGGSRKLSCAASGFTFSSFGMHWVRQAPEKGLEWVAYISSDSSAIYYADTVKGRFTISRDNPKNTLFLQMTSLRSEDTAMYYCGRGRENIYYGSRLDYWGQGTTLTVSS

SEQ ID NO: 391 - protein, 107 aa, synthetic construct
  REGION 1..107: note = Synthetic
DIAMTQSQKFMSTSVGDRVSVTCKASQNVDTNVAWYQQKPGQSPKALIYSASYRYSGVPDRFTGSGSGTDFTLTINNVQSEDLAEYFCQQYNNYPFTFGSGTKLEIK

SEQ ID NO: 392 - protein, 10 aa, synthetic construct
  REGION 1..10: note = Synthetic
SYWMQWVRQA

SEQ ID NO: 393 - protein, 11 aa, synthetic construct
  REGION 1..11: note = Synthetic
TIYPGDGDTRY

SEQ ID NO: 394 - protein, 12 aa, synthetic construct
  REGION 1..12: note = Synthetic
RGIPRLWYFDVM

SEQ ID NO: 395 - protein, 10 aa, synthetic construct
  REGION 1..10: note = Synthetic
ITCRASQDIS

SEQ ID NO: 396 - protein, 11 aa, synthetic construct
  REGION 1..11: note = Synthetic
YTSRLHSGVPS

SEQ ID NO: 397 - protein, 12 aa, synthetic construct
  REGION 1..12: note = Synthetic
QQGNTLPPFTGG

SEQ ID NO: 398 - protein, 122 aa, synthetic construct; identical to SEQ ID NO: 390
  REGION 1..122: note = Synthetic

SEQ ID NO: 399 - protein, 107 aa, synthetic construct; identical to SEQ ID NO: 391
  REGION 1..107: note = Synthetic

SEQ ID NO: 400 - protein, 10 aa, synthetic construct
  REGION 1..10: note = Synthetic
SYGMSWVRQA

SEQ ID NO: 401 - protein, 10 aa, synthetic construct
  REGION 1..10: note = Synthetic
INSGGSNTYY

SEQ ID NO: 402 - protein, 9 aa, synthetic construct
  REGION 1..9: note = Synthetic
HDGGAMDYW

SEQ ID NO: 403 - protein, 14 aa, synthetic construct
  REGION 1..14: note = Synthetic
ITCRASESIYSYLA

SEQ ID NO: 404 - protein, 7 aa, synthetic construct
  REGION 1..7: note = Synthetic
NTKTLPE

SEQ ID NO: 405 - protein, 11 aa, synthetic construct
  REGION 1..11: note = Synthetic
HHYGTPPWTFG

SEQ ID NO: 406 - protein, 117 aa, synthetic construct
  REGION 1..117: note = Synthetic
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 407 - protein, 108 aa, synthetic construct
  REGION 1..108: note = Synthetic
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 408 - protein, 10 aa, synthetic construct
  REGION 1..10: note = Synthetic
SFGMHWVRQA

SEQ ID NO: 409 - protein, 20 aa, synthetic construct
  REGION 1..20: note = Synthetic
ISSGSGTIYYADTVKGRFTI

SEQ ID NO: 410 - protein, 12 aa, synthetic construct
  REGION 1..12: note = Synthetic
HGYRYEGFDYWG

SEQ ID NO: 411 - protein, 14 aa, synthetic construct
  REGION 1..14: note = Synthetic
ITCKASQNVDTNVA

SEQ ID NO: 412 - protein, 11 aa, synthetic construct
  REGION 1..11: note = Synthetic
SASYRYSGVPS

SEQ ID NO: 413 - protein, 12 aa, synthetic construct
  REGION 1..12: note = Synthetic
QQYNNYPFTFGQ

SEQ ID NO: 414 - protein, 119 aa, synthetic construct
  REGION 1..119: note = Synthetic
EVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 415 - protein, 107 aa, synthetic construct
  REGION 1..107: note = Synthetic
DIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIK

SEQ ID NO: 416 - protein, 5 aa, synthetic construct
  REGION 1..5: note = Synthetic
NYVMH

SEQ ID NO: 417 - protein, 17 aa, synthetic construct
  REGION 1..17: note = Synthetic
YINPYNDDVKYNEKFKG

SEQ ID NO: 418 - protein, 13 aa, synthetic construct
  REGION 1..13: note = Synthetic
WGYYGSPLYYFDY

SEQ ID NO: 419 - protein, 10 aa, synthetic construct
  REGION 1..10: note = Synthetic
RASSRLIYMH

SEQ ID NO: 420 - protein, 7 aa, synthetic construct
  REGION 1..7: note = Synthetic
ATSNLAS

SEQ ID NO: 421 - protein, 9 aa, synthetic construct
  REGION 1..9: note = Synthetic
QQWNSNPPT

SEQ ID NO: 422 - protein, 122 aa, synthetic construct
  REGION 1..122: note = Synthetic
EVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 423 - protein, 106 aa, synthetic construct
  REGION 1..106: note = Synthetic
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELK

SEQ ID NO: 424 - protein, 5 aa, synthetic construct
  REGION 1..5: note = Synthetic
NYVMH

SEQ ID NO: 425 - protein, 17 aa, synthetic construct
  REGION 1..17: note = Synthetic
YINPYNDDVKYNEKFKG

SEQ ID NO: 426 - protein, 13 aa, synthetic construct
  REGION 1..13: note = Synthetic
WGYYGSPLYYFDY

SEQ ID NO: 427 - protein, 10 aa, synthetic construct
  REGION 1..10: note = Synthetic
RASSRLIYMH

SEQ ID NO: 428 - protein, 7 aa, synthetic construct
  REGION 1..7: note = Synthetic
ATSNLAS

SEQ ID NO: 429 - protein, 9 aa, synthetic construct
  REGION 1..9: note = Synthetic
QQWNSNPPT

SEQ ID NO: 430 - protein, 122 aa, synthetic construct
  REGION 1..122: note = Synthetic
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 431 - protein, 106 aa, synthetic construct
  REGION 1..106: note = Synthetic
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 432 - protein, 10 aa, synthetic construct
  REGION 1..10: note = Synthetic
GYSFTSYTIH

SEQ ID NO: 433 - protein, 17 aa, synthetic construct
  REGION 1..17: note = Synthetic
YINPNSRNTDYAQKFQG

SEQ ID NO: 434 - protein, 12 aa, synthetic construct
  REGION 1..12: note = Synthetic
YSGSTPYWYFDV

SEQ ID NO: 435 - protein, 10 aa, synthetic construct
  REGION 1..10: note = Synthetic
RASSSVSYMN

SEQ ID NO: 436 - protein, 7 aa, synthetic construct
  REGION 1..7: note = Synthetic
ATSNLAS

SEQ ID NO: 437 - protein, 9 aa, synthetic construct
  REGION 1..9: note = Synthetic
QQWSSNPLT

SEQ ID NO: 438 - protein, 121 aa, synthetic construct
  REGION 1..121: note = Synthetic
EVQLVQSGAEVKKPGSSVKVSCKASGYSFTSYTIHWVRQAPGQGLEWMGYINPNSRNTDYAQKFQGRVTLTADKSTSTAYMELSSLRSEDTAVYYCARYSGSTPYWYFDVWGQGTTVTVSS

SEQ ID NO: 439 - protein, 107 aa, synthetic construct; identical to SEQ ID NO: 231
  REGION 1..107: note = Synthetic

SEQ ID NO: 440 - protein, 10 aa, synthetic construct
  REGION 1..10: note = Synthetic
GYTFSSYWMH

SEQ ID NO: 441 - protein, 17 aa, synthetic construct
  REGION 1..17: note = Synthetic
LIHPDSGSTNYNEMFKN

SEQ ID NO: 442 - protein, 7 aa, synthetic construct
  REGION 1..7: note = Synthetic
GGRLYFD

SEQ ID NO: 443 - protein, 16 aa, synthetic construct
  REGION 1..16: note = Synthetic
RSSQSLVHSNGDTYLR

SEQ ID NO: 444 - protein, 7 aa, synthetic construct
  REGION 1..7: note = Synthetic
KVSNRFS

SEQ ID NO: 445 - protein, 9 aa, synthetic construct
  REGION 1..9: note = Synthetic
SQSTHVPYT

SEQ ID NO: 446 - protein, 117 aa, synthetic construct
  REGION 1..117: note = Synthetic
EVQLVQSGAEVKKPGSSVKVSCKASGYTFSSYWMHWVRQAPGQGLEWIGLIHPDSGSTNYNEMFKNRATLTVDRSTSTAYVELSSLRSEDTAVYFCAGGGRLYFDYWGQGTTVTVSS

SEQ ID NO: 447 - protein, 112 aa, synthetic construct
  REGION 1..112: note = Synthetic
DVVMTQSPLSLPVTPGEPASISCRSSQSLVHSNGDTYLRWYLQKPGQSPQLLIYKVSNRFSGVPDRFSGSGSGTDFTLKISRVEAEDVGVYYCSQSTHVPYTFGGGTKVEIK

SEQ ID NO: 448 - protein, 10 aa, synthetic construct
  REGION 1..10: note = Synthetic
GYTFSSYWMH

SEQ ID NO: 449 - protein, 17 aa, synthetic construct
  REGION 1..17: note = Synthetic
LIHPESGSTNYNEMFKN

SEQ ID NO: 450 - protein, 8 aa, synthetic construct
  REGION 1..8: note = Synthetic
GGRLYFDY

SEQ ID NO: 451 - protein, 16 aa, synthetic construct
  REGION 1..16: note = Synthetic
RSSQSLVHSNQDTYLR

SEQ ID NO: 452 - protein, 7 aa, synthetic construct
  REGION 1..7: note = Synthetic
KVSNRFS

SEQ ID NO: 453 - protein, 9 aa, synthetic construct
  REGION 1..9: note = Synthetic
SQSTHVPYT

SEQ ID NO: 454 - protein, 117 aa, synthetic construct
  REGION 1..117: note = Synthetic
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 455 - protein, 113 aa, synthetic construct
  REGION 1..113: note = Synthetic
DIVMTQSPLSLPVTPGEPASISCRSSQSLVHSNQDTYLRWYLQKPGQSPQLLIYKVSNRFSGVPDRFSGSGSGTDFTLKKISRVEAEDVGVYYCSQSTHVPYTFGGGTKVEIK

SEQ ID NO: 456 - protein, 12 aa, synthetic construct
  REGION 1..12: note = Synthetic
TGYSITSGYSWH

SEQ ID NO: 457 - protein, 16 aa, synthetic construct
  REGION 1..16: note = Synthetic
YIHSSGSTNYNPSLKS

SEQ ID NO: 458 - protein, 7 aa, synthetic construct
  REGION 1..7: note = Synthetic
YDDYFEY

SEQ ID NO: 459 - protein, 12 aa, synthetic construct
  REGION 1..12: note = Synthetic
KASQNVGFNVAW

SEQ ID NO: 460 - protein, 7 aa, synthetic construct
  REGION 1..7: note = Synthetic
SASYRYS

SEQ ID NO: 461 - protein, 9 aa, synthetic construct
  REGION 1..9: note = Synthetic
QQYNWYPFT

SEQ ID NO: 462 - protein, 116 aa, synthetic construct; identical to SEQ ID NO: 230
  REGION 1..116: note = Synthetic

SEQ ID NO: 463 - protein, 108 aa, synthetic construct
  REGION 1..108: note = Synthetic
DIQMTQSPSSLSASVGDRVTITCKASQNVGGFNVAWYQQKPGKSPKALIYSASYRYSGVPSRFSGSGSGTDFTLTISSLQPEDFAEYFCQQYNWYPFTFGQGTKLEIK

SEQ ID NO: 464 - protein, 5 aa, synthetic construct
  REGION 1..5: note = Synthetic
NYDIN

SEQ ID NO: 465 - protein, 20 aa, synthetic construct
  REGION 1..20: note = Synthetic
WIGWIFPGDDSTQYNEKFKG

SEQ ID NO: 466 - protein, 9 aa, synthetic construct
  REGION 1..9: note = Synthetic
QTTGTWFAY

SEQ ID NO: 467 - protein, 11 aa, synthetic construct
  REGION 1..11: note = Synthetic
RASQSISDYLY

SEQ ID NO: 468 - protein, 7 aa, synthetic construct
  REGION 1..7: note = Synthetic
YASQSIS

SEQ ID NO: 469 - protein, 9 aa, synthetic construct
  REGION 1..9: note = Synthetic
CQNGHSFPL

SEQ ID NO: 470 - protein, 118 aa, synthetic construct
  REGION 1..118: note = Synthetic
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSS

SEQ ID NO: 471 - protein, 107 aa, synthetic construct
  REGION 1..107: note = Synthetic
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELK

SEQ ID NO: 472 - protein, 119 aa, synthetic construct
  REGION 1..119: note = Synthetic
QVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSS

SEQ ID NO: 473 - protein, 109 aa, synthetic construct
  REGION 1..109: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRITFGQGTRLEIK

SEQ ID NO: 474 - protein, 5 aa, synthetic construct
  REGION 1..5: note = Synthetic
IYNVH

SEQ ID NO: 475 - protein, 17 aa, synthetic construct
  REGION 1..17: note = Synthetic
TIFPGNGDTSYNQKFKD

SEQ ID NO: 476 - protein, 10 aa, synthetic construct
  REGION 1..10: note = Synthetic
WDDGNVGFAH

SEQ ID NO: 477 - protein, 11 aa, synthetic construct
  REGION 1..11: note = Synthetic
RASENINNYLT

SEQ ID NO: 478 - protein, 7 aa, synthetic construct
  REGION 1..7: note = Synthetic
HAKTLAE

SEQ ID NO: 479 - protein, 9 aa, synthetic construct
  REGION 1..9: note = Synthetic
QHHYGTPPT

SEQ ID NO: 480 - protein, 119 aa, synthetic construct
  REGION 1..119: note = Synthetic
QVQLQQPGAELVKPGASVKMSCKASGYTFTIYNVHWIKQTPGQGLEWMGTIFPGNGDTSYNQKFKDKATLTTDKSSKTAYMQLNSLTSEDSAVYYCARWDDGNVGFAHWGQGTLVTVSA

SEQ ID NO: 481 - protein, 107 aa, synthetic construct
  REGION 1..107: note = Synthetic
DIQMTQSPASLSASVGETVTITCRASENINNYLTWFQQKQGKSPQLLVYHAKTLAEGVPSRFSGSGSGTQFSLKINSLQPEDFGSYYCQHHYGTPPTFGGGTKLEIK

SEQ ID NO: 482 - protein, 119 aa, synthetic construct
  REGION 1..119: note = Synthetic
EVQLVQSGAEVKKPGASVKVSCKASGYTFTIYNVHWVRQAPGQGLEWMGTIFPGNGDTSYNQKFKDKVTMTTDTSTSTAYMELSSLRSEDTAVYYCARWDDGNVGFAHWGQGTLVTVSS

SEQ ID NO: 483 - protein, 107 aa, synthetic construct
  REGION 1..107: note = Synthetic
DIQMTQSPSSLSASVGDRVTITCRASENINNYLTWFQQKQGKSPQLLIYHAKTLAEGVPSRFSGSGSGTDFTLTISSLQPEDFATYYCQHHYGTPPTFGGGTKVEIK

SEQ ID NO: 484 - protein, 119 aa, synthetic construct
  REGION 1..119: note = Synthetic
EVQLVQSGAEVKKPGASVKVSCKASGYTFTIYNVHWIRQAPGQGLEWMGTIFPGNGDTSYNQKFKDRATLTTDKSTKTAYMELRSLRSDDTAVYYCARWDDGNVGFAHWGQGTLVTVSS

SEQ ID NO: 485 - protein, 107 aa, synthetic construct
  REGION 1..107: note = Synthetic
DIQMTQSPSSLSASVGDRVTITCRASENINNYLTWFQQKPGKAPKLLVYHAKTLAEGVPSRFSGSGSGTQFTLTISSLQPEDFATYYCQHHYGTPPTFGQGTKLEIK

SEQ ID NO: 486 - protein, 447 aa, synthetic construct
  REGION 1..447: note = Synthetic
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 220 aa, synthetic construct
  REGION 1..220: note = Synthetic
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 488 - protein, 445 aa, synthetic construct
  REGION 1..445: note = Synthetic
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 489 - protein, 217 aa, synthetic construct
  REGION 1..217: note = Synthetic
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 490 - protein, 447 aa, synthetic construct
  REGION 1..447: note = Synthetic
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 491 - protein, 214 aa, synthetic construct
  REGION 1..214: note = Synthetic
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 449 aa, synthetic construct
  REGION 1..449: note = Synthetic
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 493 - protein, 220 aa, synthetic construct
  REGION 1..220: note = Synthetic
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 7 aa, synthetic construct
  REGION 1..7: note = Synthetic
TSNMGVG

SEQ ID NO: 495 - protein, 16 aa, synthetic construct
  REGION 1..16: note = Synthetic
HIWWDDDKYYSPSLKS

SEQ ID NO: 496 - protein, 10 aa, synthetic construct
  REGION 1..10: note = Synthetic
SNYGYAWFAY

SEQ ID NO: 497 - protein, 11 aa, synthetic construct
  REGION 1..11: note = Synthetic
KASQDIYPYLN

SEQ ID NO: 498 - protein, 7 aa, synthetic construct
  REGION 1..7: note = Synthetic
RTNRLLD

SEQ ID NO: 499 - protein, 9 aa, synthetic construct
  REGION 1..9: note = Synthetic
LQYDEFPLT

SEQ ID NO: 500 - protein, 120 aa, synthetic construct
  REGION 1..120: note = Synthetic
QITLKESGPTLVKPTQTLTLTCTFSGFSLSTSNMGVGWIRQPPGKALEWLAHIWWDDDKYYSPSLKSRLTITKDTSKNQVVLTMTNMDPVDTATYYCVRSNYGYAWFAYWGQGTLVTVSS

SEQ ID NO: 501 - protein, 107 aa, synthetic construct
  REGION 1..107: note = Synthetic
DIQMTQSPSSLSASVGDRVTITCKASQDIYPYLNWFQQKPGKAPKTLIYRTNRLLDGVPSRFSGSGSGTDFTFTISSLQPEDIATYYCLQYDEFPLTFGAGTKLEIK

SEQ ID NO: 502 - protein, 5 aa, synthetic construct
  REGION 1..5: note = Synthetic
DYAVH

SEQ ID NO: 503 - protein, 17 aa, synthetic construct
  REGION 1..17: note = Synthetic
VISTYNDYTYNNQDFKG

SEQ ID NO: 504 - protein, 10 aa, synthetic construct
  REGION 1..10: note = Synthetic
GNSYFYALDY

SEQ ID NO: 505 - protein, 15 aa, synthetic construct
  REGION 1..15: note = Synthetic
RASESVDSYGKSFMH

SEQ ID NO: 506 - protein, 7 aa, synthetic construct
  REGION 1..7: note = Synthetic
RASNLES

SEQ ID NO: 507 - protein, 9 aa, synthetic construct
  REGION 1..9: note = Synthetic
QQSNEDPWT

SEQ ID NO: 508 - protein, 119 aa, synthetic construct
  REGION 1..119: note = Synthetic
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 509 - protein, 111 aa, synthetic construct
  REGION 1..111: note = Synthetic
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 510 - protein, 5 aa, synthetic construct
  REGION 1..5: note = Synthetic
RYWMS

SEQ ID NO: 511 - protein, 17 aa, synthetic construct
  REGION 1..17: note = Synthetic
DLNPDSSAINYVDSVKG

SEQ ID NO: 512 - protein, 11 aa, synthetic construct
  REGION 1..11: note = Synthetic
ITTLVPYTMDF

SEQ ID NO: 513 - protein, 11 aa, synthetic construct
  REGION 1..11: note = Synthetic
ITNTDIDDDMN

SEQ ID NO: 514 - protein, 7 aa, synthetic construct
  REGION 1..7: note = Synthetic
EGNGLRP

SEQ ID NO: 515 - protein, 9 aa, synthetic construct
  REGION 1..9: note = Synthetic
LQSDNLPLT

SEQ ID NO: 516 - protein, 120 aa, synthetic construct
  REGION 1..120: note = Synthetic
EVQLVESGGGLVQPGGSLRLSCAASGFDFSRYWMSWVRQAPGKGLEWIGDLNPDSSAINYVDSVKGRFTISRDNAKNSLYLQMNSLRAEDTAVYYCTLITTLVPYTMDFWGQGTSVTVSS

SEQ ID NO: 517 - protein, 107 aa, synthetic construct
  REGION 1..107: note = Synthetic
ETTLTQSPAFMSATPGDKVNISCITNTDIDDDMNWYQQKPGEAAILLISEGNGLRPGIPPRFSGSGYGTDFTLTINNIESEDAAYYFCLQSDNLPLTFGSGTKLEIK

SEQ ID NO: 518 - protein, 5 aa, synthetic construct
  REGION 1..5: note = Synthetic
DYYMH

SEQ ID NO: 519 - protein, 17 aa, synthetic construct
  REGION 1..17: note = Synthetic
WIDPENGDTEYGPKFQG

SEQ ID NO: 520 - protein, 11 aa, synthetic construct
  REGION 1..11: note = Synthetic
HNAHYGTWFAY

SEQ ID NO: 521 - protein, 16 aa, synthetic construct
  REGION 1..16: note = Synthetic
RSSQSLLHSSGNTYLE

SEQ ID NO: 522 - protein, 7 aa, synthetic construct
  REGION 1..7: note = Synthetic
KISTRFS

SEQ ID NO: 523 - protein, 9 aa, synthetic construct
  REGION 1..9: note = Synthetic
FQGSHVPYT

SEQ ID NO: 524 - protein, 120 aa, synthetic construct
  REGION 1..120: note = Synthetic
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 525 - protein, 112 aa, synthetic construct
  REGION 1..112: note = Synthetic
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 526 - protein, 5 aa, synthetic construct
  REGION 1..5: note = Synthetic
DYNVN

SEQ ID NO: 527 - protein, 17 aa, synthetic construct
  REGION 1..17: note = Synthetic
VINPKYGTTRYNQKFKG

SEQ ID NO: 528 - protein, 7 aa, synthetic construct
  REGION 1..7: note = Synthetic
GLNAWDY

SEQ ID NO: 529 - protein, 11 aa, synthetic construct
  REGION 1..11: note = Synthetic
GASENIYGALN

SEQ ID NO: 530 - protein, 7 aa, synthetic construct
  REGION 1..7: note = Synthetic
GATNLED

SEQ ID NO: 531 - protein, 9 aa, synthetic construct
  REGION 1..9: note = Synthetic
QNVLTTPYT

SEQ ID NO: 532 - protein, 116 aa, synthetic construct
  REGION 1..116: note = Synthetic
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 533 - protein, 107 aa, synthetic construct
  REGION 1..107: note = Synthetic
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 534 - protein, 5 aa, synthetic construct
  REGION 1..5: note = Synthetic
GYFMN

SEQ ID NO: 535 - protein, 17 aa, synthetic construct
  REGION 1..17: note = Synthetic
LINPYNGDSFYNQKFKG

SEQ ID NO: 536 - protein, 8 aa, synthetic construct
  REGION 1..8: note = Synthetic
GLRRDFDY

SEQ ID NO: 537 - protein, 16 aa, synthetic construct
  REGION 1..16: note = Synthetic
KSSQSLLDSDGKTYLN

SEQ ID NO: 538 - protein, 7 aa, synthetic construct
  REGION 1..7: note = Synthetic
LVSELDS

SEQ ID NO: 539 - protein, 9 aa, synthetic construct
  REGION 1..9: note = Synthetic
WQGTHFPRT

SEQ ID NO: 540 - protein, 117 aa, synthetic construct
  REGION 1..117: note = Synthetic
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 541 - protein, 112 aa, synthetic construct
  REGION 1..112: note = Synthetic
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 542 - protein, 5 aa, synthetic construct
  REGION 1..5: note = Synthetic
DFGMN

SEQ ID NO: 543 - protein, 17 aa, synthetic construct
  REGION 1..17: note = Synthetic
WINTFTGEPSYGNVFKG

SEQ ID NO: 544 - protein, 10 aa, synthetic construct
  REGION 1..10: note = Synthetic
RHGNGNVFDS

SEQ ID NO: 545 - protein, 11 aa, synthetic construct
  REGION 1..11: note = Synthetic
RASQSIGSNIH

SEQ ID NO: 546 - protein, 7 aa, synthetic construct
  REGION 1..7: note = Synthetic
YTSESIS

SEQ ID NO: 547 - protein, 9 aa, synthetic construct
  REGION 1..9: note = Synthetic
QQSNSWPLT

SEQ ID NO: 548 - protein, 119 aa, synthetic construct
  REGION 1..119: note = Synthetic
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSS

SEQ ID NO: 549 - protein, 108 aa, synthetic construct
  REGION 1..108: note = Synthetic
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIKR

SEQ ID NO: 550 - protein, 5 aa, synthetic construct
  REGION 1..5: note = Synthetic
TAAIS

SEQ ID NO: 551 - protein, 17 aa, synthetic construct
  REGION 1..17: note = Synthetic
GIIPIFGKAHYAQKFQG

SEQ ID NO: 552 - protein, 14 aa, synthetic construct
  REGION 1..14: note = Synthetic
KFHFVSGSPFGMDV

SEQ ID NO: 553 - protein, 11 aa, synthetic construct
  REGION 1..11: note = Synthetic
RASQSVSSYLA

SEQ ID NO: 554 - protein, 7 aa, synthetic construct
  REGION 1..7: note = Synthetic
DASNRAT

SEQ ID NO: 555 - protein, 8 aa, synthetic construct
  REGION 1..8: note = Synthetic
QQRSNWPT

SEQ ID NO: 556 - protein, 123 aa, synthetic construct
  REGION 1..123: note = Synthetic
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 557 - protein, 106 aa, synthetic construct
  REGION 1..106: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 558 - protein, 5 aa, synthetic construct
  REGION 1..5: note = Synthetic
SYAIS

SEQ ID NO: 559 - protein, 17 aa, synthetic construct
  REGION 1..17: note = Synthetic
GIIPIFGTANYAQKFQG

SEQ ID NO: 560 - protein, 21 aa, synthetic construct
  REGION 1..21: note = Synthetic
APLRFLEWSTQDHYYYYYMDV

SEQ ID NO: 561 - protein, 11 aa, synthetic construct
  REGION 1..11: note = Synthetic
QGDSLRSYYAT

SEQ ID NO: 562 - protein, 7 aa, synthetic construct
  REGION 1..7: note = Synthetic
GENKRPS

SEQ ID NO: 563 - protein, 11 aa, synthetic construct
  REGION 1..11: note = Synthetic
KSRDGSGQHLV

SEQ ID NO: 564 - protein, 130 aa, synthetic construct
  REGION 1..130: note = Synthetic
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 565 - protein, 108 aa, synthetic construct
  REGION 1..108: note = Synthetic
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVL

SEQ ID NO: 566 - protein, 5 aa, synthetic construct
  REGION 1..5: note = Synthetic
SYWIN

SEQ ID NO: 567 - protein, 17 aa, synthetic construct
  REGION 1..17: note = Synthetic
NIYPSDSYTNYNQKFKD

SEQ ID NO: 568 - protein, 9 aa, synthetic construct
  REGION 1..9: note = Synthetic
SWRGNSFDY

SEQ ID NO: 569 - protein, 17 aa, synthetic construct
  REGION 1..17: note = Synthetic
KSSQSLLNSGNQKNYLT

SEQ ID NO: 570 - protein, 7 aa, synthetic construct
  REGION 1..7: note = Synthetic
WASTRES

SEQ ID NO: 571 - protein, 9 aa, synthetic construct
  REGION 1..9: note = Synthetic
QNDYSYPFT

SEQ ID NO: 572 - protein, 118 aa, synthetic construct
  REGION 1..118: note = Synthetic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 573 - protein, 113 aa, synthetic construct
  REGION 1..113: note = Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 574 - protein, 5 aa, synthetic construct
  REGION 1..5: note = Synthetic
NYGMN

SEQ ID NO: 575 - protein, 17 aa, synthetic construct
  REGION 1..17: note = Synthetic
WINTNTGEPTYAEEFKG

SEQ ID NO: 576 - protein, 9 aa, synthetic construct
  REGION 1..9: note = Synthetic
LGFGNAMDY

SEQ ID NO: 577 - protein, 17 aa, synthetic construct
  REGION 1..17: note = Synthetic
KSSQSLLNSGNQKNYLT

SEQ ID NO: 578 - protein, 7 aa, synthetic construct
  REGION 1..7: note = Synthetic
WASTRES

SEQ ID NO: 579 - protein, 9 aa, synthetic construct
  REGION 1..9: note = Synthetic
QNDYSYPLT

SEQ ID NO: 580 - protein, 118 aa, synthetic construct
  REGION 1..118: note = Synthetic
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 581 - protein, 113 aa, synthetic construct
  REGION 1..113: note = Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 582 - protein, 5 aa, synthetic construct
  REGION 1..5: note = Synthetic
SYNMN

SEQ ID NO: 583 - protein, 17 aa, synthetic construct
  REGION 1..17: note = Synthetic
YISSSSSTIYYADSVKG

SEQ ID NO: 584 - protein, 8 aa, synthetic construct
  REGION 1..8: note = Synthetic
AYYYGMDV

SEQ ID NO: 585 - protein, 11 aa, synthetic construct
  REGION 1..11: note = Synthetic
RASQGISGWLA

SEQ ID NO: 586 - protein, 7 aa, synthetic construct
  REGION 1..7: note = Synthetic
AASTLQS

SEQ ID NO: 587 - protein, 9 aa, synthetic construct
  REGION 1..9: note = Synthetic
QQANSFPPT

SEQ ID NO: 588 - protein, 117 aa, synthetic construct
  REGION 1..117: note = Synthetic
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 589 - protein, 107 aa, synthetic construct
  REGION 1..107: note = Synthetic
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 590 - protein, 5 aa, synthetic construct
  REGION 1..5: note = Synthetic
SYGMH

SEQ ID NO: 591 - protein, 17 aa, synthetic construct
  REGION 1..17: note = Synthetic
VIWYDGSNQYYADSVKG

SEQ ID NO: 592 - protein, 11 aa, synthetic construct
  REGION 1..11: note = Synthetic
GLTSGRYGMDV

SEQ ID NO: 593 - protein, 16 aa, synthetic construct
  REGION 1..16: note = Synthetic
RSSQSLLLSHGFNYLD

SEQ ID NO: 594 - protein, 7 aa, synthetic construct
  REGION 1..7: note = Synthetic
LGSSRAS

SEQ ID NO: 595 - protein, 9 aa, synthetic construct
  REGION 1..9: note = Synthetic
MQPLQIPWT

SEQ ID NO: 596 - protein, 120 aa, synthetic construct
  REGION 1..120: note = Synthetic
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDVWGQGTTVTVSS

SEQ ID NO: 597 - protein, 112 aa, synthetic construct
  REGION 1..112: note = Synthetic
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIK

SEQ ID NO: 598 - protein, 5 aa, synthetic construct
  REGION 1..5: note = Synthetic
SGYWN

SEQ ID NO: 599 - protein, 16 aa, synthetic construct
  REGION 1..16: note = Synthetic
YISDSGITYYNPSLKS

SEQ ID NO: 600 - protein, 11 aa, synthetic construct
  REGION 1..11: note = Synthetic
RTLATYYAMDY

SEQ ID NO: 601 - protein, 16 aa, synthetic construct
  REGION 1..16: note = Synthetic
RASQSLVHSDGNTYLH

SEQ ID NO: 602 - protein, 7 aa, synthetic construct
  REGION 1..7: note = Synthetic
RVSNRFS

SEQ ID NO: 603 - protein, 9 aa, synthetic construct
  REGION 1..9: note = Synthetic
SQSTHVPPT

SEQ ID NO: 604 - protein, 119 aa, synthetic construct
  REGION 1..119: note = Synthetic
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSS

SEQ ID NO: 605 - protein, 113 aa, synthetic construct
  REGION 1..113: note = Synthetic
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKR

SEQ ID NO: 606 - protein, 5 aa, synthetic construct
  REGION 1..5: note = Synthetic
NYAMS

SEQ ID NO: 607 - protein, 17 aa, synthetic construct
  REGION 1..17: note = Synthetic
SISGSGDYTYYTDSVKG

SEQ ID NO: 608 - protein, 9 aa, synthetic construct
  REGION 1..9: note = Synthetic
SPWGYYLDS

SEQ ID NO: 609 - protein, 11 aa, synthetic construct
  REGION 1..11: note = Synthetic
RASQGISSRLA

SEQ ID NO: 610 - protein, 7 aa, synthetic construct
  REGION 1..7: note = Synthetic
AASSLQS

SEQ ID NO: 611 - protein, 9 aa, synthetic construct
  REGION 1..9: note = Synthetic
QQYNSYPYT

SEQ ID NO: 612 - protein, 118 aa, synthetic construct
  REGION 1..118: note = Synthetic
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 613 - protein, 107 aa, synthetic construct
  REGION 1..107: note = Synthetic
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 614 - protein, 5 aa, synthetic construct
  REGION 1..5: note = Synthetic
DHAIH

SEQ ID NO: 615 - protein, 17 aa, synthetic construct
  REGION 1..17: note = Synthetic
YFSPGNDDIKYNEKFRG

SEQ ID NO: 616 - protein, 6 aa, synthetic construct
  REGION 1..6: note = Synthetic
SLSTPY

SEQ ID NO: 617 - protein, 17 aa, synthetic construct
  REGION 1..17: note = Synthetic
KSSQSLLNRGNHKNYLT

SEQ ID NO: 618 - protein, 7 aa, synthetic construct
  REGION 1..7: note = Synthetic
WASTRES

SEQ ID NO: 619 - protein, 9 aa, synthetic construct
  REGION 1..9: note = Synthetic
QNDYTYPYT

SEQ ID NO: 620 - protein, 115 aa, synthetic construct
  REGION 1..115: note = Synthetic
EVQLVQSGAEVKKPGASVKVSCKASGYTFTDHAIHWVRQAPGQGLEWMGYFSPGNDDIKYNEKFRGRVTMTADKSSSTAYMELRSLRSDDTAVYFCKRSLSTPYWGQGTLVTVSS

SEQ ID NO: 621 - protein, 113 aa, synthetic construct
  REGION 1..113: note = Synthetic
DIVMTQSPDSLAVSLGERATINCKSSQSLLNRGNHKNYLTWYQQKPGQPPKLLIYWASTRESGVPDRFSGSGSGTDFTLTISSLQAEDVAVYYCQNDYTYPYTFGQGTKVEIK

SEQ ID NO: 622 - protein, 5 aa, synthetic construct
  REGION 1..5: note = Synthetic
SYNMH

SEQ ID NO: 623 - protein, 17 aa, synthetic construct
  REGION 1..17: note = Synthetic
AIYPGNGDTSYNQKFKG

SEQ ID NO: 624 - protein, 12 aa, synthetic construct
  REGION 1..12: note = Synthetic
STYYGGDWYFNV

SEQ ID NO: 625 - protein, 10 aa, synthetic construct
  REGION 1..10: note = Synthetic
RASSSVSYIH

SEQ ID NO: 626 - protein, 7 aa, synthetic construct
  REGION 1..7: note = Synthetic
ATSNLAS

SEQ ID NO: 627 - protein, 9 aa, synthetic construct
  REGION 1..9: note = Synthetic
QQWTSNPPT

SEQ ID NO: 628 - protein, 121 aa, synthetic construct [sequence omitted]

SEQ ID NO: 629 - protein, 106 aa, synthetic construct [sequence omitted]

SEQ ID NO: 630 - protein, 5 aa, synthetic construct
  REGION 1..5: note = Synthetic
DTYIH

SEQ ID NO: 631 - protein, 17 aa, synthetic construct
  REGION 1..17: note = Synthetic
RIYPTNGYTRYADSVKG

SEQ ID NO: 632 - protein, 11 aa, synthetic construct
  REGION 1..11: note = Synthetic
WGGDGFYAMDY

SEQ ID NO: 633 - protein, 11 aa, synthetic construct
  REGION 1..11: note = Synthetic
RASQDVNTAVA

SEQ ID NO: 634 - protein, 7 aa, synthetic construct
  REGION 1..7: note = Synthetic
SASFLYS

SEQ ID NO: 635 - protein, 9 aa, synthetic construct
  REGION 1..9: note = Synthetic
QQHYTTPPT

SEQ ID NO: 636 - protein, 120 aa, synthetic construct [sequence omitted]

SEQ ID NO: 637 - protein, 107 aa, synthetic construct [sequence omitted]

SEQ ID NO: 638 - protein, 5 aa, synthetic construct
  REGION 1..5: note = Synthetic
SYWIE

SEQ ID NO: 639 - protein, 17 aa, synthetic construct
  REGION 1..17: note = Synthetic
EILPGGGDTNYNEIFKG

SEQ ID NO: 640 - protein, 8 aa, synthetic construct
  REGION 1..8: note = Synthetic
RVPIRLDY

SEQ ID NO: 641 - protein, 15 aa, synthetic construct
  REGION 1..15: note = Synthetic
KASQSVDYEGDSFLN

SEQ ID NO: 642 - protein, 7 aa, synthetic construct
  REGION 1..7: note = Synthetic
AASNLES

SEQ ID NO: 643 - protein, 9 aa, synthetic construct
  REGION 1..9: note = Synthetic
QQSNEDPLT

SEQ ID NO: 644 - protein, 117 aa, synthetic construct [sequence omitted]

SEQ ID NO: 645 - protein, 111 aa, synthetic construct [sequence omitted]

SEQ ID NO: 646 - protein, 5 aa, synthetic construct
  REGION 1..5: note = Synthetic
DFAMS

SEQ ID NO: 647 - protein, 17 aa, synthetic construct
  REGION 1..17: note = Synthetic
TIGRVAFHTYYPDSMKG

SEQ ID NO: 648 - protein, 11 aa, synthetic construct
  REGION 1..11: note = Synthetic
HRGFDVGHFDF

SEQ ID NO: 649 - protein, 16 aa, synthetic construct
  REGION 1..16: note = Synthetic
RSSETLVHSSGNTYLE

SEQ ID NO: 650 - protein, 7 aa, synthetic construct
  REGION 1..7: note = Synthetic
RVSNRFS

SEQ ID NO: 651 - protein, 9 aa, synthetic construct
  REGION 1..9: note = Synthetic
FQGSFNPLT

SEQ ID NO: 652 - protein, 120 aa, synthetic construct
  REGION 1..120: note = Synthetic
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 653 - protein, 112 aa, synthetic construct
  REGION 1..112: note = Synthetic
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIK

SEQ ID NO: 654 - protein, 6 aa, synthetic construct
  REGION 1..6: note = Synthetic
NDYAWN

SEQ ID NO: 655 - protein, 16 aa, synthetic construct
  REGION 1..16: note = Synthetic
YISYSGYTTYNPSLKS

SEQ ID NO: 656 - protein, 7 aa, synthetic construct
  REGION 1..7: note = Synthetic
WTSGLDY

SEQ ID NO: 657 - protein, 11 aa, synthetic construct
  REGION 1..11: note = Synthetic
KASDLIHNWLA

SEQ ID NO: 658 - protein, 7 aa, synthetic construct
  REGION 1..7: note = Synthetic
GATSLET

SEQ ID NO: 659 - protein, 9 aa, synthetic construct
  REGION 1..9: note = Synthetic
QQYWTTPFT

SEQ ID NO: 660 - protein, 116 aa, synthetic construct
  REGION 1..116: note = Synthetic
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 661 - protein, 107 aa, synthetic construct
  REGION 1..107: note = Synthetic
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 662 - protein, 6 aa, synthetic construct
  REGION 1..6: note = Synthetic
SDYAWN

SEQ ID NO: 663 - protein, 16 aa, synthetic construct
  REGION 1..16: note = Synthetic
YISNSGSTSYNPSLKS

SEQ ID NO: 664 - protein, 15 aa, synthetic construct
  REGION 1..15: note = Synthetic
ERNYDYDDYYYAMDY

SEQ ID NO: 665 - protein, 17 aa, synthetic construct
  REGION 1..17: note = Synthetic
KSSQSLLYRSNQKNYLA

SEQ ID NO: 666 - protein, 7 aa, synthetic construct
  REGION 1..7: note = Synthetic
WASTRES

SEQ ID NO: 667 - protein, 9 aa, synthetic construct
  REGION 1..9: note = Synthetic
QQYYNYPRT

SEQ ID NO: 668 - protein, 124 aa, synthetic construct
  REGION 1..124: note = Synthetic
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 669 - protein, 113 aa, synthetic construct
  REGION 1..113: note = Synthetic
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 670 - protein, 5 aa, synthetic construct
  REGION 1..5: note = Synthetic
NYWMH

SEQ ID NO: 671 - protein, 17 aa, synthetic construct
  REGION 1..17: note = Synthetic
ATYRGHSDTYYNQKFKG

SEQ ID NO: 672 - protein, 12 aa, synthetic construct
  REGION 1..12: note = Synthetic
GAIYDGYDVLDN

SEQ ID NO: 673 - protein, 11 aa, synthetic construct
  REGION 1..11: note = Synthetic
SASQDISNYLN

SEQ ID NO: 674 - protein, 7 aa, synthetic construct
  REGION 1..7: note = Synthetic
YTSNLHS

SEQ ID NO: 675 - protein, 9 aa, synthetic construct
  REGION 1..9: note = Synthetic
QQYRKLPWT

SEQ ID NO: 676 - protein, 121 aa, synthetic construct
  REGION 1..121: note = Synthetic
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 677 - protein, 107 aa, synthetic construct
  REGION 1..107: note = Synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 678 - protein, 5 aa, synthetic construct
  REGION 1..5: note = Synthetic
AYTMH

SEQ ID NO: 679 - protein, 17 aa, synthetic construct
  REGION 1..17: note = Synthetic
WIKPNNGLANYAQKFQG

SEQ ID NO: 680 - protein, 9 aa, synthetic construct
  REGION 1..9: note = Synthetic
SEITTEFDY

SEQ ID NO: 681 - protein, 15 aa, synthetic construct
  REGION 1..15: note = Synthetic
KSSESVDSYANSFLH

SEQ ID NO: 682 - protein, 7 aa, synthetic construct
  REGION 1..7: note = Synthetic
RASTRES

SEQ ID NO: 683 - protein, 9 aa, synthetic construct
  REGION 1..9: note = Synthetic
QQSKEDPLT

SEQ ID NO: 684 - protein, 118 aa, synthetic construct
  REGION 1..118: note = Synthetic
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 685 - protein, 111 aa, synthetic construct
  REGION 1..111: note = Synthetic
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 686 - protein, 6 aa, synthetic construct
  REGION 1..6: note = Synthetic
SDFAWN

SEQ ID NO: 687 - protein, 16 aa, synthetic construct
  REGION 1..16: note = Synthetic
YISYSGNTRYQPSLKS

SEQ ID NO: 688 - protein, 7 aa, synthetic construct
  REGION 1..7: note = Synthetic
AGRGFPY

SEQ ID NO: 689 - protein, 11 aa, synthetic construct
  REGION 1..11: note = Synthetic
HSSQDINSNIG

SEQ ID NO: 690 - protein, 7 aa, synthetic construct
  REGION 1..7: note = Synthetic
HGTNLDD

SEQ ID NO: 691 - protein, 9 aa, synthetic construct
  REGION 1..9: note = Synthetic
VQYAQFPWT

SEQ ID NO: 692 - protein, 116 aa, synthetic construct
  REGION 1..116: note = Synthetic
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 693 - protein, 107 aa, synthetic construct; identical to SEQ ID NO: 319
  REGION 1..107: note = Synthetic

SEQ ID NO: 694 - protein, 5 aa, synthetic construct
  REGION 1..5: note = Synthetic
DYYMA

SEQ ID NO: 695 - protein, 17 aa, synthetic construct
  REGION 1..17: note = Synthetic
SINYDGSSTYYVDSVKG

SEQ ID NO: 696 - protein, 8 aa, synthetic construct
  REGION 1..8: note = Synthetic
DRGYYFDY

SEQ ID NO: 697 - protein, 16 aa, synthetic construct
  REGION 1..16: note = Synthetic
RSSQSLVHSNGNTYLH

SEQ ID NO: 698 - protein, 7 aa, synthetic construct
  REGION 1..7: note = Synthetic
KVSNRFS

SEQ ID NO: 699 - protein, 10 aa, synthetic construct
  REGION 1..10: note = Synthetic
SQSTHVPPFT

SEQ ID NO: 700 - protein, 117 aa, synthetic construct
  REGION 1..117: note = Synthetic
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSS

SEQ ID NO: 701 - protein, 113 aa, synthetic construct
  REGION 1..113: note = Synthetic
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIK

SEQ ID NO: 702 - protein, 5 aa, synthetic construct
  REGION 1..5: note = Synthetic
SYGMH

SEQ ID NO: 703 - protein, 17 aa, synthetic construct
  REGION 1..17: note = Synthetic
VIWYDGSNQYYADSVKG

SEQ ID NO: 704 - protein, 11 aa, synthetic construct
  REGION 1..11: note = Synthetic
GLTSGRYGMDV

SEQ ID NO: 705 - protein, 16 aa, synthetic construct
  REGION 1..16: note = Synthetic
RSSQSLLLSHGFNYLD

SEQ ID NO: 706 - protein, 7 aa, synthetic construct
  REGION 1..7: note = Synthetic
LGSSRAS

SEQ ID NO: 707 - protein, 9 aa, synthetic construct
  REGION 1..9: note = Synthetic
MQPLQIPWT

SEQ ID NO: 708 - protein, 120 aa, synthetic construct; identical to SEQ ID NO: 596
  REGION 1..120: note = Synthetic

SEQ ID NO: 709 - protein, 112 aa, synthetic construct; identical to SEQ ID NO: 597
  REGION 1..112: note = Synthetic

SEQ ID NO: 710 - protein, 5 aa, synthetic construct
  REGION 1..5: note = Synthetic
NAWMS

SEQ ID NO: 711 - protein, 17 aa, synthetic construct
  REGION 1..17: note = Synthetic
YISSSGSTIYYADSVKG

SEQ ID NO: 712 - protein, 8 aa, synthetic construct
  REGION 1..8: note = Synthetic
EGLWAFDY

SEQ ID NO: 713 - protein, 14 aa, synthetic construct
  REGION 1..14: note = Synthetic
TGSSSNIGAGYVVH

SEQ ID NO: 714 - protein, 7 aa, synthetic construct
  REGION 1..7: note = Synthetic
DNNKRPS

SEQ ID NO: 715 - protein, 11 aa, synthetic construct
  REGION 1..11: note = Synthetic
AAWDDRLNGPV

SEQ ID NO: 716 - protein, 117 aa, synthetic construct
  REGION 1..117: note = Synthetic
EVQLLESGGGLVQPGGSLRLSCAASGFTFSNAWMSWVRQAPGKGLEWVSYISSSGSTIYYADSVKGRFTISRDNSKNTLYLQMNSLRAEDTAVYYCAREGLWAFDYWGQGTLVTVSS

SEQ ID NO: 717 - protein, 111 aa, synthetic construct
  REGION 1..111: note = Synthetic
ESVLTQPPSVSGAPGQRVTISCTGSSSNIGAGYVVHWYQQLPGTAPKLLIYDNNKRPSGVPDRFSGSKSGTSASLAISGLRSEDEADYYCAAWDDRLNGPVFGGGTKLTVL

SEQ ID NO: 718 - protein, 5 aa, synthetic construct
  REGION 1..5: note = Synthetic
GYYWS

SEQ ID NO: 719 - protein, 16 aa, synthetic construct
  REGION 1..16: note = Synthetic
EINHRGNTNDNPSLKS

SEQ ID NO: 720 - protein, 11 aa, synthetic construct
  REGION 1..11: note = Synthetic
ERGYTYGNFDH

SEQ ID NO: 721 - protein, 11 aa, synthetic construct
  REGION 1..11: note = Synthetic
RASQSVSRNLA

SEQ ID NO: 722 - protein, 7 aa, synthetic construct
  REGION 1..7: note = Synthetic
GASTRAT

SEQ ID NO: 723 - protein, 9 aa, synthetic construct
  REGION 1..9: note = Synthetic
QQYKTWPRT

SEQ ID NO: 724 - protein, 119 aa, synthetic construct
  REGION 1..119: note = Synthetic
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 725 - protein, 107 aa, synthetic construct
  REGION 1..107: note = Synthetic
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 726 - protein, 5 aa, synthetic construct
  REGION 1..5: note = Synthetic
SYAMN

SEQ ID NO: 727 - protein, 17 aa, synthetic construct
  REGION 1..17: note = Synthetic
TTSGSGASTYYADSVKG

SEQ ID NO: 728 - protein, 7 aa, synthetic construct
  REGION 1..7: note = Synthetic
IWIAFDI

SEQ ID NO: 729 - protein, 12 aa, synthetic construct
  REGION 1..12: note = Synthetic
RASQSVSSSYLA

SEQ ID NO: 730 - protein, 7 aa, synthetic construct
  REGION 1..7: note = Synthetic
GASSRAT

SEQ ID NO: 731 - protein, 9 aa, synthetic construct
  REGION 1..9: note = Synthetic
QQYGSSPYT

SEQ ID NO: 732 - protein, 116 aa, synthetic construct
  REGION 1..116: note = Synthetic
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 733 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 734 - protein, 7 aa, synthetic construct
  REGION 1..7: note = Synthetic
SFNYYWS

SEQ ID NO: 735 - protein, 16 aa, synthetic construct
  REGION 1..16: note = Synthetic
YIYYSGSTYSNPSLKS

SEQ ID NO: 736 - protein, 9 aa, synthetic construct
  REGION 1..9: note = Synthetic
GYNWNYFDY

SEQ ID NO: 737 - protein, 11 aa, synthetic construct
  REGION 1..11: note = Synthetic
RASQSVDNNLV

SEQ ID NO: 738 - protein, 7 aa, synthetic construct
  REGION 1..7: note = Synthetic
GASTRAT

SEQ ID NO: 739 - protein, 10 aa, synthetic construct [sequence omitted]

SEQ ID NO: 740 - protein, 119 aa, synthetic construct [sequence omitted]

SEQ ID NO: 741 - protein, 108 aa, synthetic construct [sequence omitted]

SEQ ID NO: 742 - protein, 5 aa, synthetic construct
  REGION 1..5: note = Synthetic
TYWMH

SEQ ID NO: 743 - protein, 17 aa, synthetic construct
  REGION 1..17: note = Synthetic
EIDPSDSYSNYNQKFKD

SEQ ID NO: 744 - protein, 11 aa, synthetic construct
  REGION 1..11: note = Synthetic
NGGLGPAWFSY

SEQ ID NO: 745 - protein, 11 aa, synthetic construct
  REGION 1..11: note = Synthetic
KASQYVGTAVA

SEQ ID NO: 746 - protein, 7 aa, synthetic construct
  REGION 1..7: note = Synthetic
SASNRYT

SEQ ID NO: 747 - protein, 9 aa, synthetic construct
  REGION 1..9: note = Synthetic
QQYSSYPWT

SEQ ID NO: 748 - protein, 120 aa, synthetic construct
  REGION 1..120: note = Synthetic
EVQLVQSGAEVKKPGSSVKVSCKASGYTFTTYWMHWVRQAPGQGLEWIGEIDPSDSYSNYNQKFKDRATLTVDKSTSTAYMELSSLRSEDTAVYYCARNGGLGPAWFSYWGQGTLVTVSS

SEQ ID NO: 749 - protein, 107 aa, synthetic construct
  REGION 1..107: note = Synthetic
DIQMTQSPSSVSASVGDRVTITCKASQYVGTAVAWYQQKPGKSPKLLIYSASNRYTGVPSRFSDSGSGTDFTLTISSLQPEDFATYFCQQYSSYPWTFGGGTKVEIK

SEQ ID NO: 750 - protein, 5 aa, synthetic construct
  REGION 1..5: note = Synthetic
SYAMS

SEQ ID NO: 751 - protein, 17 aa, synthetic construct
  REGION 1..17: note = Synthetic
AISGSGTSTYYADSVKG

SEQ ID NO: 752 - protein, 13 aa, synthetic construct
  REGION 1..13: note = Synthetic
VRYNWNHGDWFDP

SEQ ID NO: 753 - protein, 13 aa, synthetic construct
  REGION 1..13: note = Synthetic
SGSSSNIGNNYVS

SEQ ID NO: 754 - protein, 7 aa, synthetic construct
  REGION 1..7: note = Synthetic
ENYNRPA

SEQ ID NO: 755 - protein, 11 aa, synthetic construct
  REGION 1..11: note = Synthetic
SSWDDSLNYWV

SEQ ID NO: 756 - protein, 122 aa, synthetic construct
  REGION 1..122: note = Synthetic
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 757 - protein, 110 aa, synthetic construct
  REGION 1..110: note = Synthetic
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 758 - protein, 5 aa, synthetic construct
  REGION 1..5: note = Synthetic
SYGMS

SEQ ID NO: 759 - protein, 17 aa, synthetic construct
  REGION 1..17: note = Synthetic
TISSGGSYKYYVDSVKG

SEQ ID NO: 760 - protein, 11 aa, synthetic construct
  REGION 1..11: note = Synthetic
HPDYDGVWFAY

SEQ ID NO: 761 - protein, 10 aa, synthetic construct
  REGION 1..10: note = Synthetic
SVSSSVFYVH

SEQ ID NO: 762 - protein, 7 aa, synthetic construct
  REGION 1..7: note = Synthetic
DTSKLAS

SEQ ID NO: 763 - protein, 9 aa, synthetic construct
  REGION 1..9: note = Synthetic
QQWNSNPPT

SEQ ID NO: 764 - protein, 120 aa, synthetic construct
  REGION 1..120: note = Synthetic
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 765 - protein, 107 aa, synthetic construct
  REGION 1..107: note = Synthetic
DIQMTQSPSSLSASVGDRVTITCSVSSSVFYVHWYQQKPGKAPKLLIYDTSKLASSGVPSRFSGSGSGTDFTFTISSLQPEDIATYYCQQWNSNPPTFGGGTKVEIK

SEQ ID NO: 766 - protein, 5 aa, synthetic construct
  REGION 1..5: note = Synthetic
SYGMS

SEQ ID NO: 767 - protein, 17 aa, synthetic construct
  REGION 1..17: note = Synthetic
TISSGGSYTYYPDSVKG

SEQ ID NO: 768 - protein, 11 aa, synthetic construct
  REGION 1..11: note = Synthetic
HPDYDGVWFAY

SEQ ID NO: 769 - protein, 10 aa, synthetic construct
  REGION 1..10: note = Synthetic
SVSSSVFYVH

SEQ ID NO: 770 - protein, 7 aa, synthetic construct
  REGION 1..7: note = Synthetic
DTSKLAS

SEQ ID NO: 771 - protein, 9 aa, synthetic construct
  REGION 1..9: note = Synthetic
QQWNSNPPT

SEQ ID NO: 772 - protein, 120 aa, synthetic construct
  REGION 1..120: note = Synthetic
EVQLVESGGDLVKPGGSLKLSCAASGFTFNSYGMSWVRQTPDKRLEWVATISSGGSYTYYPDSVKGRFTISRDNAKNTLYLQMSSLKSEDTAMYYCARHPDYDGVWFAYWGQGTLVTVSA

SEQ ID NO: 773 - protein, 105 aa, synthetic construct
  REGION 1..105: note = Synthetic
QIVLTQSPAIMASPGEKVTMTCSVSSSVFYVHWYQQKSGTSPKRWIYDTSKLASGVPARFSGSGSGTSYSLTISSMEAEDAATYYCQQWNSNPPTFGGGTKLEIK

SEQ ID NO: 774 - protein, 5 aa, synthetic construct
  REGION 1..5: note = Synthetic
SYYMH

SEQ ID NO: 775 - protein, 17 aa, synthetic construct
  REGION 1..17: note = Synthetic
IINPSGGSTSYAQKFQG

SEQ ID NO: 776 - protein, 16 aa, synthetic construct
  REGION 1..16: note = Synthetic
DGVLRYFDWLLDYYYY

SEQ ID NO: 777 - protein, 11 aa, synthetic construct
  REGION 1..11: note = Synthetic
RASQSVGSYLA

SEQ ID NO: 778 - protein, 7 aa, synthetic construct
  REGION 1..7: note = Synthetic
DASNRAT

SEQ ID NO: 779 - protein, 8 aa, synthetic construct
  REGION 1..8: note = Synthetic
QQRANVFT

SEQ ID NO: 780 - protein, 128 aa, synthetic construct
  REGION 1..128: note = Synthetic
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGVLRYFDWLLDYYYYMDVWGKGTTVTVSS

SEQ ID NO: 781 - protein, 106 aa, synthetic construct
  REGION 1..106: note = Synthetic
EIVLTQSPATLSLSPGERATLSCRASQSVGSYLAWYQQRPGQAPRLLIYDASNRATGIPARFSGSGSGTDFTLTISSLEPEDFAVYYCQQRANVFTFGQGTKVEIK

SEQ ID NO: 782 - protein, 5 aa, synthetic construct
  REGION 1..5: note = Synthetic
SYYMH

SEQ ID NO: 783 - protein, 17 aa, synthetic construct
  REGION 1..17: note = Synthetic
IINPSGGSTSYAQKFQG

SEQ ID NO: 784 - protein, 19 aa, synthetic construct
  REGION 1..19: note = Synthetic
DAELRHFDHLLDYHYYMDV

SEQ ID NO: 785 - protein, 11 aa, synthetic construct
  REGION 1..11: note = Synthetic
RASQSVGSYLA

SEQ ID NO: 786 - protein, 7 aa, synthetic construct
  REGION 1..7: note = Synthetic
DASNRAT

SEQ ID NO: 787 - protein, 8 aa, synthetic construct
  REGION 1..8: note = Synthetic
QQRAQEFT

SEQ ID NO: 788 - protein, 128 aa, synthetic construct
  REGION 1..128: note = Synthetic
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDAELRHFDHLLDYHYYMDVWGQGTTVTVSS

SEQ ID NO: 789 - protein, 106 aa, synthetic construct
  REGION 1..106: note = Synthetic
EIVMTQSPATLSLSPGERATLSCRASQSVGSYLAWYQQKPGQAPRLLIYDASNRATGIPARFSGSGSGTDFTLTISSLQPEDFAVYYCQQRAQEFTFGQGTKVEIK

SEQ ID NO: 790 - protein, 120 aa, synthetic construct
  REGION 1..120: note = Synthetic
QVQLVQSGSELKKPGAPVKVSCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTVTVSS

SEQ ID NO: 791 - protein, 107 aa, synthetic construct
  REGION 1..107: note = Synthetic
DIQMTQSPSSLSASLGDRVTITCRASQDIRNYLNWYQQKPGKAPKLLIYYTSRLHSGVPSRFSGSGSGTDYTLTISSLQPEDFATYFCQQGHTLPPTFGQGTKLEIK

SEQ ID NO: 792 - protein, 120 aa, synthetic construct
  REGION 1..120: note = Synthetic
QIQLVQSGPELKKPGAPVKISCKASGYTFTTFGMSWVKQAPGQGLKWMGWIHTYAGVPIYGDDFKGRFVFSLDTSVSTAYLQISSVKAEDTATYFCARRSDNYRYFFDYWGQGTTLTVSS

SEQ ID NO: 793 - protein, 107 aa, synthetic construct; identical to SEQ ID NO: 791
  REGION 1..107: note = Synthetic

SEQ ID NO: 794 - protein, 5 aa, synthetic construct
  REGION 1..5: note = Synthetic
NYYMA

SEQ ID NO: 795 - protein, 17 aa, synthetic construct
  REGION 1..17: note = Synthetic
SITKGGGNTYYRDSVKG

SEQ ID NO: 796 - protein, 14 aa, synthetic construct
  REGION 1..14: note = Synthetic
QVTIAAVSTSYFDS

SEQ ID NO: 797 - protein, 15 aa, synthetic construct
  REGION 1..15: note = Synthetic
KTNQKVDYYGNSYVY

SEQ ID NO: 798 - protein, 7 aa, synthetic construct
  REGION 1..7: note = Synthetic
LASNLAS

SEQ ID NO: 799 - protein, 9 aa, synthetic construct
  REGION 1..9: note = Synthetic
QQSRNLPYT

SEQ ID NO: 800 - protein, 123 aa, synthetic construct
  REGION 1..123: note = Synthetic
EVQLVESGGGLVQSGRSIRLSCAASGFSFSNYYMAWVRQAPSKGLEWVASITKGGGNTYYRDSVKGRFTFSRDNAKSTLYLQMDSLRSEDTATYYCARQVTIAAVSTSYFDSWGQGVMVTVSS

SEQ ID NO: 801 - protein, 110 aa, synthetic construct
  REGION 1..110: note = Synthetic
DIVLTQSPALAVSLGQRATISCKTNQKVDYYGNSYVYWYQQKPGQQPKLLIYLASNLASGIPARFSGRGSGTDFTLTIDPVEADDTATYYCQQSRNLPYTFGAGTKLELK

SEQ ID NO: 802 - protein, 5 aa, synthetic construct
  REGION 1..5: note = Synthetic
DYYMK

SEQ ID NO: 803 - protein, 17 aa, synthetic construct
  REGION 1..17: note = Synthetic
DIIPSNGATFYNQKFKG

SEQ ID NO: 804 - protein, 11 aa, synthetic construct
  REGION 1..11: note = Synthetic
SHLLRASWFAY

SEQ ID NO: 805 - protein, 17 aa, synthetic construct
  REGION 1..17: note = Synthetic
KSSQSLLNSGNQKNYLT

SEQ ID NO: 806 - protein, 7 aa, synthetic construct
  REGION 1..7: note = Synthetic
WASTRES

SEQ ID NO: 807 - protein, 9 aa, synthetic construct
  REGION 1..9: note = Synthetic
QNDYSYPYT

SEQ ID NO: 808 - protein, 120 aa, synthetic construct
  REGION 1..120: note = Synthetic
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 809 - protein, 113 aa, synthetic construct
  REGION 1..113: note = Synthetic
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 810 - protein, 5 aa, synthetic construct
  REGION 1..5: note = Synthetic
DYEMH

SEQ ID NO: 811 - protein, 20 aa, synthetic construct
  REGION 1..20: note = Synthetic
WIGGIDPETGGTAYNQKFKG

SEQ ID NO: 812 - protein, 6 aa, synthetic construct
  REGION 1..6: note = Synthetic
YYSFAY

SEQ ID NO: 813 - protein, 16 aa, synthetic construct
  REGION 1..16: note = Synthetic
RSSQSIVHSNGNTYLQ

SEQ ID NO: 814 - protein, 7 aa, synthetic construct
  REGION 1..7: note = Synthetic
KVSNRFS

SEQ ID NO: 815 - protein, 9 aa, synthetic construct
  REGION 1..9: note = Synthetic
FQVSHVPYT

SEQ ID NO: 816 - protein, 115 aa, synthetic construct
  REGION 1..115: note = Synthetic
EVQLVQSGAEVKKPGATVKISCKVSGYTFTDYEMHWVQQAPGKGLEWMGGIDPETGGTAYNQKFKGRVTLTADKSTDTAYMELSSLRSEDTAVYYCGRYYSFAYWGQGTLVTVSS

SEQ ID NO: 817 - protein, 112 aa, synthetic construct
  REGION 1..112: note = Synthetic
DVVMTQSPLSLPVTLGQPASISCRSSQSIVHSNANTYLQWFQQRPGQSPRLLIYKVSNRFSGVPDRFSGSGSGTDFTLKISRVEAEDVGVYYCFQVSHVPYTFGQGTKLEIK

SEQ ID NO: 818 - protein, 5 aa, synthetic construct
  REGION 1..5: note = Synthetic
DYNVN

SEQ ID NO: 819 - protein, 17 aa, synthetic construct
  REGION 1..17: note = Synthetic
VINPKYGTTRYNQKFKG

SEQ ID NO: 820 - protein, 7 aa, synthetic construct
  REGION 1..7: note = Synthetic
GLNAWDY

SEQ ID NO: 821 - protein, 11 aa, synthetic construct
  REGION 1..11: note = Synthetic
GASENIYGALN

SEQ ID NO: 822 - protein, 7 aa, synthetic construct
  REGION 1..7: note = Synthetic
GATNLED

SEQ ID NO: 823 - protein, 9 aa, synthetic construct
  REGION 1..9: note = Synthetic
QNVLTTPYT

SEQ ID NO: 824 - protein, 116 aa, synthetic construct; identical to SEQ ID NO: 532
  REGION 1..116: note = Synthetic

SEQ ID NO: 825 - protein, 107 aa, synthetic construct; identical to SEQ ID NO: 533
  REGION 1..107: note = Synthetic

SEQ ID NO: 826 - protein, 5 aa, synthetic construct
  REGION 1..5: note = Synthetic
GYFMN

SEQ ID NO: 827 - protein, 17 aa, synthetic construct
  REGION 1..17: note = Synthetic
LINPYNGDSFYNQKFKG

SEQ ID NO: 828 - protein, 8 aa, synthetic construct
  REGION 1..8: note = Synthetic
GLRRDFDY

SEQ ID NO: 829 - protein, 8 aa, synthetic construct
  REGION 1..8: note = Synthetic
GLRRDFDY

SEQ ID NO: 830 - protein, 7 aa, synthetic construct
  REGION 1..7: note = Synthetic
LVSELDS

SEQ ID NO: 831 - protein, 9 aa, synthetic construct
  REGION 1..9: note = Synthetic
WQGTHFPRT

SEQ ID NO: 832 - protein, 117 aa, synthetic construct; identical to SEQ ID NO: 540
  REGION 1..117: note = Synthetic

SEQ ID NO: 833 - protein, 112 aa, synthetic construct; identical to SEQ ID NO: 541
  REGION 1..112: note = Synthetic

SEQ ID NO: 834 - protein, 5 aa, synthetic construct
  REGION 1..5: note = Synthetic
TAAIS

SEQ ID NO: 835 - protein, 17 aa, synthetic construct
  REGION 1..17: note = Synthetic
GIIPIFGKAHYAQKFQG

SEQ ID NO: 836 - protein, 14 aa, synthetic construct
  REGION 1..14: note = Synthetic
KFHFVSGSPFGMDV

SEQ ID NO: 837 - protein, 11 aa, synthetic construct
  REGION 1..11: note = Synthetic
RASQSVSSYLA

SEQ ID NO: 838 - protein, 7 aa, synthetic construct
  REGION 1..7: note = Synthetic
DASNRAT

SEQ ID NO: 839 - protein, 8 aa, synthetic construct
  REGION 1..8: note = Synthetic
QQRSNWPT

SEQ ID NO: 840 - protein, 123 aa, synthetic construct; identical to SEQ ID NO: 556
  REGION 1..123: note = Synthetic

SEQ ID NO: 841 - protein, 106 aa, synthetic construct; identical to SEQ ID NO: 557
  REGION 1..106: note = Synthetic

SEQ ID NO: 842 - protein, 9 aa, synthetic construct
  REGION 1..9: note = Synthetic
GTFSSYAIS

SEQ ID NO: 843 - protein, 17 aa, synthetic construct
  REGION 1..17: note = Synthetic
SIIPIFGTANYAQKFQG

SEQ ID NO: 844 - protein, 18 aa, synthetic construct
  REGION 1..18: note = Synthetic
ARGPSEVGAILGYVWFDP

SEQ ID NO: 845 - protein, 16 aa, synthetic construct
  REGION 1..16: note = Synthetic
RSSQSLLHSNGYNYLD

SEQ ID NO: 846 - protein, 7 aa, synthetic construct
  REGION 1..7: note = Synthetic
LGSNRAS

SEQ ID NO: 847 - protein, 9 aa, synthetic construct
  REGION 1..9: note = Synthetic
MQARRIPIT

SEQ ID NO: 848 - protein, 125 aa, synthetic construct
  REGION 1..125: note = Synthetic
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 849 - protein, 112 aa, synthetic construct
  REGION 1..112: note = Synthetic
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 850 - protein, 12 aa, synthetic construct
  REGION 1..12: note = Synthetic
GYTFTDHAIHWV

SEQ ID NO: 851 - protein, 10 aa, synthetic construct
  REGION 1..10: note = Synthetic
FSPGNDDIKY

SEQ ID NO: 852 - protein, 8 aa, synthetic construct
  REGION 1..8: note = Synthetic
KRSLSTPY

SEQ ID NO: 853 - protein, 12 aa, synthetic construct
  REGION 1..12: note = Synthetic
QSLLNRGNHKNY

SEQ ID NO: 854 - protein, 7 aa, synthetic construct
  REGION 1..7: note = Synthetic
WASTRES

SEQ ID NO: 855 - protein, 9 aa, synthetic construct
  REGION 1..9: note = Synthetic
QNDYTYPYT

SEQ ID NO: 856 - protein, 115 aa, synthetic construct; identical to SEQ ID NO: 620
  REGION 1..115: note = Synthetic

SEQ ID NO: 857 - protein, 113 aa, synthetic construct; identical to SEQ ID NO: 621
  REGION 1..113: note = Synthetic

SEQ ID NO: 858 - protein, 5 aa, synthetic construct
  REGION 1..5: note = Synthetic
NYDIN

SEQ ID NO: 859 - protein, 17 aa, synthetic construct
  REGION 1..17: note = Synthetic
WIYPGDGSTKYNEKFKA

SEQ ID NO: 860 - protein, 8 aa, synthetic construct
  REGION 1..8: note = Synthetic
GYEDAMDY

SEQ ID NO: 861 - protein, 11 aa, synthetic construct
  REGION 1..11: note = Synthetic
KASQDINSYLS

SEQ ID NO: 862 - protein, 7 aa, synthetic construct
  REGION 1..7: note = Synthetic
RANRLVD

SEQ ID NO: 863 - protein, 9 aa, synthetic construct
  REGION 1..9: note = Synthetic
LQYDEFPLT

SEQ ID NO: 864 - protein, 117 aa, synthetic construct
  REGION 1..117: note = Synthetic
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 865 - protein, 105 aa, synthetic construct
  REGION 1..105: note = Synthetic
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVE

SEQ ID NO: 866 - protein, 5 aa, synthetic construct
  REGION 1..5: note = Synthetic
NYGMN

SEQ ID NO: 867 - protein, 17 aa, synthetic construct
  REGION 1..17: note = Synthetic
WINTYSGEPRYADDFKG

SEQ ID NO: 868 - protein, 9 aa, synthetic construct
  REGION 1..9: note = Synthetic
DYGRWYFDV

SEQ ID NO: 869 - protein, 10 aa, synthetic construct
  REGION 1..10: note = Synthetic
RASSSVSHMH

SEQ ID NO: 870 - protein, 7 aa, synthetic construct
  REGION 1..7: note = Synthetic
ATSNLAS

SEQ ID NO: 871 - protein, 9 aa, synthetic construct
  REGION 1..9: note = Synthetic
QQWSSTPRT

SEQ ID NO: 872 - protein, 118 aa, synthetic construct; identical to SEQ ID NO: 158
  REGION 1..118: note = Synthetic

SEQ ID NO: 873 - protein, 106 aa, synthetic construct
  REGION 1..106: note = Synthetic
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIK

SEQ ID NO: 874 - protein, 5 aa, synthetic construct
  REGION 1..5: note = Synthetic
DYYIH

SEQ ID NO: 875 - protein, 17 aa, synthetic construct
  REGION 1..17: note = Synthetic
YINPNSGYTNYAQKFQG

SEQ ID NO: 876 - protein, 12 aa, synthetic construct
  REGION 1..12: note = Synthetic
YMWERVTGFFDF

SEQ ID NO: 877 - protein, 11 aa, synthetic construct
  REGION 1..11: note = Synthetic
LASEDISDDLA

SEQ ID NO: 878 - protein, 7 aa, synthetic construct
  REGION 1..7: note = Synthetic
TTSSLQS

SEQ ID NO: 879 - protein, 9 aa, synthetic construct
  REGION 1..9: note = Synthetic
QQTYKFPPT

SEQ ID NO: 880 - protein, 121 aa, synthetic construct
  REGION 1..121: note = Synthetic
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 881 - protein, 107 aa, synthetic construct
  REGION 1..107: note = Synthetic
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIK

SEQ ID NO: 882 - protein, 8 aa, synthetic construct
  REGION 1..8: note = Synthetic
GFTFSNYA

SEQ ID NO: 883 - protein, 8 aa, synthetic construct
  REGION 1..8: note = Synthetic
ISGSGDYT

SEQ ID NO: 884 - protein, 11 aa, synthetic construct
  REGION 1..11: note = Synthetic
ARSPWGYYLDS

SEQ ID NO: 885 - protein, 6 aa, synthetic construct
  REGION 1..6: note = Synthetic
QGISSR

SEQ ID NO: 886 - skipped

SEQ ID NO: 887 - protein, 9 aa, synthetic construct
  REGION 1..9: note = Synthetic
QQYNSYPYT

SEQ ID NO: 888 - protein, 118 aa, synthetic construct; identical to SEQ ID NO: 612
  REGION 1..118: note = Synthetic

SEQ ID NO: 889 - protein, 107 aa, synthetic construct; identical to SEQ ID NO: 613
  REGION 1..107: note = Synthetic